SEQUENCE LISTING (85 sequences)

SEQ ID NO: 1 - DNA, 418 bp, artificial sequence
  T-DNA Left Border sequence
ctgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattcacatccgtttgatacttgtctaaaattggctgatttcgagtgcatctatgcataaaaacaatctaatgacaattattaccaagcatcaatgagatgatgtgtgtgtctatgtgtaaatattgcgcggagtcattacagttataattattttacgagttatttaaagttataaatacatttatataccaagatatatatactattataaatatgaaacttatataagcaattcaatattacagagaatagatagatatcataaaaa

SEQ ID NO: 2 - DNA, 279 bp, artificial sequence
  T-DNA Right Border sequence
ccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagg

SEQ ID NO: 3 - DNA, 1345 bp, artificial sequence
  Culiflower mosaic virus 35s promoter
tcgacgaattaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacac

SEQ ID NO: 4 - DNA, 976 bp, artificial sequence
  maize ubiquitin promoter
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaa

SEQ ID NO: 5 - DNA, 1233 bp, artificial sequence
  rice actin promoter
cctcgaggtcattcatatgcttgagaagagagtcgggatagtccaaaataaaacaaaggtaagattacctggtcaaaagtgaaaacatcagttaaaaggtggtataagtaaaatatcggtaataaaaggtggcccaaagtgaaatttactcttttctactattataaaaattgaggatgttttgtcggtactttgatacgtcatttttgtatgaattggtttttaagtttattcgcgatttggaaatgcatatctgtatttgagtcggtttttaagttcgttgcttttgtaaatacagagggatttgtataagaaatatctttaaaaaacccatatgctaatttgacataatttttgagaaaaatatatattcaggcgaattccacaatgaacaataataagattaaaatagcttgcccccgttgcagcgatgggtattttttctagtaaaataaaagataaacttagactcaaaacatttacaaaaacaacccctaaagtcctaaagcccaaagtgctatgcacgatccatagcaagcccagcccaacccaacccaacccaacccaccccagtgcagccaactggcaaatagtctccacccccggcactatcaccgtgagttgtccgcaccaccgcacgtctcgcagccaaaaaaaaaaaaagaaagaaaaaaaagaaaaagaaaaacagcaggtgggtccgggtcgtgggggccggaaaagcgaggaggatcgcgagcagcgacgaggcccggccctccctccgcttccaaagaaacgccccccatcgccactatatacatacccccccctctcctcccatccccccaaccctaccaccaccaccaccaccacctcctcccccctcgctgccggacgacgagctcctcccccctccccctccgccgccgccggtaaccaccccgcccctctcctctttctttctccgttttttttttcgtctcggtctcgatctttggccttggtagtttgggtgggcgagagcggcttcgtcgcccagatcggtgcgcgggaggggcgggatctcgcggctggcgtctccgggcgtgagtcggcccggatcctcgcggggaatggggctctcggatgtagatcttctttctttcttctttttgtggtagaatttgaatccctcagcattgttcatcggtagtttttcttttcatgatttgtgacaaatgcagcctcgtgcggagcttttttgtaggtaga

SEQ ID NO: 6 - DNA, 824 bp, artificial sequence
  Arabidopsis thaliana rd29A promoter region
cgactcaaaacaaacttacgaaatttaggtagaacttatatacattatatgtgtaattttttgtaacaaaatgtttttattattattatagaattttactggttaaattaaaaatgaatagaaaaggtgaattaagaggagagaggaggtaaacattttcttctattttttcatattttcaggataaattattgtagaagtttaaaagatttccatttgactagtgtaaatgaggaatattctctagtaagatcattatttcatctacttcttttatcttctaccagtagaggaataaacaatatttagctcctttgtaaatacaaattaattttcgttcttgacatcattcaattttaattttacgtataaaataaaagatcatacctattagaacgattaaggagaaatacaattcgaatgagaaggatgtgccgtttgttataataaacagccacacgacgtaaacgtaaaatgaccacatgatgggccaatagacatggaccgactactaataatagtaagttacattttaggatggaataaatatcataccgacatcagtttgaaagaaaagggaaaaaaagaaaaaataaataaaagatatactaccgacatgagttccaaaaagcaaaaaaaaagatcaagccgacacagacacgcgtagagagcaaaatgactttgacgtcacaccacgaaaacagacgcttcatacgtgtccctttatctctctcagtctctctataaacttagtgagaccctcctctgttttactcacaaatatgcaaactagaaaacaatcatcaggaataaagggtttgatt

SEQ ID NO: 7 - DNA, 1033 bp, artificial sequence
  Barley ASI promoter
actgggctcgaaactaaaataagaacatggaaaaagagcgttatcgtatgcatttgaattacgtaggtcttctgcatggtgtttagtttgcttactagagcatcttcaacagttcgtatgttcaatcgttgttataagtgttcacattatcaaccaacatcacatcatacaagctctttaatagagtgtatgttaaccatatgtaaaataactaagtgggtccatcaaatgttgaagtaataatcatgtttgcctcggagcttgtgcatgaaccgttgcttcaagttcatacggtttcattctctctcttcttttattatatgtcatgtcatcaaaatcatctatgtgaaaattttatcaatgatgatcataccaccatcgaagatgccctaagaacgtatccatttactggttggctactgtggctgcatgcatgcattcccgactgcttccccgtcattgtgtcgcacaatttcgtcgagattggtagtacaattcaaacgcttgattcgcatatggttattttttttgtatatgggaggatgtggaacgcagatagtgacacttgagactgtgagagtctcacaaaggtgaagccaactactccctccgtttttaaatatttctcttttttagaaattttagtataaactatatacaaatatatatatatatagataattagagtgtaaattcaatcattttgcgatgtatgtagttcatagttaaatatctaaaatgataaatatttaagaacagaaggagtggtaggatataacattgctctgtatagatctgcaccgcatgcgataatcggtgtacgcacttactggatgccactgagggatgtaaaggaacaggcttcacatcacgcaatccaccagaagaaaagaatgcaagcaaagcaactctacttccagatcactataaatacggacatgaagcactcgtgatgcctcacccgagccaccgaagcacaccctagcttgcagtctcacttgacctcgaggacactccagcagaggtttcagtc

SEQ ID NO: 8 - DNA, 552 bp, Streptomyces hygroscopicus
  CDS (1)..(552)
atgggcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctga

SEQ ID NO: 9 - protein, 183 aa, Streptomyces hygroscopicus
MGPERRPADIRRATEADMPAVCTIVNHYIETSTVNFRTEPQEPQEWTDDLVRLRERYPWLVAEVDGEVAGIAYAGPWKARNAYDWTAESTVYVSPRHQRTGLGSTLYTHLLKSLEAQGFKSVVAVIGLPNDPSVRMHEALGYAPRGMLRAAGFKHGNWHDVGFWQLDFSLPVPPRPVLPVTEI

SEQ ID NO: 10 - DNA, 795 bp, artificial sequence
  Neomycin phosphtransferase
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 11 - protein, 264 aa, artificial sequence
  Synthetic Construct
MIEQDGLHAGSPAAWVERLFGYDWAQQTIGCSDAAVFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKARMPDGDDLVVTHGDACLPNIMVENGRFSGFIDCGRLGVADRYQDIALATRDIAEELGGEWADRFLVLYGIAAPDSQRIAFYRLLDEFF

SEQ ID NO: 12 - DNA, 1026 bp, artificial sequence
  Hygromycin phosphotransferase
atgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggcattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatag

SEQ ID NO: 13 - protein, 341 aa, artificial sequence
  Synthetic Construct
MKKPELTATSVEKFLIEKFDSVSDLMQLSEGEESRAFSFDVGGRGYVLRVNSCADGFYKDRYVYRHFASAALPIPEVLDIGAFSESLTYCISRRAQGVTLQDLPETELPAVLQPVAEAMDAIAAADLSQTSGFGPFGPQGIGQYTTWRDFICAIADPHVYHWQTVMDDTVSASVAQALDELMLWAEDCPEVRHLVHADFGSNNVLTDNGRITAVIDWSEAMFGDSQYEVANIFFWRPWLACMEQQTRYFERRHPELAGSPRLRAYMLRIGLDQLYQSLVDGNFDDAAWAQGRCDAIVRSGAGTVGRTQIARRSAAVWTDGCVEVLADSGNRRPSTRPRAKE

SEQ ID NO: 14 - DNA, 660 bp, artificial sequence
  Chloramphenicol acetyl transferase
atggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaa

SEQ ID NO: 15 - protein, 219 aa, artificial sequence
  Synthetic Construct
MEKKITGYTTVDISQWHRKEHFEAFQSVAQCTYNQTVQLDITAFLKTVKKNKHKFYPAFIHILARLMNAHPEFRMAMKDGELVIWDSVHPCYTVFHEQTETFSSLWSEYHDDFRQFLHIYSQDVACYGENLAYFPKGFIENMFFVSANPWVSFTSFDLNVANMDNFFAPVFTMGKYYTQGDKVLMPLAIQVHHAVCDGFHVGRMLNELQQYCDEWQGGA

SEQ ID NO: 16 - DNA, 1332 bp, Salmonella typhimurium
  CDS (27)..(1310)
tttctgttttttgagagttgagtttcatggaatccctgacgttacaacccatcgcgcgggtcgatggcgccattaatttacctggctccaaaagtgtttcaaaccgtgctttgctcctggcggctttaccttgtggtaaaaccgctctgacgaatctgctggatagcgatgacgtccgccatatgctcaatgccctgagcgcgttggggatcaattacaccctttctgccgatcgcacccgctgtgatatcacgggtaatggcggcgcattacgtgcgccaggcgctctggaactgtttctcggtaatgccggaaccgcgatgcgtccgttagcggcagcgctatgtctggggcaaaatgagatagtgttaaccggcgaaccgcgtatgaaagagcgtccgataggccatctggtcgattcgctgcgtcagggcggggcgaatattgattacctggagcaggaaaactatccgcccctgcgtctgcgcggcggttttaccggcggcgacattgaggttgatggtagcgtttccagccagttcctgaccgctctgctgatgacggcgccgctggcccctaaagacacaattattcgcgttaaaggcgaactggtatcaaaaccttacatcgatatcacgctaaatttaatgaaaacctttggcgtggagatagcgaaccaccactaccaacaatttgtcgtgaagggaggtcaacagtatcactctccaggtcgctatctggtcgagggcgatgcctcgtcagcgtcctattttctcgccgctggggcgataaaaggcggcacggtaaaagtgaccggaattggccgcaaaagtatgcagggcgatattcgttttgccgatgtgctggagaaaatgggcgcgaccattacctggggcgatgattttattgcctgcacgcgcggtgaattgcacgccatagatatggatatgaaccatattccggatgcggcgatgacgattgccaccacggcgctgtttgcgaaaggaaccacgacgttgcgcaatatttataactggcgagtgaaagaaaccgatcgcctgttcgcgatggcgaccgagctacgtaaagtgggcgctgaagtcgaagaagggcacgactatattcgtatcacgccgccggcgaagctccaacacgcggatattggcacgtacaacgaccaccgtatggcgatgtgcttctcactggtcgcactgtccgatacgccagttacgatcctggaccctaaatgtaccgcaaaaacgttccctgattatttcgaacaactggcgcgaatgagtacgcctgcctaagtcttctgttgcgccagtcgac

SEQ ID NO: 17 - protein, 427 aa, Salmonella typhimurium
MESLTLQPIARVDGAINLPGSKSVSNRALLLAALPCGKTALTNLLDSDDVRHMLNALSALGINYTLSADRTRCDITGNGGALRAPGALELFLGNAGTAMRPLAAALCLGQNEIVLTGEPRMKERPIGHLVDSLRQGGANIDYLEQENYPPLRLRGGFTGGDIEVDGSVSSQFLTALLMTAPLAPKDTIIRVKGELVSKPYIDITLNLMKTFGVEIANHHYQQFVVKGGQQYHSPGRYLVEGDASSASYFLAAGAIKGGTVKVTGIGRKSMQGDIRFADVLEKMGATITWGDDFIACTRGELHAIDMDMNHIPDAAMTIATTALFAKGTTTLRNIYNWRVKETDRLFAMATELRKVGAEVEEGHDYIRITPPAKLQHADIGTYNDHRMAMCFSLVALSDTPVTILDPKCTAKTFPDYFEQLARMSTPA

SEQ ID NO: 18 - DNA, 1323 bp, Streptomyces viridochromogenes
  CDS (1)..(1323)
atgaccatccacaaccccgaggaaccggagtacttccccgaggtcttcccccgcgacgccttcccccggtacacctgggacgacggcatgcggccgctcaccctgccgcatgaggtctggctgtcggagaccacccaccgcgacggccagcagggcggactgcccctctccctggacaccagccggaagatctacgacatcctctgcgagatcaccggcgactccacggcgatccggcacgccgagttcttcccgtaccgcgactccgaccgcaacgccctgatctacgcactggaacggcaccgcgacggcgccccgatcgagcccaccacctggatccgcgcccgccgcgaggacgtcgagctgatcaagcggatcggcgtcaccgagaccggtctgctcagctcctcctccgactaccacaccttccacaagttcggctcgggcggccggaccgaggccgccgcgatgtacctcgacgcggtcaccatggctctggaccacggcatcaagccccgcgtccacctggaggacaccacccgctccgacccggacttcgtgcgcgctctggtcgaggaggtcctgcggatcgccgccccgtaccccgccgaactccagccgcgcttccgggtctgcgacaccctcggcatcggcctgcccttcgacgacgtcgccctgccgcgcagcgtcccccgctggatccggctgctgcggggcttcggtctgacgccctcccagatcgagctccacccgcacaacgacacctggctgaccgtcgccaactgcctggccgcgatccgcgagggctgcggagtgatcagcggaacgacgctcggcaccggcgaacgcaccggcaacgcgccgctggaagccgtcatggtgcacctgctcggcatgggctactggcccgagggcggcgtcgacctgactgccgtcaacaagctcgtcgagctctacgacggcatcggcgccggcccgtcgcagaagtacccgctcttcggccgtgacgcctacgtcacccgggccggcatccacgccgacggcctcaacaagttctggtggatgtacgcgccgttcaacgcccccctgctcaccggccgggaactggacgtcgccctcaccaaggactcgggccaggcgggtctgctcttcgtgctgaacaagcggctcgggctgcggctggagaagggcgatccgcgggtcgtggacctgctcgcgtggatggacgagcagtgggacgccggccgggtctccgcgatcgagtggagcgaactcgaaccggtcgtcgagaaggtcttcgccaccgaggaagaggcgagctga

SEQ ID NO: 19 - protein, 440 aa, Streptomyces viridochromogenes
MTIHNPEEPEYFPEVFPRDAFPRYTWDDGMRPLTLPHEVWLSETTHRDGQQGGLPLSLDTSRKIYDILCEITGDSTAIRHAEFFPYRDSDRNALIYALERHRDGAPIEPTTWIRARREDVELIKRIGVTETGLLSSSSDYHTFHKFGSGGRTEAAAMYLDAVTMALDHGIKPRVHLEDTTRSDPDFVRALVEEVLRIAAPYPAELQPRFRVCDTLGIGLPFDDVALPRSVPRWIRLLRGFGLTPSQIELHPHNDTWLTVANCLAAIREGCGVISGTTLGTGERTGNAPLEAVMVHLLGMGYWPEGGVDLTAVNKLVELYDGIGAGPSQKYPLFGRDAYVTRAGIHADGLNKFWWMYAPFNAPLLTGRELDVALTKDSGQAGLLFVLNKRLGLRLEKGDPRVVDLLAWMDEQWDAGRVSAIEWSELEPVVEKVFATEEEAS

SEQ ID NO: 20 - DNA, 2457 bp, artificial sequence
  Glycine max Cp4esps
tggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgacaagctgactctagcagatctttcaagaatggcacaaattaacaacatggcacaagggatacaaacccttaatcccaattccaatttccataaaccccaagttcctaaatcttcaagttttcttgtttttggatctaaaaaactgaaaaattcagcaaattctatgttggttttgaaaaaagattcaatttttatgcaaaagttttgttcctttaggatttcagcatcagtggctacagcctgcatgcttcacggtgcaagcagccggcccgcaaccgcccgcaaatcctctggcctttccggaaccgtccgcattcccggcgacaagtcgatctcccaccggtccttcatgttcggcggtctcgcgagcggtgaaacgcgcatcaccggccttctggaaggcgaggacgtcatcaatacgggcaaggccatgcaggccatgggcgccaggatccgtaaggaaggcgacacctggatcatcgatggcgtcggcaatggcggcctcctggcgcctgaggcgccgctcgatttcggcaatgccgccacgggctgccgcctgaccatgggcctcgtcggggtctacgatttcgacagcaccttcatcggcgacgcctcgctcacaaagcgcccgatgggccgcgtgttgaacccgctgcgcgaaatgggcgtgcaggtgaaatcggaagacggtgaccgtcttcccgttaccttgcgcgggccgaagacgccgacgccgatcacctaccgcgtgccgatggcctccgcacaggtgaagtccgccgtgctgctcgccggcctcaacacgcccggcatcacgacggtcatcgagccgatcatgacgtgcgatcatacggaaaagatgctgcagggctttggcgccaaccttaccgtcgagacggatgcggacggcgtgcgcaccatccgcctggaaggccgcggcaagctcaccggccaagtcatcgacgtgccgggcgacccgtcctcgacggccttcccgctggttgcggccctgcttgttccgggctccgacgtcaccatcctcaacgtgctgatgaaccccacccgcaccggcctcatcctgacgctgcaggaaatgggcgccgacatcgaagtcatcaacctgcgccttgccggcggcgaagacgtggcggacctgcgcgttcgctcctccacgctgaagggcgtcacggtgccggaagaccgcgcgcctccgatgatcgacgaatatccgattctcgctgtcgccgccgccttcgcggaaggggcgaccgtgatgaacggtctggaagaactccgcgtcaaggaaagcgaccgcctctcggccgtcgccaatggcctcaagctcaatggcgtggattgcgatgagggcgagacgtcgctcgtcgtgcgtggccgccctgacggcaaggggctcggcaacgcctcgggcgccgccgtcgccacccatctcgatcaccgcatcgccatgagcttcctcgtcatgggcctcgtgtcggaaaaccctgtcacggtggacgatgccacgatgatcgccacgagcttcccggagttcatggacctgatggccgggctgggcgcgaagatcgaactctccgatacgaaggctgcctgatgagctcgaattcgagctcggtaccggatccaattcccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggggatcgatcccccaccggtccttcatgttcggcggtctcgcgagcggtgaaacgcgcatcaccggccttctggaaggcgaggacgtcatcaatacgggcaaggccatgcaggccatgggcgccaggatccgtaaggaaggcgacacctggatcatcgatggcgtcggcaatggcggcctcctggcgcctgaggcgccgctcgatttcggcaatgccgccacgggctgccgcctgaccatgggcctcgtcggggtctacgatttcaagcgcatcatgctgggaa

SEQ ID NO: 21 - protein, 527 aa, artificial sequence
  Synthetic Construct
MAQINNMAQGIQTLNPNSNFHKPQVPKSSSFLVFGSKKLKNSANSMLVLKKDSIFMQKFCSFRISASVATACMLHGASSRPATARKSSGLSGTVRIPGDKSISHRSFMFGGLASGETRITGLLEGEDVINTGKAMQAMGARIRKEGDTWIIDGVGNGGLLAPEAPLDFGNAATGCRLTMGLVGVYDFDSTFIGDASLTKRPMGRVLNPLREMGVQVKSEDGDRLPVTLRGPKTPTPITYRVPMASAQVKSAVLLAGLNTPGITTVIEPIMTCDHTEKMLQGFGANLTVETDADGVRTIRLEGRGKLTGQVIDVPGDPSSTAFPLVAALLVPGSDVTILNVLMNPTRTGLILTLQEMGADIEVINLRLAGGEDVADLRVRSSTLKGVTVPEDRAPPMIDEYPILAVAAAFAEGATVMNGLEELRVKESDRLSAVANGLKLNGVDCDEGETSLVVRGRPDGKGLGNASGAAVATHLDHRIAMSFLVMGLVSENPVTVDDATMIATSFPEFMDLMAGLGAKIELSDTKAA

SEQ ID NO: 22 - DNA, 441 bp, artificial sequence
  Glyphosate N-acetyl transferase
atgatagaggtaaaaccgattaacgcagaggatacctatgacctaaggcatagagtcctcagaccaaaccagccgatagaagcgtgtatgtttgaaagcgatttaacgcgtagtgcatttcacttaggcggcttctacgggggcaaactgatttccgtcgcttcattccaccaggccgagcactcggaacttcaaggcaagaaacagtaccagcttcgaggtgtggctaccttggaaggttatcgtgagcagaaggcgggttccagtctagttaaacacgctgaagaaattctacgtaagaggggggcggacatgatttggtgtaatgcgcggacatctgcctcaggctactacagaaagttaggcttcagcgagcagggagaggtattcgacacgccgccagtaggacctcacatcctgatgtataaaaggatcacataa

SEQ ID NO: 23 - protein, 146 aa, artificial sequence
  Synthetic Construct
MIEVKPINAEDTYDLRHRVLRPNQPIEACMFESDLTRSAFHLGGFYGGKLISVASFHQAEHSELQGKKQYQLRGVATLEGYREQKAGSSLVKHAEEILRKRGADMIWCNARTSASGYYRKLGFSEQGEVFDTPPVGPHILMYKRIT

SEQ ID NO: 24 - DNA, 1176 bp, Escherichia coli
  CDS (1)..(1176)
atgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaagtctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggcgctcgacagtcagcagggtgaaccgtggcaaacgattcgtttaatttctgaattttacccggatgacagcggcctgttctctccgctgttgctgaatgtggtgaaattaaatcctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaagttcgaagccaaaccggctaaccagttgttgacccagccggtgaaacaaggtgcagaactggacttcccgattcctgttgatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcacgggtttacaacaagctgtaa

SEQ ID NO: 25 - protein, 391 aa, Escherichia coli
MQKLINSVQNYAWGSKTALTELYGMENPSSQPMAELWMGAHPKSSSRVQNAAGDIVSLRDVIESDKSTLLGEAVAKRFGELPFLFKVLCAAQPLSIQVHPNKHKSEIGFAKENAAGIPMDAAERNYKDPNHKPELVFALTPFLAMNAFREFSEIVSLLQPVAGAHPAIAHFLQQPDAERLSELFASLLNMQGEEKSRALAILKSALDSQQGEPWQTIRLISEFYPDDSGLFSPLLLNVVKLNPGEAMFLFAETPHAYLQGVALEVMANSDNVLRAGLTPKYIDIPELVANVKFEAKPANQLLTQPVKQGAELDFPIPVDDFAFSLHDLSDKETTISQQSAAILFCVEGDATLWKGSQQLQLKPGESAFIAANESPVTVKGHGRLARVYNKL

SEQ ID NO: 26 - DNA, 765 bp, Aspergillus fumigatus
  CDS (1)..(765)
atgaataccgtcctcgaccccgtaacaacatacggcttaaccgccgtcccctcctcctgtaccaccctcttcaacggcaacatccccagctcaccaccccctttcgtcccaaccactcaaactcccatcccatcgacccccctcgcaactcgcatagacaactacgcccgcgaaaatctctccgagcaaacctaccaccactccctccgcgtctaccacttcggcctcgccatcaaacgccacgcctttcccacctggtccttcacagacgagacctacttcctcgcctgcctgctccatgacctcggcacaacagacaagaacacgcgcaagacccggctgagcttcgagttctacggcggccttctcgccctcgaggtcctgcagtcgagcgcggagcatcccgccaaccattcggggtacggcgcagccaccgggaccgtcgcgccgagggagcaggcggagagtgtcgccgaggcgatcgtccggcaccaggatttgtgcgagaagggaatgatcacggcgctggggcggttgttgcagttggcgacgttgctggataataccggcgccaatgagcacctcgtcaatccgcagacgatcaaggatgtctgcgagaattatccccggaagcagtggagcagttgctttgcgggggtcattcgcaaggagaacgggctgaagccgtgggcgcattccaccacgctgggggaggagtttccagcgaagatcatggggaataagctgatggcgccgtatgagtga

SEQ ID NO: 27 - protein, 254 aa, Aspergillus fumigatus
MNTVLDPVTTYGLTAVPSSCTTLFNGNIPSSPPPFVPTTQTPIPSTPLATRIDNYARENLSEQTYHHSLRVYHFGLAIKRHAFPTWSFTDETYFLACLLHDLGTTDKNTRKTRLSFEFYGGLLALEVLQSSAEHPANHSGYGAATGTVAPREQAESVAEAIVRHQDLCEKGMITALGRLLQLATLLDNTGANEHLVNPQTIKDVCENYPRKQWSSCFAGVIRKENGLKPWAHSTTLGEEFPAKIMGNKLMAPYE

SEQ ID NO: 28 - DNA, 1160 bp, Rhodotorula gracilis
  CDS (1)..(1107)
atgcactcgcagaagcgcgtcgttgtcctcggatcaggcgttatcggtctgagcagcgccctcatcctcgctcggaagggctacagcgtgcatattctcgcgcgcgacttgccggaggacgtctcgagccagactttcgcttcaccatgggctggcgcgaattggacgcctttcatgacgcttacagacggtcctcgacaagcaaaatgggaagaatcgactttcaagaagtgggtcgagttggtcccgacgggccatgccatgtggctcaaggggacgaggcggttcgcgcagaacgaagacggcttgctcgggcactggtacaaggacatcacgccaaattaccgccccctcccatcttccgaatgtccacctggcgctatcggcgtaacctacgacaccctctccgtccacgcaccaaagtactgccagtaccttgcaagagagctgcagaagctcggcgcgacgtttgagagacggaccgttacgtcgcttgagcaggcgttcgacggtgcggatttggtggtcaacgctacgggacttggcgccaagtcgattgcgggcatcgacgaccaagccgccgagccaatccgcgggcaaaccgtcctcgtcaagtccccatgcaagcgatgcacgatggactcgtccgaccccgcttctcccgcctacatcattccccgaccaggtggcgaagtcatctgcggcgggacgtacggcgtgggagactgggacttgtctgtcaacccagagacggtccagcggatcctcaagcactgcttgcgcctcgacccgaccatctcgagcgacggaacgatcgaaggcatcgaggtcctccgccacaacgtcggcttgcgacctgcacgacgaggcggaccccgcgttgaggcagaacggatcgtcctgcctctcgaccggacaaagtcgcccctctcgctcggcaggggcagcgcacgagcggcgaaggagaaggaggtcacgcttgtgcatgcgtatggcttctcgagtgcgggataccagcagagttggggcgcggcggaggatgtcgcgcagctcgtcgacgaggcgttccagcggtaccacggcgcggcgcgggagtcgaagttgtagggcgggatttgtggctgtattgcgggcatctacaagaaaaaaaaaaaaaaaaa

SEQ ID NO: 29 - protein, 368 aa, Rhodotorula gracilis
MHSQKRVVVLGSGVIGLSSALILARKGYSVHILARDLPEDVSSQTFASPWAGANWTPFMTLTDGPRQAKWEESTFKKWVELVPTGHAMWLKGTRRFAQNEDGLLGHWYKDITPNYRPLPSSECPPGAIGVTYDTLSVHAPKYCQYLARELQKLGATFERRTVTSLEQAFDGADLVVNATGLGAKSIAGIDDQAAEPIRGQTVLVKSPCKRCTMDSSDPASPAYIIPRPGGEVICGGTYGVGDWDLSVNPETVQRILKHCLRLDPTISSDGTIEGIEVLRHNVGLRPARRGGPRVEAERIVLPLDRTKSPLSLGRGSARAAKEKEVTLVHAYGFSSAGYQQSWGAAEDVAQLVDEAFQRYHGAARESKL

SEQ ID NO: 30 - DNA, 1947 bp, Mus musculus
  CDS (1)..(1947)
atgtccctaaaatggagtgcgtgttgggtcgcgctgggccagctgctgtgcagctgcgcgctggctctgaagggcgggatgctgttcccgaaggagagcccgtcgcgggagctcaaggcgctggacggactgtggcacttccgcgccgacctctcgaacaaccggctgcagggtttcgagcagcaatggtaccggcagccgctacgggagtcgggcccagtcttggacatgcctgtcccttctagcttcaatgacatcacccaagaagcagcccttcgggactttattggctgggtgtggtatgaacgggaagcaatcctgccacggcgatggacccaagataccgacatgagagtggtgttgaggatcaacagtgcccattattatgcagttgtgtgggtgaatgggattcatgtggtggaacatgagggaggtcacctcccctttgaggctgacattagcaagctggtccagagtgggcccctgaccacctgccggatcacgattgccatcaacaacacactgacccctcatacccttccgccggggaccatcgtctacaagactgacacctccatgtatcccaagggttactttgtccaggacacaagctttgacttcttcaactatgcgggactgcatcgatctgtggtcctctataccacccctaccacttacatcgatgatatcactgtgatcactaatgtggagcaagacatcgggctggtgacctactggatttctgtgcagggcagtgaacatttccagctagaagtgcaacttttggatgaggatggcaaagtcgtggcccatgggacagggaaccagggtcaacttcaggttcccagtgccaacctctggtggccttacctgatgcatgagcatccagcctacatgtactccttggaggtgaaggtgacaacaactgagtctgtgactgactactacacccttcctatcgggattcgaacagtggctgtcacaaagagcaagttcctcataaacgggaagcccttctatttccaaggggtcaataagcacgaggattcagatatccgagggaaaggcttcgactggccgctgctggtaaaggatttcaacctgctccgttggctcggggcaaattcctttcgtaccagccactatccctactcagaggaggtacttcagctctgtgaccgatacgggattgtggtcatcgatgagtgtcccggtgtgggcattgtgctacctcagagttttggcaacgagtcacttcggcaccacctagaggtgatggaggagctggttcgccgggacaaaaatcaccctgcggttgtgatgtggtctgtggccaatgagccttcctctgctctgaaacccgccgcatattactttaagacgctgatcacccacaccaaagccctggacctcacccgtcccgtgacctttgtgagcaacgccaaatatgatgcagacctgggggccccgtacgtggatgttatctgtgtaaacagctacttttcttggtatcatgactatgggcatttggaggtgattcagccacagctgaatagccagtttgagaactggtataagacgcatcagaagccgattatccagagcgagtatggagcagacgcaatcccagggatccacgaggacccgcctcgcatgttcagtgaggagtaccagaaggctgttctggagaattaccattcagttctggatcagaaacgtaaagaatacgtggtcggagagctcatctggaatttcgccgacttcatgacgaaccagtcaccgctgagagtaatcggaaacaagaaggggatcttcactcgccagagacagcccaaaacttcggcctttattttgcgagagagatactggaggattgccaacgaaaccggaggtcacggttcagggccgagaacccagtgtttcggaagcagaccgttcacgttctaa

SEQ ID NO: 31 - protein, 648 aa, Mus musculus
MSLKWSACWVALGQLLCSCALALKGGMLFPKESPSRELKALDGLWHFRADLSNNRLQGFEQQWYRQPLRESGPVLDMPVPSSFNDITQEAALRDFIGWVWYEREAILPRRWTQDTDMRVVLRINSAHYYAVVWVNGIHVVEHEGGHLPFEADISKLVQSGPLTTCRITIAINNTLTPHTLPPGTIVYKTDTSMYPKGYFVQDTSFDFFNYAGLHRSVVLYTTPTTYIDDITVITNVEQDIGLVTYWISVQGSEHFQLEVQLLDEDGKVVAHGTGNQGQLQVPSANLWWPYLMHEHPAYMYSLEVKVTTTESVTDYYTLPIGIRTVAVTKSKFLINGKPFYFQGVNKHEDSDIRGKGFDWPLLVKDFNLLRWLGANSFRTSHYPYSEEVLQLCDRYGIVVIDECPGVGIVLPQSFGNESLRHHLEVMEELVRRDKNHPAVVMWSVANEPSSALKPAAYYFKTLITHTKALDLTRPVTFVSNAKYDADLGAPYVDVICVNSYFSWYHDYGHLEVIQPQLNSQFENWYKTHQKPIIQSEYGADAIPGIHEDPPRMFSEEYQKAVLENYHSVLDQKRKEYVVGELIWNFADFMTNQSPLRVIGNKKGIFTRQRQPKTSAFILRERYWRIANETGGHGSGPRTQCFGSRPFTF

SEQ ID NO: 32 - DNA, 1653 bp, artificial sequence
  Luciferase
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 33 - protein, 550 aa, artificial sequence
  Synthetic Construct
MEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 34 - DNA, 3075 bp, artificial sequence
  Beta galactosidase
atgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgaactcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 35 - protein, 1024 aa, artificial sequence
  Synthetic Construct
MTMITDSLAVVLQRRDWENPGVTQLNRLAAHPPFASWRNSEEARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAEFQLSAGRYHYQLVWCQK

SEQ ID NO: 36 - DNA, 678 bp, artificial sequence
  Mutant discosoma red fluorescent protein
atggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctgcttcatctacaaggtgaagttcatcggcgtgaacttcccctccgacggccccgtaatgcagaagaagactatgggctgggagccctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcaccgagggacgccaccatctgttcctttag

SEQ ID NO: 37 - protein, 225 aa, artificial sequence
  Synthetic Construct
MASSEDVIKEFMRFKVRMEGSVNGHEFEIEGEGEGRPYEGTQTAKLKVTKGGPLPFAWDILSPQFQYGSKVYVKHPADIPDYKKLSFPEGFKWERVMNFEDGGVVTVTQDSSLQDGCFIYKVKFIGVNFPSDGPVMQKKTMGWEPSTERLYPRDGVLKGEIHKALKLKDGGHYLVEFKSIYMAKKPVQLPGYYYVDSKLDITSHNEDYTIVEQYERTEGRHHLFL

SEQ ID NO: 38 - DNA, 941 bp, Aequorea coerulescens
  CDS (67)..(783)
attcaaaacactgcagaattttggatagattttcctgctacttcacacgcataaaagacaagaaagatgagtaaaggagcagaacttttcactggagttgtcccaattcttattgaattaaatggtgatgttaatgggcacaaattctctgtcagtggagagggcgaaggtgatgcgacatacggaaagttaacccttaaatttatttgcactacaggaaaactacctgttccatggccaacacttgtcactactttctcttatggtgttcaatgcttttcaagatatccagatcatatgaaacagcatgacttcttcaagagtgccatgcctgaaggttatatacaggaaagaactatatttttcaaagatgacgggaactacaagtcgcgtgctgaagtcaagttcgaaggtgataccctggttaatagaattgagttaacaggtactgattttaaagaagatggaaacatccttggaaataaaatggaatacaactataacgcacataatgtatacatcatgacagacaaagcaaaaaatggaatcaaagttaacttcaaaattagacacaacattgaagatggaagcgttcaacttgcagaccattatcaacaaaatactccaattggcgatggccctgtccttttaccagataaccattacctgtccacacaatctaccctttccaaagatcccaacgaaaagagagatcacatgatctattttgagtttgtaacagctgctgcgattacacatggcatggatgaattatacaaataaatgtatagacttcaagttgacactaacgtgtccgaacaattactaaaatctcagggttcctggttaaaatcaggctgagatattatttacatattatagattcattagaattatttaaatactttatagatgttattgataggggttattttcttatt

SEQ ID NO: 39 - protein, 238 aa, Aequorea coerulescens
MSKGAELFTGVVPILIELNGDVNGHKFSVSGEGEGDATYGKLTLKFICTTGKLPVPWPTLVTTFSYGVQCFSRYPDHMKQHDFFKSAMPEGYIQERTIFFKDDGNYKSRAEVKFEGDTLVNRIELTGTDFKEDGNILGNKMEYNYNAHNVYIMTDKAKNGIKVNFKIRHNIEDGSVQLADHYQQNTPIGDGPVLLPDNHYLSTQSTLSKDPNEKRDHMIYFEFVTAAAITHGMDELYK

SEQ ID NO: 40 - DNA, 2885 bp, Triticum aestivum
  CDS (265)..(2757)
agaccgtccaaaaatctgttttacaaagctccaattgctccttgcttatccagctttttttgtgttggcaaactacactttttcaaccgattttgttcttctcacactttcttcttaggctaaacaaaccttaccgtgcacgcagccatggtcctgaatcttcacctcgtccctataaaagcctagccaaccttcacaatctcttcatcacccacaacaccgagcatcacaaactagagatcaattcaccgacagtccaccgagatgactaagcggttggttctttttgcggcggtagtcgtcgcccttgtggctctcaccgctgctgaaggtgaggcctctgggcaactacagtgtgagcgcgagctccaggaacactcgcttaaggcatgccgacaggtcgtagaccagcagctccgagacgttagccccgagtgccaacccgtcggcggcggcccggtcgcgagacaatatgagcagcaagtcgtggtgccgcccaagggtggatctttctaccccggcgagaccacgccaccacagcaactccaacaaagtatactttggggaatacctgcactactaagaaggtattacctaagtgtaacttctccgcaacaggtttcatactatccaggccaagcttcttcgcaacggccaggacaaggtcagcagccaggacaaggacaacaagaatactacctaacttctccgcaacagtcaggacaatggcaacaaccgggacaagggcaagcagggtactacccaacttctccgcagcagtcaggacaagagcaaccagggtactatccaacttctccatggcagccagaacaattgcaacaaccaacacaagggcaacaaagacagcaaccaggacaaggtcagcaactaagacaaggacaacaaggtcagcagtcaggacaagggcaaccaagatactatccaacttcttcgcagcagccaggacaattgcaacaactagcacaaggccaacaagggcagcaaccagaacgagggcaacaaggccaacagtcaggacaagggcaacaactaggacaagggcaacaaggtcagcagccaggacaaaagcaacaatcaggacaaggacaacaagggtactacccaatttctccgcaacagttaggacaagggcaacagtcaggacaagggcaactagggtactacccaacttctccgcagcagtcaggacaaggacaatcaggatactatccaacttctgcgcagcagccaggacaattgcaacaatcaacacaagagcagcaattaggacaagagcaacaagatcagcaatcaggacaagggcgacaaggtcaacagtcaggacaaaggcaacaagatcagcagtcaggacaagggcagcaaccgggacaaaggcagccagggtactactcaacttctccgcaacaattaggacaagggcaaccaaggtactacccaacttctccgcagcagccaggacaagagcagcagccaagacaattgcaacaaccagaacaagggcaacaaggtcagcagccagaacaagggcagcaaggtcagcagccaggacaaggggagcaaggtcagcagccaggacaagggcaacaagggcagcaaccgggacaagggcagccagggtactacccaacttctccgcagcagtcgggacaagggcaaccagggtactacccaacttctccacagcagtcaggacaattgcaacaaccagcacaagggcagcaaccaggacaagagcaacaaggtcaacagccaggacaagggcaacaaggtcaacagccaggacaagggcagcaaccgggacaagggcagccagggtactacccaacttctccgcagcagtcaggacaagagcaacagctagaacaatggcaacagtcaggacaggggcaaccagggcactacccaacttctccgttgcagccaggacaagggcaaccagggtactacccaacttctccacaacagataggacaagggcagcagccaggacaattgcaacaaccaacacaagggcaacaagggcagcaaccaggacaagggcaacaaggtcaacagccaggacaagggcaacaaggtcagcagccaggacaagggcagcaaccaggacaagggcagccagggtactacccaacttctttgcagcagtcaggacaagggcaacagccaggacaatggcaacaaccaggacaaggactaccagggtactacccaacttcttcgttgcagccagaacaagggcaacaagggtactacccaacttctcagcagcaaccaggacaagggccgcaaccaggacaatggcaacaatcaggacaagggcaacaagggtactacccaacttctccgcagcagtcaggacaagggcaacagccaggacaatggttgcaaccaggacaatggctgcaatcagggtactacctaacttctccgcagcagttaggacaagggcaacagccaagacaatggctgcaaccaagacaagggcaacaaggatactacccaacttctccgcagcagtcaggacaagggcaacaattaggacaagggcaacaaggatactacccaacttctccgcagcagtcaggacaagggcaacaaggctacgacagcccataccatgttagcgcggagcaccaggcggccagcctaaaggtggcaaaggcacagcagctcgcggcacagctgccggcaatgtgccggctagagggcggcgacgcattgttggccagccagtgatagaactctctgcagctcgcttggtgcttaggcatgcatgcaccttagctatacaataaatgtggcgtgtgttcaagtttttcatgtaactaatgtaaagcccagtaatgatgcaaaatgaaaagctt

SEQ ID NO: 41 - protein, 830 aa, Triticum aestivum
MTKRLVLFAAVVVALVALTAAEGEASGQLQCERELQEHSLKACRQVVDQQLRDVSPECQPVGGGPVARQYEQQVVVPPKGGSFYPGETTPPQQLQQSILWGIPALLRRYYLSVTSPQQVSYYPGQASSQRPGQGQQPGQGQQEYYLTSPQQSGQWQQPGQGQAGYYPTSPQQSGQEQPGYYPTSPWQPEQLQQPTQGQQRQQPGQGQQLRQGQQGQQSGQGQPRYYPTSSQQPGQLQQLAQGQQGQQPERGQQGQQSGQGQQLGQGQQGQQPGQKQQSGQGQQGYYPISPQQLGQGQQSGQGQLGYYPTSPQQSGQGQSGYYPTSAQQPGQLQQSTQEQQLGQEQQDQQSGQGRQGQQSGQRQQDQQSGQGQQPGQRQPGYYSTSPQQLGQGQPRYYPTSPQQPGQEQQPRQLQQPEQGQQGQQPEQGQQGQQPGQGEQGQQPGQGQQGQQPGQGQPGYYPTSPQQSGQGQPGYYPTSPQQSGQLQQPAQGQQPGQEQQGQQPGQGQQGQQPGQGQQPGQGQPGYYPTSPQQSGQEQQLEQWQQSGQGQPGHYPTSPLQPGQGQPGYYPTSPQQIGQGQQPGQLQQPTQGQQGQQPGQGQQGQQPGQGQQGQQPGQGQQPGQGQPGYYPTSLQQSGQGQQPGQWQQPGQGLPGYYPTSSLQPEQGQQGYYPTSQQQPGQGPQPGQWQQSGQGQQGYYPTSPQQSGQGQQPGQWLQPGQWLQSGYYLTSPQQLGQGQQPRQWLQPRQGQQGYYPTSPQQSGQGQQLGQGQQGYYPTSPQQSGQGQQGYDSPYHVSAEHQAASLKVAKAQQLAAQLPAMCRLEGGDALLASQ

SEQ ID NO: 42 - DNA, 1404 bp, Aspergillus niger
  CDS (1)..(1404)
atgggcgtctctgctgttctacttcctttgtatctcctgtctggagtcacctccggactggcagtccccgcctcgagaaatcaatccagttgcgatacggtcgatcaggggtatcaatgcttctccgagacttcgcatctttggggtcaatacgcaccgttcttctctctggcaaacgaatcggtcatctcccctgaggtgcccgccggatgcagagtcactttcgctcaggtcctctcccgtcatggagcgcggtatccgaccgactccaagggcaagaaatactccgctctcattgaggagatccagcagaacgcgaccacctttgacggaaaatatgccttcctgaagacatacaactacagcttgggtgcagatgacctgactcccttcggagaacaggagctagtcaactccggcatcaagttctaccagcggtacgaatcgctcacaaggaacatcgttccattcatccgatcctctggctccagccgcgtgatcgcctccggcaagaaattcatcgagggcttccagagcaccaagctgaaggatcctcgtgcccagcccggccaatcgtcgcccaagatcgacgtggtcatttccgaggccagctcatccaacaacactctcgacccaggcacctgcactgtcttcgaagacagcgaattggccgataccgtcgaagccaatttcaccgccacgttcgtcccctccattcgtcaacgtctggagaacgacctgtccggtgtgactctcacagacacagaagtgacctacctcatggacatgtgctccttcgacaccatctccaccagcaccgtcgacaccaagctgtcccccttctgtgacctgttcacccatgacgaatggatcaactacgactacctccagtccttgaaaaagtattacggccatggtgcaggtaacccgctcggcccgacccagggcgtcggctacgctaacgagctcatcgcccgtctgacccactcgcctgtccacgatgacaccagttccaaccacactttggactcgagcccggctacctttccgctcaactctactctctacgcggacttttcgcatgacaacggcatcatctccattctctttgctttaggtctgtacaacggcactaagccgctatctaccacgaccgtggagaatatcacccagacagatggattctcgtctgcttggacggttccgtttgcttcgcgtttgtacgtcgagatgatgcagtgtcaggcggagcaggagccgctggtccgtgtcttggttaatgatcgcgttgtcccgctgcatgggtgtccggttgatgctttggggagatgtacccgggatagctttgtgagggggttgagctttgctagatctgggggtgattgggcggagtgttttgcttag

SEQ ID NO: 43 - protein, 467 aa, Aspergillus niger
MGVSAVLLPLYLLSGVTSGLAVPASRNQSSCDTVDQGYQCFSETSHLWGQYAPFFSLANESVISPEVPAGCRVTFAQVLSRHGARYPTDSKGKKYSALIEEIQQNATTFDGKYAFLKTYNYSLGADDLTPFGEQELVNSGIKFYQRYESLTRNIVPFIRSSGSSRVIASGKKFIEGFQSTKLKDPRAQPGQSSPKIDVVISEASSSNNTLDPGTCTVFEDSELADTVEANFTATFVPSIRQRLENDLSGVTLTDTEVTYLMDMCSFDTISTSTVDTKLSPFCDLFTHDEWINYDYLQSLKKYYGHGAGNPLGPTQGVGYANELIARLTHSPVHDDTSSNHTLDSSPATFPLNSTLYADFSHDNGIISILFALGLYNGTKPLSTTTVENITQTDGFSSAWTVPFASRLYVEMMQCQAEQEPLVRVLVNDRVVPLHGCPVDALGRCTRDSFVRGLSFARSGGDWAECFA

SEQ ID NO: 44 - DNA, 1203 bp, Hepatitis B virus
  CDS (1)..(1203)
atgggaggttggtcttccaaacctcgacaaggcatggggacgaatctttctgttcccaatcctctgggattctttcccgatcaccagttggaccctgcgttcggagccaactcaaacaatccagattgggacttcaaccccaacaaggatcattggccagaggcaaatcaggtaggagcgggagcattcgggccagggttcaccccaccacacggcggtcttttggggtggagccctcaggctcagggcatattgacaacagtgccagcagcacctcctcctgcctccaccaatcggcagtcaggaagacagcctactcccatctctccacctctaagagacagtcatcctcaggccatgcagtggaactccacgacattccaccaagctctgctagatcccagagtgaggggcctatattttcctgctggtggctccagttccggaacagtaaaccctgttccgactactgcctcacccatatcgtcaatcttctcgaggactggggaccctgcaccgaacatggagagcacaacatcaggattcctaggacccctgctcgtgttacaggcggggtttttcttgttgacaagaatcctcacaataccacagagtctagactcgtggtggacttctctcaattttctagggggagcacccacgtgtcctggccaaaattcgcagtccccaacctccaatcactcaccaacctcttgtcctccaacttgtcctggctatcgctggatgtgtctgcggcgttttatcatattcctcttcatcctgctgctatgcctcatcttcttgttggttcttctggactaccaaggtatgttgcccgtttgtcctctacttccaggaacatcaactaccagcacgggaccatgcaagacctgcacgattcctgctcaaggaacctctatgtttccctcttgttgctgtacaaaaccttcggacggaaactgcacttgtattcccatcccatcatcctgggctttcgcaagattcctatgggagtgggcctcagtccgtttctcctggctcagtttactagtgccatttgttcagtggttcgtagggctttcccccactgtttggctttcagttatatggatgatgtggtattgggggccaagtctgtacaacatcttgagtccctttttacctctattaccaattttcttttgtctttgggtatacatttga

SEQ ID NO: 45 - protein, 400 aa, Hepatitis B virus
MGGWSSKPRQGMGTNLSVPNPLGFFPDHQLDPAFGANSNNPDWDFNPNKDHWPEANQVGAGAFGPGFTPPHGGLLGWSPQAQGILTTVPAAPPPASTNRQSGRQPTPISPPLRDSHPQAMQWNSTTFHQALLDPRVRGLYFPAGGSSSGTVNPVPTTASPISSIFSRTGDPAPNMESTTSGFLGPLLVLQAGFFLLTRILTIPQSLDSWWTSLNFLGGAPTCPGQNSQSPTSNHSPTSCPPTCPGYRWMCLRRFIIFLFILLLCLIFLLVLLDYQGMLPVCPLLPGTSTTSTGPCKTCTIPAQGTSMFPSCCCTKPSDGNCTCIPIPSSWAFARFLWEWASVRFSWLSLLVPFVQWFVGLSPTVWLSVIWMMWYWGPSLYNILSPFLPLLPIFFCLWVYI

SEQ ID NO: 46 - DNA, 336 bp, Bacillus amyloliquefaciens
  CDS (1)..(336)
atggtaccggttatcaacacgtttgacggggttgcggattatctgcagacatatcataagctacctgataattacattacaaaatcagaagcacaagccctcggctgggtggcatcaaaagggaaccttgcagacgtcgctccggggaaaagcatcgtcggagacatcttctcaaacagggaaggcaaactcccgggcaaaagcggacgaacatggcgtgaagcggatattaactatacatcaggcttcagaaattcagaccggattctttactcaagcgactggctgatttacaaaacaacggaccattatcagacctttacaaaaatcagataa

SEQ ID NO: 47 - protein, 111 aa, Bacillus amyloliquefaciens
MVPVINTFDGVADYLQTYHKLPDNYITKSEAQALGWVASKGNLADVAPGKSIVGDIFSNREGKLPGKSGRTWREADINYTSGFRNSDRILYSSDWLIYKTTDHYQTFTKIR

SEQ ID NO: 48 - DNA, 870 bp, wheat streak mosaic virus
  CDS (1)..(810)
caatctaataacgtatctgtcatggctggccttgacacgggaggagctaagacaggtcaaggatcaggatcaaaagggacgggtggttcattcacatcgaatcccgtgcgaactggaggccgagcaacggatgtgcaagatcagacaccaggtttagtatttccagcaccaaagatcacaacaaaggccatatacatgccaaacactgtacgcgacaagataaagcctgaaatgataaatgacatgatcaaataccaaccgcgtgcggaacttatcgacaacagatatgcaacaactgaacaactcaacacctggataaaagaggcatctgaagggcttgacgtgacagaggatgttttcataaacaccttactaccaggatgggtctaccactgcataatcaacacaacgagcccagagaacagagcactaggaacttggcgtgttgtgaataatgcaggcaaggacaatgagcagcaactcgagtttaacactgacccaatgtacaacgctgcgaagccatcacttcgagccattatgcgccactttggcgaaggagctccagtgatgatcgaggagagtgttcggattggaaaacctatcataccaaggggcttcgacaaggccggtgtgctaagcatcaacaatattgtggcagcgtgtgatttcatcatgcgcggtgcagatgacacaccaaattttgtgcaagtgcagaacagcgttgcagtgaacaggctacgcggaatacaaaacaagttgtttgcacaggcacgactgagtgcgggtactaatgaggacaactcacgtcatgatgcagatgatgtgagggagaacacgcacagtttcaatggtgtaaacgctcttgcgtga

SEQ ID NO: 49 - protein, 270 aa, wheat streak mosaic virus
QSNNVSVMAGLDTGGAKTGQGSGSKGTGGSFTSNPVRTGGRATDVQDQTPGLVFPAPKITTKAIYMPNTVRDKIKPEMINDMIKYQPRAELIDNRYATTEQLNTWIKEASEGLDVTEDVFINTLLPGWVYHCIINTTSPENRALGTWRVVNNAGKDNEQQLEFNTDPMYNAAKPSLRAIMRHFGEGAPVMIEESVRIGKPIIPRGFDKAGVLSINNIVAACDFIMRGADDTPNFVQVQNSVAVNRLRGIQNKLFAQARLSAGTNEDNSRH

SEQ ID NO: 50 - DNA, 925 bp, triticum aestivum
  CDS (67)..(741)
cgagggcacgcacacaagctaccgtcacaagtcgtactcccgggaacaacaagagcggcatcatccatggcgtccactcgcgtcctccacctcatcgccctcgtcctcgccgtcgccaccgccgcagatgcggccaccatcaccgtcgtcaaccgttgctcctacacggtgtggccgggcgcgctcccaggcggcggcgtgcgtctcgacccgggccagtcttgggcgctgaacatgcccgccggcaccgcgggcgccagggtgtggccgcgcaccgggtgcaccttcgacggcagcggccggggccggtgcatcacgggcgactgcggcggcacgctggcctgcagggtgtccggccagcagcccaccacgctggccgagtacaccctgggccagggcgggaacaaggacttcttcgacctgtccgtcatcgacgggttcaacgtgcccatgaacttcgagcccgtcggcggttcgtgccgcgctgcgcgctgcgccacggacatcaccaaggagtgcctcaaggagctgcaggtgcccggagggtgcgcgagcgcgtgcggcaaattcggcggcgacacctattgctgccggggccagttcgagcacaactgcccgccgaccaactactcgaagttcttcaaggggaagtgccccgacgcctacagctacgccaaggacgaccagaccagcaccttcacatgcccagccggaaccaactaccagatcgtcctctgcccttagattaggacgcgttgaaggatcaaaagtataataactgggatcaataaggaacaaggactactcttagtagtctacttggatgtatgtgccatgtcacatatatgcatatgaatgactgtgccttgtatgttgaccaataataaataaatggttgaatgttaaaaaaaaaaaaaaaaaaaaa

SEQ ID NO: 51 - protein, 225 aa, triticum aestivum
MASTRVLHLIALVLAVATAADAATITVVNRCSYTVWPGALPGGGVRLDPGQSWALNMPAGTAGARVWPRTGCTFDGSGRGRCITGDCGGTLACRVSGQQPTTLAEYTLGQGGNKDFFDLSVIDGFNVPMNFEPVGGSCRAARCATDITKECLKELQVPGGCASACGKFGGDTYCCRGQFEHNCPPTNYSKFFKGKCPDAYSYAKDDQTSTFTCPAGTNYQIVLCP

SEQ ID NO: 52 - DNA, 1170 bp, Triticum aestivum
  CDS (1)..(1167)
atgtcgcaccgtaagttcgagcacccgaggcacggatccctcggtttcctccccaggaagcggtgctcgcgccaccgcggaaaggtgaaggcctttcccagagatgaccaatccaagaaatgccaccttactgccttccttggctacaaggctgggatgacccacattgtgcgtgaggttgagaagcctggttccaagctacacaagaaggagacatgtgaggctgtcaccattgttgagacacccccgattgttattgttggactggttgcctatgtgaagactcctcgtggccttcgtactctcaactctgtctgggcacagcatctcagcgaagatgtcaggagaaggttctacaagaactggtgcaagagcaagaagaaggccttcaccaagtatgctctgaagtatgacagtgatgctggcaagaaagaaatccagatgcagcttgagaagatgaagaagtatgctactgttgtccgtgttatcgcccatacccagatcaggaagatgaagggtttgaagcagaagaaggctcacctcatggagatccagatcaatggcggcaccattgccgataaggtcgactatggttacaacttctttgagaaggaagtccccattgatgcagtcttccaaaaggatgagatgattgacatcatcggagttaccaagggtaagggttacgaaggtgttgtgacacgttggggtgtcacccgtcttccccgcaagacccacagaggtcttcgcaaggttgcctgtattggtgcctgccatcctgctagggtgtcctacactgttgctcgtgctggtcagaatggataccaccaccgaactgagatgaacaagaaggtctacaagattggcaaggttggacaggaaactcatgatgcctctaccgagttcgacaggaccgagaaggacatcactcccatgggtggcttccctcactatggtgtggtgaaggctgactacctgatgatcaagggatgctgtgttggccccaagaagcgtgtggtgaccctccgccagtccctgctgaagcagacctctcgtcttgccctggaggagatcaagctcaagttcgtcgacacctcttccaagttcgggcacggtcgcttccagaccacggacgagaagcagaggttctacggcaagctcaaggcttga

SEQ ID NO: 53 - protein, 389 aa, Triticum aestivum
MSHRKFEHPRHGSLGFLPRKRCSRHRGKVKAFPRDDQSKKCHLTAFLGYKAGMTHIVREVEKPGSKLHKKETCEAVTIVETPPIVIVGLVAYVKTPRGLRTLNSVWAQHLSEDVRRRFYKNWCKSKKKAFTKYALKYDSDAGKKEIQMQLEKMKKYATVVRVIAHTQIRKMKGLKQKKAHLMEIQINGGTIADKVDYGYNFFEKEVPIDAVFQKDEMIDIIGVTKGKGYEGVVTRWGVTRLPRKTHRGLRKVACIGACHPARVSYTVARAGQNGYHHRTEMNKKVYKIGKVGQETHDASTEFDRTEKDITPMGGFPHYGVVKADYLMIKGCCVGPKKRVVTLRQSLLKQTSRLALEEIKLKFVDTSSKFGHGRFQTTDEKQRFYGKLKA

SEQ ID NO: 54 - DNA, 1356 bp, Fusarium graminearum
  CDS (1)..(1353)
atggctttcaagatacagctcgacaccctcggccagctaccaggcctcctttcgatctacacccaaatcagtctcctctaccccgtctctgattcctctcaatatcccactattgtcagcaccttcgagcaaggtcttaagcgcttctccgaagccgtcccatgggtcgcaggccaggtcaaagccgagggcattagcgagggaaacacaggaacttcctttatcgtcccttttgaggacgttcctcgtgttgtagtgaaagacctccgcgatgatccttcagcgcccacgatcgagggtatgagaaaggcgggataccctatggcgatgtttgacgagaacatcatcgcgccaaggaagacgttacctattggacctggtactggtcccgacgacccaaagcctgtaattctattgcagctcaacttcatcaagggcggactcatcctcactgtcaacggacagcacggtgctatggatatggtaggccaagatgcggtgatccgtctactctccaaggcgtgccgtaacgacccattcaccgaagaggaaatgacggccatgaacctcgatcgcaagacgatagttccttaccttgaaaactatacgattggccccgaggtagatcatcagattgtcaaagctgatgtagctggtggtgacgctgttctcacgccggtcagtgcaagctgggcgttcttcacattcagccccaaggccatgtcagagctcaaggatgctgctaccaagactcttgacgcatcaacaaagttcgtgtcgactgacgatgctctttcggcgttcatctggaaatcggcctctcgcgtgcgtctcgaaagaatcgatggctctgcacctaccgagttctgccgtgctgttgatgctcgaccggcaatgggtgtctcgaacaactacccaggccttcttcaaaacatgacctaccacaactcgaccatcggcgaaatcgccaacgagtcactcggcgcaacagcatcacgccttcgttcagaactcgaccccgcgagcatgcgccagcgaacaagaggtctcgcgacgtacctgcacaacaaccccgacaagtccaacgtatccctgacggctgatgcggacccatctaccagcgtcatgctgagttcttgggccaaggtgggactctgggattacgactttgggctcggactgggtaagcccgagactgtgagacggccaatctttgagcctgttgagagcttgatgtactttatgcccaagaagcctgatggcgagttctgtgcggcgctttctctgagggatgaggatatggaccgattgaaggcggataaggagtggaccaagtatgcgcagtacgttggttag

SEQ ID NO: 55 - protein, 451 aa, Fusarium graminearum
MAFKIQLDTLGQLPGLLSIYTQISLLYPVSDSSQYPTIVSTFEQGLKRFSEAVPWVAGQVKAEGISEGNTGTSFIVPFEDVPRVVVKDLRDDPSAPTIEGMRKAGYPMAMFDENIIAPRKTLPIGPGTGPDDPKPVILLQLNFIKGGLILTVNGQHGAMDMVGQDAVIRLLSKACRNDPFTEEEMTAMNLDRKTIVPYLENYTIGPEVDHQIVKADVAGGDAVLTPVSASWAFFTFSPKAMSELKDAATKTLDASTKFVSTDDALSAFIWKSASRVRLERIDGSAPTEFCRAVDARPAMGVSNNYPGLLQNMTYHNSTIGEIANESLGATASRLRSELDPASMRQRTRGLATYLHNNPDKSNVSLTADADPSTSVMLSSWAKVGLWDYDFGLGLGKPETVRRPIFEPVESLMYFMPKKPDGEFCAALSLRDEDMDRLKADKEWTKYAQYVG

SEQ ID NO: 56 - DNA, 1025 bp, Hordeum vulgare
  CDS (120)..(761)
gtgccggtagtaaatcatgagcatctcttgcgactcgaaacgtagtacagcaacagcctaaagcgagtccgagtggtgattccagttcgtgtttgtttgagctagatcgtgagacgaagatggcctccaaccagaaccaggggagctaccacgccggcgagaccaaggcccgcaccgaggagaagaccgggcagatgatgggcgccaccaagcagaaggcggggcagaccaccgaggccaccaagcagaaggccggcgagacggccgaggccaccaagcagaagaccggcgagacggccgaggccgccaagcagaaggccgccgaggccaaggacaagacggcgcagacggcgcaggcggccaaggacaagacgtacgagacggcgcaggcggccaaggagcgcgccgcccagggcaaggaccagaccggcagcgccctcggcgagaagacggaggcggccaagcagaaggccgccgagacgacggaggcggccaagcagaaggccgccgaggcaaccgaggcggccaagcagaaggcgtccgacacggcgcagtacaccaaggagtccgcggtggccggcaaggacaagaccggcagcgtcctccagcaggccggcgagacggtggtgaacgccgtggtgggcgccaaggacgccgtggcaaacacgctgggcatgggaggggacaacaccagcgccaccaaggacgccaccaccggcgccaccgtcaaggacaccaccaccaccaccaggaatcactagacgcatgcgttcgcgcttaatttccgttcctttagtcgtgtttggtcgttcgagggccttctacatatttcatatttgtatgtttccactctttcatgatttccgctcatttagtgtaagtttgcctccgatttgatgtactcgtctctggttctgtaatgagttataatccatgggctttggtgtaaatggataacgaggacactcgaaggcggcaataaagttgtatgtgatcggtacaccaccaccaccaggaatcactag

SEQ ID NO: 57 - protein, 213 aa, Hordeum vulgare
MASNQNQGSYHAGETKARTEEKTGQMMGATKQKAGQTTEATKQKAGETAEATKQKTGETAEAAKQKAAEAKDKTAQTAQAAKDKTYETAQAAKERAAQGKDQTGSALGEKTEAAKQKAAETTEAAKQKAAEATEAAKQKASDTAQYTKESAVAGKDKTGSVLQQAGETVVNAVVGAKDAVANTLGMGGDNTSATKDATTGATVKDTTTTTRNH

SEQ ID NO: 58 - DNA, 651 bp, Arabidopsis thaliana
  CDS (1)..(651)
atgaactcattttctgctttttctgaaatgtttggctccgattacgagtcttcggtttcctcaggcggtgattatattccgacgcttgcgagcagctgccccaagaaaccggcgggtcgtaagaagtttcgtgagactcgtcacccaatatacagaggagttcgtcggagaaactccggtaagtgggtttgtgaggttagagaaccaaacaagaaaacaaggatttggctcggaacatttcaaaccgctgagatggcagctcgagctcacgacgttgccgctttagcccttcgtggccgatcagcctgtctcaatttcgctgactcggcttggagactccgaatcccggaatcaacttgcgctaaggacatccaaaaggcggcggctgaagctgcgttggcgtttcaggatgagatgtgtgatgcgacgacggatcatggcttcgacatggaggagacgttggtggaggctatttacacggcggaacagagcgaaaatgcgttttatatgcacgatgaggcgatgtttgagatgccgagtttgttggctaatatggcagaagggatgcttttgccgcttccgtccgtacagtggaatcataatcatgaagtcgacggcgatgatgacgacgtatcgttatggagttattaa

SEQ ID NO: 59 - protein, 216 aa, Arabidopsis thaliana
MNSFSAFSEMFGSDYESSVSSGGDYIPTLASSCPKKPAGRKKFRETRHPIYRGVRRRNSGKWVCEVREPNKKTRIWLGTFQTAEMAARAHDVAALALRGRSACLNFADSAWRLRIPESTCAKDIQKAAAEAALAFQDEMCDATTDHGFDMEETLVEAIYTAEQSENAFYMHDEAMFEMPSLLANMAEGMLLPLPSVQWNHNHEVDGDDDDVSLWSY

SEQ ID NO: 60 - DNA, 1818 bp, triticum aestivum
  CDS (1)..(1818)
atggcggctctggtcacgtcgcagctcgccacctccggcaccgtcctcggcatcaccgacaggttccggcgtgcaggttttcagggtgtgaggccccggagcccggcagatgcgccgctcggcatgaggactaccggagcgagcgccgccccgaagcaacaaagccggaaagcgcaccgcgggacccggcggtgcctctccatggtggtgcgcgccacgggcagcgccggcatgaacctcgtgttcgtcggcgccgagatggcgccctggagcaagaccggcggcctcggcgacgtcctcgggggcctccccccagccatggccgccaacggtcaccgggtcatggtcatctccccgcgctacgaccagtacaaggacgcctgggacaccagcgtcgtctccgagatcaaggtcgcggacgagtacgagagggtgaggtacttccactgctacaagcgcggggtggaccgcgtgttcgtcgaccacccgtgcttcctggagaaggtccggggcaagaccaaggagaagatctacgggcccgatgccggcacggactacgaggacaaccagctacgcttcagcctgctctgccaggcagcgcttgaggcacccaggatcctcgacctcaacaacaacccatacttctccggaccctacggggaagacgtggtgttcgtgtgcaacgactggcacacgggccttctggcctgctacctcaagagcaactaccagtccagtggcatctataggacggccaaggtagcgttctgcatccacaacatctcgtatcagggccgcttctccttcgacgacttcgcgcagctcaacctgcccgacaggttcaagtcgtccttcgacttcatcgacggctacgacaagccggtggaggggcgcaagatcaactggatgaaggccgggatcctgcaggccgacaaggtgctcacggtgagcccctactacgcggaggagctcatctccggcgaagccaggggctgcgagctcgacaacatcatgcgcctcacgggcatcaccggcatcgtcaacggcatggacgtcagcgagtgggaccccgccaaggacaagttcctcgccgccaactacgacgtcaccaccgcgttggaggggaaggcgctgaacaaggaggcgctgcaggccgaggtggggctgccggtggaccggaaggtgcccctggtggccttcatcggcaggctggaggagcagaagggccccgacgtgatgatcgccgccatcccggagatcttgaaggaggaggacgtccagatcgttctcctgggcaccgggaagaagaagtttgagcggctgctcaagagcgtggaggagaagttcccgagcaaggtgagggccgtggtcaggttcaacgcgccgctggctcaccagatgatggccggcgccgacgtgctcgccgtcaccagccgcttcgagccctgcggcctcatccagctccaggggatgcgctacggaacgccgtgcgcgtgcgcgtccaccggcgggctcgtcgacacgatcatggagggcaagaccgggttccacatgggccacctcagcgtcgactgcaacgtggtggagccggccgacgtgaagaaggtggtgaccaccctgaagcgcgccgtcaaggtcgtcggcacgccagcctaccatgagatggtcaagaactgcatgatccaggatctctcctggaaggggccagccaagaactgggaggacgtgcttctggaactgggggtcgaggggagcgagccaggggtcatcggcgaggagattgcgccgctcgccatggagaacgtcgccgctccctga

SEQ ID NO: 61 - protein, 605 aa, triticum aestivum
MAALVTSQLATSGTVLGITDRFRRAGFQGVRPRSPADAPLGMRTTGASAAPKQQSRKAHRGTRRCLSMVVRATGSAGMNLVFVGAEMAPWSKTGGLGDVLGGLPPAMAANGHRVMVISPRYDQYKDAWDTSVVSEIKVADEYERVRYFHCYKRGVDRVFVDHPCFLEKVRGKTKEKIYGPDAGTDYEDNQLRFSLLCQAALEAPRILDLNNNPYFSGPYGEDVVFVCNDWHTGLLACYLKSNYQSSGIYRTAKVAFCIHNISYQGRFSFDDFAQLNLPDRFKSSFDFIDGYDKPVEGRKINWMKAGILQADKVLTVSPYYAEELISGEARGCELDNIMRLTGITGIVNGMDVSEWDPAKDKFLAANYDVTTALEGKALNKEALQAEVGLPVDRKVPLVAFIGRLEEQKGPDVMIAAIPEILKEEDVQIVLLGTGKKKFERLLKSVEEKFPSKVRAVVRFNAPLAHQMMAGADVLAVTSRFEPCGLIQLQGMRYGTPCACASTGGLVDTIMEGKTGFHMGHLSVDCNVVEPADVKKVVTTLKRAVKVVGTPAYHEMVKNCMIQDLSWKGPAKNWEDVLLELGVEGSEPGVIGEEIAPLAMENVAAP

SEQ ID NO: 62 - DNA, 20 bp, artificial sequence
  oligonucleotide for PCR amplification of Knotted 1 4D allele of wheat
caacaggagagccagaaggt

SEQ ID NO: 63 - DNA, 20 bp, artificial sequence
  oligonucleotide for PCR amplification of Knotted 1 4D allele of wheat
aggtcaccggtaacggtaag

SEQ ID NO: 64 - DNA, 30 bp, artificial sequence
  Oligonucleotide for amplification of D-amino acid oxidase from R. gracilis
attagatcttactactcgaaggacgccatg

SEQ ID NO: 65 - DNA, 30 bp, artificial sequence
  Oligonucleotide for amplification of D-amino acid oxidase from R. gracilis
attagatctacagccacaattcccgcccta

SEQ ID NO: 66 - DNA, 18 bp, artificial sequence
  Oligonucleotide for PCR amplification of mutant discosoma red fluorescent protein designated attB1
atggcctcctccgaggac

SEQ ID NO: 67 - DNA, 20 bp, artificial sequence
  Oligonucleotide for PCR amplification of mutant discosoma red fluorescent protein designated attB2
gccaccatctgttcctttag

SEQ ID NO: 68 - DNA, 37 bp, artificial sequence
  Oligonucleotide for amplifying 2175bp of 5′ untranslated promotersequence, act1D (act1D) from rice designated attB4
atcgactagtcccatccctcagccgcctttcactatc

SEQ ID NO: 69 - DNA, 42 bp, artificial sequence
  Oligonucleotide for amplifying 2175bp of 5′ untranslated promotersequence, act1D (act1D) from rice designated attB1
atcggcggccgccccatcctcggcgctcagccatcttctacc

SEQ ID NO: 70 - DNA, 39 bp, artificial sequence
  Oligonucleotide for amplifying CaMV35s polyadenylation signal designated attB2
atcgccaccgcggtggagtccgcaaaaatcaccagtctc

SEQ ID NO: 71 - DNA, 40 bp, artificial sequence
  Oligonucleotide for amplifying CaMV35s polyadenylation signal designated attB3
atcgccaccgcggtggaggtcactggattttggttttagg

SEQ ID NO: 72 - DNA, 20 bp, artificial sequence
  Oligonucleotide for amplyfying probe for detecting GusA gene by Southern hybridization
catcctcgacgatagcaccc

SEQ ID NO: 73 - DNA, 20 bp, artificial sequence
  Oligonucleotide for amplyfying probe for detecting GusA gene by Southern hybridization
tcatgtttgccaaagccctt

SEQ ID NO: 74 - DNA, 27 bp, artificial sequence
  Oligonucleotide for amplyfying probe for detecting hph gene bySouthern hybridization
cgcataacagcggtcattgactggagc

SEQ ID NO: 75 - DNA, 26 bp, artificial sequence
  Oligonucleotide for amplyfying probe for detecting hph gene bySouthern hybridization
gctggggcgtcggtttccactatcgg

SEQ ID NO: 76 - DNA, 20 bp, artificial sequence
  Oligonucleotide for amplyfying probe for detecting GusA gene by Southern hybridization
atgaactgtgcgtcacagcc

SEQ ID NO: 77 - DNA, 19 bp, artificial sequence
  Oligonucleotide for amplyfying probe for detecting GusA gene by Southern hybridization
ttgtcacgcgctatcagcc

SEQ ID NO: 78 - DNA, 19 bp, artificial sequence
  Oligonucleotide for amplyfying probe for detecting bar gene bySouthern hybridization
gtctgcaccatcgtcaacc

SEQ ID NO: 79 - DNA, 20 bp, artificial sequence
  Oligonucleotide for amplyfying probe for detecting bar gene bySouthern hybridization
gaagtccagctgccagaaac

SEQ ID NO: 80 - DNA, 20 bp, artificial sequence
  Oligonucleotide for amplyfying probe for detecting DsRed2 gene by Southern hybridization
ctgtccccccagttccagta

SEQ ID NO: 81 - DNA, 22 bp, artificial sequence
  Oligonucleotide for amplyfying probe for detecting DsRed2 gene by Southern hybridization
cgatggtgtagtcctcgttgtg

SEQ ID NO: 82 - DNA, 20 bp, artificial sequence
  Oligonucleotide for amplyfying probe for detecting dsd A gene by Southern hybridization
gtgggctcaaccggaaatct

SEQ ID NO: 83 - DNA, 21 bp, artificial sequence
  Oligonucleotide for amplyfying probe for detecting dsd A gene by Southern hybridization
gcagttgttctgcgctgaaac

SEQ ID NO: 84 - DNA, 20 bp, artificial sequence
  Oligonucleotide for amplyfying probe for detecting dao 1 gene by Southern hybridization
acatcacgccaaattaccgc

SEQ ID NO: 85 - DNA, 20 bp, artificial sequence
  Oligonucleotide for amplyfying probe for detecting dao 1 gene by Southern hybridization
gccccaactctgctggtatc

(19) United States
(12) Patent Application Publication
(10) Pub. No.: US 2009/0191636 A1
(43) Pub. Date: Jul. 30, 2009

(54) Method of Producing Transgenic Graminaceous Cells and Plants

(76) Inventors: Carl McDonald Ramage, Victoria (AU); German Spangenberg, Victoria (AU); Dalia Vishnudasan, Victoria (AU)

Correspondence Address: COOPER & DUNHAM, LLP, 30 Rockefeller Plaza, 20th Floor, NEW YORK, NY, 10112, US

(21) Appl. No.: 12/087,634

(22) PCT Filed: Jan. 11, 2007
(86) PCT No.: PCT/AU2007/000021

§ 371 Date: Jan. 22, 2009

Related U.S. Application Data
(60) Provisional application No. 60/757,994, filed on Jan. 11, 2006.

(30) Foreign Application Priority Data
Feb. 20, 2006 (AU) 2006900826

(51) Int. Cl. C12N 15/82; C12N 5/10

(52) U.S. Cl. 435/469; 435/419

(57) ABSTRACT

The present invention provides a method for producing a transgenic graminaceous plant cell, said method comprising: (i) obtaining embryonic cells from a mature graminaceous grain; and (ii) contacting said embryonic cells with a bacterium capable of transforming a plant cell, said bacterium comprising transfer-nucleic acid to be introduced into the embryonic cells, said contacting being for a time and under conditions sufficient for said bacterium to introduce said transfer-nucleic acid into one or more of the embryonic cells, thereby producing a transgenic graminaceous plant cell. The present invention also provides a method for producing a transgenic graminaceous plant. The present invention also provides a transgenic graminaceous plant cell and/or a transgenic graminaceous plant produced by said method. The present invention also provides a method for expressing a nucleic acid in a transgenic graminaceous plant cell or a transgenic graminaceous plant.

### RELATED APPLICATION DATA

This application claims Convention Priority from U.S. Patent Application No. 60/757,994 filed on Jan. 11, 2006 and from Australian Patent Application No. 2006900826 filed on Feb. 20, 2006, the contents of which are incorporated by reference.

### FIELD OF THE INVENTION

The present invention relates to a method for producing a transgenic cell from a graminaceous plant and transgenic tissues, organs, plants and seeds derived therefrom. The invention also relates to the use of such transgenic cells, tissues, organs, plants and seeds in agriculture, plant breeding and for industrial applications.

### BACKGROUND OF THE INVENTION

### General

This specification contains nucleotide and amino acid sequence information prepared using PatentIn Version 3.3. Each nucleotide sequence is identified in the sequence listing by the numeric indicator <210> followed by the sequence identifier (e.g. <210>1, <210>2, <210>3, etc). The length and type of sequence (DNA, protein (PRT), etc), and source organism for each nucleotide sequence, are indicated by information provided in the numeric indicator fields <211>, <212> and <213>, respectively. Nucleotide sequences referred to in the specification are defined by the term “SEQ ID NO:”, followed by the sequence identifier (e.g. SEQ ID NO: 1 refers to the sequence in the sequence listing designated as <400>1).

The designation of nucleotide residues referred to herein are those recommended by the IUPAC-IUB Biochemical Nomenclature Commission, wherein A represents Adenine, C represents Cytosine, G represents Guanine, T represents thymine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

As used herein the term “derived from” shall be taken to indicate that a specified integer may be obtained from a particular source albeit not necessarily directly from that source.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each embodiment described herein is to be applied mutatis mutandis to each and every other embodiment unless specifically stated otherwise.

Furthermore, each embodiment described herein in respect of a graminaceous plant or a graminaceous or a part thereof (e.g., a grain or seed) or a progeny thereof, shall be taken to apply mutatis mutandis to wheat (e.g., a wheat plant or a wheat plant part or progeny of a wheat plant).

The invention described herein with respect to any embodiment in so far as it refers to one or more graminaceous plants, plant species or varieties of plant species is capable of being separately directed to and claimed for one specific graminaceous plant, plant species or variety, and divisible from any other graminaceous plant, plant species or variety/varieties, without specific recitation of embodiments directed to that one specific graminaceous plant, plant species or variety. This is subject to the proviso that said graminaceous plant, plant species or variety claimed is specifically referred to herein in accordance with any embodiment of the invention described.

The invention described herein with respect to any embodiment in so far as it refers to the use of a bacterium is capable of being separately directed to and claimed for one bacterium, and divisible from any other bacterium, without specific recitation of embodiments directed to that one specific bacterium. This is subject to the proviso that said bacterium claimed is specifically referred to herein in accordance with any embodiment of the invention described.

The invention described herein with respect to any embodiment in so far as it refers to any method for introducing nucleic acid into embryonic cell(s) is capable of being separately directed to and claimed for one specific method for introducing nucleic acid into embryonic cell(s), and divisible from any other method for introducing nucleic acid into embryonic cell(s), without specific recitation of embodiments directed to that one specific method for introducing nucleic acid into embryonic cell(s). This is subject to the proviso that said method for introducing nucleic acid into embryonic cell(s) claimed is specifically referred to herein in accordance with any embodiment of the invention described.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purpose of exemplification only. Functionally-equivalent products, compositions and methods are clearly within the scope of the invention, as described herein.

The present invention is performed without undue experimentation using, unless otherwise indicated, conventional techniques of molecular biology, microbiology, virology, recombinant DNA technology, peptide synthesis in solution, solid phase peptide synthesis, and immunology. Such procedures are described, for example, in the following texts that are incorporated by reference:
1. Sambrook, Fritsch & Maniatis, Molecular Cloning: A Laboratory Manual, Cold Spring Harbor Laboratories, New York, Second Edition (1989), whole of Vols I, II, and III;
2. DNA Cloning: A Practical Approach, Vols. I and II (D. N. Glover, ed., 1985), IRL Press, Oxford, whole of text;
3. Oligonucleotide Synthesis: A Practical Approach (M. J. Gait, ed., 1984) IRL Press, Oxford, whole of text, and particularly the papers therein by Gait, pp 1-22; Atkinson et al., pp 35-81; Sproat et al., pp 83-115; and Wu et al., pp. 135-151;
4. Nucleic Acid Hybridization: A Practical Approach (B. D. Hames & S. J. Higgins, eds., 1985) IRL Press, Oxford, whole of text;
5. Perbal, B., A Practical Guide to Molecular Cloning (1984);
6. Methods in Plant Biochemistry and Molecular Biology (W. V. Dashek, ed., 1997) CRC Press, whole of text; and
7. Methods of Molecular Biology: Plant Cell and Tissue Culture (J. Polland, ed., 1990) Humana Press, whole of text

### DESCRIPTION OF THE RELATED ART

Wheat is one of the most abundant sources of energy and nourishment for humans. To date, the majority of beneficial traits contributing to improved plant productivity and/or nutritional value of wheat have been introduced into wheat using traditional breeding techniques e.g., introgression from one line into another line accompanied by selection and backcrossing over several generations.

Because wheat is an important broad acre crop plant, and because traditional plant breeding approaches to crop improvement are time-consuming, the production of genetically-engineered wheat (i.e., transgenic wheat) expressing phenotypes of interest is an attractive outcome. However, current methods for producing transgenic dicotyledonous plants either do not work or work inefficiently or unreliably when applied to monocotyledonous plants and, in particular, different varieties of wheat.

The skilled artisan will understand that the term “transgenic” means a plant or plant cell or plant part (e.g., a plant tissue or a plant organ) that comprises genetic material additional to the naturally occurring nucleic acid within the plant, cell or part. For example, the genome of a transgenic plant or plant cell or plant part may comprise nucleic acid from a different organism such as an animal, insect, bacterium, fungus or different plant species or variety. Alternatively, the genome of a transgenic plant or plant cell or plant part may comprise one or more additional copies of nucleic acid that occur naturally in the same plant species or variety. Alternatively, the genome of a transgenic plant or plant cell or plant part may comprise nucleic acid that does not occur in nature e.g., RNAi. The genome of a transgenic plant or plant cell or plant part may also contain a deletion relative to the genome of an isogenic or near-isogenic naturally-occurring plant e.g., as a result of homologous recombination or recombinase-induced recombination.

The term “plant part” is understood to mean a tissue or organ of a plant, including any reproductive material e.g., seed.

Generally, the production of a transgenic plant or plant cell or plant part comprises:
(i) transformation, wherein nucleic acid is introduced into the nuclear genome of a plant protoplast or plant cell to produce a transformed cell; and
(ii) regeneration, wherein plant tissues, organs or whole plants carrying the introduced nucleic acid in the genome of their cells are regenerated from the transformed cell whether by a process of organogenesis or embryogenesis.

As used herein, the term “org anogenesis” shall be taken to mean a process by which shoots and roots are developed sequentially from meristematic centres.

As used herein, the term “embryogenesis” shall be taken to mean a process by which shoots and roots develop together in a concerted fashion (not sequentially), whether from somatic cells or gametes.

The present invention provides a method that specifically provides for improved transformation of wheat which, when coupled to existing methods for achieving regeneration, provide the means for reliably improving this valuable crop plant.

Methods for Introducing Nucleic Acid into Wheat

Uptake of nucleic acid into protoplasts, particle bombardment-mediated transformation and Agrobacterium-mediated transformation have been disclosed for transforming wheat. These methods generally involve the use of protoplasts, inflorescences, embryonic callus, or immature embryos as starting material for the transformation.

The skilled artisan will be aware that the term “protoplast” refers to a plant cell in which the cell wall has been removed artificially, e.g., by enzymic digestion using a combination of cellulase, hemicullulase and pectinase.

In the present context, an “inflorescence” refers to floral structures, generally immature or developing buds.

The term “callus” refers to a cluster or group of undifferentiated cells produced by incubation of a plant tissue or organ for a time and under conditions sufficient for cell division to occur in the absence of regeneration. In the art of plant tissue culture, callus is generally considered to be non-naturally-occurring tissue.

The term “embryonic callus” refers to callus derived from embryos in tissue culture, commonly at the linear grain filling stage of seed development.

The term “embryo” refers to that part of the seed that on germination gives rise to a seedling. The skilled artisan will be aware that an embryo from a wheat grain comprises an embryonic root (radicle) enclosed within a coleorhiza, and a shoot apex enclosed within a coleoptile, in addition to a scutellum.

The term “immature embryo” is understood in the art to mean an embryo derived from a wheat seed at about 10-18 days post-anthesis (d.p.a.) and more commonly from a wheat seed at about 14-15 d.p.a. (see, for example, Weeks et al., Plant Physiol., 102: 1077-1084, 1993; Delporte et al., Plant Cell, Tissue and Organ Culture 80: 139-149, 2005 and Published International Application No. WO 97/48814). At this stage of development, the wheat seed is characterized by one or more of the following: (i) rapid cell division of cells of the endosperm e.g., as determined by mitotic index; (ii) endoreduplication in the endosperm e.g., as determined by DAPI staining; (iii) increasing DNA content in the endosperm e.g., as determined by DAPI staining; (iv) increasing fresh weight of seed; (v) increasing water content of the endosperm; and (vi) increasing starch content in the endosperm. In brief, the seed is in the grain filling phase of development. Such plant material has been considered to be most useful for transformation purposes because cells of the embryo are rapidly dividing in this phase.

Uptake of DNA into Protoplasts

To produce a protoplast, it is necessary to remove the cell wall from a plant cell. Methods for producing protoplasts are known in the art and described, for example, by Potrykus and Shillito, Methods in Enymology 118, 449-578, 1986.

Naked nucleic acid (i.e., nucleic acid that is not contained within a carrier, vector, cell, bacteriophage or virus) is introduced into a plant protoplast by physical or chemical permeabilization of the plasma membrane of the protoplast (Lörz et al., Mol. Gen. Genet. 199: 178-182, 1985 and Fromm et al., Nature, 319: 791-793, 1986).

The preferred physical means for introducing nucleic acid into protoplasts is electroporation, which comprises the application of brief, high-voltage electric pulses to the protoplast, thereby forming nanometer-sized pores in the plasma membrane. Nucleic acid is taken up through these pores and into the cytoplasm. Alternatively, the nucleic acid may be taken up through the plasma membrane as a consequence of the redistribution of membrane components that accompanies closure of the pores. From the cytoplasm, the nucleic acid is transported to the nucleus where it is incorporated into the genome.

The preferred chemical means for introducing nucleic acid into protoplasts utilizes polyethylene glycol (PEG). PEG-mediated transformation generally comprises treating a protoplast with nucleic acid of interest in the presence of a PEG solution for a time and under conditions sufficient to permeabilize the plasma membranes of the protoplast. The nucleic acid is then taken up through pores produced in the plasma membrane and either maintained as an episomal plasmid or incorporated into the genome of the protoplast.

Unfortunately, these physical and chemical means reduce the viability of protoplasts and impede their mitotic capability, thereby resulting in very low transformation efficiencies. Moreover, successful and reliable regeneration from transformed protoplasts has been achieved for only a narrow range of genotypes in those plant species tested. The extended culture conditions required for protoplast-mediated transformation also induces mutations, including somaclonal variation, that often result in the regeneration of infertile plants.

### Particle Bombardment-Mediated Transformation (Biolistic Transformation)

Particle bombardment-mediated transformation also delivers naked nucleic acid into plant cells (Sanford et al., J. Part. Sci. Technol. 5: 27, 37, 1987). This technique involves the acceleration of dense nucleic acid-coated microparticles, e.g., gold or tungsten particles, to a sufficient velocity to penetrate the plant cell wall and nucleus. The introduced nucleic acid is then incorporated into the plant genome, thereby producing a transgenic plant cell. This cell is then used to regenerate a transgenic plant.

However, transformation efficiencies using particle bombardment have remained low for most cultivated wheat varieties, generally about 0.1% to about 2.5% (Patnaik and Khurana, BMC Plant Biology, 3: 5-15, 2003). This means that large numbers of immature embryos and/or explants are required to produce even a few transformed plants. This increases production costs.

Furthermore, particle bombardment-mediated transformation regularly results in the incorporation of multiple copies of the introduced nucleic acid into the genome of the plant cell. Such multiple copies are associated with undesirable down-regulation of expression of the introduced nucleic acid by suppression or co-suppression (Rakoczy-Trojanowska, Cell and Molecular Biology Letters, 7: 849-858, 2002). The presence of multiple copies of exogenously-introduced nucleic acid is also generally unacceptable to national regulatory authorities, the approval of which is important for commercialization. This is partly to ensure that the transgenic plants can be fully characterized with respect to the insertion site of the introduced nucleic acid and heritability thereof. Accordingly, the presence of multiple copies of an introduced nucleic acid is undesirable.

Particle bombardment techniques are also expensive as they require the use of specialized equipment.

Patnaik and Khurana (BMC Plant Biology, 3: 5-15, 2003) have also transformed embryonic callus from mature wheat embryos, using particle-mediated transformation. In this case, embryos were isolated from grain in which the scutellum had hardened, and cultured for about two weeks to generate callus. The calli were then physically separated from the hardened scutellum and cultured for an additional week. Calli, not embryos, were transformed and transgenic plants regenerated from the transformed calli. A disadvantage of this technique is the significant time required to produce calli from the embryos prior to transformation.

### Arobacterium-Mediated Transformation

Agrobacterium tumefaciens is the causative agent of crown gall disease, predominantly in dicotyledonous plants. During infection, a fragment of a tumor inducing or Ti plasmid borne by the bacterium is transferred to the plant genome where it is stably integrated into the genome of the host plant (Hooykas and Beijersbergen, Ann. Rev. Phytopathol., 32: 157-179, 1994). Nucleic acid transferred to the plant cell is then transcribed by the host RNA polymerase II (Kahl and Schell (1982), Molecular Biology of Plant Tumors, Academic Press, New York).

Studies of gene transfer from A. tumefaciens to plants have facilitated the development of genetically-modified strains of the bacterium that permit gene transfer without the development of disease. For example, Horsch (Science, 227: 1229-1231, 1985) demonstrated successful transfer of a foreign nucleic acid to tobacco using A. tumefaciens lacking the genes causing crown gall disease. Since that report, A. tumefaciens has been used to produce transgenic cells from a variety of dicotyledonous plants, from which transgenic plants have been produced. The Agrobacterium system for transforming plants provides several advantages over other transformation methods, such as, for example, rapid production of transgenic plants, use of any of a variety of plant cells for transformation, and a relatively easy method that is inexpensive to perform.

However, Agrobacterium-mediated transformation has not been readily applied to the monocotyledonous plants, and wheat has proven to be especially recalcitrant to transformation by this method e.g., Birch Annu. Rev. Plant Physiol., 48: 793-797, 1997. For example, whilst Mooney et al., Plant Cell, Tissue and Organ Culture, 25: 209-218, 1991 reported the Agrobacterium-mediated transformation of immature wheat embryos from seeds at about 12-16 d.p.a., the authors were unable to regenerate, any transgenic plants. Similarly, whilst Ishida et al., Nature Biotechnology 14: 745-750, 1996 and EP 0 672752 reported the Agrobacterium-mediated transformation of immature embryos from maize and rice, they did not demonstrate successful transformation of other cereal crops, especially wheat. A further disadvantage of both of these methods is the requirement for immature embryonic tissue. Such tissue is not readily available year-round and requires the use of specialized equipment and the availability of adequate resources, e.g., labor to ensure a continuous supply of starting material.

Amoah et al., Journal of Experimental Botany. 52: 1135-1142, 2001 disclosed Agrobacterium-mediated transformation of callus derived from wheat inflorescences, however were not able to obtain transgenic cells when inflorescence tissue was used without a pre-culture to form callus. In this report, transgenic cells were found in callus tissue and not in inflorescence tissue. Furthermore, Amoah et al. failed to regenerate any transformed plants from the transgenic calli.

It follows that there is a clear need in the art for rapid and inexpensive means for producing transgenic wheat cells capable of being regenerated into transgenic tissues, organs or whole plants having desired phenotypes, such as, for example, improved yield and/or pest resistance and/or drought tolerance.

### SUMMARY OF INVENTION

The present invention provides a reliable and efficient bacterial-mediated method for transforming cells of graminaceous plants (i.e., graminaceous plant cells), which is applicable to a wide range of different plants, including, for example, wheat. The inventors have discovered that embryos from mature grain can be used directly as starting material for the bacterial-mediated transformation of cells from graminaceous plants, thereby overcoming the need for tissue culture steps to produce embryogenic callus. In so doing, the inventors have demonstrated against conventional wisdom that callus formation per se is not required for successful transformation of graminaceous plant cells. By avoiding such steps, the inventors also reduce the chance of somaclonal variation in transgenic cells and plants associated with tissue culture required for callus formation. Moreover, by virtue of using mature seeds which are in abundant supply compared to immature embryos or callus, the present invention provides significant time and cost savings over the prior art methods. The inventors have also demonstrated the general applicability of this bacterial-mediated transformation method to a diverse range of wheat varieties and barley, rice and maize thereby showing that this is a robust system useful for transforming graminaceous plants independent of their genotype.

In this regard, the inventors have used wheat as a model system for graminaceous plants generally as wheat plants have until now proved to be resistant to bacterial-mediated transformation, in particular, Agrobacterium-mediated transformation.

The inventors have also demonstrated the general applicability of the method for transforming graminaceous plants by producing transgenic wheat cells, transgenic barley cells, transgenic rice cells and transgenic maize cells.

The transformed graminaceous plant cells produced in accordance with the inventive method described herein are capable of undergoing subsequent regeneration to regenerate into plant parts, plantlets and whole plants carrying the introduced nucleic acid i.e., transformed plant parts and transformed whole plants. As will be apparent to the skilled artisan, the method of the present invention is useful for generating breeding populations, germplasm, etc expressing one or more desirable phenotypes e.g., enhanced tolerance to drought and/or a fungal pathogen; such as by virtue of having modified expression of an endogenous gene or conferred expression of an introduced gene.

Accordingly, the present invention provides a method for producing a transgenic graminaceous plant cell, said method comprising:
(i) obtaining embryonic cells from a mature graminaceous grain; and
(ii) contacting said embryonic cells with a bacterium capable of transforming a plant cell, said bacterium comprising transfer-nucleic acid to be introduced into the embryonic cells, said contacting being for a time and under conditions sufficient for said bacterium to introduce said transfer-nucleic acid into one or more cells thereof,
   thereby producing a transgenic graminaceous plant cell.

As used herein, the term “graminaceous” shall be taken in its broadest context to mean any monocotyledonous true grass or part thereof, preferably from the family Graminaceae, Gramineae or Poaceae. Suitable species of plant will be apparent to the skilled artisan. Examples of suitable graminaceous plants include, for example, a plant from the genus Aegilops, Agropyron, Agrostis, Alopecuris, Andropogon, Arrhenatherum, Arundo, Avena, Bromus, Bouteloua, Buchloe, Calamagrostis, Cenchrus, Chloris, Cortaderia, Cynodon, Dactylis, Dactyloctenium, Digitaria, Echinocloa, Eleusine, Elymus, Eragrostis, Erianthus, Festuca, Glyceria, Holcus, Hordeum, Leymus, Lolium, Muhlenbergia, Oryza, Oryzopsis, Panicum, Paspalum, Pennisetum, Phalarus, Phleum, Pseudosasa, Racemobambos, Sasa, Schizostachium, Spinifex, Stipa, Teinostachyum, Thamnocalamus, Triodia, Triticum, Yushania or Zea. Additional suitable genera will be apparent to the skilled artisan. For example, the graminaceous plant is a ryegrass (i.e., of the genus Lolium) or barley (i.e., of the genus Hordeum) or rice (e.g., of the genus Oryza) or maize (e.g., of the genus Zea) or wheat.

Accordingly, the present invention provides a method for producing a transgenic ryegrass cell, said method comprising:
(i) obtaining embryonic cells from a mature ryegrass grain; and
(ii) contacting said embryonic cells with a bacterium capable of transforming a plant cell, said bacterium comprising transfer-nucleic acid to be introduced into the embryonic cells, said contacting being for a time and under conditions sufficient for said bacterium to introduce said transfer-nucleic acid into one or more cells thereof,
   thereby producing a transgenic ryegrass cell.

As used herein, the term “ryegrass” shall be taken to mean any plant of the genus Lolium or tufted grasses, belonging to the grass family Poaceae. Ryegrasses are generally diploid, with 2n=14, and are closely related to the fescues Festuca. Lolium species are generally divided into outbreeding species, e.g., L. multiflorum or L. perenne and inbreeding species, e.g., L. teinulentum or L. persicum.

The present invention also provides a method for producing a transgenic barley cell or barley cell, said method comprising:
(i) obtaining embryonic cells from a mature barley grain; and
(ii) contacting said embryonic cells with a bacterium capable of transforming a plant cell, said bacterium comprising transfer-nucleic acid to be introduced into the embryonic cells, said contacting being for a time and under conditions sufficient for said bacterium to introduce said transfer-nucleic acid into one or more cells thereof,
   thereby producing a transgenic barley cell.

As used herein, the term “barley” shall be taken to mean any plant of the genus Hordeum. Hordeum species are annual or perennial with ploidy level ranges from 2×, 4× to 6× with basic chromosome number x=7. The term Hordeum includes such species as, for example, H. bulbosum, H. murinum, H. brachyantherum, H. patagonicum H. euclaston, H. fleruosum or H. vulgare. Preferably, the Hordeum plant is H. vulgare.

The present invention also provides a method for producing a transgenic rice cell, said method comprising:
(i) obtaining embryonic cells from a mature rice grain; and
(ii) contacting said embryonic cells with a bacterium capable of transforming a plant cell, said bacterium comprising transfer-nucleic acid to be introduced into the embryonic cells, said contacting being for a time and under conditions sufficient for said bacterium to introduce said transfer-nucleic acid into one or more cells thereof,
   thereby producing a transgenic rice cell.

As used herein, the term “rice” shall be taken to mean grass of the genus Oryza or Zizania. The term rice includes such species as, for example, O. sativa, O. rufipogon, O. alta, O. australiensis, O. barthii, O. brachyanth, O. eichingeri, O. glaberrima, O. grandiglumis, O. granulata, O. latifolia, O. longigumis, O. longistaminata, O. minuta, O. nivara, O. officinalis, O. punctata, O. ridleyi, Z. palustris, Z. aquatica, Z. texana or Z. latifolia. Preferably, the rice is O. sativa.

The present invention also provides a method for producing a transgenic maize cell, said method comprising:
(i) obtaining embryonic cells from a mature maize grain; and
(ii) contacting said embryonic cells with a bacterium capable of transforming a plant cell, said bacterium comprising transfer-nucleic acid to be introduced into the embryonic cells, said contacting being for a time and under conditions sufficient for said bacterium to introduce said transfer-nucleic acid into one or more cells thereof,
   thereby producing a transgenic maize cell.

As used herein, the term “maize” shall be taken to mean grass of the genus Zea. Preferably, the term mays encompasses any plant of the species Zea mays. The term maize includes such species as, for example, Z. mays indurata, Z. mays indenta, Z. mays everta, Z. mays saccharata, Z. mays amylacea, Z. mays tunicata and/or Z. mays Ceratina Kulesh.

The present invention also provides a method for producing a transgenic wheat cell, said method comprising:
(i) obtaining embryonic cells from a mature wheat grain; and
(ii) contacting said embryonic cells with a bacterium capable of transforming a plant cell, said bacterium comprising transfer-nucleic acid to be introduced into the embryonic cells, said contacting being for a time and under conditions sufficient for said bacterium to introduce said transfer-nucleic acid into one or more cells thereof,
   thereby producing a transgenic wheat cell.

In one example, the present invention provides a method for producing a transgenic wheat cell, said method comprising:
(i) obtaining embryonic cells from a mature wheat grain; and
(ii) contacting said embryonic cells with an Agrobacterium comprising a nucleic acid construct that comprises transfer-nucleic acid to be introduced into the embryonic cells for a time and under conditions sufficient for said Agrobacterium to introduce said transfer-nucleic acid into one or more cells thereof,
   thereby producing a transgenic wheat cell.

As used herein, the term “wheat” is to be taken in its broadest context to mean an annual or biennial grass capable of producing erect flower spikes and light brown grains and belonging to the Aegilops-Triticum group including Triticum sp. and Aegilops sp. Suitable species and/or cultivars will be apparent to the skilled artisan based on the description herein.

The term “wheat” also includes any tetraploid, hexaploid and allopolyploid (e.g., allotetraploid and allohexaploid) Aegilops sp. or Triticum sp. which carries the A genome and/or the B genome and/or D genome of the allohexaploid Triticum aestivum or a variant thereof. This includes A genome diploids (e.g., T. monococcum and T. urartu), B genome diploids (e.g., Aegilops speltoides and T. searsii) and closely-related S genome diploids (e.g., Aegilops sharonensis), D genome diploids (e.g., T. tauschii and Aegilops squarrosa), tetraploids (e.g., T. turgidum and T. dicoccum (AABB), Aegilops tauschii (AADD)), and hexaploids (e.g., T. aestivum and T. compactum). The term “wheat” may encompass varieties, cultivars and lines of Aegilops sp. or Triticum sp. but is not to be limited to any specific variety, cultivar or line thereof unless specifically stated otherwise. In one example, the wheat is a winter wheat. In this respect, a winter wheat is a wheat that sprouts before winter (e.g., before soil freezing occurs), then becomes dormant until the soil warm in spring. In another example, the wheat is a summer wheat or spring wheat. In this respect, a summer wheat or spring wheat is a wheat that is sown in spring and that matures over the following summer. The skilled artisan will be aware of varieties of winter wheat (e.g., Tennant or Brennan or Warbler or Currawong or Whistler) and/or summer wheat (e.g., Satu or Turbo or Nandu or Opal or Gaby).

In the present context, the term mature grain shall be taken to mean a grain in which grain filling is complete or nearly complete. For example, the term “mature wheat grain” refers to a wheat grain or seed in which grain-filling is complete or nearly complete and preferably, further characterized by:
(i) the presence of endosperm cells that are not detectably dividing (e.g., the mitotic index is 0 or nearly 0); and/or
(ii) endosperm cells that have ceased endoreduplication; and/or
(iii) endosperm cells having low water content in the endosperm i.e., desiccation of the seed has commenced.

For example, the term “mature barley grain” refers to a barley grain or seed in which grain-filling is complete or nearly complete and preferably, further characterized by:
(i) the presence of endosperm cells that are not detectably dividing (e.g., the mitotic index is 0 or nearly 0); and/or
(ii) endosperm cells having low water content in the endosperm i.e., desiccation of the seed has commenced; and/or
(ii) a kernel moisture content of no more than about 40%.

The term “mature rice grain” refers to a rice grain or seed in which grain-filling is complete or nearly complete and preferably, further characterized by:
(i) the color of the panicle or of the grain is yellow; and/or
(ii) endosperm cells having low water content in the endosperm i.e., desiccation of the seed has commenced

The term “mature maize grain” refers to a maize grain or seed or kernel in which grain-filling is complete or nearly complete and preferably, further characterized by:
(i) the presence of endosperm cells that are not detectably dividing (e.g., the mitotic index is 0 or nearly 0); and/or
(ii) formation of a layer of black colored cells within the maize grain or seed or kernel; and/or
(iii) a kernel moisture content of no more than about 35%.

It is to be understood that to be useful in the inventive method, it is not essential for a mature grain not have actually completed grain filling and/or undergone senescence of the pericarp and/or possess a hard scutellum, or otherwise be capable of achieving germination. In fact, one example of the present invention clearly encompasses the use of a mature grain that has not completed grain filling. In the case of wheat, such grain will be generally characterized by a rounded appearance indicating that grain filling is nearly complete and preferably further characterized by a green pericarp.

It will be apparent to the skilled artisan that the term “mature wheat grain” in the present context is generally aged at least about 30 d.p.a., and preferably at least about 35 d.p.a., or at least about 40 d.p.a., when the grain filling phase of seed development is completed or nearly completed; The term “mature barley grain” in the present context is generally aged at least about 30 d.p.a., and preferably at least about 35 d.p.a., or at least about 40 d.p.a., when the grain filling phase of seed development is completed or nearly completed. The term “mature rice grain” in the present context is generally aged at least about 25 d.p.a., and preferably at least about 30 d.p.a., or at least about 35 d.p.a., when the grain filling phase of seed development is completed or nearly completed. The term “mature maize grain” in the present context is generally aged at least about 35 d.p.a., and preferably at least about 40 d.p.a., or at least about 45 d.p.a., when the grain filling phase of seed development is completed or nearly completed.

In one example, the method of the present invention utilizes a mature grain consisting of a dried grain or seed. In dried seed, the accumulation of storage protein and starch is complete, the pericarp has commenced fusion with the maternal epidermis, the cells of the seed coat are compressed and the aleurone has commenced producing proteins associated with osmoprotection and/or dessication tolerance.

The skilled artisan will be aware of characteristics of mature grain from other graminaceous plants, such as, Lolium.

Mature seed or grain will be readily identifiable and distinguishable from immature seed using the description provided herein.

In the present context, the term “embryonic cells from a mature grain” shall be taken to include any number of embryonic cells, or whole embryos, with or without surrounding non-embryonic tissues e.g., pericarp, endosperm, aleurone. Preferably, the term “embryonic cells from a mature grain” shall be taken to include any number of embryonic cells, or whole embryos, substantially free of pericarp and/or endosperm and/or aleurone. By “substantially free” in this context is meant less than about 5-10% contamination by weight, preferably than about 10-20% contamination by weight, more preferably than about 20-40% contamination by weight.

Preferred embryonic cells for use in accordance with the present invention are cells from the epiblast or the scutellum. Accordingly, the present invention clearly contemplates the use of embryonic tissue comprising epiblast and/or scutellum cells or tissues.

The term “embryonic cells from a mature grain” shall also be taken in this context to mean naturally-occurring embryonic cells i.e. not produced directly by means of tissue culture. Accordingly, such embryonic cells are present in an embryo in the absence of steps taken to induce callus formation or to de-differentiate an embryonic cell or to produce an undifferentiated cell from an embryonic cell. Accordingly, in one example, embryonic cells from a mature seed are contacted with a bacterium for a time and under conditions that are not sufficient to permit callus formation from said embryonic cells. This means, for example, that the embryonic tissue used in the present invention is not pre-incubated or maintained in media containing a synthetic auxin such as 2,4-dichlorophenoxyacetic acid for prolonged periods e.g., of at least about two weeks. This does not exclude maintenance of the mature seed or embryonic cells therefrom in tissue culture for a shortened period of time prior to contacting with the a bacterium, e.g., for less than about 3 days, preferably less than about 2 days, more preferably less than about 1 day, and still more preferably less than about 8 hours.

As used herein, the term “obtaining embryonic cells from a mature grain” shall be taken to include isolation or separation of embryonic cells from the cells of a mature grain as defined herein above. Preferred means for obtaining embryonic cells include, for example, excision of embryonic tissue. In one example, the method of the invention comprises excising an embryonic tissue (e.g., an epiblast and/or scutellum or fragment thereof) from a mature seed, e.g., using a scalpel.

A suitable bacterium capable of introducing nucleic acid into a plant cell will be apparent to the skilled artisan. Preferably, the bacterium is a soil-borne bacterium capable of introducing nucleic acid into a plant cell and/or transforming a plant cell. In this respect, the term “soil-borne” merely requires that the species or genus of bacterium was originally identified in or isolated from a soil source or occurs naturally in soil. This term does not require that the bacterium used in the transformation method of the invention actually be in soil.

Preferably, the bacterium is any one bacterium such as a bacterium of the genus Agrobacterium or Rhizobium or Sinorhizobium or Mesorhizobium. Preferably, the bacterium is Agrobacterium sp. Many species or strains of “Agrobacterium” are suitable for use in performing the present invention without undue experimentation provided that they are capable of delivering a transfer-nucleic acid to a plant cell. Preferred species include A. tumefaciens and A. rhizogenes. Preferred strains of Agrobacterium will be apparent to the skilled artisan based on the description herein.

By “contacting” is meant that the bacterium, e.g., the Agrobacterium is brought into physical contact or co-cultivated with the embryonic cells of the mature grain. Such means include dipping the tissue into a solution comprising bacterium, or dripping the bacterium onto the embryonic cells of the mature grain. All art-recognized means for inoculating plant tissue with bacterium, in particular, Agrobacterium, including subsequent co-cultivation of the plant tissue with the bacterium, are encompassed herein subject to the proviso that the embryonic cells have not been subjected to tissue culture steps to induce callus formation prior to their inoculation with the bacterium.

Preferred conditions that are sufficient for a bacterium to introduce transfer-nucleic acid into an embryonic cell comprise contacting the embryonic cells with the bacterium for a time and under conditions sufficient for said bacterium to bind to or attach to said embryonic cells. In one example, such conditions are also sufficient for said bacterium to introduce the transfer-nucleic acid to an embryonic cell (i.e., co-culture). Suitable methods of co-culture are known in the art and/or described herein.

As used herein, the term “nucleic acid construct” shall be taken to mean any nucleic acid comprising a transfer-nucleic acid capable of being delivered by a bacterium to an embryonic cell of a mature grain. For example, the nucleic acid construct may comprise a vector, such as, for example, Ti vector or a Ri vector comprising a transgene of interest.

As used herein, the term “transfer-nucleic acid” refers to the region or component of a nucleic acid construct that is introduced into a plant cell by a bacterium, preferably, an Agrobacterium. For example, a transfer nucleic acid may comprise transfer DNA (T-DNA) from a Ti vector or a Ri vector i.e., that part of the Ti vector or Ri vector that is transferred to the plant cell during transformation. Generally, a transfer-nucleic acid is positioned between a Left Border (LB) and a Right Border (RB) of a Ti vector or Ri vector, and optionally includes LB and/or RB sequences and the intervening DNA comprising a so-called “transgene”. The skilled artisan will be aware that multiple copies of a LB and/or a RB may be introduced to a plant cell during bacterial-mediated transformation. Accordingly, transfer-nucleic acid may comprise multiple copies of a LB and/or a RB.

For the purposes of nomenclature a nucleotide sequence of a Left Border is set forth in SEQ ID NO: 1 and a nucleotide sequence of a Right Border is set forth in SEQ ID NO: 2.

As used herein, the term “transgene” shall be taken to mean a region of a transfer-nucleic acid that is desired to be introduced into a graminaceous plant cell to thereby produce a transgenic graminaceous plant cell. The general applicability of the present invention is not to be limited by the nature of the transgene or by whether or not it is expressed or even produces or modifies a phenotype. Suitable transgenes will be apparent to the skilled artisan based on the description herein.

It is to be understood that a transgene need not be expressed in a transgenic cell or plant into which it is introduced. For example, a transgene may comprise a sequence of nucleotides capable of inducing transcriptional gene silencing (e.g., transcriptional homology-dependent gene-silencing), or consist of a molecular tag e.g., a specific DNA sequence, to assist in varietal identification.

In one example, expression of the transgene at the protein or RNA level may confer, induce or enhance a phenotype of the transgenic cell or plant. Exemplary transgenes are capable of expressing interfering RNA, an abzyme or a ribozyme that is capable of reducing or preventing expression of a gene in a plant cell. Alternatively, a transgene is capable of expressing a peptide, polypeptide or protein e.g., a reporter molecule or selectable marker or simply a tag to assist in varietal identification. As used herein, the term “express” or “expressed” or “expressing” shall be taken to mean at least the transcription of a nucleotide sequence to produce a RNA molecule. In some examples of the invention, the term “express” or “expressed” or “expressing” further means the translation of said RNA molecule to produce a peptide, polypeptide of protein.

In the case of an expressible transgene, it is preferred that the transgene is linked to a promoter that is operable in a graminaceous plant cell, and preferably, a wheat cell.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid (e.g., a transgene), e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid (e.g., a transgene and/or a selectable marker gene and/or a detectable marker gene) to which it is operably linked. Preferred promoters can contain additional copies of one or more specific regulatory elements to further enhance expression and/or alter the spatial expression and/or temporal expression of said nucleic acid.

As used herein, the term “in operable connection with” “in connection with” or “operably linked to” means positioning a promoter relative to a nucleic acid (e.g., a transgene) such that expression of the nucleic acid is controlled by the promoter. For example, a promoter is generally positioned 5′ (upstream) to the nucleic acid, the expression of which it controls. To construct heterologous promoter/nucleic acid combinations (e.g., promoter/transgene and/or promoter/selectable marker gene combinations), it is generally preferred to position the promoter at a distance from the gene transcription start site that is approximately the same as the distance between that promoter and the nucleic acid it controls in its natural setting, i.e., the gene from which the promoter is derived. As is known in the art, some variation in this distance can be accommodated without loss of promoter function.

As exemplified herein, the present inventors have enhanced the transformation efficiency of the present method by removing the aleurone and/or seed coat from the embryonic cells prior to transformation. Accordingly, in one example, the method of the invention additionally comprises removing the seed coat and/or aleurone from the embryonic cells prior to contacting said cells with a bacterium. The skilled artisan will be aware of suitable methods of scarification or seed coat removal, such as for example, acid etching or mechanical removal. If it is desired to specifically transform scutellar cells, this may require the use of seed that do not have a hard scutellum, to permit retention of such cells when the seed coat is removed. Such considerations are not significant when transforming the epiblast.

As exemplified herein, the inventors have additionally increased transformation efficiency by including a nitrogen source, e.g., isolated from soybean, in the inoculation and/or co-culture medium i.e. the culture medium in which the bacterium is inoculated and/or co-cultured with the embryonic cells. Accordingly, it is preferred for inoculation and/or co-culture to be performed in the presence of a compound that provides a nitrogen source that a bacterium, and preferably, an Agrobacterium can utilize. Preferred nitrogen sources in this context include e.g., a peptone, i.e., an enzymic digest or acid hydrolysate of plant or animal protein. For example, the inoculation and/or co-culture is performed in the presence of a peptone derived from soy, e.g., Soytone. Additional peptones will be apparent to the skilled artisan and include, for example, a peptone produced from protein derived from or isolated from a plant that an Agrobacterium is capable of infecting.

In one example, the method of the invention additionally comprises providing, producing or obtaining the bacterium comprising the nucleic acid construct. For example, the method of the invention comprises introducing the nucleic acid construct into the bacterium using a method known in the art, such as, for example, electroporation or tri-parental mating.

Alternatively, or in addition, the method of the invention additionally comprises providing, producing or obtaining the nucleic acid construct, e.g., using a method known in the art and/or described herein. For example, the method of the invention additionally comprises placing a transgene in operable connection with a promoter operable in a graminaceous plant cell. Such a transgene is then inserted, e.g., cloned into a suitable nucleic acid construct, e.g., a Ti vector or a Ri vector.

Preferably, the method of the invention additionally comprises detecting and/or selecting a transgenic graminaceous plant cell. To facilitate such selection and/or detection, a transfer-nucleic acid introduced into a graminaceous plant cell preferably comprises a selectable marker gene and/or a detectable marker gene operable in a cell of a graminaceous plant. Alternatively, the transfer-nucleic acid is transformed with (i.e., co-transformed) a further transfer-nucleic acid comprising a detectable and/or selectable marker gene. Suitable detectable and/or selectable markers will be apparent to the skilled artisan based on the description herein.

For example, the selectable marker may facilitate growth of a graminaceous plant cell or plant in the presence of a D-amino acid, such as, for example, D-alanine and/or D-serine (e.g., the selectable marker is a D-amino acid oxidase; DAAO). A graminaceous plant cell expressing such a marker is selected by growing said cell in the presence of D-alanine and/or D-serine, both of which are toxic to a plant cell not expressing a D-amino acid oxidase.

The present invention additionally provides a transgenic graminaceous plant cell produced directly by the method of the present invention as described herein according to any embodiment.

The present inventors have also exemplified the expression of a heterologous nucleic acid in a transgenic cell of a graminaceous plant following transformation using the method of the invention. Accordingly, the present invention additionally provides for the use of the method of the invention for producing a transgenic graminaceous plant cell that expresses a transgene. For example, the present invention additionally provides a method for expressing a transgene in a graminaceous plant cell, said method comprising:
(i) producing a transgenic graminaceous plant cell comprising a transgene in operable connection with a promoter operable in a graminaceous plant cell, said transgenic graminaceous plant cell produced by performing a method described herein according to any embodiment; and
(ii) maintaining said transgenic cell for a time and under conditions sufficient for said transgene to be expressed.

As will be apparent to the skilled artisan based on the foregoing description, the present invention also provides a method for producing a transgenic wheat cell or a transgenic barley cell or a transgenic rice cell or a transgenic maize cell, said method comprising:

(i) obtaining embryonic cells from a mature wheat grain or from a mature barley grain or from a mature rice grain or from a mature maize kernel;
(ii) contacting the embryonic cells with an Agrobacterium comprising a nucleic acid construct that comprises transfer-nucleic acid to be introduced into the embryonic cells for a time and under conditions sufficient for said Agrobacterium to bind to or attach to said embryonic cells; and
(iii) maintaining the embryonic cells and the bound Agrobacterium for a time and under conditions sufficient for said Agrobacterium to introduce the transfer-nucleic acid into one or more cells thereof,
thereby producing a transgenic wheat cell or a transgenic barley cell or a transgenic rice cell or a transgenic maize cell.

The present invention also provides a method for producing a transgenic wheat cell or a transgenic barley cell or a transgenic rice cell or a transgenic maize cell, said method comprising:

(i) obtaining embryonic cells from a mature wheat grain or from a mature barley grain or from a mature rice grain or from a mature maize kernel;
(ii) removing the seed coat and/or aleurone from the embryonic cells;
(iii) contacting the embryonic cells with an Agrobacterium comprising a nucleic acid construct that comprises transfer-nucleic acid to be introduced into the embryonic cells for a time and under conditions sufficient for said Agrobacterium to bind to or attach to said embryonic cells; and
(iv) maintaining the embryonic cells and the bound Agrobacterium for a time and under conditions sufficient for said Agrobacterium to introduce the transfer-nucleic acid into one or more cells thereof,
thereby producing a transgenic wheat cell or a transgenic barley cell or a transgenic rice cell or a transgenic maize cell.

Furthermore, the present invention provides a method for producing a transgenic wheat cell or a transgenic barley cell or a transgenic rice cell or a transgenic maize cell, said method comprising:

(i) obtaining embryonic cells from a mature wheat grain or from a mature barley grain or from a mature rice grain or from a mature maize kernel;
(ii) removing the seed coat and/or aleurone from the embryonic cells;
(iii) contacting the embryonic cells with an Agrobacterium comprising a nucleic acid construct that comprises transfer-nucleic acid to be introduced into the embryonic cells for a time and under conditions sufficient for said Agrobacterium to bind to or attach to said embryonic cells, wherein said contacting is performed in the presence of a peptone; and
(iv) maintaining the embryonic cells and the bound Agrobacterium for a time and under conditions sufficient for said Agrobacterium to introduce the transfer-nucleic acid into one or more cells thereof wherein said maintaining is performed in the presence of a peptone,
thereby producing a transgenic wheat cell or a transgenic barley cell or a transgenic rice cell or a transgenic maize cell.

The present invention additionally provides a transgenic wheat cell produced directly by the method of the present invention as described herein according to any embodiment.

The present inventors have also clearly exemplified the expression of a heterologous nucleic acid in a transgenic wheat cell following transformation using the method of the invention. Accordingly, the present invention additionally provides for the use of the method of the invention for producing a transgenic wheat cell that expresses a transgene. For example, the present invention additionally provides a method for expressing a transgene in a wheat cell, said method comprising:
(i) producing a transgenic wheat cell comprising a transgene in operable connection with a promoter operable in a wheat cell, said transgenic wheat cell produced by performing a method described herein according to any embodiment; and
(ii) maintaining said transgenic cell for a time and under conditions sufficient for said transgene to be expressed.

Suitable conditions for expressing a transgene in a graminaceous plant cell will depend on, for example, the promoter used and/or the graminaceous plant cell and/or the transgene and will be apparent to the skilled artisan, e.g., based on the description herein.

The skilled artisan will be aware of suitable transgenes. For example, a suitable transgene encodes a peptide, polypeptide or protein that induces or confers a desirable characteristic, such as, for example, improved drought tolerance and/or fungal resistance in a graminaceous plant, e.g., a wheat plant. Alternatively, or in addition, the transgene encodes a peptide, polypeptide or protein that improves plant productivity or confers resistance to an insecticide or herbicide.

The present invention additionally provides for the use of the method of the present invention to modulate expression of a nucleic acid in a graminaceous plant cell. For example, the present invention provides a method for modulating the expression of a nucleic acid in a graminaceous plant cell, said method comprising:
(i) producing a transgenic graminaceous plant cell comprising a transgene capable of modulating the expression of the nucleic acid, said transgenic cell produced by performing a method described herein according to any embodiment; and
(ii) maintaining said transgenic cell for a time and under conditions sufficient for the expression of the nucleic acid to be modulated.

For example, the transgene is capable of expressing a nucleic acid that inhibits expression of a nucleic acid in a graminaceous plant cell (e.g., an endogenous gene or a transgene in the cell). In accordance with some examples of the invention, the transgenic graminaceous plant cell expresses nucleic acid that induces co-suppression of an endogenous gene and/or expresses nucleic acid encoding a short interfering RNA (siRNA) and/or expresses hairpin RNA and/or expresses microRNA. In one example, the method comprises maintaining the transgenic graminaceous plant cell for a time and under conditions sufficient for expression of the transgene to thereby modulate expression of the nucleic acid.

However, the transgene need not necessarily be expressed in the graminaceous plant cell to thereby modulate expression of a nucleic acid in a graminaceous plant cell. For example, as discussed supra, the present invention encompasses the introduction of a transgene capable of inducing transcriptional gene silencing (e.g., transcriptional homology-dependent gene silencing) into a plant cell.

In accordance with these examples of the invention, the method optionally additionally comprises detecting expression of the transgene and/or selecting a cell comprising and/or expressing said transgene.

The present invention is also clearly useful for producing a transgenic graminaceous plant or plantlet or plant part (e.g., a transgenic wheat plant or plantlet or plant part). Accordingly, in one example, the present invention provides a method for producing a transgenic graminaceous plant or plantlet or plant part, said method comprising:
(i) producing a transgenic graminaceous plant cell by performing a method of the invention as described herein according to any embodiment;
(ii) regenerating a transgenic graminaceous plant or plantlet or plant part from the transgenic graminaceous plant cell produced at (i), thereby producing a transgenic plant or plantlet or plant part.

In one example, the method comprises contacting the transgenic graminaceous plant cell so formed with a compound that induces callus formation and/or induces dedifferentiation of the transgenic cell (or a cell derived therefrom) and/or induces the production of an undifferentiated cell from said transgenic cell for a time and under conditions sufficient to produce a callus and/or dedifferentiated cell and/or undifferentiated cell. A suitable compound will be apparent to the skilled artisan e.g., a compound is selected from the group consisting of 2,4-dichlorophenoxyacetic acid; 3,6-dichloro-o-anisic acid; 4-amino-3,5,6-thrichloropicolinic acid; and mixtures thereof.

Preferably, the callus and/or dedifferentiated cell and/or undifferentiated cell is contacted with a compound that induces shoot formation for a time and under conditions sufficient for a shoot to develop thereby producing a plantlet.

Preferably, the callus and/or dedifferentiated cell and/or undifferentiated cell is additionally and/or alternatively contacted with a compound that induces root formation for a time and under conditions sufficient to initiate root growth, thereby producing a plantlet. In this respect, the callus and/or dedifferentiated cell and/or undifferentiated cell may be contacted with a compound that induces shoot formation and a compound that produces root formation simultaneously, or consecutively.

Preferably, a compound that induces shoot formation and/or root formation is selected from the group consisting of indole-3-acetic acid, benzyladenine, indole-butyric acid, zeatin, α-naphthaleneacetic acid, 6-benzyl aminopurine, thidiazuron, kinetin, 2iP and mixtures thereof.

Preferably, the method for producing a transgenic plant additionally comprises maintaining the plantlet under conditions sufficient for the plantlet to develop into a whole plant (e.g., grow roots or shoots or grow to maturity).

It is preferred to select a cell comprising and/or expressing the transgene at the time of or during plant regeneration. Accordingly, in one example, the method for producing a transgenic graminaceous plant additionally comprises selecting a cell comprising the transfer-nucleic acid, and preferably, the transgene. For example, a cell comprising the transfer-nucleic acid is selected following transformation and/or at least about 1 week, or 3 weeks or 5 weeks following transformation. For example, a cell comprising the transfer-nucleic acid is selected at least about 1 week following transformation. For example, a cell comprising the transfer-nucleic acid is selected at least about 3 weeks following transformation. For example, a cell comprising the transfer-nucleic acid is selected at least about 5 weeks following transformation. Methods for selecting a cell comprising a transgene will be apparent to the skilled artisan based on the description herein.

As the transformation method of the present invention preferentially introduces transfer-nucleic acid into a cell of the epiblast or scutellum, such cells are preferably isolated to reduce the number of untransformed cells in a culture prior to or during selection. In this respect, these cells are isolated during transformation of the graminaceous plant cell (e.g., following inoculation) or following transformation (e.g., following co-cultivation) or prior to or during regeneration. Accordingly, the method for producing a transgenic plant of the present invention preferably additionally comprises isolating an epiblast cell and/or a scutellum cell following obtaining embryonic cells from the mature seed and/or following inoculation of said embryonic cells and/or following co-culture of said embryonic cells.

Preferably, a method of the present invention as described herein for producing a transgenic graminaceous plant additionally comprises selecting a transgenic graminaceous plant cell or callus or plantlet or plant in which a single transfer-nucleic acid or transgene has integrated into the genome of said cell, or cells of said callus, plantlet or plant. As discussed herein, a transgenic plant comprising cells having a single copy of a transgene (or a transfer-nucleic acid) is preferred by regulatory bodies for breeding and/or, growth for example, by farmers. Methods for selecting a transgenic plant cell or callus or plantlet or plant comprising cells having a single copy of the transfer-nucleic acid or transgene will be apparent to the skilled artisan. For example, a Southern hybridization is performed to determine the number of copies of said transfer nucleic acid or transgene in the genome of said cell, or cells of said callus, plantlet or plant.

As will be apparent to the skilled artisan from the description herein, the present invention provides a process for producing a transgenic wheat plant or a transgenic barley plant or a transgenic rice plant or a transgenic maize plant, said process comprising:

(i) producing a transgenic wheat cell or transgenic barley cell or transgenic rice cell or transgenic maize cell by performing a method comprising:
(a) obtaining embryonic cells from a mature wheat grain or from a mature barley grain or from a mature rice grain or from a mature maize kernel; and
(b) contacting said embryonic cells with an Agrobacterium comprising a nucleic acid construct that comprises transfer-nucleic acid to be introduced into the embryonic cells for a time and under conditions sufficient for said Agrobacterium to introduce said transfer-nucleic acid into one or more cells thereof, thereby producing a transgenic wheat cell or transgenic barley cell or transgenic rice cell or transgenic maize cell; and
   (ii) regenerating a wheat plant or barley plant or rice plant or maize plant from the transgenic wheat cell or transgenic barley cell or transgenic rice cell or transgenic maize cell produced at (i) by performing a method comprising:
(a) contacting the transgenic wheat cell or transgenic barley cell or transgenic rice cell or transgenic maize cell with a compound that induces callus formation for a time and under conditions sufficient to produce a callus;
(b) contacting the callus with a compound that induces shoot formation for a time and under conditions sufficient for a shoot to develop;
(c) contacting the callus with a compound that induces root formation for a time and under conditions sufficient to initiate root growth, thereby producing a plantlet; and
(d) growing the plantlet for a time and under conditions sufficient to produce a transgenic wheat plant or a transgenic barley plant or a transgenic rice plant or a transgenic maize plant.

The present invention also provides a process for producing a transgenic wheat plant or a transgenic barley plant or a transgenic rice plant or a transgenic maize plant, said process comprising:

(i) producing a transgenic wheat cell or transgenic barley cell or transgenic rice cell or transgenic maize cell by performing a method comprising:
(a) obtaining embryonic cells from a mature wheat grain or from a mature barley grain or from a mature rice grain or from a mature maize kernel; and
(b) removing the seed coat and/or aleurone from the embryonic cells;
(c) contacting the embryonic cells with an Agrobacterium comprising a nucleic acid construct that comprises transfer-nucleic acid to be introduced into the embryonic cells for a time and under conditions sufficient for said Agrobacterium to bind to or attach to said embryonic cells; and
(d) maintaining the embryonic cells and the bound Agrobacterium for a time and under conditions sufficient for said Agrobacterium to introduce said transfer-nucleic acid into one or more cells thereof, thereby producing a transgenic wheat cell or transgenic barley cell or transgenic rice cell or transgenic maize cell; and
   (ii) regenerating a wheat plant or barley plant or rice plant or maize plant from the transgenic wheat cell or transgenic barley cell or transgenic rice cell or transgenic maize cell produced at (i) by performing a method comprising:
(a) contacting the transgenic wheat cell or transgenic barley cell or transgenic rice cell or transgenic maize cell with a compound that induces callus formation for a time and under conditions sufficient to produce a callus;
(b) contacting the callus with a compound that induces shoot formation for a time and under conditions sufficient for a shoot to develop;
(c) contacting the callus with a compound that induces root formation for a time and under conditions sufficient to initiate root growth, thereby producing a plantlet; and
(d) growing the plantlet for a time and under conditions sufficient to produce a transgenic wheat plant or a transgenic barley plant or a transgenic rice plant or a transgenic maize plant.

The present invention also provides a process for producing a transgenic wheat plant or a transgenic barley plant or a transgenic rice plant or a transgenic maize plant, said process comprising:

(i) producing a transgenic wheat cell or transgenic barley cell or transgenic rice cell or transgenic maize cell by performing a method comprising:
(a) obtaining embryonic cells from a mature wheat grain or from a mature barley grain or from a mature rice grain or from a mature maize kernel;
(b) removing the seed coat and/or aleurone from the embryonic cells;
(c) contacting the embryonic cells with an Agrobacterium comprising a nucleic acid construct that comprises transfer-nucleic acid to be introduced into the embryonic cells for a time and under conditions sufficient for said Agrobacterium to bind to or attach to said embryonic cells, wherein said contacting is performed in the presence of a peptone; and
(d) maintaining the embryonic cells and the bound Agrobacterium for a time and under conditions sufficient for said Agrobacterium to introduce said transfer-nucleic acid into one or more cells thereof wherein said maintaining is performed in the presence of a peptone, thereby producing a transgenic wheat cell or transgenic barley cell or transgenic rice cell or transgenic maize cell; and
   (ii) regenerating a wheat plant or barley plant or rice plant or maize plant from the transgenic wheat cell produced at (i) by performing a method comprising:
(a) contacting the transgenic wheat cell or transgenic barley cell or transgenic rice cell or transgenic maize cell with a compound that induces callus formation for a time and under conditions sufficient to produce a callus;
(b) contacting the callus with a compound that induces shoot formation for a time and under conditions sufficient for a shoot to develop;
(c) contacting the callus with a compound that induces root formation for a time and under conditions sufficient to initiate Toot growth, thereby producing a plantlet; and
(d) growing the plantlet for a time and under conditions sufficient to produce a transgenic wheat plant or a transgenic barley plant or a transgenic rice plant or a transgenic maize plant.

The present invention is also useful for producing a transgenic graminaceous plant having a desirable characteristic. For example, the transgenic graminaceous plant comprises a transgene that encodes a peptide, polypeptide or protein that induces and/or enhances and/or confers said desirable characteristic. Alternatively, or in addition, the transgene modulates expression of a nucleic acid in a graminaceous plant associated with said characteristic. Methods for producing a transgenic graminaceous plant using the method of the invention as described in any embodiment are to be taken to apply mutatis mutandis to this embodiment of the invention.

For example, the transgene encodes a protein associated with improved productivity of a graminaceous plant, e.g., wheat, e.g., by conferring and/or inducing and/or enhancing resistance to a plant pathogen in a graminaceous plant in which the transgene is expressed (e.g., the protein is a wheat thaumatin-like protein or a wheat streak mosaic virus coat protein).

Alternatively, the transgene induces and/or enhances and/or confers drought tolerance and/or dessication tolerance and/or salt tolerance and/or cold tolerance in a graminaceous plant (e.g., wheat) in which the transgene is expressed. For example, the transgene is an Arabidopsis DREB1A gene.

Alternatively, or in addition, the transgene encodes a protein that improves or modifies a nutritional quality of a product from a transgenic graminaceous plant in which said transgene is expressed, e.g., the transgene improves or modifies a nutritional quality of flour produced from a transgenic wheat plant in which said transgene is expressed. For example, the transgene is a high molecular weight glutenin subunit 1Ax1 gene.

Alternatively, or in addition, the transgene expresses a nucleic acid that modifies a nutritional quality of a product from a graminaceous plant. For example, the transgene expresses a siRNA that reduces or prevents expression of a wheat granule-bound starch synthase I gene.

In a further alternative, the transgene confers a nutraceutical quality on a product from a graminaceous plant in which said transgene is expressed. As used herein, the term “nutraceutical” shall be taken to mean any substance that may be considered a food or part of a food and provides a medical or health benefit, including the prevention and treatment of disease.

For example, the transgene encodes a hepatitis B surface antigen.

In one example, the method of producing a transgenic graminaceous plant of the present invention additionally comprises growing the transgenic plant for a time and under conditions sufficient for seed to be produced. Preferably, the method additionally comprises obtaining said seed. Accordingly, the present invention additionally provides a method for producing a transgenic seed from a graminaceous plant, and, preferably from a wheat plant.

In another example, the method of producing a transgenic graminaceous plant of the present invention additionally comprises obtaining a plant part (e.g., reproductive material or propagating material or germplasm) from said plant.

In one example, a method for producing a transgenic graminaceous plant additionally comprises providing said plant and/or progeny thereof and/or seed thereof and/or propagating material thereof and/or reproductive material thereof and/or germplasm thereof.

The present invention additionally encompasses a method for producing progeny of a transgenic graminaceous plant. Accordingly, the present invention additionally provides a method for breeding a transgenic graminaceous plant, said method comprising:
(i) producing a transgenic graminaceous plant by performing a method described herein according to any embodiment; and
(ii) breeding the transgenic plant produced at (i) to thereby produce progeny of said plant.

In this respect, the transgenic plant may be bred with a transgenic or non-transgenic plant, i.e., the progeny produced may be homozygous or hemizygous for the transgene.

Preferably, the method comprises selecting or identifying a progeny of the transgenic plant comprising a transfer-nucleic acid as defined herein, and, preferably, comprising a transgene.

Clearly, the present invention additionally encompasses a transgenic plant, progeny of a transgenic plant, a seed of a transgenic plant or propagating material of a transgenic plant or reproductive material of a transgenic plant or germplasm of a transgenic plant produced using a method of the present invention as described herein according to any embodiment. Preferably, the plant is a wheat plant.

The present invention additionally encompasses a method for breeding a transgenic graminaceous plant, said method comprising:
(i) producing a transgenic graminaceous plant or progeny of the transgenic graminaceous plant or a seed of the transgenic graminaceous plant or propagating material of the transgenic graminaceous plant using a method described herein according to any embodiment; and
(ii) providing the plant, progeny, seed or propagating material for breeding purposes.

In another example, the present invention provides a method for breeding a transgenic graminaceous plant, said method comprising:
(i) obtaining a transgenic graminaceous plant or progeny of the transgenic graminaceous plant produced by performing a method described herein according to any embodiment; and
(ii) breeding the transgenic plant or progeny.

Alternatively, the method comprises:
(i) obtaining a seed of a transgenic graminaceous plant or propagating material of a transgenic graminaceous plant produced by performing a method described herein according to any embodiment;
(ii) growing or producing a transgenic plant using the seed or propagating material; and
(iii) breeding the transgenic plant produced at (ii).

Methods for breeding a graminaceous plant will be apparent to the skilled artisan and/or described herein.

The present invention also provides for the use of a method for producing a transgenic graminaceous plant cell or a transgenic graminaceous plant described herein in any embodiment in plant breeding. Preferably, the graminaceous plant is a wheat plant.

As will be apparent to the skilled artisan, a method for producing a transgenic graminaceous plant is also useful for expressing a transgene in a plant. Accordingly, the present invention provides a process for expressing a transgene in a graminaceous plant, said process comprising:

(i) producing a transgenic graminaceous plant or progeny thereof comprising a transgene operably linked to a promoter operable in a graminaceous plant cell, said plant or progeny produced by performing a method described herein according to any embodiment; and
(ii) maintaining said transgenic plant for a time and under conditions sufficient for said transgene to be expressed.

Suitable transgenes are described herein and are to be taken to apply mutatis mutandis to the present embodiment of the invention.

The present invention also provides a process for modulating the expression of a nucleic acid in a graminaceous plant, said process comprising:

(i) producing a transgenic graminaceous plant or progeny thereof comprising a transgene capable of modulating the expression of said nucleic acid, said plant or progeny produced by performing the method described herein according to any embodiment; and
(ii) maintaining said transgenic plant for a time and under conditions sufficient to modulate expression of said nucleic acid.

In one example, the transgene is placed in operable connection with a promoter and expresses a nucleic acid capable of modulating expression of a nucleic acid (e.g., a siRNA or a micro-RNA). In accordance with this embodiment, the method comprises maintaining the transgenic plant for a time and under conditions sufficient for the transgene to be expressed thereby modulating expression of the nucleic acid.

Suitable transgenes are described herein and are to be taken to apply mutatis mutandis to the present embodiment of the invention.

As will be apparent to the skilled artisan, a method for expressing a transgene in a graminaceous plant, or a method for modulating expression of a nucleic acid in a graminaceous plant, is also useful for conferring a phenotype on a graminaceous plant or modulating a characteristic in a graminaceous plant. Accordingly, the present invention also provides for the use of the method for expressing a tmmsgene in a graminaceous plant as described herein according to any embodiment to confer a characteristic on a graminaceous plant or modulate a characteristic in a graminaceous plant. For example, the present invention provides a process for conferring a characteristic on a graminaceous plant or modulating a characteristic in a graminaceous plant, said process comprising:

(i) producing a transgenic graminaceous plant or progeny thereof comprising a transgene capable of conferring or modulating said characteristic, said plant produced by performing the method described herein according to any embodiment; and
(ii) maintaining said transgenic plant for a time and under conditions sufficient to confer or modulate the characteristic.

For example, the transgene expresses a peptide, polypeptide or protein capable of conferring or improving or enhancing the characteristic. In accordance with this embodiment, the method comprises maintaining the transgenic plant for a time and under conditions sufficient for said transgene to be expressed thereby conferring or modulating the characteristic.

Alternatively, the transgene is capable of modulating expression of a nucleic acid in a graminaceous plant associated with the characteristic.

Preferred characteristics include, for example, productivity of a graminaceous plant e.g., a wheat plant, drought tolerance of a graminaceous plant, resistance to a pathogen, nutritional quality of a product from a graminaceous plant, e.g., bran or a nutraceutical quality of a graminaceous plant. Transgenes associated with these qualities are described herein and are to be taken to apply mutatis mutandis to the present embodiment of the invention.

The present invention also provides a process for improving the productivity of a graminaceous plant, said method comprising:

(i) producing a transgenic graminaceous plant or progeny thereof comprising a transgene encoding a protein associated with improved productivity, said transgene operably linked to a promoter operable in a graminaceous plant cell, said plant produced by performing the method described herein according to any embodiment;
(ii) maintaining said transgenic plant for a time and under conditions sufficient for said transgene to be expressed; and
(iii) growing said transgenic plant for a time and under conditions sufficient to produce grain, thereby enhancing the productivity of a graminaceous plant.

The present invention additionally provides a process for improving the nutritional quality of grain from a graminaceous plant said process comprising:

(i) producing a transgenic graminaceous plant or progeny thereof comprising a transgene encoding a nutritional protein, said transgene operably linked to a promoter operable in a graminaceous plant cell, said plant produced by performing the method described herein according to any embodiment;
(ii) maintaining said transgenic plant for a time and under conditions sufficient for said transgene to be expressed; and
(iii) obtaining a grain from said plant, said grain having an improved nutritional quality.

Furthermore, the present invention provides process for modulating the nutritional quality of grain from a graminaceous plant said process comprising:

(i) producing a transgenic graminaceous plant or progeny thereof comprising a transgene capable of modulating expression of a nucleic acid associated with a nutritional quality of a graminaceous plant, said plant produced by performing the method described herein according to any embodiment;
(ii) maintaining said transgenic plant for a time and under conditions sufficient for the expression of said nucleic acid to be modulated; and
(iii) obtaining a grain from said plant, said grain having an improved nutritional quality.

The present invention also provides a process for conferring a nutraceutical quality on a graminaceous plant, said method comprising:

(i) producing a transgenic graminaceous plant or progeny thereof comprising a transgene encoding a therapeutic or prophylactic or immunogenic protein, said transgene operably linked to a promoter operable in a graminaceous plant cell, said plant produced by performing the method described herein according to any embodiment;
(ii) maintaining said transgenic plant for a time and under conditions sufficient for said transgene to be expressed; and
(iii) obtaining a plant part in which the transgene is expressed, thereby enhancing the nutraceutical quality of the graminaceous plant.

Optionally, the method of the present embodiment additionally comprises feeding the obtained plant part to a subject (e.g., an animal or human subject).

As graminaceous plants, for example, wheat, are a major source of products for consumption (e.g., by humans), the present invention additionally encompasses a product comprising plant matter from a transgenic plant of the present invention or produced using a method of the present invention. Preferably, said product is labeled so as to indicate the nature of the product.

As used herein, the term “labeled so as to indicate the nature of the product” shall be taken to mean that the product is labeled so as to indicate that it comprises a transgenic graminaceous plant, e.g., wheat or plant matter derived therefrom, or that the product comprises plant matter from a transgenic graminaceous plant produced using bacterium-mediated transformation, e.g., Agrobacterium-mediated transformation or that the product comprises plant matter from a transgenic graminaceous plant produced using a method of the invention.

### BRIEF DESCRIPTION OF THE FIGURES

FIG. 1 is a schematic representation showing one example of a method for transforming a wheat embryo as described herein according to any embodiment. Briefly, the depicted method comprises surface sterilizing a mature wheat grain, isolating an embryo from the grain, inoculating the embryo with a suitable strain of Agrobacterium and co-cultivating the embryo with the Agrobacterium.

FIG. 2A is a copy of a photographic representation showing mature wheat grains from which embryos are isolated for use in a method for producing a transgenic wheat cell or transgenic wheat plant as described herein according to any embodiment.

FIG. 2B is a copy of a photographic representation showing a magnified image of a mature wheat grain from which an embryo is isolated for use in a method for producing a transgenic wheat cell or transgenic wheat plant as described herein according to any embodiment.

FIG. 2C is a copy of a photographic representation showing embryonic tissue (indicated by the arrow) excised from dried caryopsis of a wheat grain. The isolated embryo is then used for inoculation and co-cultivation, e.g., as depicted in FIG. 1.

FIG. 2D is a copy of a photographic representation showing an embryo transformed with the vector pCAMBIA1305.2 using a method as depicted in FIG. 1 and stained to detect gusA activity 3 days following inoculation. Dark staining cells express gusA (indicated by the arrow).

FIG. 2E is a copy of a photographic representation showing an embryo transformed with the vector pLM301 (pSB1_Ubi1::DsRed2-nos) using a method as depicted in FIG. 1.

FIG. 2F is a copy of a photographic representation showing DsRed2 expression in the embryo shown in FIG. 2E. DsRed2 expressing cells are shown as grey regions, examples of which are indicated by arrows.

FIG. 2G is a copy of a photographic representation showing an embryo transformed with the vector pLM301 (pSB1_Ubi1::DsRed2-nos) using a method as depicted in FIG. 1.

FIG. 2H is a copy of a photographic representation showing DsRed2 expression in the embryo shown in FIG. 2E. DsRed2 expressing cells are shown as grey regions, an example of which is indicated by an arrow.

FIG. 3A is a schematic representation showing an example of a method for regenerating a wheat plant from a transformed a wheat embryo as described herein according to any embodiment. Briefly, the depicted method comprises inducing callus induction in a callus induction medium as described; inducing regeneration in a regeneration medium described and inducing root induction in a root induction medium described.

FIG. 3B is a copy of a photographic representation showing wheat plants undergoing regeneration.

FIG. 3C is a copy of a photographic representation showing T₀ wheat plants undergoing root induction.

FIG. 3D is a copy of a photographic representation showing a T₁ wheat plant growing in nursery mix.

FIG. 3E is a copy of a photographic representation showing T₁ wheat plants growing in nursery mix.

FIG. 4A is a graphical representation showing the results of a quantitative polymerase chain reaction (PCR) to detect the presence of a hygromycin selectable marker in T₁ plants. Plants from the T1 line SE36 were assayed and results from those assays are labeled on the right-hand side of the figure. Results from positive and negative controls are also indicated on the right-hand side of the figure. The number of cycles performed is indicated on the X-axis and fluorescence units indicated on the Y-axis.

FIG. 4B is a graphical representation showing the results of a quantitative polymerase chain reaction (PCR) to detect the presence of the vir C gene from Agrobacterium strain EHA105 in T₁ plants. Plants from the T1 line SE36 were assayed and results from those assays are labeled on the right-hand side of the figure. Results from positive and negative controls are also indicated on the right-hand side of the figure. The number of cycles performed is indicated on the X-axis and fluorescence units indicated on the Y-axis.

FIG. 4C is a graphical representation showing the results of a quantitative polymerase chain reaction (PCR) to detect the presence of a hygromycin selectable marker in T₁ plants. Plants from the T1 line DV92-88 were assayed and results from those assays are labeled on the right-hand side of the figure. Results from positive and negative controls are also indicated on the right-hand side of the figure. The number of cycles performed is indicated on the X-axis and fluorescence units indicated on the Y-axis.

FIG. 4D is a graphical representation showing the results of a quantitative polymerase chain reaction (PCR) to detect the presence of a hygromycin selectable marker in T₁ plants. Plants from the T1 line DV100-92 were assayed and results from those assays are labeled on the right-hand side of the figure. Results from positive and negative controls are also indicated on the right-hand side of the figure. The number of cycles performed is indicated on the X-axis and fluorescence units indicated on the Y-axis.

FIG. 5 is a graphical representation showing the percentage of explants from a variety of wheat genotypes transformed using the method described in Example 1 in which gusA expression foci were detected. The name of each genotype (i.e., wheat variety) is indicated on the X-axis. The percentage of explants having gusA expression foci is indicated on the Y-axis.

FIG. 6A is a copy of a photographic representation showing a wheat embryo (Carinya variety) transformed with the vector pCAMBIA1305.2 and stained to detect gusA activity. Dark staining cells express gusA (indicated by the arrow).

FIG. 6B is a copy of a photographic representation showing a wheat embryo (Chara variety) transformed with the vector pCAMBIA1305.2 and stained to detect gusA activity. Dark staining cells express gusA (indicated by the arrow).

FIG. 6C is a copy of a photographic representation showing a wheat embryo (Diamondbird variety) transformed with the vector pCAMBIA1305.2 and stained to detect gusA activity. Dark staining cells express gusA (indicated by the arrows).

FIG. 6D is a copy of a photographic representation showing a wheat embryo (Sapphire variety) transformed with the vector pCAMBIA1305.2 and stained to detect gusA activity. Dark staining cells express gusA (indicated by the arrows).

FIG. 6E is a copy of a photographic representation showing a wheat embryo (W12332 variety) transformed with the vector pCAMBIA1305.2 and stained to detect gusA activity. Dark staining cells express gusA (indicated by the arrows).

FIG. 6F is a copy of a photographic representation showing a wheat embryo (RAC1262 variety) transformed with the vector pCAMBIA1305.2 and stained to detect gusA activity. Dark staining cells express gusA (indicated by the arrow).

FIG. 6G is a copy of a photographic representation showing a wheat embryo (Krichauff variety) transformed with the vector pCAMBIA1305.2 and stained to detect gusA activity. Dark staining cells express gusA (indicated by the arrows).

FIG. 6H is a copy of a photographic representation showing a wheat embryo (Ventura variety) transformed with the vector pCAMBIA1305.2 and stained to detect gusA activity. Dark staining cells express gusA (indicated by the arrows).

FIG. 7 is a graphical representation showing the frequency of plant regeneration of a variety of wheat genotypes using a method as depicted in FIG. 3A. The regeneration frequency is calculated based on the proportion of explants with regenerating whole plants. The wheat genotype (i.e., variety) is shown on the X-axis and the percentage regeneration frequency is shown on the Y-axis.

FIG. 8A is a copy of a photographic representation showing wheat explants of the Bobwhite variety undergoing regeneration according to a method depicted in FIG. 3A,

FIG. 8B is a copy of a photographic representation showing a wheat explant of the Fame variety undergoing regeneration according to a method depicted in FIG. 3A.

FIG. 8C is a copy of a photographic representation showing a wheat explant of the Carinya variety undergoing regeneration according to a method depicted in FIG. 3A.

FIG. 8D is a copy of a photographic representation showing wheat explants of the Kirchauff variety undergoing regeneration according to a method depicted in FIG. 3A.

FIG. 8E is a copy of a photographic representation showing a wheat explants of the Ventura variety undergoing regeneration according to a method depicted in FIG. 3A.

FIG. 9 is a graphical representation showing the effect of Soytone™ on transformation efficiency. Wheat embryos were inoculated and co-cultured with Agrobacterium carrying the pCAMBIA1305.2 vector in various concentrations of Soytone™ and the number of foci staining positive for gusA expression 3 days after inoculation determined. The concentration of Soytone™ is indicated at the base of the graph.

FIG. 10 is a graphical representation showing the effect of Soytone™ and/or seed coat removal on transformation efficiency. Wheat embryos were inoculated and co-cultured with Agrobacterium carrying the pCAMBIA1305.2 vector under various conditions (with or without seed coat and/or in the presence of Soytone™ or in the presence of a sugar) and the number of foci staining positive for gusA expression 3 days after inoculation determined. The treatment used in indicated at the base of the graph.

FIG. 11A is a copy of a photographic representation showing mature barley grains from which embryos are isolated for use in a method for producing a transgenic barley cell or transgenic barley plant as described herein according to any embodiment.

FIG. 11B is a copy of a photographic representation showing a magnified image of a mature barley grain from which an embryo is isolated for use in a method for producing a transgenic barley cell or transgenic barley plant as described herein according to any embodiment.

FIG. 11C is a copy of a photographic representation showing embryonic tissue (indicated by the arrow) excised from dried caryopsis of a barley grain. The isolated embryo is then used for inoculation and co-cultivation with a suitable bacterium to thereby produce a transgenic barley cell.

FIG. 11D is a copy of a photographic representation showing embryonic tissue (indicated by the arrow) excised from dried caryopsis of a barley grain. The isolated embryo is then used for inoculation and co-cultivation with a suitable bacterium to thereby produce a transgenic barley cell.

FIG. 11E is a copy of a photographic representation showing barley embryonic tissue that has been directly inoculated with an Agrobacterium suspension and co-cultivated.

FIG. 11F is a copy of a photographic representation showing a barley embryo transformed with the vector pCAMBIA1305.2 and stained to detect gusA activity. Dark staining cells express gusA (indicated by the arrows).

FIG. 12 is a copy of a photographic representation showing regeneration of barley plants from mature barley embryos transformed using an Agrobacterium-mediated transformation method.

FIG. 13A is a copy of a photographic representation showing mature rice grains from which embryos are isolated for use in a method for producing a transgenic rice cell or transgenic rice plant as described herein according to any embodiment.

FIG. 13B is a copy of a photographic representation showing a magnified image of a mature rice grain from which an embryo is isolated for use in a method for producing a transgenic rice cell or transgenic rice plant as described herein according to any embodiment.

FIG. 13C is a copy of a photographic representation showing rice embryonic tissue (indicated by the arrow) excised from dried caryopsis of a rice grain. The isolated embryo is then used for inoculation and co-cultivation with a suitable bacterium to thereby produce a transgenic rice cell.

FIG. 13D is a copy of a photographic representation showing rice embryonic tissue (indicated by the arrow) excised from dried caryopsis of a rice grain. The isolated embryo is then used for inoculation and co-cultivation with a suitable bacterium to thereby produce a transgenic rice cell.

FIG. 13E is a copy of a photographic representation showing barley embryonic tissue that has been directly inoculated with an Agrobacterium suspension and co-cultivated.

FIG. 13F is a copy of a photographic representation showing a barley embryo transformed with the vector pCAMBLA1305.2 and stained to detect gusA activity. Dark staining cells express gusA (indicated by the arrow).

FIG. 14A is a copy of a photographic representation showing mature maize kernel (grain) from which embryos are isolated for use in a method for producing a transgenic maize cell or transgenic maize plant as described herein according to any embodiment.

FIG. 14B is a copy of a photographic representation showing a magnified image of a mature maize Kernel (grain) from which an embryo is isolated for use in a method for producing a transgenic maize cell or transgenic maize plant as described herein according to any embodiment.

FIG. 14C is a copy of a photographic representation showing maize embryonic tissue (indicated by the arrow) excised from a dried maize kernel. The isolated embryo is then bisected and used for inoculation and co-cultivation with a suitable bacterium to thereby produce a transgenic maize cell.

FIG. 14D is a copy of a photographic representation showing a bisected maize embryo. The bisected embryo is then used for inoculation and co-cultivation with a suitable bacterium to thereby produce a transgenic maize cell.

FIG. 14E is a copy of a photographic representation showing a regenerating maize explant following transformation with the vector LM227.

FIG. 14F is a copy of a photographic representation showing the level of DsRed2 expression in the explant shown in FIG. 14E. DsRed2 expressing tissue is shown in the lighter cells, examples of which are indicated by arrows.

FIG. 15 is a graphical representation of the pBPS0054 vector. This vector comprises Left Border (LB) and Right Border (RB) regions flanking a maize ubiquitin promoter that drives expression of the bialaphos resistance gene (bar). The bar gene is in operable connection with the nos polyadenylation signal. The pBPS0054 vector also comprises the spectinomycin resistance gene for selection in bacteria. Restriction endonuclease cleavage sites are indicated.

FIG. 16 is a graphical representation of the pBPS0055 binary vector. This vector comprises Left Border (LB) and Right Border (RB) regions flanking a rice actin 1 1D promoter that drives expression of the gusA reporter gene. The gusA gene is in operable connection with the cauliflower mosaic virus 35S polyadenylation signal. The pBPS0055 vector also comprises the spectinomycin resistance gene for selection in bacteria. Restriction endonuclease cleavage sites are indicated.

FIG. 17 is a graphical representation of the pBPS0056 binary vector. This vector comprises Left Border (LB) and Right Border (RB) regions flanking a rice actin 1 1D promoter that drives expression of the improved green fluorescent protein (sGFP) reporter gene. The sGFP gene is in operable connection with the cauliflower mosaic virus 35S polyadenylation signal. The pBPS0056 vector also comprises the spectinomycin resistance gene for selection in bacteria. Restriction endonuclease cleavage sites are indicated.

FIG. 18 is a graphical representation of the pBPS0057 binary vector. This vector comprises Left Border-(LB) and Right Border (RB) regions flanking a rice actin 1 1D promoter that drives expression of the improved gusA reporter gene. The gusA gene is in operable connection with the cauliflower mosaic virus 35S polyadenylation signal. Also between the LB and RB is the plant selectable bar gene placed in operable connection with the maize ubiquitin promoter and nos terminator. The pBPS0057 vector also comprises the spectinomycin resistance gene for selection in bacteria. Restriction endonuclease cleavage sites are indicated.

FIG. 19 is a graphical representation of the pBPS0058 binary vector. This vector comprises Left Border (LB) and Right Border (RB) regions flanking a rice actin 1 1D promoter that drives expression of the improved sGFP reporter gene. The sGFP gene is in operable connection with the cauliflower mosaic virus 35S polyadenylation signal. Also between the LB and RB is the plant selectable bar gene placed in operable connection with the maize ubiquitin promoter and nos terminator. The pBPS0058 vector also comprises the spectinomycin resistance gene for selection in bacteria. Restriction endonuclease cleavage sites are indicated.

FIG. 20 is a graphical representation of the pPZPMV T2 R4R3 binary base vector. This vector comprises two separate T-DNAs and has been constructed to facilitate marker excision. One T-DNA contains a multiple cloning site suitable for modular expression cassettes and the other contains an R4R3 multi-site recombination cassette. The multiple cloning site consists of 13 hexanucleotide restriction sites, 6 octanucleotide restriction sites and 5 rare homing endonuclease sites to facilitate modularization.

FIG. 21 is a graphical representation showing the pBPS0059 vector. This vector comprises Left Border (LB) and Right Border (RB) regions flanking a maize ubiquitin promoter that drives expression of the bar resistance gene. The bar gene is in operable connection with the nos terminator. The pBPS0059 vector also comprises the spectinomycin resistance gene for selection in bacteria and a region for homologous recombination into the super binary acceptor vector pSB1. Restriction endonuclease cleavage sites are indicated.

FIG. 22 is a graphical representation showing the pBPS0060 vector. This vector comprises Left Border (LB) and Right Border (RB) regions flanking a rice actin 1D promoter that drives expression of the gusA reporter gene. The gusA gene is in operable connection with the cauliflower mosaic virus 35S terminator. The pBPS0060 vector also comprises the spectinomycin resistance gene for selection in bacteria and a region for homologous recombination into the super binary acceptor vector pSB1. Restriction endonuclease cleavage sites are indicated.

FIG. 23 is a graphical representation showing the pBPS0061 vector. This vector comprises Left Border (LB) and Right Border (RB) regions flanking a rice actin 1D promoter that drives expression of the sGFP reporter gene. The sGFP gene is in operable connection with the cauliflower mosaic virus 35S terminator. The pBPS0061 vector also comprises the spectinomycin resistance gene for selection in bacteria and a region for homologous recombination into the super binary acceptor vector pSB1. Restriction endonuclease cleavage sites are indicated.

FIG. 24 is a graphical representation showing the pBPS0062 vector. This vector comprises Left Border (LB) and Right Border (RB) regions flanking a rice actin 1D promoter that drives expression of the improved gusA reporter gene. The gusA gene is in operable connection with the cauliflower mosaic virus 35S polyadenylation signal. Also between the LB and RB is the plant selectable bar gene placed in operable connection with the maize ubiquitin promoter and nos terminator. The pBPS0062 vector also comprises the spectinomycin resistance gene for selection in bacteria and a region for homologous recombination into the super binary acceptor vector pSB1. Restriction endonuclease cleavage sites are indicated.

FIG. 25 is a graphical representation showing the pBS0063 vector. This vector comprises Left Border, (LB) and Right Border (RB) regions flanking a rice actin 1D promoter that drives expression of the improved sGFP reporter gene. The sGFP gene is in operable connection with the cauliflower mosaic virus 35S polyadenylation signal. Also between the LB and RB is the plant selectable bar gene placed in operable connection with the maize ubiquitin promoter and nos terminator. The pBS0063 vector also comprises the spectinomycin resistance gene for selection in bacteria and a region for homologous recombination into the super binary acceptor vector pSB1. Restriction endonuclease cleavage sites are indicated.

FIG. 26 is a graphical representation of the superbinary vector pSB1. This vector comprises a set of virulence genes (virG, virB and virC) derived from the pTiBo542 plasmid from Agrobacterium strain A281. This vector is capable of recombining with any of pBPS0059 to pBPS0063 in Agrobacterium tumefaciens to produce a hybrid vector. The pSB1 vector also comprises the tetracycline resistance gene for selection in bacteria. Restriction endonuclease sites are indicated

FIG. 27 is a graphical representation showing the pSB11 T2 R4R3 super-binary donor base vector containing two separate T-DNAs. One T-DNA contains a multiple cloning site suitable for selectable marker cassettes and the other contains an R4R3 multi-site recombination cassette. Restriction endonuclease cleavage sites are indicated.

FIG. 28 is a graphical representation showing the pSB11ubnT2R4R3 super-binary donor base vector containing two separate T-DNAs. One T-DNA contains a multiple cloning site suitable for selectable marker cassettes and the other contains an R4R3 multi-site recombination cassette. The ubi::bar-nos selectable marker cassette has been cloned into the multiple cloning site of this vector. Restriction endonuclease cleavage sites are indicated.

FIG. 29 is a graphical representation showing the pPZP200 ubi::bar-nos R4R3 base vector. This vector comprises Left border (LB) and Right Border (RB) regions flanking a maize ubiquitin promoter that drives expression of the bialaphos resistance gene (bar). The bar gene is in operable connection with the nos polyadenylation signal. The pPZP200 ubi::bar-nos R4R3 vector also contains an R4R3 multi-site recombination cassette and the spectinomycin resistance gene for selection in bacteria. Restriction endonuclease sites are indicated.

FIG. 30 is a graphical representation showing the pPZP200 ubi::dao1-nos R4R3 base vector. This vector comprises Left border (LB) and Right Border (RB) regions flanking a maize ubiquitin promoter that drives expression of the D-amino oxidase gene (dao1) from the yeast R. gracilis. The dao1 gene in is operable connection with the nos polyadenylation signal. The pPZP200 ubi::bar-nos R4R3 vector also contains an R4R3 multi-site recombination cassette and the spectinomycin resistance gene for selection in bacteria. Restriction endonuclease sites are indicated.

FIG. 31 is a graphical representation showing the pPZP200ubidao1-nos_act1D::rfa-RGA2-rfa(as)-35ST RNAi base vector. This vector comprises Left Border (LB) and Right Border (RB) regions flanking a ubi::dao1-nos selectable marker cassette and an act1D::rfa-RGA2-rfa(as)-35ST cassette. RGA2 is a wheat intron sequence and rfa and rfa(as) are recombination sites for both sense and antisense cloning of a sequence for RNAi silencing.

### DETAILED DESCRIPTION OF THE PREFERRED EMBODIMENTS

### 1. Suitable Plant Strains and Cultivars

The present inventors have demonstrated that the method for producing a transgenic graminaceous plant cell or plant described herein according to any embodiment is generally applicable to a variety of strains of graminaceous plants. Accordingly, the present invention encompasses any species/strain/line/variety/cultivar of graminaceous plant.

For example, the present invention encompasses the production of a transgenic plant or cell from a genus selected from the group consisting of Acamptoclados, Achlaena, Achnatherum, Aciachne, Acidosasa, Acostia, Acrachne, Acritochaete, Acroceras, Actinocladum, Aegilops, Aegopogon, Aeluropus, Afrotrichloris, Agenium, Agnesia, Agropyron, Agropyropsis, Agrostis, Aira, Airopsis, Alexfloydia, Alloeochaete, Allolepis, Alloteropsis, Alopecurus, Alvimia, Amblyopyrum, Ammochloa, Ammophila, Ampelodesmos, Amphibromus, Amphicarpum, Amphipogon, Anadelphia, Anadelphia, Ancistrachne, Ancistragrostis, Andropogon, Andropterum, Anemanthele, Aniselytron, Anisopogon, Anomochloa, Anthaenantiopsis, Anthenantia, Anthephora, Anthochloa, Anthoxanthum, Antinoria, Apera, Aphanelytrum, Apluda, Apochiton, Apoclada, Apocopis, Arberella, Arctagrostis, Arctophila, Aristida, Arrhenatherum, Arthragrostis, Arthraxon, Arthropogon, Arthrostylidium, Arundinaria, Arundinella, Arundo, Arundoclaytonia, Asthenochloa, Astrebla, Athroostachys, Atractantha, Aulonemia, Australopyrum, Austrochloris, Austrodanthonia, Austrofestuca, Austrostipa, Avellinia, Avena, Axonopus, Bambusa, Baptorhachis, Bealia, Beckeropsis, Beckmannia, Bellardiochloa, Bewsia, Bhidea, Blepharidachne, Blepharoneuron, Boissiera, Boivinella, Borinda, Bothriochloa, Bouteloua, Brachiaria, Brachyachne, Brachychloa, Brachyelytrum, Brachypodium, Briza, Bromuniola, Bromus, Brylkinia, Buchloe, Buchlomimus, Buergersiochloa, Calamagrostis, Calamovilfa, Calderonella, Calosteca, Calyptochloa, Camusiella, Capillipedium, Castellia, Catabrosa, Catabrosella, Catalepis, Catapodium, Cathestechum, Cenchrus, Centotheca, Centrochloa, Centropodia, Cephalostachyum, Chaboissaea, Chaetium, Chaetobromus, Chaetopoa, Chaetopogon, Chaetostichium, Chamaeraphis, Chandrasekharania, Chasechloa, Chasmanthium, Chasmopodium, Chevalierella, Chikusichloa, Chimonobambusa, Chionachne, Chionochloa, Chloachne, Chloris, Chlorocalymma, Chrysochloa, Chrysopogon, Chumsriella, Chusquea, Cinna, Cladoraphis, Clausospicula, Cleistachne, Cleistochloa, Cliffordiochloa, Cockaynea, Coelachne, Coelachyropsis, Coelachynum, Coelorachis, Coix, Colanthelia, Coleanthus, Colpodium, Commelinidium, Cornucopiae, Cortaderia, Corynephorus, Cottea, Craspedorhachis, Crinipes, Crithopsis, Crypsis, Cryptochloa, Ctenium, Ctenopsis, Cutandia, Cyathopus, Cyclostachya, Cymbopogon, Cymbosetaria, Cynodon, Cynosurus, Cyperochloa, Cyphochlaena, Cypholepis, Cyrtococcum, Dactylis, Dactyloctenium, Daknopholis, Dallwatsonia, Danthonia, Danthoniastrum, Danthonidium, Danthoniopsis, Dasyochloa, Dasypoa, Dasypyrum, Davidsea, Decaryella, Decaryochloa, Dendrocalamus, Dendrochloa, Deschainpsia, Desmazeria, Desmostachya, Deyeuxia, Diandrochloa, Diandrolyra, Diandrostachya, Diarrhena, Dichaetaria, Dichanthelium, Dichanthium, Dichelachne, Diectomis, Dielsiochloa, Digastrium, Digitaria, Digitariopsis, Dignathia, Diheteropogon, Dilophotriche, Dimeria, Dimorphochloa, Dinebra, Dinochloa, Diplachne, Diplopogon, Dissanthelium, Dissochondrus, Distichlis, Drake-Brochnania, Dregeochloa, Drepanostachyum, Dryopoa, Dupontia, Duthiea, Dybowskia, Eccoilopus, Eccoptocarpha, Echinaria, Echinochloa, Echinolaena, Echinopogon, Ectrosia, Ectrosiopsis, Ehrharta, Ekmanochloa, Eleusine, Elionurus, Elymandra, Elymus, Elytrigia, Elytrophorus, Elytrostachys, Enneapogon, Enteropogon, Entolasia, Entoplocamia, Eragrostiella, Eragrostis, Ereinium, Eremochloa, Eremopoa, Eremopogon, Eremopyrum, Eriachne, Erianthecium, Erianthus, Eriochloa, Eriochrysis, Erioneuron, Euchlaena, Euclasta, Eulalia, Eulaliopsis, Eustachys, Euthryptochloa, Exotheca, Fargesia, Farrago, Fasciculochloa, Festuca, Festucella, Festucopsis, Fingerhuthia, Froesiochloa, Garnotia, Gastridium, Gaudinia, Gaudiniopsis, Germainia, Gerritea, Gigantochloa, Gilgiochloa, Glaziophyton, Glyceria, Glyphochloa, Gouinia, Gouldochloa, Graphephorum, Greslania, Griffithsochloa, Guaduella, Gymnachne, Gyrnnopogon, Gynerium, Habrochloa, Hackelochloa, Hainardia, Hakonechloa, Halopyrum, Harpachne, Harpochloa, Helictotrichon, Helleria, Hemarthria, Hemisorghum, Henrardia, Hesperostipa, Heterachne, Heteranthelium, Heteranthoecia, Heterocarpha, Heteropholis, Heteropogon, Hibanobambusa, Hickelia, Hierochloe, Hilaria, Hitchcockella, Holcolemma, Holcus, Homolepis, Homopholis, Homozeugos, Hookerochloa, Hordelymus, Hordeum, Hubbardia, Hubbardochloa, Humbertochloa, Hyalopoa, Hydrochloa, Hydrothauma, Hygrochloa, Hygroryza, Hylebates, Hymenachne, Hyparrhenia, Hyperthelia, Hypogynium, Hypseochloa, Hystrix, Ichnanthus, Imperata, Indocalamus, Indopoa, Indosasa, Isachne, Isalus, Ischaemum, Ischnochloa, Ischnurus, Iseilema, Ixophorus, Jansenella, Jardinea, Jouvea, Joycea, Kainpochloa, Kaokochloa, Karroochloa, Kengia, Kengyilia, Kerriochloa, Koeleria, Lagurus, Lamarckia, Lamprothyrsus, Lasiacis, Lasiorhachis, Lasiurus, Lecomtella, Leersia, Lepargochloa, Leptagrostis, Leptaspis, Leptocarydion, Leptochloa, Leptochlopsis, Leptocoryphium, Leptoioma, Leptosaccharum, Leptothrium, Lepturella, Lepturidium, Lepturopetium, Leptirus, Leucophrys, Leucopoa, Leymus, Libyella, Limnas, Limnodea, Limnopoa, Lindbergella, Linkagrostis, Lintonia, Lithachne, Littledalea, Loliolum, Lolium, Lombardochloa, Lophacnie, Lophatherum, Lopholepis, Lophopogon, Lophopyrum, Lorenzochloa, Loudetia, Loudetiopsis, Louisiella, Loxodera, Luziola, Lycochloa, Lycurus, Lygeum, Maclurolyra, Maillea, Malacurus, Maltebrunia, Manisuris, Megalachne, Megaloprotachne, Megastachya, Melanocenchris, Melica, Melinis, Melocalamus, Melocanna, Merostachys, Merxmnuellera, Mesosetum, Metasasa, Metcalfia, Mibora, Micraira, Microbriza, Microcalamus, Microchloa, Microlaena, Micropyropsis, Micropyrum, Microstegium, Mildbraediochloa, Milium, Miscanthidium, Miscanthus, Mnesithea, Mniochloa, Molinia, Monachather, Monanthochloe, Monelytrum, Monium, Monocladus, Monocyinbium, Monodia, Mosdenia, Muhlenbergia, Munroa, Myriocladus, Myriostachya, Narduroides, Nardus, Narenga, Nassella, Nastus, Neeragrostis, Neesiochloa, Nematopoa, Neobouteloua, Neohouzeaua, Neostapfla, Neostapfiella, Nephelochloa, Neurachne, Neurolepis, Neyraudia, Notochloe, Notodanthonia, Ochlandra, Ochthochloa, Odontelytrum, Odyssea, Olmeca, Olyra, Ophiochloa, Ophiuros, Opizia, Oplismenopsis, Oplismenus, Orcuttia, Oreobambos, Oreochloa, Orinus, Oropetium, Ortachne, Orthoclada, Oryza, Oryzidium, Oryzopsis, Otachyrium, Otatea, Ottochloa, Oxychloris, Oxyrhachis, Oxytenanthera, Panicum, Pappophoruin, Parafestuca, Parahyparrhenid, Paraneurachne, Parapholis, Paratheria, Parectenium, Pariana, Parodiolyra, Pascopyrum, Paspalidium, Paspalum, Pennisetum, Pentameris, Pentapogon, Pentarrhaphis, Pentaschistis, Pereilema, Periballia, Peridictyon, Perotis, Perrierbambus, Perulifera, Petriella, Peyritschia, Phacelurus, PhaenanthQecium, Phaenospemma, Phalaris, Pharus, Pheidochloa, Phippsia, Phleum, Pholiurus, Phragmites, Phyllorhachis, Phyllostachys, Pilgerochloa, Piptatherum, Piptochaetium, Piptophyllum, Piresia, Piresiella, Plagiantha, Plagiosetum, Planichloa, Plectrachne, Pleiadelphia, Pleioblastus, Pleuropogon, Plinthanthesis, Poa—Bluegrass (grass), Pobeguinea, Podophorus, Poecilostachys, Pogonachne, Pogonarthria, Pogonatherum, Pogoneura, Pogonochloa, Pohlidium, Poidium, Polevansia, Polliniopsis, Polypogon, Polytoca, Polytrias, Pommereulla, Porteresia, Potamophila, Pringleochloa, Prionanthium, Prosphytochloa, Psammagrostis, Psammochloa, Psathyrostachys, Pseudanthistiria, Pseudarrhenatherum, Pseudechinolaena, Pseudobromus, Pseudochaetochloa, Pseudocoix, Pseudodanthonia, Pseudodichanthium, Pseudopentameris, Pseudophleum, Pseudopogonatherum, Pseudoraphis, Pseudoroegneria, Pseudosasa, Pseudosorghum, Pseudostachyum, Pseudovossia, Pseudoxytenanthera, Pseudozoysia, Psilathera, Psilolemma, Psilurus, Pterochloris, Ptilagrostis, Puccinellia, Puelia, Racemobambos, Raddia, Raddiella, Ratzeburgia, Redfieldia, Reederochloa, Rehia, Reimarochloa, Reitzia, Relchela, Rendlia, Reynaudia, Rhipidocladum, Rhizocephalts, Rhomboelytrum, Rhynchelytrum, Rhynchoryza, Rhytachne, Richardsiella, Robynsiochloa, Rottboellia, Rytidosperma, Saccharum, Sacciolepis, Sartidia, Sasa, Sasaella, Sasamorpha, Saugetia, Schafflerella, Schedonnardus, Schenckochloa, Schismus, Schizachne, Schizachyrium, Schizostachyum, Schmidtia, Schoenefeldia, Sclerachne, Sclerochloa, Sclerodactylon, Scleropogon, Sclerostachya, Scolochloa, Scribneria, Scrotochloa, Scutachne, Secale, Sehima, Semiarundinaria, Sesleria, Sesleriella, Setaria, Setariopsis, Shibataea, Silentvalleya, Simplicia, Sinarundinaria, Sinobambusa, Sinochasea, Sitanion, Snowdenia, Soderstromia, Sohnsia, Sorghastrum, Sorghum, Spartina, Spartochloa, Spathia, Sphaerobambos, Sphaerocaryum, Spheneria, Sphenopholis, Sphenopus, Spinifex, Spodiopogon, Sporobolus, Steinchisma, Steirachne, Stenotaphrum, Stephanachine, Stereochlaena, Steyernarkochloa, Stiburus, Stilpnophleum, Stipa, Stipagrostis, Streblochaete, Streptochaeta, Streptogyna, Streptolophus, Streptostachys, Styppeiochloa, Sucrea, Suddia, Swallenia, Swallenochloa, Symplectrodia, Taeniatherum, Taeniorhachis, Tarigidia, Tatianyx, Teinostachyum, Tetrachaete, Tetrachne, Tetrapogon, Tetrarrhena, Thamnocalamus, Thaumastochloa, Thelepogon, Thellungia, Theineda, Thinopyrum, Thrasya, Thrasyopsis, Thuarea, Thyridachne, Thyridolepis, Thyrsia, Thyrsostachys, Thysanolaena, Torreyochloa, Tovarochloa, Trachypogon, Trachys, Tragus, Tribolium, Tricholaena, Trichoneura, Trichopteiyx, Tridens, Trikeraia, Trilobachne, Triniochloa, Triodia, Triplachne, Triplasis, Triplopogon, Tripogon, Tripsacum, Triraphis, Triscenia, Trisetum, Tristachya, Triticum, Tsvelevia, Tuctoria, Uniola, Uranthoecium, Urelytrum, Urochloa, Urochondra, Vahlodea, Vaseyochloa, Ventenata, Vetiveria, Vietnamochloa, Vietnamosasa, Viguierella, Vossia, Vulpia, Vulpiella, Wangenheimia, Whiteochloa, Willkommia, Xerochloa, Yakirra, Ystia, Yushania, Yvesia, Zea, Zenkeria, Zeugites, Zingeria, Zizania, Zizaniopsis, Zonotriche, Zoysia, Zygochlo.

In one example, the graminaceous plant is of the genus Hordeum. Suitable species of plants in the genus Hordeum will be apparent to the skilled artisan and include, for example, H. chilense, H. cordobense, H. euclaston, H. flexuosum, H. intercedens, H. muticum, H. pusillum, H. stenostachys, H. arizonicum, H. comosum, H. jubatum, H. lechleri, H. procenum, H. pubiflorum, H bulbosum, H bulbosum, H bulbosum, H. bulbosum, H. murinum ssp glaucum, H. inurinum ssp leporinum, H. murinum ssp murinum, H. vulgare ssp spontaneum, H. vulgare ssp vulgareH. bogdanii, H. brachyantherum ssp brachyantheruin, H. brachyantherum ssp californicum, H. brevisubulatum ssp brevisubulatum, H. capense, H. depressum, H. erectifolium, H. guatemalense, H. marinum ssp marinum, H. marinum ssp gussoneanum, H. parodii, H. patagonicum ssp magellanicum, H. patagonicum ssp mustersii, H. patagonicum ssp patagonicum, H. patagonicum ssp santacrucense, H. patagonicum ssp setifolium, H. roshevitzii, H secalinum or H. tetraploïdum.

In another example, the graminaceous plant is a ryegrass. Again, a suitable species of ryegrass will be apparent to the skilled artisan. For example, suitable species of ryegrass include, L. perenne, L. multiflorum, L. rigidum or L. temulentum.

In another example, the graminaceous plant is a rice. A suitable species and/or variety of rice will be apparent to the skilled artisan. For example, a suitable variety of rice includes, koshihikari, opus, millin, amaroo, jarrah, illabong, langi, doongara, kyema, basmati, bombia, camaroli, baldo, roma, nero or Arborio.

In another example, the graminaceous plant is a maize. A suitable species and/or variety of maize will be apparent to the skilled artisan. For example, a suitable variety of maize includes, algans, aldante, avenir, Hudson, loft, tasilo, GH128, GH390, QK694 and Hycorn 1, General and PX75.

Preferably, the graminaceous plant is wheat. For example, the wheat is a diploid wheat, such as, for example, Triticum monococcum.

Alternatively, the wheat is a tetraploid wheat, such as, for example, T. turgidum (e.g., var. durum, polonicum, persicum, turanicum or turgidum) or T. durum.

Preferably, the wheat is a hexaploid wheat. For example, the wheat strain/line/variety/cultivar is a winter wheat strain/line/variety/cultivar or a spring wheat strain/line/variety/cultivar.

In one example, the wheat strain/line/variety/cultivar is a strain/line/variety/cultivar grown in or produced in, for example, Australia. For example; the wheat strain or cultivar is selected from the group consisting of Halberd, Cranbrook, Chuan Mai 18 (Cm18), Vigour 18 (V18), Gba Sapphire, Wyalkatchem, Annuello, Wawht2499, Ega Eagle Rock, Gba Ruby, Gba Shenton, Carnamah, Arrino, Babbler, Barunga, Batavia, Baxter, Blade Older, Brookton, Cadoux, Calingiri, Camm, Carnamah, Cascades, Chara, Condor, Cunningham, Dollarbird, Diamondbird, Eradu, Excalibur, Frame, Goldmark, Goroke, H45, Hartog, Hybrid Mercury, Janz, Kelalac, Kennedy, Krichauff, Lang, Machete, Meering, Mitre, Ouyen, Petrie, Silverstar, Spear Older, Stiletto, Strzelecki, Sunbri, Sunbrook, Sunco, Sunlin, Sunstate, Sunvale, Trident, Westonia, Whistler, Worrakatta, Wylah, Yitpi and crosses and hybrids thereof.

In another example, the wheat strain/line/variety/cultivar is a strain/line/variety/cultivar generally grown in northern America, such as, for example, Fielder, Wawawai, Zak, Scarlet, Tara, Neeley, UC 1036, Karl, Jagger, Tam106, Bobwhite, Crocus, Columbus, Kyle, Chinese Spring, Alpowa, Hank, Edwall, Penawawa, Calorwa, Winsome, Butte86, Challis, Maron, Eden, WPB926, WA7839, WA7859, WA7860, WA7875, WA7877, WA7883, WA7884, WA7886, WA7887, WA7890, WA7892, WA7893, WA7900, WA7901, WA7904, WA7914 or WA7915.

In another example, the wheat strain/line/variety/cultivar is a strain/line/variety/cultivar generally grown in Europe, such as, for example, Terra, Brigadier And Hussar, Hunter, Riband, Mercia, Hereward, Spark, Pastiche, Talon, Rialto, Shiraz, FAP75141, Boval, Renan, Derenb Silber, FAP75337, lena, Cappelle, Champlein, Roazon, VPM, Kanzler, Monopol, Carstacht, Vuka, Tamaro, M. Huntsman, Rektor, Bernina, Greif, Caribo, Ares, Kraka, Kronjuwel, Granada, Apollo, Basalt, FAP75527, FAP75507, Galaxie, Obelisk, Formo, Heinevii, Kormoran, Merlin, Bussard, Sperber, FAP75517, Arina, Zenith, FAP754561, Probus, FAP75468, FAP62420, Bezostaja, Kavkas, Timmo, Maris Butler, Sicco, Broom Highbury, Avalon, Fenman, Bounty, Copain, Baron, Norman, Hustler, Kador, Sentry, Flanders, Armada, Brigand or Rapier.

In a further example, the wheat strain/line/variety/cultivar is an elite strain/line/variety/cultivar. In this respect, an “elite” strain/line/variety/cultivar generally displays an improved growth characteristic, such as, for example, resistance to a plant pathogen or drought or desiccation tolerance.

In another example, the wheat strain/line/variety/cultivar is a synthetic derivative of wheat. Such a synthetic derivative is produced, for example, by crossing a cultivated wheat with an uncultivated wheat to thereby improve or enhance the genetic diversity of said wheat. A large number of synthetic wheat derivatives are known in the art and include, for example, a cross between Triticum turgidum and T. taschii. Such a cross mimics the cross that occurred in nature to produce the hexaploid bread wheats of the present day. Suitable sources of such synthetic wheat derivatives will be apparent to the skilled artisan and include, for example, CIMMYT (International Centre for the Improvement of Maize and Wheat; Km. 45, Carretera Mexico-Veracruz. El Batan, Texcoco, Edo. de Mexico, CP 56130 México)

Examples of synthetic wheat derivatives include, for example, CIGM90.590, CIGM88.1536-0B, CIGM90.897, CIGM93.183, CIGM87.2765, CIGM87.2767, CIGM90.561, CIGM88.1239, CIGM88.1344, CIGM92.1727, CIGM90.845, CIGM90.846, CIGM 90.257-1, CIGM 91.61-1, CIGM 90.462, CIGM 90.248-1, CIGM 90.250-2, CIGM 90.412, CIGM90.590, CIGM87.2765-1B-0PR-0B, CIGM88.1175-0B, CIGM87.2767-1B-0PR-0B, CIGM87.2775-1B-0PR-0B, CIGM87.2768-1B-0PR-0B, CIGM86.946-1B-0B-0PR-0B, CIGM87.2770-1B-0PR-0B, CIGM88.1194-0B, CIGM87.2771-1B-0PR-0B, CIGM88.1197-0B, CIGM88.1200-0B, CIGM86.959-1M-1Y-0B-0PR-0B, CIGM88.1209-0B, CIGM90-561, CIGM86.1211-0B, CIGM86.940-1B-0B-0PR-0B, CIGM87.2760-0B-0PR-0B, CIGM88.1212-0B, CIGM86.953-1M-1Y-0B-0PR-0B, CIGM87.2761-1B-0PR-0B, CIGM88.1214-0B, CIGM88.1216-0B, CIGM88.1219-0B, CIGM86.950-1M-1Y-0B-0PR-0B, CIGM86.942-1B-0PR-0B, CIGM90.525, CIGM88.1270-0B, CIGM88.1273-0Y, CIGM88.1288-0B, CIGM88.1313, CIGM88.1313, CIGM88.1344-0B, CIGM88.1273-0Y, CIGM88.1363-0B, CIGM88.1362-0Y, CIGM90.566, CIGM90-590, CIGM89.506-0Y, CIGM89.525-0Y, CIGM89.537-0Y, CIGM89.538-0Y, CIGM90.686, CIGM90.760, CIGM89.559, CIGM89.559, CIGM89.479-0Y, CIGM89.561-0Y, CIGM90.543, CIGM89.564-0Y, CIGM86.944-1B-0Y, 0B-0PR-0B, CIGM86-3277-1B-0B-0PR-0B, CIGM88.1239-2B, CIGM89.567-1B, CIGM90.799, CIGM90.808, CIGM90.812, CIGM90.815, CIGM90.818, CIGM90.820, CIGM90.824, CIGM90.845, CIGM90.846, CIGM90.863, CIGM90.864, CIGM90.865, CIGM90.869, CIGM90.871, CIGM90.878, CIGM90.897, CIGM90.898, CIGM90.906, CIGM90.911, CIGM90.910, CIGM92.1647, CIGM92.1665, CIGM92.1666, CIGM92.1667, CIGM92.1682, CIGM92.1713, CIGM92.1721, CIGM92.1723, CIGM92.1727, CIGM92.1871, CIGM93.183, CIGM93.229, CIGM93.237, CIGM93.388, CIGM93.261, CIGM93.395, CIGM93.266, CIGM93.297, CIGM93.300, CIGM93.406, CIGM93.306, CIGM88.1182-0Y, CIGM87.2754-1B-0PR-0B, CIGM86.951-1RB-0B-0PR-0B, CIGM86.955-1M-1Y-0B-0PR-0B, CIGM88.1217-0B, CIGM88.1228-0B, CIGM88.1240-0B, CIGM90.809, CIGM90.826, CIGM90.896, CIGM93.377, CIGM93.299, CIGM93.302, CASW94Y00092S, CASW94Y00095S, CASW94Y00116S, CASW94Y00130S, CASW94Y00144S, CASW94Y0015 SS, CASW94Y00155S, CASW94Y00156S, CASW94Y00230S, CASW95Y00102S, CASW96Y00555S, CASW96Y00568S, CASW96Y00573S, CASW98B00011S, CASW98B00031S, CASW98B00032S, CASW98B00036S, CASW98B00044S, CASW98B00049S or CASW98B00061S. In this respect, the CIGM number or CASW number referred to supra corresponds to the Cross Identification Number applied to the wheat strain as applied by CIMMYT. Alternatively, synthetic wheat derivatives are described, for example, in Oliver et al., Crop Science, 45:1353-1360, 2005.

In another example, the wheat variety or cultivar (or genotype) is selected from the group consisting of Bobwhite, Chara, Camm, Krichauff, Diamondbird, Yitpi, Wedgetail, Wyalkatchem, Calingiri, Babbler, Silverstar, Sapphire, Frame, Aus29597, Aus29614, Canon, Sunco, Chemnya, Ventura, Tammarin Rock, Kukri, Janz, Sunco, Tasman, Cranbrook, Halberd DH, a CIMMYT non-synthetic derivative, an advanced breeding line generated by, for example, Australian wheat breeding enterprises such as the Department of Agriculture in Western Australia and Australian Grain Technologies Pty Ltd, and crosses and hybrids thereof.

In a further example, the wheat variety or cultivar (or genotype) is selected from the group consisting of Bobwhite, Chara, Camm, Kricbauff, Diamondbird, Yitpi, Wedgetail, Wyalkatchem, Calingiri, Babbler, Silverstar, Sapphire, Frame, Aus29597, Aus29614 and crosses and hybrids thereof.

The skilled artisan will be capable of determining any additional wheat strain, variety/cultivar or breeding line that may be transformed using the method of the invention.

2. Obtaining an Embryo from a Mature Grain

The skilled artisan will be aware of methods for determining the stage of development of a grain from a graminaceous plant, e.g., for determining a grain that has completed grain filling. For example, a wheat seed that is mature comprises approximately 35% moisture. Accordingly, by selecting a wheat seed having about 35% or less moisture a mature grain is selected. The moisture in a wheat seed is determined, for example, using a moisture meter (e.g., as available from Perten Instruments, Springfield, Ill., USA) or using radiofrequency monitoring (e.g., as described in, for example, Lawrence and Nelson, Sensor Update, 7: 377-392, 2001).

Alternatively, the level of endoreduplication in cells of the endosperm of a grain is determined. Suitable methods for determining the level of endoreduplication in cells of the endosperm will be apparent to the skilled artisan and include, for example, those described in Dilkes et al., Genetics, 160: 1163-1177, 2002. For example, endosperm from a wheat grain is isolated (e.g., dissected) and homogenized in a buffer suitable for lysing a plant cell. The level of nucleic acid in a previously determined number of nuclei is then determined using flow cytometry, e.g., by detecting the level of 4′,6-diamidino-2-phenylindole bound to nucleic acid in each nucleus. Endosperm in which no cells or few cells are undergoing endoreduplication are considered to be from a mature grain.

Alternatively, the level of starch in the endosperm of a grain, e.g., a wheat grain, is determined to identify a mature grain. For example, the level of starch in the endosperm of a grain is determined using an amyloglucosidase/α-amylase-based method (such as, for example, the Megazyme total starch assay procedure). Generally, such a method comprises hydrolyzing and solubilizing starch from endosperm of a graminaceous plant using amyloglucosidase and/or α-amylase. The starch dextrins are hydrolyzed to form glucose, which is then quantified using, for example, a glucose oxidase-horseradish peroxidase reaction using 4-aminoantipyrine. Such a method is described, for example, in McLeary et al., J. Cereal Sci., 20: 51-58, 1994.

Alternatively, a mature grain is determined using visual inspection. For example, a wheat grain in which the glumes and peduncle are no longer green and little green coloring remains in the plant is considered a mature wheat grain. Similarly, a wheat grain in which the kernel is hard, but can still be dented with a thumbnail- and/or that is derived from a plant that is completely yellow is considered a mature grain.

In another example, a grain is harvested from a plant that is suspected of comprising mature grain. For example, the maturity of wheat grain is estimated using the growing degree calculation proposed by Bauer, Fanning, Enz and Eberlein. (1984, Use of growing-degree days to determine spring wheat growth stages. North Dakota Coop. Ext. Ser. EB-37. Fargo, N. Dak.).

Following isolation of a mature grain, embryonic cells are isolated therefrom, e.g., by excising or dissecting the embryonic cells away from the grain. Methods for obtaining embryonic cells from a mature grain will be apparent to the skilled artisan and/or described, for example, in Delporte et al., Plant Cell Tiss. Organ Cult. 67: 73-80, 2001. For example, the embryo is excised using a blade (e.g., a scalpel blade).

In one example, the seed is imbibed for a period of time (e.g., 1-2 hours) in water to facilitate obtaining the embryonic cells therefrom.

In one example, the seed coat is removed from the mature embryo. Methods for removing the seed coat will be apparent to the skilled artisan. For example, the seed coat is excised from the mature embryo, e.g., using a blade (e.g., a scalpel blade). Alternatively, the seed coat is removed by cracking or scratching the seed coat with a knife or abrasive materials. The seed coat may also be removed by, for example, contacting the embryo with an acid (e.g., sulfuric acid), or a solvent (e.g., acetone or alcohol) for a time sufficient to remove the seed coat.

3. Transformation of Mature Embryo with a Bacterium

### 3.1 Suitable Strains of Bacteria

Suitable bacteria for introducing a nucleic acid into a graminaceous plant cell will be apparent to the skilled artisan. For example, Broothaerts et al., (Nature 433: 629633, 2005) describe the production of transgenic plants using Rhizobium spp. NGR234 or Sinorhizobium meliloti or Mesorhizobium loti. Accordingly, it is preferable that the transformation method of the invention as described in any embodiment is performed using any one of these bacteria or with Agrombacterium sp. In each case the nucleic acid transferred to the transgenic plant was carried within a Ti vector. Furthermore, the transformation protocols were similar to those used for Agrobacterium. Accordingly, the description provided herein with respect to vectors and transformation procedures for Agrobacterium shall be taken to apply mutatis mutandis to transformation using one or more of the previously described bacteria.

In an example of the invention, nucleic acid is introduced into a graminaceous plant cell using Agrobacterium. Members of the genus Agrobacterium are soil-borne in their native environment, Gram-negative, rod-shaped phytopathogenic bacteria that cause crown gall disease or hairy root disease. The term “Agrobacterium” includes, but is not limited to, strains Agrobacterium tumefaciens, (that typically causes crown gall in infected plants), and Agrobacterium rhizogenes (that typically cause hairy root disease in infected host plants). Infection of a plant cell with Agrobacterium generally results in the production of opines (e.g., nopaline, agropine, octopine etc.) by the infected cell. Thus, Agrobacterium strains which cause production of nopaline (e.g., strain LBA4301, C58, A208, GV3101) are referred to as “nopaline-type” Agrobacterium; Agrobacterium strains that cause production of octopine (e.g., strain LBA4404, Ach5, B6) are referred to as “octopine-type” Agrobacterium; and Agrobacterium strains that cause production of agropine (e.g., strain EHA105, EHA101, A281) are referred to as “agropine-type” Agrobacterium.

In one example, nucleic acid is introduced into a graminaceous plant using A. tumefaciens or A. rhizogenes. Preferably, the A. tumefaciens or A. rhizogenes is a disarmed Agrobacterium. In this respect, a disarmed Agrobacterium comprises the genes required to infect a plant cell (e.g., vir genes), however lacks the nucleic acid required to cause plant disease, e.g., crown gall disease.

A. tumefaciens strains are generally defined by their chromosomal background and the resident or endogenous Ti plasmid found in the strain. Examples of suitable Agrobacterium strains and their chromosomal background and Ti plasmid are set forth in Table 1:

TABLE 1
Disarmed A. tumefaciens strains described by Agrobacterium chromosomal background and Ti plasmid they comprise

| | Chromosomal | | Ti Plasmid | | | |
|---|---|---|---|---|---|---|
| Agrobacterium Strain | Background | Marker Gene | | Marker gene | Opine | Reference |
| LBA4404 | TiAch5 | rif | pAL4404 | Spec and | Octopine | Hoekema et al., |
| | | | | strep | | Nature, 303: 179-180 |
| GV2260 | C58 | rif | pGV2260 | carb | Octopine | McBride and Summerfelt |
| | | | (pTiB6S3Δ | | | Plant Mol. Biol. 14: 269-276 |
| | | | T-DNA) | | | |
| C58C1 | C58 | — | Cured | — | Nopaline | Deblaere et al., Nucleic Acids |
| | | | | | | Res., 13, 4777-1778, 1985 |
| GV3100 | C58 | — | Cured | — | Nopaline | Holsters et al., Plasmid, |
| | | | | | | 3: 212-230, 1980 |
| A136 | C58 | Rif and nal | Cured | — | Nopaline | Watson et al., J. Bacteriol., |
| | | | | | | 123: 255-264, 1975 |
| GV3101 | C58 | rif | Cured | — | Nopaline | Holsters et al., Plasmid, |
| | | | | | | 3: 212-230, 1980 |
| GV3850 | C58 | rif | pGV3850 | carb | Nopaline | Zambryski et al., EMBO J. |
| | | | (pTiC58Δon | | | 2: 2143-2150, 1983 |
| | | | c genes) | | | |
| GV3101::pMP90 | C58 | rif | pMP90 | gent | Nopaline | Koncz and Schell Mol. Gen. |
| | | | (pTiC58ΔT0 | | | Genet. 204: 383-396, 1986 |
| | | | DNA) | | | |
| GV3101::pMP90 | C58 | rif | pMP90RK | Gent and | Nopaline | Koncz and Schell Mol. Gen. |
| RK | | | (pTiC58ΔT0 | kan | | Genet. 204: 383-396, 1986 |
| | | | DNA) | | | |
| EHA101 | C58 | rif | pEHA101 | kan | Nopaline | Hood et al., J. Bacteriol, |
| | | | (pTiBo542Δ | | | 168: 1291-1301, 1986 |
| | | | T-DNA) | | | |
| EHA105 | C58 | rif | pEHA105 | — | Succinamopine | Hood et al., Transgenic Res. |
| | | | (pTiBo542Δ | | | 2: 208-218, 1993 |
| | | | T-DNA) | | | |
| AGL-1 | C58, RecA | rif, carb | pTiBo542Δ | — | Succinamopine | Lazo et al., Biotechnology, |
| | | | T-DNA | | | 9: 963-967, 1991 |

In one example, the A. tumefaciens used in the method of the present invention has an improved ability to infect a plant cell. Suitable strains having improved infectivity are known in the art. For example, strains comprising an increased level of virG or an increased level of active virG have been produced (Zupan et al., Plant J, 23: 11-28, 2000). Increasing the level of virG expression or activation results in increased expression of the remaining genes in the vir cluster, thereby enhancing the infectivity of A. tumefaciens.

Alternatively, or in addition, the A. tumefaciens strain comprises enhanced virE1 expression (Zupan et al., supra). virE1 encodes a single-stranded DNA binding protein that binds to the transferred T-strand of the T-DNA thereby enhancing introduction of the T-DNA into the plant cell.

Additional strains of A. tumefaciens will be apparent to the skilled artisan and include, for example, A281 (Hood et al, J. Bacteriol. 168: 1291-1301, 1986.

Suitable strains of A. rhizogenes will be apparent to the skilled artisan. For example, the strain is selected from the group consisting of R1601, R1000, ATCC15834, MAFF03-01724, A4RS, LBA 9402 and LMG 1500 (Han et al., Can. J. For. Res., 27: 464-470, 1997 or Bais et al., Current Science, 80: 83-87, 2001). Suitable sources of A. rhizogenes strains will be apparent to the skilled artisan. The skilled artisan will also be aware that following introduction of a nucleic acid into a plant cell using A. rhizogenes, roots are induced to form. These roots are then used to regenerate a plantlet (e.g., to produce a shoot) using a method known in the art and/or described herein.

### 3.2 Nucleic Acid Constructs

As the transformation method of the present invention makes use of bacterium, and preferably, Agrobacterium, a suitable nucleic acid construct generally comprises or consists of a Ti plasmid (in the case of A. tumefaciens) or a Ri plasmid (in the case of A. rhizogenes). In the context of the present invention, such a vector generally comprises a transgene of interest within a transfer-nucleic acid that is introduced to a plant cell. Suitable transgenes are described in greater detail infra. The current section describes suitable constructs for introducing said transgene into a plant cell.

In an example, the nucleic acid construct comprises a transgene of interest flanked by or delineated by imperfect repeat DNA (also known as the left border (LB) and the right border (RB)). Nucleotide sequences of exemplary LB and RB are set forth in SEQ ID NOs: 1 and 2, respectively. Preferably, a suitable nucleic acid construct for use in the method of the present invention comprises a suitable LB and RB.

### 3.2.1 Promoters

In another example, the nucleic acid construct comprises a transgene and/or a selectable marker gene and/or a detectable marker gene placed in operable connection with a suitable promoter.

In another example, the transgene of interest and/or the selectable/detectable marker gene is/are operably linked to a promoter that is operable in a plant cell. In this respect, the promoter need not necessarily be operable in the plant cell that is initially transformed using the method of the invention; rather the promoter may be inducible and/or operable in a particular cell type or developmental stage.

Promoters suitable for use in a nucleic acid construct (e.g., to drive expression of a transgene and/or a detectable/selectable marker gene) for expression in plants include, for example, those promoters derived from the genes of viruses, yeasts, moulds, bacteria, insects, birds, mammals and plants which are capable of functioning in graminaceous plant cells. The promoter may regulate gene expression constitutively, or differentially with respect to the tissue in which expression occurs, or with respect to the developmental stage at which expression occurs, or in response to external stimuli such as physiological stresses, pathogens, or metal ions, amongst others.

Examples of promoters useful in performance of the present invention include the CaMV 35S promoter (SEQ ID NO: 3), a maize ubiquitin promoter (SEQ ID NO: 4), a rice actin 1 promoter (SEQ ID NO: 5), a maize alcohol dehydrogenase 1 promoter, a pEMU synthetic promoter (Last et al., Theor. Appl. Genet. 81, 581-588, 1991), rd29a stress inducible promoter from Arabidopsis (SEQ ID NO: 6), ScBV promoter from sugarcane bacilli virus (SEQ ID NO: 6), basi promoter from barley (SEQ ID NO: 7) or a cad2 promoter from ryegrass.

In addition to the specific promoters identified herein, cellular promoters for so-called housekeeping genes, including the actin promoters, or promoters of histone-encoding genes, are useful.

Alternatively, an inducible promoter is used. An inducible promoter is a promoter induced by a specific stimulus such as stress conditions comprising, for example, light, temperature, chemicals, drought, high salinity, osmotic shock, oxidant conditions or in case of pathogenicity.

3.2.2 Selectable and/or Detectable Markers

In another example, the nucleic acid construct comprises one or more selectable and/or detectable markers that facilitate selection and/or detection of a bacterial cell and/or a plant cell comprising said nucleic acid construct or fragment thereof.

Bacterial Selectable Markers

In one example, a nucleic acid construct comprises a nucleic acid encoding a selectable and/or a detectable marker operable in a bacterial cell. Such a selectable and/or a detectable marker facilitates the selection or identification of a bacterial cell that comprises the nucleic acid construct. However, as discussed supra several bacterial strains, e.g., strains of Agrobacterium, also comprise a gene encoding a selectable and/or a detectable reporter. In this respect, it is preferable that the selectable and/or detectable reporter gene within the nucleic acid construct differs to that in the bacterial strain used.

Generally, the nucleic acid construct comprises a selectable marker that confers resistance to a cytotoxic compound to a bacterial cell. For example, the nucleic acid construct comprises a selectable marker encoding a polypeptide that confers resistance to kanamycin, gentamycin, tetracycline, streptomycin or spectinomycin.

Plant Selectable and/or Detectable Markers

In a further example, the nucleic acid construct comprises a nucleic acid encoding a selectable and/or a detectable marker operable in a plant cell. Such a selectable and/or detectable marker facilitates the selection and/or identification of a plant cell that has been transformed using the method of the invention. As will be apparent to the skilled artisan, such a selectable and/or detectable marker gene is preferably located within the transfer-nucleic acid of the construct to thereby facilitate introduction into the plant cell.

In one example, the nucleic acid construct comprises a selectable marker operable in a plant. Suitable selectable markers will be apparent to the skilled artisan. For example, the selectable marker is a bar gene (bialaphos resistance gene) (SEQ ID NO: 8) that encodes phosphinothricin acetyl transferase (pat) (SEQ ID NO: 9).

Alternatively, the selectable marker provides resistance to an antibiotic. For example, the selectable marker is encoded by the bacterial neomycin phosphotransferase II (nptII) gene (SEQ ID NO: 10) that provides resistance to aminoglycoside antibiotics. Alternatively, the selectable marker is encoded by a hygromycin phosphotransferase gene (SEQ ID NO: 12) (providing resistance to hygromycin B) or an aacC3 gene or an aacC4 gene (providing resistance to gentamycin) or a chloramphenicol acetyl transferase gene (SEQ ID NO: 14) (conferring resistance to chloramphenicol).

In another example, the selectable marker confers resistance to a herbicide. For example, the selectable marker is a gene encoding 5-enolpyruvyl-shikimate-3-phosphate synthase (SEQ ID NO: 16) or phosphinothricin synthase (SEQ ID NO: 18), which provide tolerance to glyphosate and/or glufosinate ammonium herbicides, respectively. The enolpyruylshikimate-phosphate synthase (CP4) (SEQ ID NO: 20) gene from Agrobacterium strain 4 and the glyphosate oxidoreductase (GOX) gene (SEQ ID NO: 22) also encode polypeptides that provide tolerance to glyphosate ammonium herbicides (Zhou et al., Plant Cell Reports, 15: 159-163, 1995).

In another example, the selectable marker confers the ability to survive and/or grow in the presence of a compound in which an untransformed plant cell cannot grow and/or survive. For example, the selectable marker is a mannose-6-phosphate isomerase (MPI) encoded by the mana gene (SEQ ID NO: 24) from Escherichia coli (Hansen and Wright, Trends in Plant Sciences, 4: 226-231, 1999). MPI permits transformed cells to grow in the presence of mannose as the sole carbon source.

Alternatively, the selectable marker is encoded by the cyanamide hydratase (Cah) gene (SEQ ID NO: 26) (as described in U.S. Ser. No. 09/518,988). Cyanamide hydratase permits a transformed plant cell to grow in the presence of cyanamide, by converting cyanamide to urea.

In one example, the selectable marker is a D-amino oxidase, (DAAO) e.g., encoded by a nucleic acid comprising a nucleotide sequence set forth in SEQ ID NO: 28. As discussed supra, DAAO permits a transformed plant cell or plant to grow in the presence of D-alanine and/or D-serine. Suitable methods for producing a nucleic acid construct comprising DAAO as a selectable marker are known in the art and/or described in Erikson et al., Nature Biotechnology, 22: 455-458, 2004 or in International Publication No. WO2003/060133. Other suitable selectable markers for selection using D-amino acids will be apparent to the skilled artisan based on the description in WO2003/060133. For example, the selectable marker is encoded by a D-amino acid ammonia-lyase, for example, from Escherichia coli.

In another example, the nucleic acid construct comprises a detectable marker gene, preferably, the transfer-nucleic acid comprises a detectable marker gene. Suitable detectable marker gene include, for example, a β-glucuronidase gene (GUS; the expression of which is detected by the metabolism of 5-bromo-4-chloro-3-indolyl-1-glucuronide to produce a blue precipitate) (SEQ ID NO: 30); a bacterial luciferase gene (SEQ ID NO: 32); a firefly luciferase gene (detectable following contacting a plant cell with luciferin); or a β-galactosidase gene (the expression of which is detected by the metabolism of 5-bromo-4-chloro-3-indolyl-β-D-galactopyranoside to produce a blue precipitate) (SEQ ID NO: 34).

In another example, the detectable marker is a fluorescent marker. For example, the fluorescent marker is a monomeric discosoma red fluorescent protein (dsRED; SEQ ID NO: 36) or a monomeric GFP from Aequorea coerulescens (SEQ ID NO: 38). Preferably, the marker is dsRED. Methods for detecting a fluorescent protein will be apparent to the skilled artisan and include, for example, exposing a plant cell or plant to a light of suitable wavelength to excite said fluorescent protein and detecting light emitted from said plant cell or plant.

### 3.2.3 Production of Nucleic Acid Constructs

Methods for producing nucleic acid constructs are known in the art and/or described in Ausubel et al (In: Current Protocols in Molecular Biology. Wiley Interscience, ISBN 047 150338, 1987), Sambrook et al (In: Molecular Cloning: Molecular Cloning: A Laboratory Manual, Cold Spring Harbor Laboratories, New York, Third Edition 2001).

Typically, the nucleic acid encoding the constituent components of the nucleic acid construct is/are isolated using a known method, such as, for example, amplification (e.g., using PCR or splice overlap extension) or isolated from nucleic acid from an organism using one or more restriction enzymes or isolated from a library of nucleic acids. Methods for such isolation will be apparent to the ordinary skilled artisan.

Alternatively, nucleic acid encoding a nucleic acid constituent of a construct for use in the method of the present invention is isolated using polymerase chain reaction (PCR). Methods of PCR are known in the art and described, for example, in Dieffenbach (ed) and Dveksler (ed) (In: PCR Primer: A Laboratory Manual, Cold Spring Harbour Laboratories, NY, 1995). Generally, for PCR two non-complementary nucleic acid primer molecules comprising at least about 20 nucleotides in length, and more preferably at least 25 nucleotides in length are hybridized to different strands of a nucleic acid template molecule, and specific nucleic acid copies of the template are amplified enzymatically. Preferably, the primers hybridize to nucleic acid adjacent to the nucleic acid of interest (e.g., a transgene, a promoter and/or a nucleic acid encoding a detectable marker or selectable marker), thereby facilitating amplification of the nucleic acid. Following amplification, the amplified nucleic acid is isolated using a method known in the art and, preferably cloned into a suitable vector, e.g., a vector described herein.

Other methods for the production of a nucleic acid of the invention will be apparent to the skilled artisan and are encompassed by the present invention. For example, a nucleic acid construct is produced by cloning a transgene of interest into a binary vector.

### 3.2.4 Binary Vectors

In one example, the nucleic acid construct is a Ti plasmid or a Ri plasmid comprising the transgene of interest.

Preferably, the Ti plasmid or Ri plasmid comprises each of the vir genes required for introduction of nucleic acid into a plant cell by A. tumefaciens.

Preferably, the nucleic acid construct is a binary Ti plasmid or Ri plasmid. Binary Ti plasmids or Ri plasmids are produced based on the observation that the T-DNA (nucleic acid transferred to a plant cell) and the vir genes required for transferring the T-DNA may reside on separate plasmids (Hoekema et al., Nature, 303: 179-180, 1983). In this respect, the vir function are generally provided by a disarmed Ti plasmid resident in or endogenous to the Agrobacterium strain used to transform a plant cell (e.g., an Agrobacterium strain described supra).

Accordingly, a binary Ti plasmid or Ri plasmid comprises a transgene located within transfer-nucleic acid (e.g., T-DNA). Such transfer-nucleic acid comprising the transgene is generally flanked by or delineated by a LB and a RB.

Suitable binary plasmids are known in the art and/or commercially available. For example, a selection of binary Ti vectors is described in Table 2.

TABLE 2
Binary Ti plasmids useful for Agrobacterium-mediated transformation

| | Bacterial | Origin of replication | | |
|---|---|---|---|---|
| Vector | selection | Agrobacterium | E. coli | Reference |
| pBIN19 | kan | pRK2 | pRK2 | Bevan et al., Nucleic Acids Res., |
| | | | | 12: 8711-8721, 1984 |
| pC22 | Amp, strep, | pRi | ColE1 | Simoens et al., Nucleic Acids |
| | spect | | | Res. 14: 8073-8090, 1986 |
| pGA482 | tet | pRK2 | ColE1 | An et al., EMBO J. 4: 277-284, |
| | | | | 1985 |
| pPCV001 | amp | pRK2 | ColE1 | Koncz and Schell Mol. Gen. |
| | | | | Genet. 204: 383-396, 1986 |
| pCGN1547 | gent | pRi | ColE1 | McBride and Summerfelt 14: |
| | | | | 269-276, 1990 |
| pJJ1881 | tet | pRK2 | pRK2 | Jones et al., Transgenic Res. 1: |
| | | | | 285-297, 1992 |
| pPZP111 | chloro | pVS1 | ColE1 | Hajukiewicz et al., Plant Mol. |
| | | | | Biol. 25: 989-994, 1994 |
| pGreen0029 | kan | pSa | pUC | Hellens et al., Plant Mol. Biol., |
| | | | | 42: 819-832, 2000 |

Additional binary vectors are described in, for example, Hellens and Mullineaux Trends in Plant Science 5: 446-451, 2000.

Suitable Ri plasmids are also known in the art and include, for example, pRiA4b (Juouanin Plasmid, 12: 91-102, 1984), pRi1724 (Moriguchi et al., J. Mol. Biol. 307:771-784, 2001), pRi2659 (Weller et al., Plant Pathol. 49:43-50, 2000) or pRi1855 (O'Connell et al., Plasmid 18:156-163, 1987).

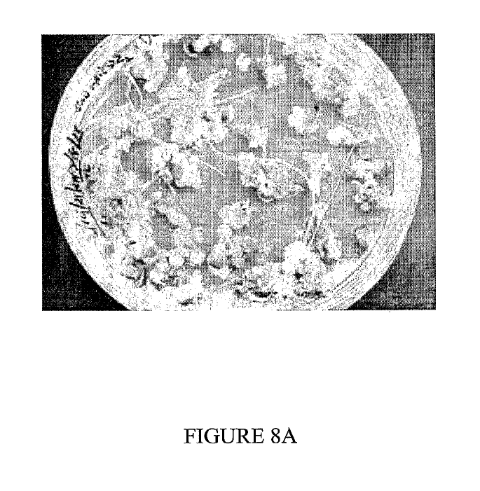

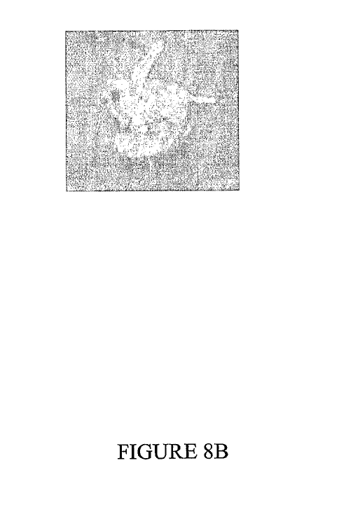

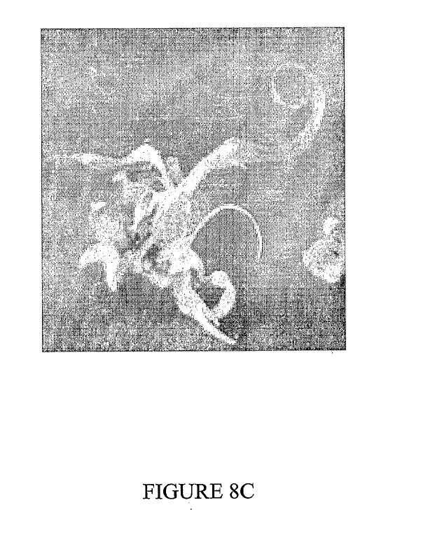

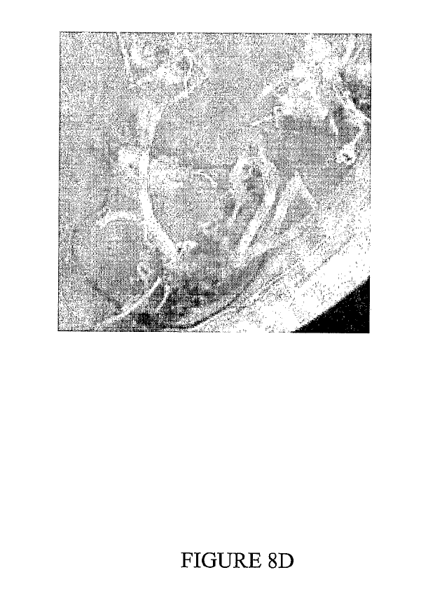

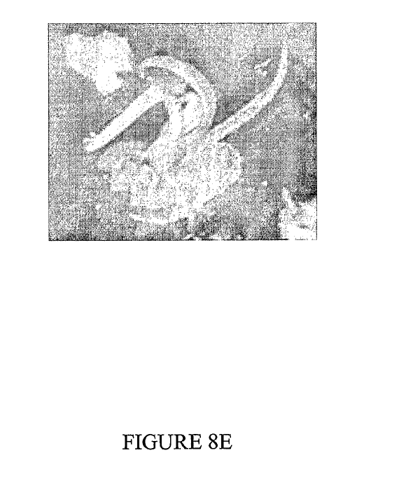

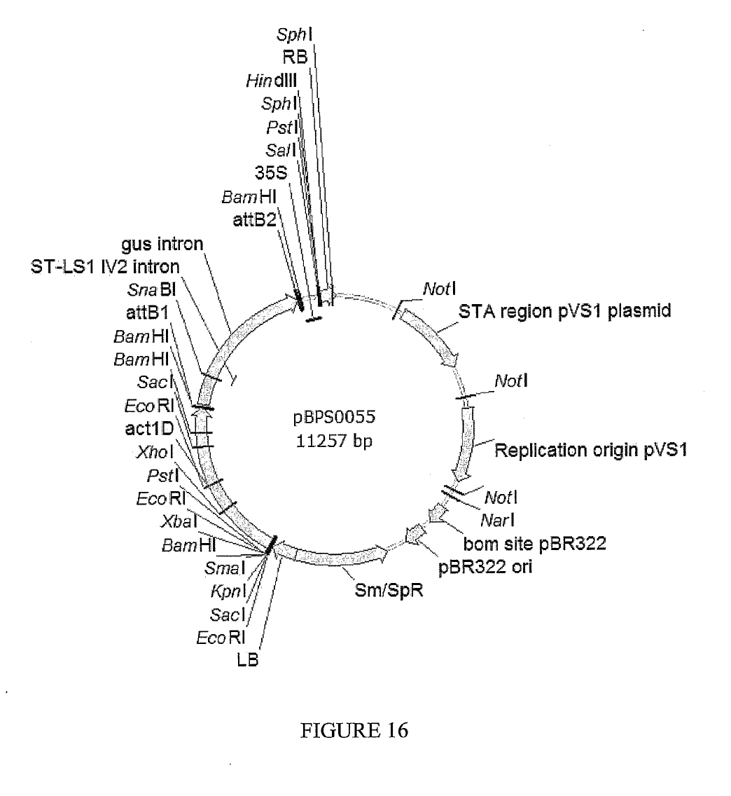

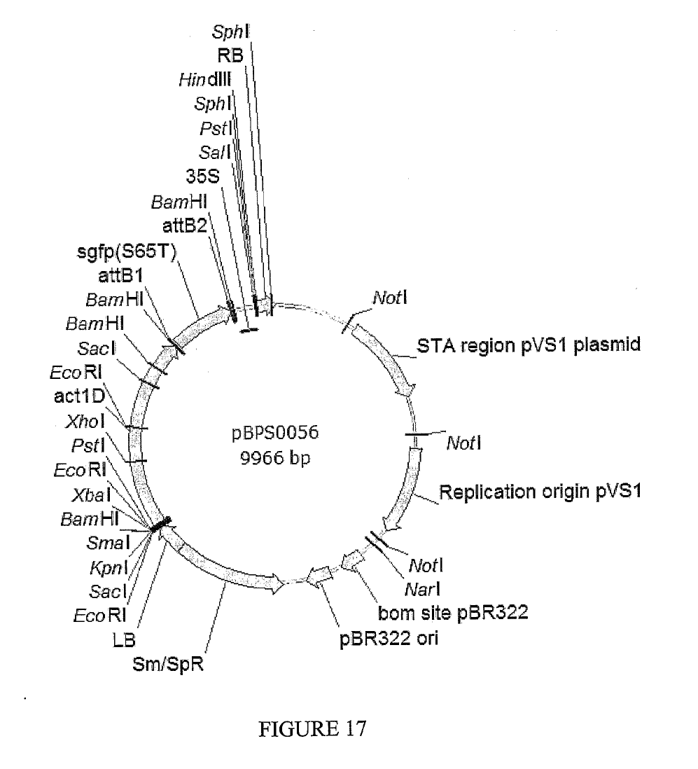

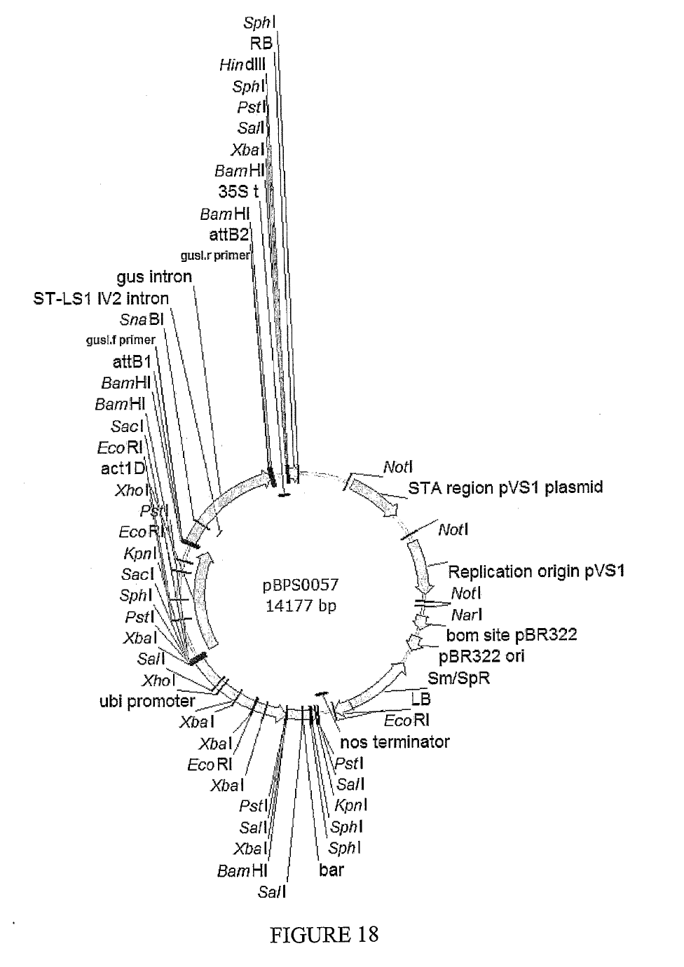

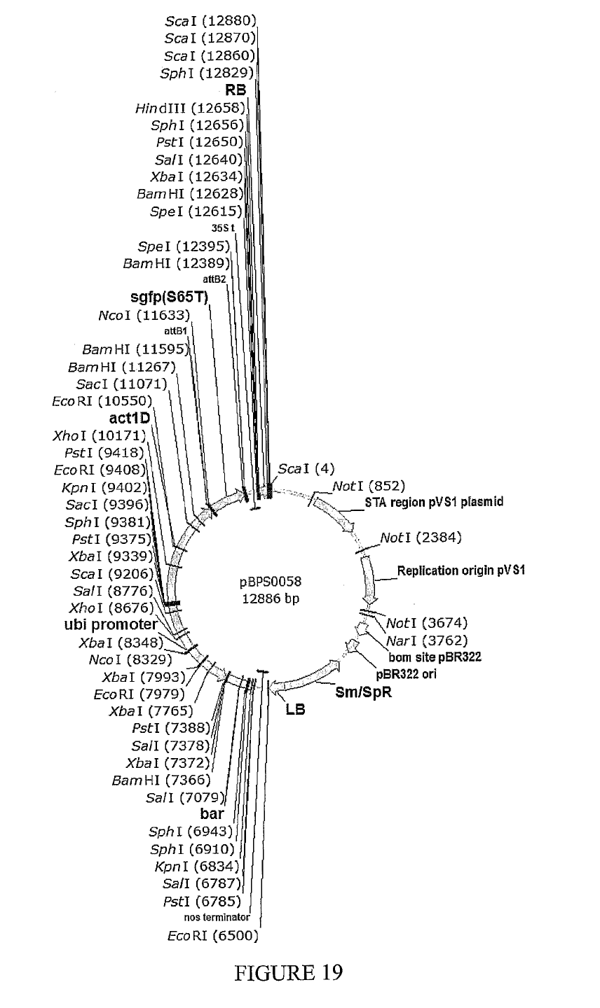

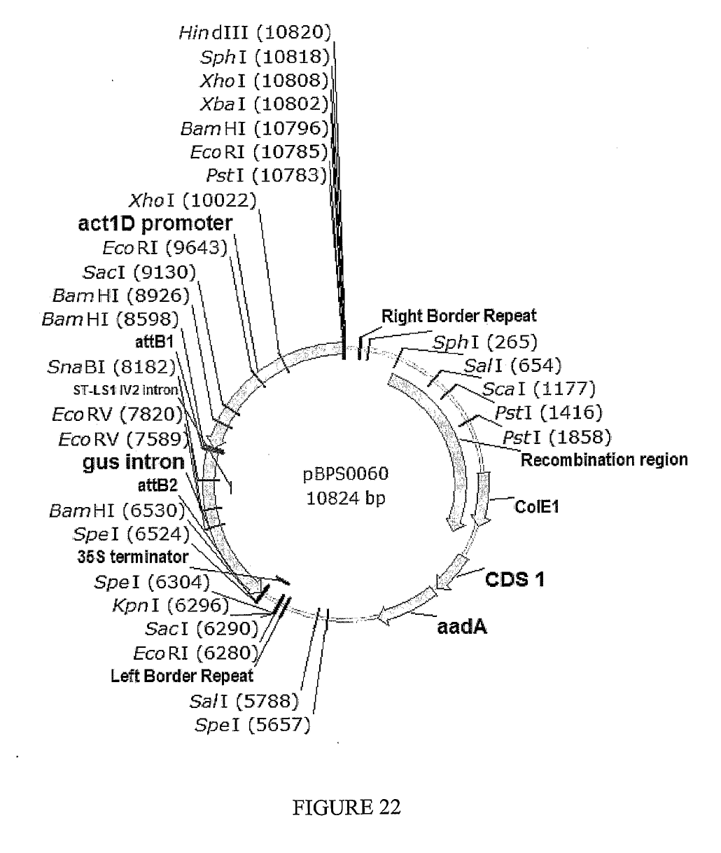

The present inventors additionally provide a number of binary vectors suitable for transforming a nucleic acid (e.g., a reporter gene) into a plant. Alternatively, or in addition, these vectors are suitable for modification for transforming a nucleic acid of interest into a plant. Vector maps for each vector are depicted in FIGS. 8 to 22.

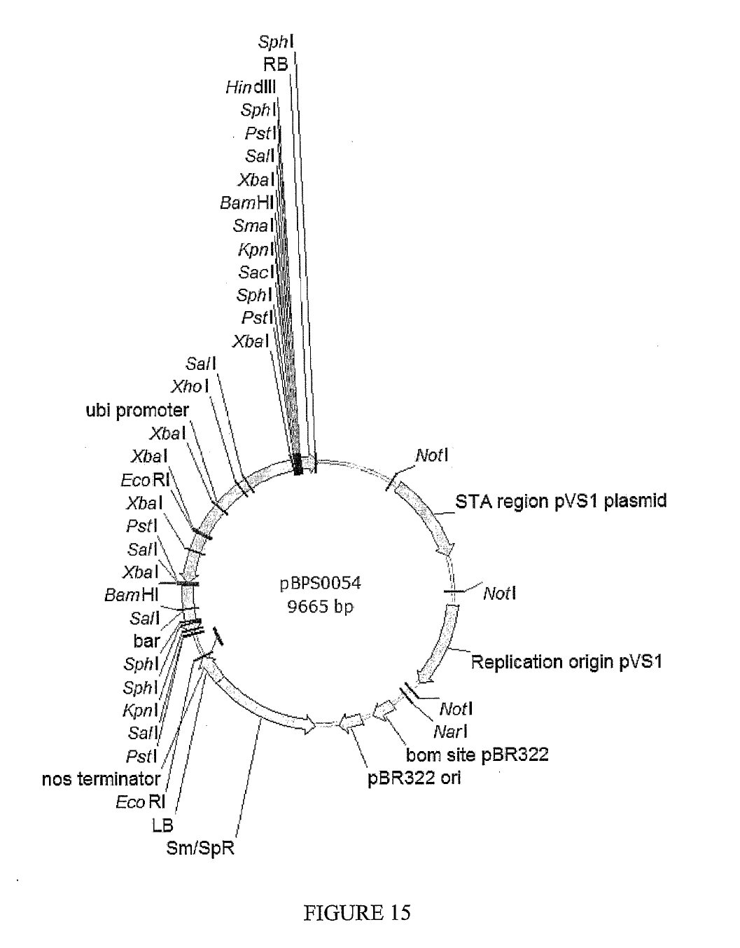

In particular, the inventors provide five binary vectors for bacterial-mediated transformation of a graminaceous plant (see Table 3 and FIGS. 15-19). Each vector has a pPZP200 vector backbone (Hajdukiewicz et al., Plant Mol. Biol. 25:989-94, 1994) and contains either chimeric act1D::gusA or act1D::sgfp with or without a chimeric ubi::bar selectable marker-cassette.

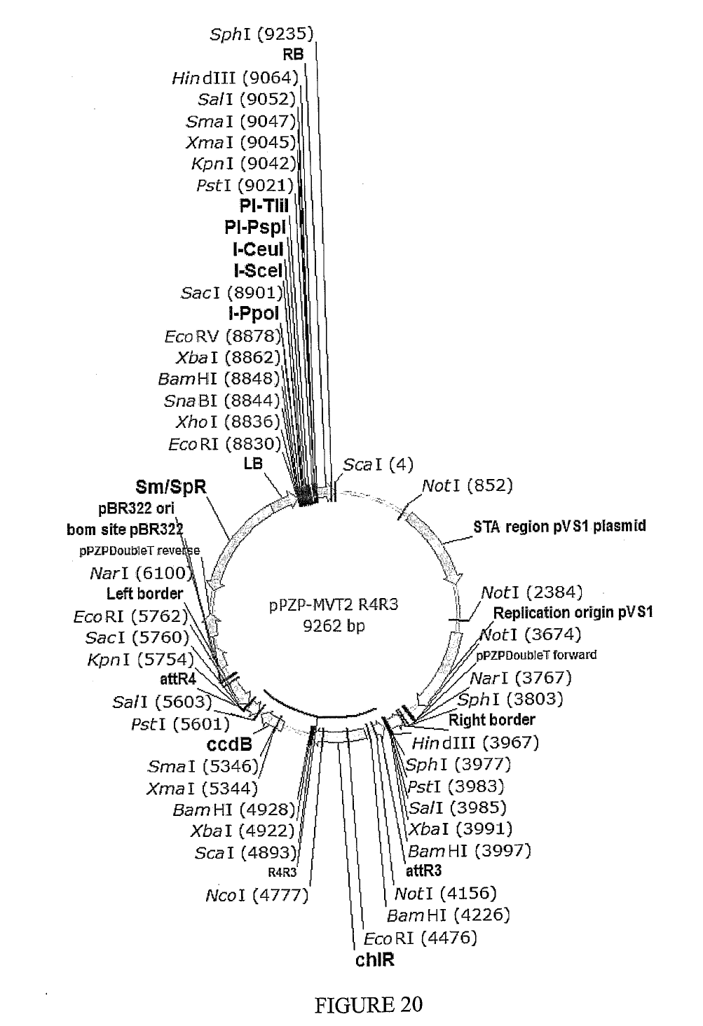

The inventors also provide a binary base vector containing two separate T-DNAs to facilitate marker excision (FIG. 20). One T-DNA contains a multiple cloning site suitable for modular expression cassettes and the other contains an R4R3 multi-site recombination cassette suitable for a selectable marker cassette. The multiple cloning site consists of 13 hexanucleotide restriction sites, 6 octanucleotide restriction sites and 5 rare homing endonuclease sites to facilitate modularization (as described in Goderis et al., Plant Mol. Biol. 50: 17-27, 2002). With this modular system up to six different expression cassettes can be cloned into the one binary vector.

TABLE 3
Bacterial binary vectors.

| Vector | Selectable marker | Reporter gene expression | Refered to |
|---|---|---|---|
| backbone | cassette | cassette | herein |
| pPZP200 | ubi::bar-nos | — | pBPS0054 |
| pPZP200 | — | act1D::gusi-35S | pBPS0055 |
| pPZP200 | — | act1D::sgfp-35S | pBPS0056 |
| pPZP200 | ubi::bar-nos | act1D::gusi-35S | pBPS0057 |
| pPZP200 | ubi::bar-nos | act1D::sgfp-35S | pBPS0058 |

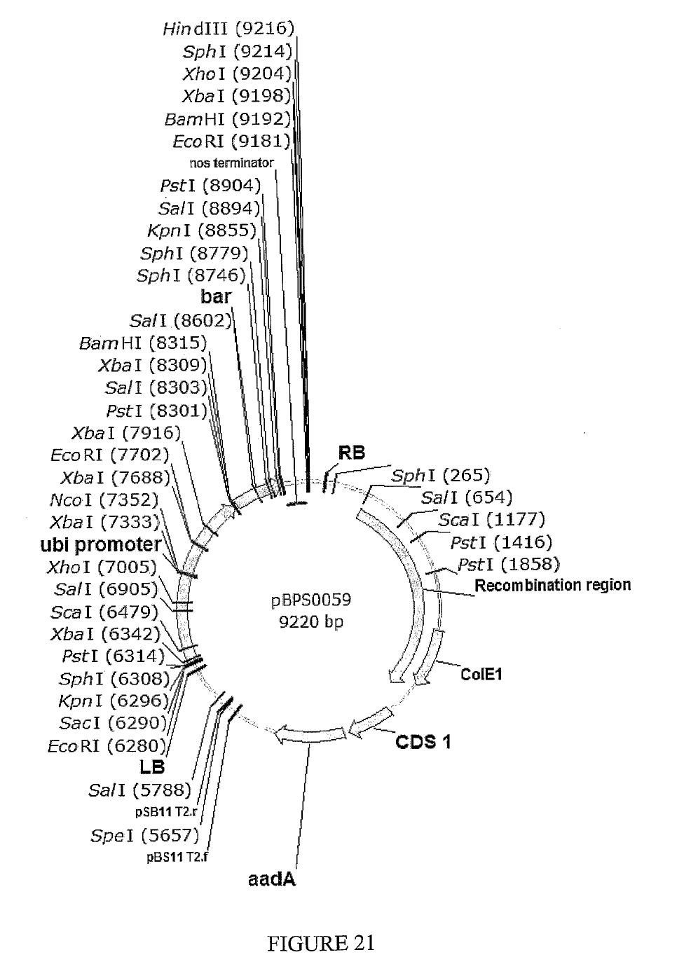

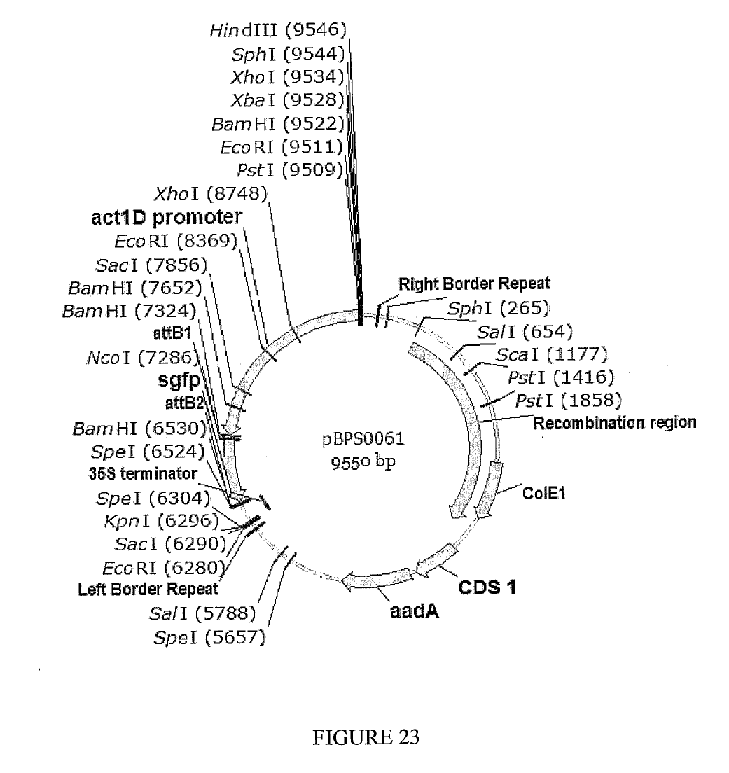

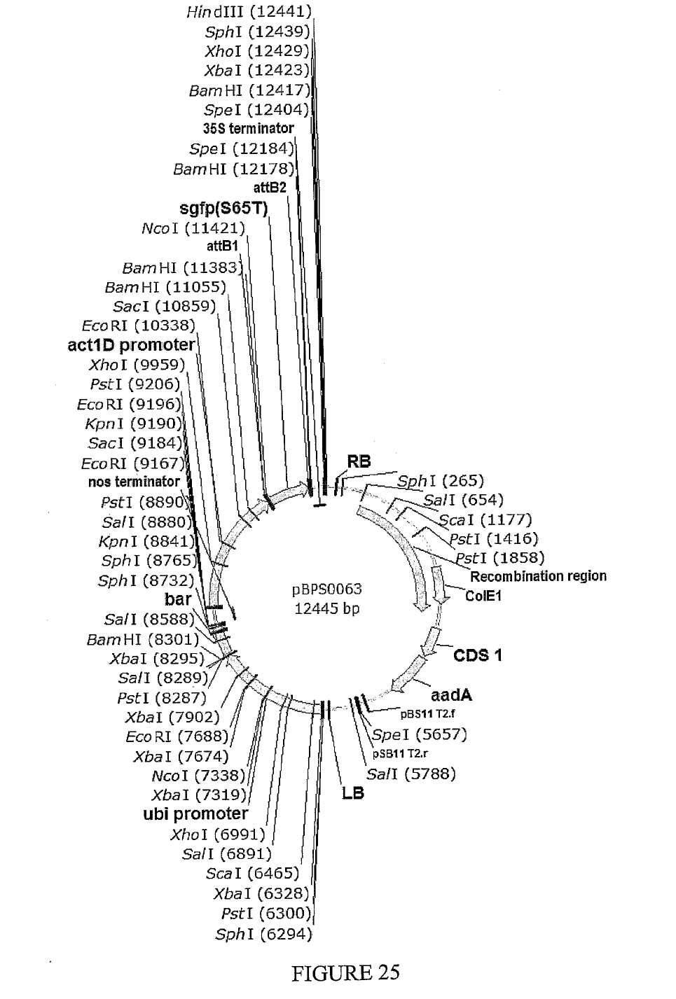

The present inventors also provide five super-binary donor and one super-binary acceptor vectors for bacterial-mediated transformation of graminaceous plant cells, e.g., wheat cells (FIGS. 21-25). Each donor vector consists of a pSB11 vector backbone (Komari et al., Plant J 10: 165-174, 1996) containing either chimeric act1D::gusA or act1D::sgfp with or without a chimeric ubi::bar selectable marker cassette.

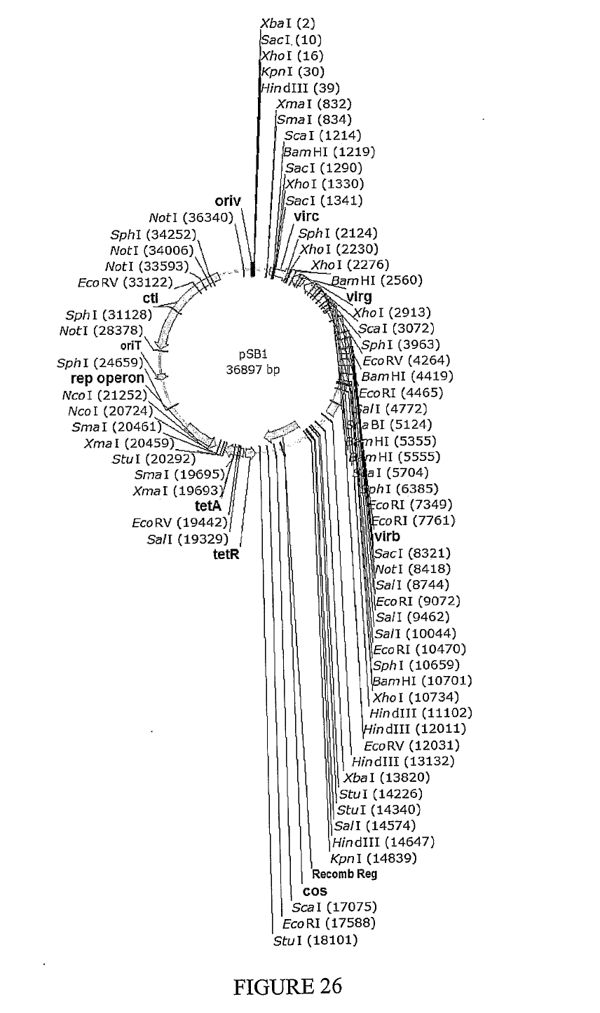

The pSB1 acceptor vector (FIG. 26) contains a set of virulence genes (virG, virB and virC) derived from the pTiBo542 plasmid from Agrobacterium strain A281 (Komari, supra). Both the donor and acceptor vectors share a 2.7 Kb fragment and homologous recombination (single cross-over) takes place in this region in a bacterium, e.g., Agrobacterium tumefaciens resulting in a hybrid vector.

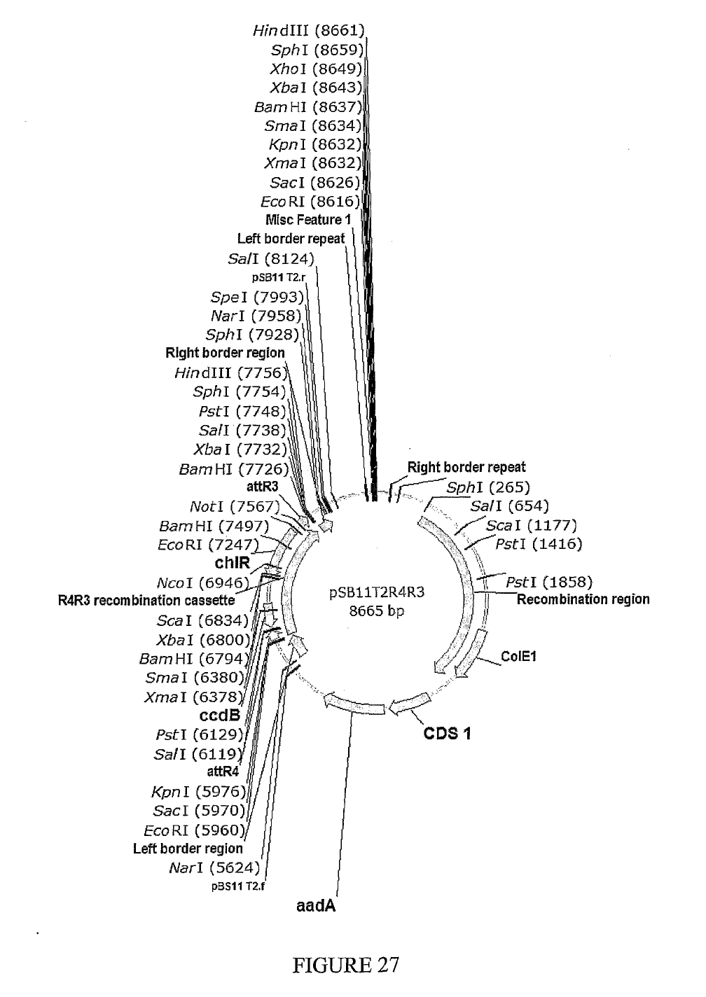

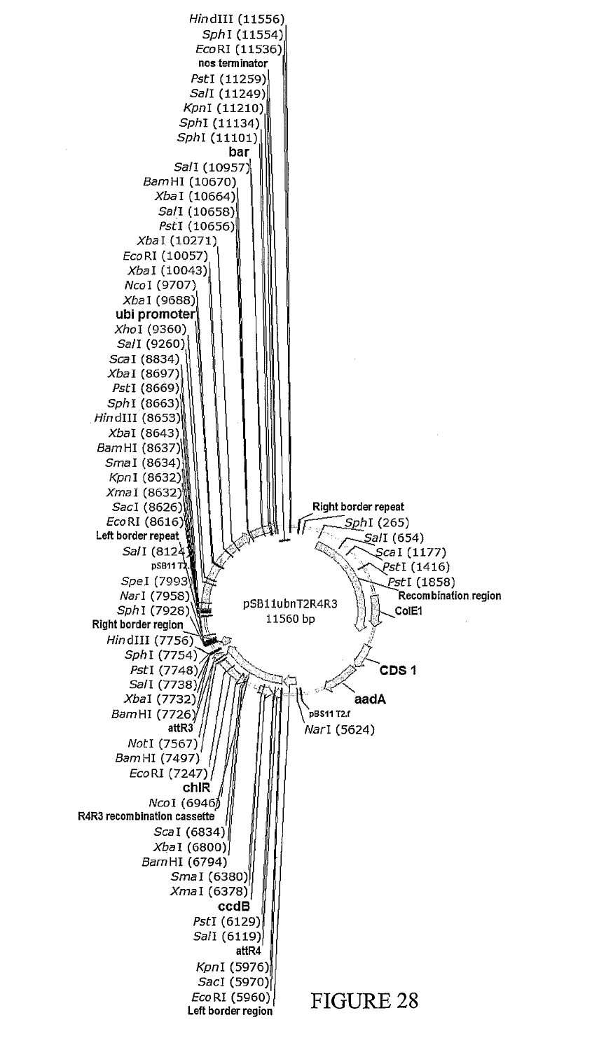

The present inventors also provide a super-binary donor base vector containing two separate T-DNAs to facilitate marker excision (FIG. 27). One T-DNA contains a multiple cloning site suitable for selectable marker cassettes (e.g. chimeric ubi::bar) and the other contains an R4R3 multi-site recombination cassette suitable for the chimeric act1D::gusA or act1D::sgfp. The ubi::bar-nos selectable marker cassette has been cloned into this base vector (FIG. 28).

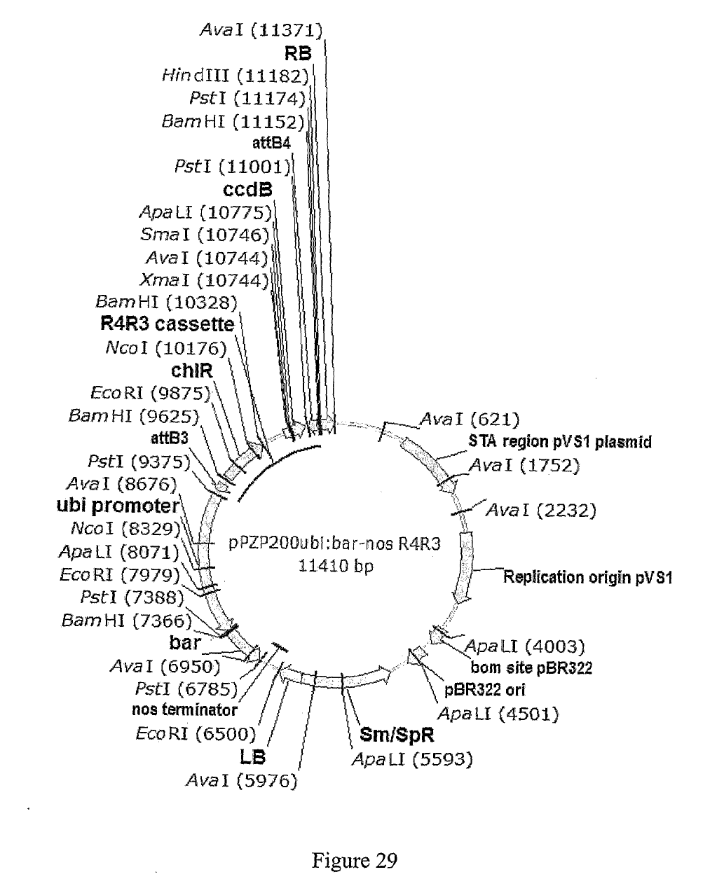

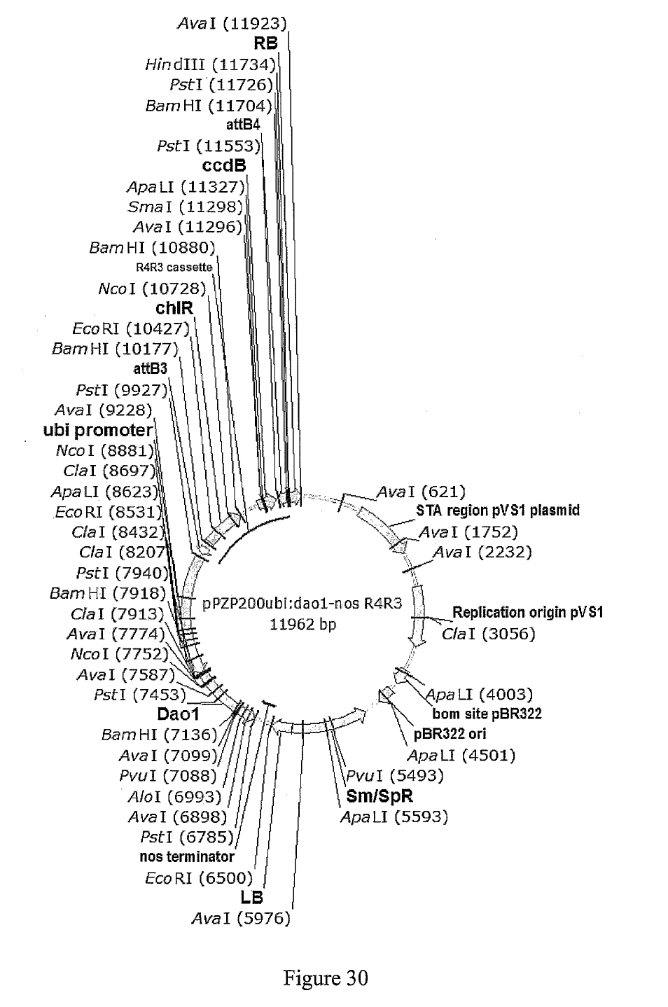

Furthermore, the present inventors provide two binary base vectors (FIGS. 29 and 30). The T-DNA contains a multiple cloning site, a chimeric selectable marker and an R4R3 multi-site recombination cassette. The vector pPZP200 ubi::bar-nos R4R3 vector (FIG. 29) comprises the bar gene in operable connection with the maize ubiquitin promoter in the multiple cloning site. The vector pPZP200 ubi::dao1-nos R4R3 (FIG. 30) comprises the D-amino oxidase gene (dao1) from the yeast R. gracilis in operable connection with the maize ubiquitin promoter in the multiple cloning site.

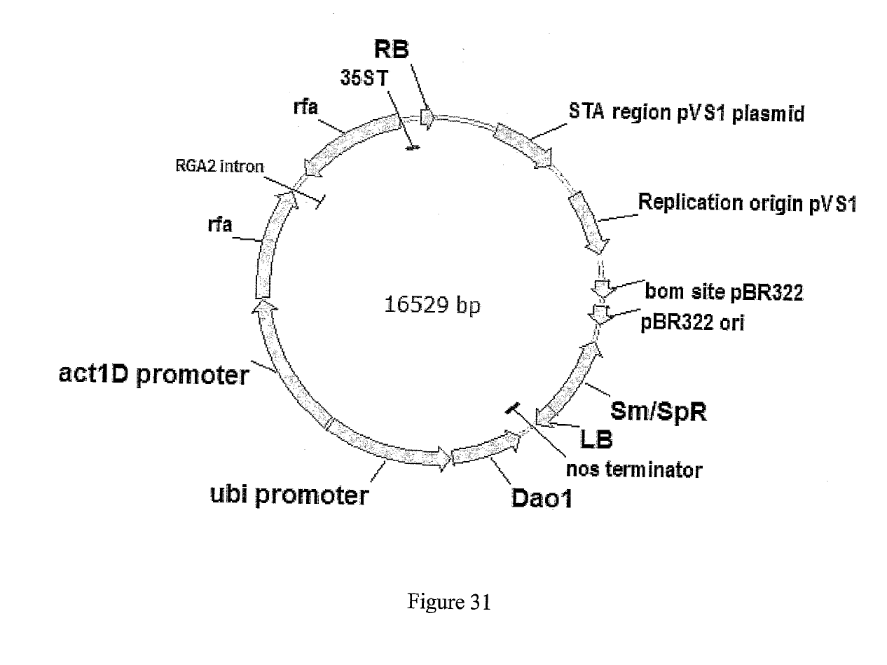

The present inventors additionally provide a binary base vector for the expression of an inhibitory RNA (e.g., RNAi) (as depicted in FIG. 31). This vector comprises a T-DNA comprising a ubi::dao1-nos selectable marker cassette. The vector additionally comprises rfa and rfa(as) recombination sites for cloning a nucleic acid in a sense and an antisense orientation for the expression of an RNAi molecule.

Methods for producing additional binary vectors are also described, for example, in each of the references described in Table 2.

3.3 Introducing a Nucleic Acid Construct into Bacterium

Methods for introducing a nucleic acid construct into bacteria are known in the art. For example, in one example, the nucleic acid construct is introduced into or transformed into bacteria using electroporation. In accordance with this embodiment, transformation-competent bacteria may be prepared using a method known in the art. The cells are then contacted with the nucleic acid construct and exposed to an electric pulse for a time and under conditions to disrupt the membrane of the cells. Following a suitable period of time to enable expression of a reporter gene functional in bacteria, those cells comprising an expression vector are selected, e.g., by growing the cells in the presence of an antibiotic. Methods for transforming bacteria using electroporation are known in the art and/or described in den Dulk-Ras and Hooykaas, Methods Mol. Biol. 55:63-72, 1995 or Tzfira et al., Plant Molecular Biology Reporter, 15: 219-235, 1997.

In another example, a nucleic acid construct is introduced into bacteria using tri-parental mating. Briefly, tri-parental mating comprises culturing three bacterial cell types together to facilitate transferal of the nucleic acid construct from one to another. For example, a nucleic acid construct is produced in E. coli. However, E. coli and, for example, A. tumefaciens are not able to mate. Accordingly a helper cell that is capable of mating with both cell types is used thereby facilitating mobilization or transferal of the nucleic acid construct from E. coli to A. tumefaciens.

In another example, the nucleic acid construct is introduced into bacteria using a freeze-thaw method, e.g., as described by Gynheung Methods in Enzymol., 153: 292-305, 1987). For example, bacteria are contacted with the nucleic acid construct and frozen, for example, using liquid nitrogen for a period of time, such as, for example, one minute. Cells are then cultured for a time and under conditions sufficient to induce expression of a selectable marker contained therein, and those cells comprising the construct selected.

### 3.4 Inoculation and Co-Culture of a Bacterium and an Embryo

In one example, the method of transformation comprises contacting the embryonic cells with a bacterium comprising a nucleic acid construct for a time and under conditions sufficient for said bacterium to bind to or attach to said embryonic cells (i.e., inoculation).

For example, the embryonic cells are completely or partially immersed in a culture medium in which bacteria comprising the nucleic acid construct have been grown for a time and under conditions sufficient for the bacteria to bind to or attach to said embryonic cells.

Accordingly, in one example, the embryonic cells are inoculated with a bacterium comprising a nucleic acid construct as described herein by performing a method comprising:
(i) growing said bacterium in a culture medium for a time and under conditions sufficient to produce a population of bacteria comprising the nucleic acid construct; and
(ii) completely or partially immersing the embryonic cells in said culture medium following growth of said population,
   wherein said embryonic cells are immersed in said medium for a time and under conditions sufficient for said bacteria to bind to or attach to said embryonic cells.

The skilled artisan will be aware of suitable conditions for inoculating a plant cell with bacteria.

For example, the embryonic cells are contacted with the bacteria for a period ranging from about 5 minutes (Cheng et al., In Vitro Cell Dev Biol-Plant. 39: 595-604, 2003) to about 2 days. More preferably, the embryonic cells are contacted with the bacteria for a period ranging from about 30-60 minutes (Weir et al., Aust J Plant Physiol. 28: 807-818, 2001) to about 1 day. Even more preferably, the embryonic cells are contacted with the bacteria for about 60 minutes to about 4 hours, more preferably for about 3 hours (as exemplified herein and/or described in Cheng et al., Plant Physiol. 115: 971-980, 1997).

Preferably, the inoculation is performed at a temperature of about 21° C. to about 28° C. More preferably, the inoculation is performed at a temperature of about 23° C. to about 26° C. Even more preferably, the inoculation is performed at about room temperature.

Generally, the inoculation is performed in a culture medium that supports growth and/or survival of both the embryonic cells and bacteria. Suitable culture media are known in the art and include, for example, Murashige and Skoog (Murashige and Skoog Physiol. Plant, 15: 473-497, 1962) or a dilution form thereof.

In one example, the embryonic cells are inoculated using a medium comprising a phenolic inducer, such as, for example, acetosyringone, coniferyl alcohol or syringaldehyde. In this respect, acetosyringone has been shown to markedly increased T-DNA delivery by Agrobacterium (Wu et al., Plant Cell Reports; 21:659-668, 2003). Preferably, graminaceous embryonic cells are inoculated using a medium comprising about 100 μM to about −500 μM acetosyringone. More preferably, embryonic cells are inoculated using a medium comprising about 200 μM to about 400 μM acetosyringone. Even more preferably, embryonic cells are inoculated using a medium comprising about 200 μM acetosyringone.

Surfactants have also been reported to increase T-DNA delivery by bacteria, for example, Agrobacterium (Wu et al., supra). Accordingly, in one example, embryonic cells are inoculated using a medium comprising a surfactant. Examples of suitable surfactants include, Silwet® (Monsanto) or Tween 20. Preferably, the medium comprises from about 0.01% surfactant to about 0.5% surfactant, more preferably, for about 0.1% to about 0.4% surfactant.

In one example, inoculation is performed in the dark. Alternatively, inoculation is performed under light.

The present inventors have also clearly demonstrated that inoculation performed in the presence of a bacterial nitrogen source dramatically increases the transformation efficiency of mature embryonic cells. Accordingly, in one example of the invention, the graminaceous embryonic cells are inoculated using a medium comprising a bacterial nitrogen source. For example, a suitable nitrogen source is an enzymatic digest of a protein extract from a plant or animal or a water soluble fraction produced by partial hydrolysis of an extract from a plant or an animal, e.g., a peptone. For example, the peptone is from a plant, such as, for example, soybean, broadbean, wheat or potato. Alternatively, the peptone is from an animal or animal product, such as, for example, porcine skin, meat or casein. Suitable commercial sources of peptones will be apparent to the skilled artisan and include, for example, Sigma Aldrich, Organo Technie, GE Healthcare or Novogen.

In one example, the graminaceous embryonic cells are inoculated using a medium comprising a soybean peptone (e.g., Soytone™). For example, the graminaceous embryonic cells are inoculated using a medium comprising from about 0.001% to about 0.1% peptone (w/v), more preferably from about 0.01% to about 0.05% peptone (w/v) and more preferably about 0.02% peptone (w/v). For example, the graminaceous embryonic cells are inoculated using a medium comprising 0.02% soybean peptone (w/v).

Without being bound by theory or mode of action, the increased transformation efficiency in the presence of a peptone may be a result of increased production of cellulose microfibrils by the bacteria, e.g., Agrobacterium thereby increasing the ability of said bacteria to bind to the plant embryonic cells. Accordingly, in one example, the embryonic graminaceous plant cells are inoculated using a medium comprising a compound that induces production of a cellulose microfibril by a bacterium, e.g., a soil-borne bacterium, preferably an Agrobacterium.

Following inoculation, culture medium and any unbound bacteria are generally removed using, for example, a vacuum or by pipetting. Embryonic graminaceous plant cells are then co-cultured with bacteria bound thereto following inoculation. Accordingly, in one example, the method of the invention comprises maintaining the embryonic cells and the bacteria comprising the nucleic acid construct under conditions sufficient for said bacteria to infect a cell of said embryonic cells or for said bacteria to thereby introduce a transfer-nucleic acid from said nucleic acid construct into a cell of said embryonic cells.

The skilled artisan will be aware of suitable conditions for co-culturing a plant cell with bacteria.

For example, the embryonic graminaceous plant cells and bound bacteria are maintained in or on a culture medium suitable for growth and/or survival of said embryonic graminaceous plant cells and bound bacteria for a period of time ranging from about 1 day to about 5 days (Wu et al., Plant Cell Reports. 21: 659-668, 2003). Preferably, the embryonic graminaceous plant cells and bound bacteria are co-cultured for a period from about 2 days to about 3 days (Weir et al., supra). In an example of the invention, the embryonic graminaceous plant cells and bound bacteria are co-cultured for a period of about 3 days.

The vir genes required for successful transformation mediated by a bacterium, e.g., Agrobacterium, are optimally expressed at a temperature less than about 28° C. (Mörbe et al., Molecular Plant-Microbe Interactions, 2: 301-308, 1989. Accordingly, co-cultivation is preferably performed at a temperature less than about 28° C. Preferably, the co-cultivation is performed at a temperature ranging from about 23° C. to about 28° C. More preferably, the temperature ranges from about 23° C. to about 26° C. More preferably, the co-cultivation is performed at room temperature.

Alternatively, the co-cultivation is performed at a plurality of temperatures. For example, co-cultivation is performed at about 27° C. for one day and at about 22° C. for about 2 days (Khanna and Daggard, Plant Cell Reports. 21: 429-436, 2003).

The vir genes required for successful transformation mediated by a bacterium, such as, Agrobacterium, are optimally expressed when bacteria are grown under acid conditions (Mörbe et al., supra). Accordingly, co-cultivation is preferably performed under acidic conditions. For example, co-cultivation is performed at a pH less than about pH 6.5, more preferably, less than about pH 6, more preferably, less than about pH 5.5.

In one example, the co-cultivation is performed in the presence of a phenolic inducer (e.g., acetosyringone) and/or a surfactant. Suitable surfactants and/or concentrations of acetosyringone or surfactant are described supra, and are to be taken to apply mutatis mutandis to the present embodiment of the invention.

In one example, the co-cultivation is performed in the presence of a phenolic inducer and glycine betaine. The inclusion of glycine betaine has been shown to enhance induction of Agrobacterium vir genes in the presence of acetosyringone (Vernade et al., J. Bacteriol., 170: 5822-5829, 1988).

Other factors shown to increase expression of vir genes and/or increase transformation efficiency include, for example, sugar (e.g., sucrose) (Andenbauer et al., Journal of Bacteriology 172: 6442-6446, 1990). Accordingly, in an example of the invention, the co-culture is performed in the presence of sucrose, e.g., from about 0.1% sucrose to about 4% sucrose, more preferably from about 0.2% sucrose to about 2% sucrose.

The present inventors have also clearly demonstrated increased transformation efficiency when co-cultivation is performed in the presence of a peptone. In this respect, suitable peptones are described supra, and are to be taken to apply mutatis mutandis to this embodiment of the invention.

In one example, inoculation and/or co-culture are performed under conditions sufficient to select for bacteria comprising the nucleic acid construct. For example, as described supra, it is preferable to include a selectable marker that is active in bacteria in a nucleic acid construct. Alternatively, several bacterial strains comprise a selectable marker and inoculation and/or co-culture is performed, for example, using an antibiotic to which the bacterium is resistant (and that does not inhibit or prevent the growth and/or survival of the embryonic cells).

Roberts et al., (Proc. Natl. Acad. Sci. USA, 100: 6634-6639, 2003) demonstrated that the inhibition of purine synthesis in plants prior to bacterium mediated transformation increased transformation efficiencies. Accordingly, in one example, embryonic graminaceous plant cells are contacted with a compound that inhibits purine synthesis prior to inoculation and/or co-culture. In this: respect, it is preferable that the purine synthesis inhibitor is not washed from the embryonic graminaceous plant cells prior to inoculation. Suitable purine synthesis inhibitors will be apparent to the skilled artisan and include, for example, azaserine or acivicin or mizoribine.

In another example, the embryonic graminaceous plant cells are wounded prior to inoculation and/or co-cultivation with a bacterium. Bidney et al., (Plant Mol. Biol., 18: 301-313, 1992) showed that wounding using microparticle bombardment dramatically increased transformation efficiency compared to unwounded cells. Suitable methods for wounding embryonic graminaceous cells will be apparent to the skilled artisan.

Following co-culture it is preferred to remove any bacteria that remain bound to the embryonic graminaceous plant cells. This may be achieved, for example, by washing the embryonic cells. Preferably, the embryonic cells are washed with a solution comprising, for example, an antibiotic that is toxic to α-bacterium, such as, Agrobacterium but is not toxic to a plant cell. For example, the embryonic cells are washed with cefotaxime or carbenicillin (Matthias and Boyd, Plant Sci. 46: 217-233, 1986).

### 4. Regeneration of Transgenic Plants or Parts Thereof

### 4.1 Callus Induction

In an example, a plant or a plant part or a plantlet is regenerated using the transformed embryonic graminaceous plant cells produced using a method described herein.

Preferably, a transformed graminaceous embryonic cell is contacted with a compound that induces callus formation for a time and under conditions sufficient for callus formation.

Alternatively, or in addition, a transgenic embryonic graminaceous plant cell is contacted with a compound that induces cell de-differentiation for a time and under conditions sufficient for a cell to de-differentiate. Alternatively, or in addition, a transgenic embryonic graminaceous plant cell is contacted with a compound that induces growth of an undifferentiated cell for a time and under conditions sufficient for an undifferentiated cell to grow.

Compounds that induce callus formation and/or induce production of undifferentiated and/or de-differentiated cells will be apparent to the skilled artisan and include, for example, an auxin, e.g., 2,4-D, 3,6-dichloro-o-anisic acid (dicambia), 4-amino-3,5,6-thrichloropicolinic acid (picloram) or thidiazuron (TDZ).

In this respect, a transformed embryonic cell is preferably maintained on a callus inducing or promoting medium.

Such a medium may additionally comprise one or more compound that facilitates callus formation/de-differentiation or growth of undifferentiated cells. For example, Mendoza and Kaeppler (In vitro Cell Dev. Biol., 38: 39-45, 2002) found that media comprising maltose rather than sucrose enhanced the formation of calli in the presence of 2,4-D.

Alternatively, or in addition, the embryonic cell is additionally contacted with myo-inositol. Studies have indicated that myo-inositol is useful for maintaining cell division in a callus (Biffen and Hanke, Biochem. J. 265: 809-814, 1990).

Similarly, casein hydrolysate appears to induce cell division in a callus and maintain callus morphogenetic responses. Accordingly, in another example, the embryonic graminaceous plant cell is additionally contacted with casein hydrolysate.

Suitable culture medium and methods for inducing callus formation and/or cell de-differentiation and/or the growth of undifferentiated cells from mature embryonic graminaceous plant cells are known in the art and/or described in Mendoza and Kaeppler, In vitro Cell Dev. Biol., 38: 3945, 2002, Özgen et al., Plant Cell Reports, 18: 331-335, 1998; Patnaik and Khurana BMC Plant Biology, 3: 1-11, Zale et al., Plant Cell, Tissue and Organ Culture, 76: 277-281, 2004 and Delporte et al., Plant Cell, Tissue and Organ Culture, 80: 139-149, 2005.

4.2 Shoot and/or Root Formation

Following callus induction, cell de-differentiation and/or growth of undifferentiated cells, the embryonic graminaceous plant cells and/or a cell derived therefrom (e.g., a callus derived therefrom or a de-differentiated or undifferentiated cell thereof) is contacted with a compound that induces shoot formation for a time and under conditions sufficient for a shoot to develop. Suitable compounds and methods for inducing shoot formation are known in the art and/or described, for example, in Mendoza and Kaeppler, In vitro Cell Dev. Biol., 38: 39-45, 2002, Özgen et al., Plant Cell Reports, 18: 331-335, 1998, Patnaik and Khurana BMC Plant Biology, 3: 1-11, Zale et al., Plant Cell, Tissue and Organ Culture, 76: 277-281, 2004, Murashige and Skoog, Plant Physiol., 15: 473-479, 1962 or Kasha et al., (In: Gene manipulation in plant improvement II, Gustafson ed., Plenum Press, 1990).

For example, a callus or an undifferentiated or de-differentiated cell is contacted with one or more plant growth regulator(s) that induces shoot formation. Examples of suitable compounds (i.e., plant growth regulators) include indole-3-acetic acid (IAA), benzyladenine (BA), indole-butyric acid (IBA), zeatin, a-naphthaleneacetic acid (NAA), 6-benzyl aminopurine (BAP), thidiazuron, kinetin, 2iP or combinations thereof.

Suitable sources of media comprising compounds for inducing shoot formation are known in the art and include, for example, Sigma.

Alternatively, or in addition, the callus or an undifferentiated or de-differentiated cell is maintained in or on a medium that does not comprise a plant growth modulator for a time and under conditions sufficient to induce shoot formation and produce a plantlet.

At the time of shoot formation or following shoot formation the callus or an undifferentiated or de-differentiated cell is preferably contacted with a compound that induces root formation for a time and under conditions sufficient to initiate root growth and produce a plantlet.

Suitable compounds that induce root formation are known to the skilled artisan and include a plant growth regulator, e.g., as described supra.

Suitable methods for inducing root induction are known in the art and/or described in Mendoza and Kaeppler, In vitro Cell Dev. Biol., 38: 39-45, 2002, Özgen et al., Plant Cell Reports, 18: 331-335, 1998, Patnaik and Khurana BMC Plant Biology, 3: 1-11, Zale et al., Plant Cell, Tissue and Organ Culture, 76: 277-281, 2004, Murashige and Skoog, Plant Physiol., 15: 473-479, 1962 or Kasha et al., (In: Gene manipulation in plant improvement-II, Gustafson ed., Plenum Press, 1990).

In an example of the invention, a callus and/or de-differentiated cell and/or undifferentiated cell is contacted with media comprising zeatin for a time and under conditions sufficient to induce shoot formation and contacted with medium comprising NAA for a time and under conditions sufficient to induce root formation.

Plantlets are then grown for a period of time sufficient for root growth before being potted (e.g., in potting mix and/or sand) and being grown.

### 4.3 Selection

During plant regeneration it is preferable to apply a selection to the transformed embryonic cells to thereby reduce bacterial, e.g., Agrobacterium growth and to prevent growth of a plant cell that does not comprise a nucleic acid construct. To facilitate such selection, it is preferable that the nucleic acid construct comprises a nucleic acid encoding a suitable selectable marker. Suitable selectable markers are known in the art and/or described herein.

For example, the selectable marker confers resistance to an antibiotic or a herbicide when expressed. During callus induction and/or plant regeneration, the transformed embryonic graminaceous plant cells are contacted with said antibiotic or herbicide. As a consequence, only those cells expressing said selectable marker will survive and/or grow in the presence of the selectable marker, thereby producing a transgenic plant (e.g., a clonal transformant).

In one example, the selectable marker facilitates growth of a plant or plant cell in the presence of a compound that is toxic to a non-transformed cell or plant. Preferably, the selectable marker gene encodes a protein that facilitates growth of a plant in the presence of a D-amino acid oxidase. For example, the selectable marker gene encodes a D-amino acid oxidase (DAAO), e.g., as described herein. Other suitable selectable marker genes will be apparent to the skilled artisan ad/or described herein and/or described in Published International Application No. WO2003/060133. For example, the selectable marker gene expresses a protein selected from the group consisting of a D-serine ammonia lyase, a D-glutamate oxidase, a D-aspartate oxidase, a D-glutamate racemase and a D-alanine transaminase. A plant or plant cell expressing such a selectable marker gene is capable of metabolizing a D-amino acid, such as, for example, D-alanine or D-serine. In contrast, a plant or plant cell that does not express the selectable marker gene is unable to grow in the presence of such a D-amino acid. In fact, at some concentrations a D-amino acid is toxic to a plant or plant cell that does not express a suitable selectable marker gene. To select a transgenic cell and/or plant, a transformed embryonic graminaceous plant cell is contacted with a D-amino acid, e.g., D-alanine and/or D-serine, for a time and under conditions to prevent an untransformed cell from growing or to induce said cell to die. For example, the cell or callus or plant is maintained in the presence of at least about 2 mM D-amino acid or at least about 3 mM D-amino acid or at least about 4 mM D-amino acid or at least about 5 mM D-amino acid. Such selection is applied, for example, during callus induction and/or during plant regeneration.

In another example, a cell or callus comprising the nucleic acid construct is identified, e.g., by detecting a detectable marker expressed by said construct. Suitable detectable markers are described herein. For example, a callus expressing the dsRED marker is detected, isolated (e.g., by excision) and used to regenerate a transgenic plant.

In one example, the selection of a transformed cell is performed at the time of callus induction and/or plant regeneration.

In another example, selection of a transformed cell is commenced following commencement of plant regeneration. For example, selection is commenced approximately 2 weeks or 3 weeks or 4 weeks or 5 weeks after the commencement of callus induction.

In one example, a cell that is or is likely to have been transformed using the method of the invention is isolated. In this respect, the method of the invention generally results in nucleic acid being incorporated into the epiblast and/or scutellum of an embryo. Accordingly, in one example, the method of the invention comprises isolating an epiblast and/or scutellum cell prior to callus induction, during callus induction and/or during plant regeneration. Such a cell is then used to regenerate a transgenic plant.

An epiblast cell or scutellum cell that comprises the nucleic acid construct may be identified by detecting a detectable marker expressed by said construct (e.g., dsRED) and said cell isolated and used to regenerate a plant.

### 4.4 Plant Breeding

The regenerated transformed plants may be propagated by any of a variety of means, such as by clonal propagation or classical breeding techniques. For example, a first generation (or T1) transformed plant is selfed to produce a homozygous second generation (or T2) transformant, and the T2 plants further propagated through classical breeding techniques. Alternatively, the first generation is bred by classical breeding techniques to produce hemizygous plants which are then interbred to produce homozygous plants.

The regenerated transformed plants contemplated herein may take a variety of forms. For example, they may be chimeras of transformed cells and non-transformed cells or clonal transformants (e.g., all cells transformed to contain the transfer-nucleic acid or transgene).

In one example a regenerated transformed plant or progeny thereof is grown to maturity and a seed or propagating material (e.g., reproductive tissue) obtained from the mature plant.

The present invention clearly contemplates the progeny of a plant produced using the method of the invention, and/or the seed or germplasm or propagating material of a plant produced according to the present invention. Methods for producing such progeny, seed, germplasm or propagating material will be apparent to the skilled artisan based on the description herein.

### 5. Examples of a Method for Producing a Transgenic Graminaceous Cell or Regenerating a Plant

In one example, the present invention provides a method for producing a transgenic graminaceous cell, said method comprising:

(i) obtaining embryonic cells from a dried graminaceous grain, for example, a wheat grain or a barley grain or a rice grain or a maize grain
(ii) removing the seed coat and/or aleurone from the embryonic cells;
(iii) contacting the embryonic cells with an Agrobacterium comprising a nucleic acid construct that comprises transfer-nucleic acid to be introduced into the embryonic cells for a time and under conditions sufficient for said Agrobacterium to bind to or attach to said embryonic cells, wherein said contacting is performed in the presence of a peptone and wherein said contacting is performed without first inducing callus formation from said embryonic cells; and
(iv) maintaining the embryonic cells and the bound Agrobacterium for a time and under conditions sufficient for said Agrobacterium to introduce the transfer-nucleic acid into one or more cells thereof wherein said maintaining is performed in the presence of a peptone,
thereby producing a transgenic graminaceous cell.

For example, the method comprises:

(i) excising embryonic cells from a dried graminaceous grain, for example, a wheat grain or a barley grain or a rice grain or a maize grain
(ii) removing the seed coat and/or aleurone from the embryonic cells, e.g., by excision;
(iii) contacting the embryonic cells with an Agrobacterium tumefaciens comprising a nucleic acid construct that comprises transfer-nucleic acid to be introduced into the embryonic cells for at least about 3 hours and in the presence of acetosyringone, a peptone from soybean and 2,4-dichlorophenoxyacetic acid, wherein said contacting is performed without first inducing callus formation from said embryonic cells; and
(iv) maintaining the embryonic cells and the bound Agrobacterium for at least about 3 days in the presence of acetosyringone and 2,4-dichlorophenoxyacetic acid to thereby permit the Agrobacterium to introduce the transfer-nucleic acid into one or more of the embryonic cells,
thereby producing a transgenic graminaceous cell.

In accordance with each of the previous embodiments, it is preferred that the step of contacting the embryonic cells with an Agrobacterium is performed in the presence of about 0.01% to about 0.04% (w/v) peptide, for example, in the presence of about 0.02% of peptone.

It is also preferred that the steps of contacting the embryonic cells with an Agrobacterium and maintaining the embryonic cells and the bound Agrobacterium are performed in the presence of from about 1 mg/L 2,4-D to about 4 mg/L 2,4-D, for example, about 2 mg/L 2,4-D.

It is also preferred that the steps of contacting the embryonic cells with an Agrobacterium and maintaining the embryonic cells and the bound Agrobacterium are performed in the presence of from about 100 μM acetosyringone to about 400 μM acetosyringone, for example, about 200 μM acetosyringone.

The present invention also provides a method for regenerating a plant from a plant cell. For example, such a method comprises:

(a) contacting a plant cell with a compound that induces callus formation for a time and under conditions sufficient to produce a callus;
(b) contacting the callus with a compound that induces shoot formation for a time and under conditions sufficient for a shoot to develop;
(c) contacting the callus with a compound that induces root formation for a time and under conditions sufficient to initiate root growth, thereby producing a plantlet; and
(d) growing the plantlet for a time and under conditions sufficient to produce a plant.

For example, the method comprises:

(i) contacting a transgenic cell with a solution comprising 2,4-D or Dicambia or TDZ and picloram such that a callus is produced; and
(ii) contacting the callus produced at (i) with a solution comprising zeatin and/or TDZ such that a shoot develops;
(iii) contacting the shoot produced at (ii) with a solution comprising a naphthaleneacetic acid such that root growth commences, thereby producing a plantlet;

For example, the transgenic cell is contacted with a solution comprising from about 1 mg/L 2,4-D to about 4 mg/L 2,4-D, for example, about 2 mg/L 2,4-D. Alternatively, the transgenic cell is contacted with a solution comprising from about 2 mg/L Dicambia to about 8 mg/L Dicambia, for example, about 4 mg/L Dicambia. Alternatively, the transgenic cell is contacted with a solution comprising from about 1 mg/L TDZ to about 6 mg/L TDZ and about 1 mg/L picloram to about 4 mg/L picloram, for example, about 3 mg/L TDZ and about 2 mg/L picloram.

In another example, the callus is contacted with a solution comprising from about 1 mg/L zeatin to about 4 mg/L zeatin, for example, about 2 mg/L zeatin. Alternatively, the callus is contacted with a solution comprising from about 0.25 mg/L TDZ to about 2 mg/L TDZ, for example, about 1 mg/L TDZ.

In a further example, the shoot is contacted with a solution comprising from about 0.25 mg/L NAA to about 2 mg/L NAA, for example, about 1 mg/L NAA.

Additional suitable compounds will be apparent to the skilled artisan based on the description herein and shall be take to apply mutatis mutandis to the present embodiment of the invention.

As will be apparent to the skilled artisan, any of the methods for regenerating a plant discussed in the previous paragraphs is also useful for regenerating a transgenic plant, e.g., from a transgenic cell produced according to a method described herein according to any embodiment.

### 6. Modulation of a Plant Phenotype

As will be apparent to the skilled artisan from the foregoing, the present invention provides a method for expressing a transgene or modulating the expression of a gene in a graminaceous plant. For example, such a method comprises:
(i) producing a transgenic graminaceous plant cell using a method described herein according to any embodiment, wherein said transgenic cell comprises a transgene operably linked to a promoter operable in a graminaceous plant cell;
(ii) regenerating a transgenic plant from said cell; and
(ii) maintaining said transgenic plant for a time and under conditions sufficient for said transgene to be expressed.

In another example, the invention provides a method for modifying expression of a nucleic acid in a graminaceous plant, said method comprising:
(i) producing a transgenic graminaceous plant cell using a method according to any embodiment, wherein said transgenic cell comprises a transgene capable of modulating the expression of the nucleic acid;
(ii) regenerating a plant from said transgenic cell; and
(ii) maintaining said transgenic plant for a time and under conditions sufficient to modulate expression of said nucleic acid.

Clearly, such a method is useful for, for example, modulating a phenotype of a plant or plant cell, e.g., by expressing a gene that confers a desirable phenotype or by suppressing expression of a gene that confers an undesirable phenotype.

### 6.1 Expression a Transgene in a Plant

In one example, the present invention provides a method for modulating a phenotype in a plant or a seed thereof or propagating material thereof, said method comprising expressing a transgene that modulates said phenotype in the plant seed or propagating material using a method described herein according to any embodiment. Alternatively, the method comprises enhancing or inducing or conferring a characteristic on a plant.

For example, the present invention provides a method for producing a graminaceous plant having an improved nutritional quality, said method comprising:
(i) transforming a graminaceous plant cell with a nucleic acid construct that comprises a transgene that encodes a protein associated with an improved nutritional quality by performing a method described herein according to any embodiment;
(ii) regenerating a transgenic plant from said cell; and
(ii) maintaining said transgenic plant for a time and under conditions sufficient for said transgene to be expressed, thereby producing a plant having an improved nutritional quality.

Alternatively, the present invention provides a method for producing a graminaceous plant expressing a pharmaceutically useful protein or nutraceutically useful protein, said method comprising:
(i) transforming a graminaceous plant cell with a nucleic acid construct that comprises a transgene that encodes a pharmaceutically useful protein or nutraceutically useful protein by performing a method described herein according to any embodiment;
(ii) regenerating a transgenic plant from said cell; and
(ii) maintaining said transgenic plant for a time and under conditions sufficient for said transgene to be expressed, thereby producing a plant expressing a pharmaceutically useful protein or nutraceutically useful protein.

Preferably, the method of the invention additionally comprises producing or providing an expression construct comprising the transgene and/or producing and/or providing a bacterium, e.g., an Agrobacterium comprising said expression construct. In this respect, the skilled artisan will be aware of suitable methods for producing such an expression construct and/or a bacterium comprising such a nucleic acid construct based on the description herein.

Clearly, the present invention also encompasses a graminaceous plant or progeny thereof or seed thereof or germplasm thereof having an improved nutritional or pharmaceutical quality. For example, a graminaceous plant, progeny, seed or germplasm produced according to a method described herein according to any embodiment.

For example, the present invention is useful for producing a transgenic plant that expresses a pharmaceutically, immunologically or nutritionally useful protein, or an enzyme that is required for production of a pharmaceutically, immunologically or nutritionally useful secondary product, or a protein capable of modifying the utilization of a substrate in a secondary metabolic pathway. Such proteins are known to those skilled in the art and include, for example, a range of structurally and functionally diverse antigenic proteins (e.g., an antigenic protein derived from a pathogen that infects a human or animal to be fed on a product of the grain), a sulphur-rich protein (e.g., Brazil Nut Protein, sunflower seed albumin, 2S protein, Asp I synthetic protein), a calcium-binding protein (e.g., calmodulin, calreticulin, or calsequestrin), an iron-binding protein (e.g., hemoglobin), and a biosynthetic enzyme that is required for the production of an osmoprotectant such as betaine (e.g., choline oxidase, betaine aldehyde dehydrogenase), a fatty acid (e.g., delta-12 desaturase), a phytosterol (e.g., S-adenosyl-L-methionine-Δ²⁴-sterol methyl transferases (SMT_{I} or SMT_{II}), a C-4 demethylase, a cycloeucalenol to obtusifoliol-isomerase, a 14α-methyl demethylase, a ΔA⁸ to Δ⁷-isomerase, a Δ⁷-sterol-C-5-desaturase, or a 24,25-reductase), an anthocyanin or other pigment (proanthocyaninidin reductase), lignin (e.g., cinnamoyl alcohol dehydrogenase, caffeic acid O-methyl-transferase, or phenylalanine ammonia lyase), an anti-nutritional protein, an enzyme capable of altering a substrate in the phenylpropanoid pathway (e.g., choline oxidase, betaine aldehyde dehydrogenase, ferulic acid decarboxylase), a choline metabolizing enzyme capable of acting upon choline to modify the use of choline by other enzymes in the phenylpropanoid pathway (e.g., choline oxidase, betaine aldehyde dehydrogenase, ferulic acid decarboxylase), an enzyme involved in the malting process (e.g., high pI α-amylase, low pI α-amylase, EII-(1-3, 1-4)-p-glucanase, Cathepsin β-like proteases, α-glucosidase, xylanase or arabinofuranosidase), an enzyme capable of acting upon a sugar alcohol, or an enzyme capable of acting upon myo-inositol, etc. Nucleic acids encoding such proteins are publicly available and/or described in the scientific literature. The structures (e.g., sequence) of such nucleic acids and their encoded proteins are fully described in the database of the National Center for Biotechnology Information of the US National Library of Medicine, 8600 Rockville Pike, Bethesda, Md. 20894, USA. As will be apparent to the skilled artisan, such a nucleic acid is a suitable transgene for use in the method of the present invention.

In one exemplified embodiment, the method of the invention is used to produce a transgenic plant expressing a hybrid high molecular weight glutenin subunit (HMW-GS) under control of native HMW-GS regulatory sequences, e.g., as described in Blechl and Anderson Nature Biotechnology, 14: 875-879, 1996.

Alternatively, or in addition, a transgene encoding the HMW-GS 1Ax1 gene (SEQ ID NO: 40) is introduced into a wheat cell using the method of the invention as described herein according to any embodiment, which is then used to produce a transgenic wheat plant. Preferably, the HMW-GSAx1 gene is placed operably under control of its endogenous promoter in the nucleic acid construct. By increasing the level of HMW-GS in a wheat grain, the elasticity of dough produced using the wheat is enhanced thereby enhancing the breadmaking properties of the flour from the wheat.

Grain from graminaceous plants is also widely used as an animal feed for non-ruminant animals. The phytase of Aspergillus niger (SEQ ID NO: 42) is used as a supplement in animal feeds to improve the digestability and also improve the bioavailability of phosphate and minerals. In one example, the method of the invention is used to produce a transgenic graminaceous plant that expresses the phyA gene from A. niger constitutively, or in the endosperm of the grain or seed.

In another example, the method of the invention is used to produce a graminaceous plant that expresses a therapeutic protein, such as, for example, a vaccine or an antibody fragment. Improved ‘plantibody’ vectors (e.g., as described in Hendy et al. J. Immunol. Methods 231:137-146, 1999) and purification strategies render such a method a practical and efficient means of producing recombinant immunoglobulins, not only for human and animal therapy, but for industrial applications as well (e.g., catalytic antibodies). Moreover, plant produced antibodies have been shown to be safe and effective and avoid the use of animal-derived materials and therefore the risk of contamination with a transmissible spongiform encephalopathy (TSE) agent. Furthermore, the differences in glycosylation patterns of plant and mammalian cell-produced antibodies have little or no effect on antigen binding or specificity. In addition, no evidence of toxicity or HAMA has been observed in patients receiving topical oral application of a plant-derived secretory dimeric IgA antibody (see Larrick et al. Res. Immunol. 149:603-608, 1998).

Various methods may be used to express recombinant antibodies in transgenic plants. For example, antibody heavy and light chains can be independently cloned into a nucleic acid construct, followed by the transformation of plant cells in vitro using the method of the invention. Subsequently, whole plants expressing individual chains are regenerated followed by their sexual cross, ultimately resulting in the production of a fully assembled and functional antibody (see, for example, Hiatt et al. Nature 342:76-87, 1989). In various examples, signal sequences may be utilized to promote the expression, binding and folding of unassembled antibody chains by directing the chains to the appropriate plant environment.

In this respect, a nucleic acid encoding an antibody fragment, e.g., the heavy and light chain of an antibody of interest is cloned into an expression construct described herein. The construct is then introduced into a bacterium, which is then use to produce a transgenic plant expressing the antibody fragment. Such a fragment may then be isolated from the plant, e.g., from a seed, using standard methods.

In another example, a peptide or polypeptide capable of eliciting an immune response in a host is expressed in a plant. For example, a transgene encoding Hepatitis B surface antigen (SEQ ID NO: 44) is inserted into a nucleic acid construct described herein and used to produce a transgenic graminaceous plant using a method described herein according to any embodiment. In accordance with this embodiment, a food product produced using the graminaceous plant or a part thereof (e.g., the bran from wheat) is then administered to humans (e.g., fed to a human) as a medicinal foodstuff or oral vaccine.

In another example, the method of the invention is used to produce a male sterile plant to thereby facilitate production of hybrid plants. In this respect, a male sterile plant is unable to self-fertilize thereby facilitating the production of plant lines. For example, a nucleic acid construct is produced that comprises a barnase transgene (SEQ ID NO: 46) under control of a suitable promoter (e.g., a tapetum specific promoter). The construct is then introduced into a bacterium and a transgenic graminaceous plant produced using a method described herein according to any embodiment. The expression of this gene prevents pollen development at specific stages of anther development thereby producing a male sterile plant.

In a further example, the method of the invention is used to produce a transgenic plant having resistance to a biotic stress (e.g., a fungal pathogen). Accordingly, in another example, the present invention provides a method for producing a transgenic graminaceous plant having resistance to a biotic stress, said method comprising:
(i) producing a transgenic graminaceous plant cell comprising a transgene that encodes a protein that confers or enhances resistance to a biotic stress using a method described herein according to any embodiment;
(ii) regenerating a transgenic plant from said cell; and
(ii) maintaining said transgenic plant for a time and under conditions sufficient for said transgene to be expressed, thereby producing a transgenic graminaceous plant having resistance to a biotic stress.

In one example, the method described supra applies mutatis mutandis to a method for improving or enhancing the resistance of a plant to a biotic stress.

In another example, the biotic stress is a plant pathogen, such as, for example, a fungus, a virus, a bacterium, or an insect that feeds on a graminaceous plant or a part of a graminaceous plant (e.g., a seed or grain of a graminaceous plant). Proteins that confer resistance to such a plant pathogen are known to those skilled in the art and include, for example, a range of structurally and functionally diverse plant defense proteins or pathogenesis-related proteins (e.g., chitinase, in particular acid chitinase or endochitinase; β-glucanase in particular β-1,3-glucanase; ribosome-inactivating protein (RIP); γ-kafirin; wheatwin or WPR4); thionin, in particular γ-thionin; thaumatin or thaumatin-like protein such as zeamatin; a proteinase inhibitor such as, for example, trypsin or chymotrypsin; or sormatin), virus coat proteins, and proteins that convert one or more pathogen toxins to non-toxic products. Nucleic acids encoding such proteins are publicly available and/or described in the scientific literature. The structures (i.e., sequence) of such nucleic acids and their encoded proteins are fully described in the database of the National Center for Biotechnology Information of the US National Library of Medicine, 8600 Rockville Pike, Bethesda, Md. 20894, USA. Such nucleic acids are suitable transgenes for use in the method of the present invention.

For example, a nucleic acid construct is produced that encodes a coat protein of wheat streak mosaic virus (SEQ ID NO: 48) that is then used to produce a transgenic wheat plant. Preferably, the gene is expressed in the seed of wheat, however, constitutive expression is also contemplated. Such expression confers resistance against wheat stripe mosaic virus.

In another example, a protein that confers or enhances resistance of a wheat plant to Fusarium graminearum (head scab) is used in the production of a wheat plant using a method described herein according to any embodiment. In accordance with this embodiment, the protein conferring or enhancing protection against F. graminearum is selected from the group consisting of: (i) a wheat thaumatin-like protein that confers protection against the fungal pathogen Fusarium graminearum (head scab) in wheat (i.e. SEQ ID NO: 50); (ii) a modified ribosomal protein L3 of wheat (i.e. wRPL3:Cys 258; SEQ ID NO: 52) that is resistant to the action of a trichothecene produced by F. graminearum; and (iii) a polypeptide having trichothecene O-acetyl transferase activity and capable of converting trichothecene produced by F. graminearum into a non-toxic product (i.e. SEQ ID NO: 54).

Alternatively, a chitinase gene from barley is used in the production of a transgenic wheat plant having resistance against Erisiphe graminis.

Alternatively, a killer protein from Ustilago maydis infecting virus is used in the production of transgenic wheat having resistance against Tilletia tritici.

Alternatively, a barley trypsin inhibitor-CMe is used in the production of a transgenic wheat plant having resistance against seed-feeding insect larvae.

In a still further example, the present invention provides a method for producing a transgenic graminaceous plant having resistance to an abiotic stress, said method comprising:

(i) producing a transgenic graminaceous plant cell comprising a transgene that encodes a protein that confers or enhances resistance to an abiotic stress using a method described herein according to any embodiment;
(ii) regenerating a transgenic plant from said cell; and
(ii) maintaining said transgenic plant for a time and under conditions sufficient to induce expression of said nucleic acid, thereby producing a transgenic graminaceous plant having resistance to an abiotic stress.

Preferably, the method described supra applies mutatis mutandis to a method for improving or enhancing the resistance of a plant to an abiotic stress.

In a further example, the abiotic stress is drought or dessication. A transgene that expresses a late embryogenesis protein that accumulates during seed desiccation and in vegetative tissues when plants experience water loss is useful for producing a transgenic graminaceous plant having drought or dessication resistance or tolerance. For example, a nucleic acid encoding barley HVA1 (SEQ ID NO: 56) is used to produce an expression construct described herein. This expression construct is then used to produce a transgenic plant by a method described herein according to any embodiment.

In another example, a transgene encoding an Arabidopsis DREB1A (SEQ ID NO: 58) is used to produce a transgenic graminaceous plant having improved drought tolerance in addition to tolerance to low temperatures and/or salinity.

### 6.2 Modulating Expression in a Graminaceous Plant

It is to be understood that the present invention also extends to the production of transgenic plants that express transgenes that do not encode a protein. For example, the transgene encodes an interfering RNA, a ribozyme, an abzyme, co-suppression molecule, gene-silencing molecule or gene-targeting molecule, which prevents or reduced the expression of a nucleic acid of interest.

Suitable methods for producing interfering RNA or a ribozyme, or an abzyme are known in the art.

For example, a number of classes of ribozymes have been identified. One class of ribozymes is derived from a number of small circular RNAs that are capable of self-cleavage and replication in plants. Examples include RNAs from avocado sunblotch viroid and the satellite RNAs from tobacco ringspot virus, lucerne transient streak virus, velvet tobacco mottle virus, solanum nodiflorum mottle virus and subterranean clover mottle virus. The design and use of transgenes encoding a ribozyme capable of selectively cleaving a target RNA is described, for example, in Haseloff et al. Nature, 334:585-591 (1988).

Alternatively, a transgene expresses a nucleic acid capable of inducing sense suppression of a target nucleic acid. For example, a transgene is produced comprising nucleic acid configured in the sense orientation as a promoter of a target nucleic acid. Such a method is described, for example, in Napoli et al., The Plant Cell 2:279-289 1990; or U.S. Pat. No. 5,034,323.

To reduce or prevent expression of a nucleic acid by sense suppression, the transgene need not be absolutely identical to the nucleic acid. Furthermore, the transgene need not comprise the complete sequence of the nucleic acid to reduce or prevent expression of said nucleic acid by sense-suppression.

RNA interference is also useful for reducing or preventing expression of a nucleic acid. Suitable methods of RNAi are described in Marx, Science, 288:1370-1372, 2000. Exemplary methods for reducing or preventing expression of a nucleic acid are described in WO 99/49029, WO 99/53050 and WO0/75164. Briefly a transgene is produced that expresses a nucleic acid that is complementary to a sequence of nucleotides in the target nucleic acid. The transgene additionally expresses nucleic acid substantially identical to said sequence of nucleotides in the target nucleic acid. The two nucleic acids expressed by the transgene are capable of hybridizing and reducing or preventing expression of the target nucleic acid, presumably at the post-transcriptional level.

For example, it may be desirable to express, for example, an inhibitory RNA that reduces or prevents expression of a fungal nucleic acid required for infection of a graminaceous plant. For example, S-adenosyl-L-methionine-Δ²⁴-sterol methyl transferases (SMT_{I} or SMT_{II}) is required for the life cycle of many insects and fungal pathogens to be completed, and expression of inhibitory RNA against this enzyme can prevent the pathogen from maturing into an adult, thereby preventing pathogen spread within the graminaceous plant.

Alternatively, a transgene encoding an inhibitory RNA molecule that reduces or prevents expression of the movement protein of wheat streak mosaic virus (WSMV) is expressed in wheat to inhibit virus movement from the pericarp through the vasculature of the plant.

In another example, the transgene encodes an inhibitory RNA, a ribozyme, an abzyme, co-suppression molecule, gene-silencing molecule or gene-targeting molecule to thereby enhance or alter the nutritional characteristics of a graminaceous plant. For example, wheat grain is predominantly composed of starch that is a mixture of two polymers: almost linear amylose and heavily-branched amylopectin. By altering the ratio of amylopectin to amylase, the physico-chemical properties and/or end-use of wheat is altered. To alter the ratio of amylopectin to amylase, an inhibitory RNA that reduces or prevents expression of the granule-bound starch synthase I gene (encoding GBSSI or WAXY protein) is expressed in a transgenic wheat plant to thereby alter the level of amylose in said plant. Wheat flour from a plant expressing such a transgene and having a reduced level of amylose relative to amylopectin is desirable for noodle making as it improves noodle texture. Accordingly, by reducing expression of the granule-bound starch synthase I gene the noodle making qualities of wheat is improved.

The present invention clearly extends to a plant, progeny, seed, propagating material having an altered phenotype or altered gene expression described herein. Preferably, such a plant, progeny, seed, propagating material is produced according to the method of the present invention.

### 8. Additional Methlods

The present invention also provides a method for regenerating a plant or plantlet or plant part from a plant cell. For example, such a method comprises:

(a) contacting a plant cell with a compound that induces callus formation for a time and under conditions sufficient to produce a callus;
(b) contacting the callus with a compound that induces shoot formation for a time and under conditions sufficient for a shoot to develop;
(c) contacting the callus with a compound that induces root formation for a time and under conditions sufficient to initiate root growth, thereby producing a plantlet; and
(d) growing the plantlet for a time and under conditions sufficient to produce a plant or plantlet or plant part.

For example, the method comprises:

(i) contacting a transgenic cell with a solution comprising 2,4-D or Dicambia or TDZ and picloram such that a callus is produced; and
(ii) contacting the callus produced at (i) with a solution comprising zeatin and/or TDZ such that a shoot develops;
(iii) contacting the shoot produced at (ii) with a solution comprising a naphthaleneacetic acid such that root growth commences, thereby producing a plantlet.

For example, the transgenic cell is contacted with a solution comprising from about 1 mg/L 2,4-D to about 4 mg/L 2,4-D, for example, about 2 mg/L 2,4-D. Alternatively, the transgenic cell is contacted with a solution comprising from about 2 mg/L Dicambia to about 8 mg/L Dicambia, for example, about 4 mg/L Dicambia. Alternatively, the transgenic cell is contacted with a solution comprising from about 1 mg/L TDZ to about 6 mg/L TDZ and about 1 mg/L picloram to about 4 mg/L picloram, for example, about 3 mg/L TDZ and about 2 mg/L picloram.

In another example, the callus is contacted with a solution comprising from about 1 mg/L zeatin to about 4 mg/L zeatin, for example, about 2 mg/L zeatin. Alternatively, the callus is contacted with a solution comprising from about 0.25 mg/L TDZ to about 2 mg/L TDZ, for example, about 1 mg/L TDZ.

In a further example, the shoot is contacted with a solution comprising from about 0.25 mg/L NAA to about 2 mg/L NAA, for example, about 1 mg/L NAA.

Additional suitable compounds will be apparent to the skilled artisan based on the description herein and shall be take to apply mutatis mutandis to the present embodiment of the invention.

In one example, the method of regenerating a plant or plantlet or plant part is for regenerating a transgenic plant or plantlet or plant part, wherein the transgenic plant or plantlet or plant part express a selectable marker, and the method additionally comprises selecting a transgenic plant or plantlet or plant part or a transgenic plant cell expressing said selectable marker.

Suitable methods of selection are described herein and apply mutatis mutandis to the present embodiment of the invention.

The present invention also provides a method of selecting a transgenic plant or plantlet or plant part or a transgenic plant cell expressing a selectable marker gene, wherein said selectable marker gene converts a toxic substrate into a non-toxic substrate and/or permits a plant or plantlet or plant part or plant cell expressing said selectable marker gene to grow in the presence of a toxic substrate, said method comprising contacting said transgenic plant or plantlet or plant part or plant cell with said toxic substrate for a time and under conditions sufficient to kill or prevent growth of a plant or plantlet or plant part or plant cell that does not express the selectable marker gene, thereby selecting a transgenic plant or plantlet or plant part or a transgenic plant cell.

Suitable selectable marker genes and methods of selection are described herein and are to be taken to apply mutatis mutandis to the present embodiment of the invention.

The present invention also provides a method of detecting or identifying a transgenic plant or plantlet or plant part or a transgenic plant cell expressing a detectable marker gene, wherein said detectable marker gene produces a detectable signal when expressed in a plant, plantlet, plant part or plant cell, said method comprising detecting said detectable signal in a plant or plantlet or plant part or plant cell,

thereby detecting or identifying a transgenic plant or plantlet or plant part or a transgenic plant cell.

In one example, the method additionally comprises selecting the plant or plantlet or plant part or plant cell expressing the detectable marker gene.

Suitable detectable marker genes and methods of detection or identification are described herein and are to be taken to apply mutatis mutandis to the present embodiment of the invention.

The present invention is further described by reference to the following non-limiting examples.

### Example 1

### Transformation of Wheat Embryonic Cells

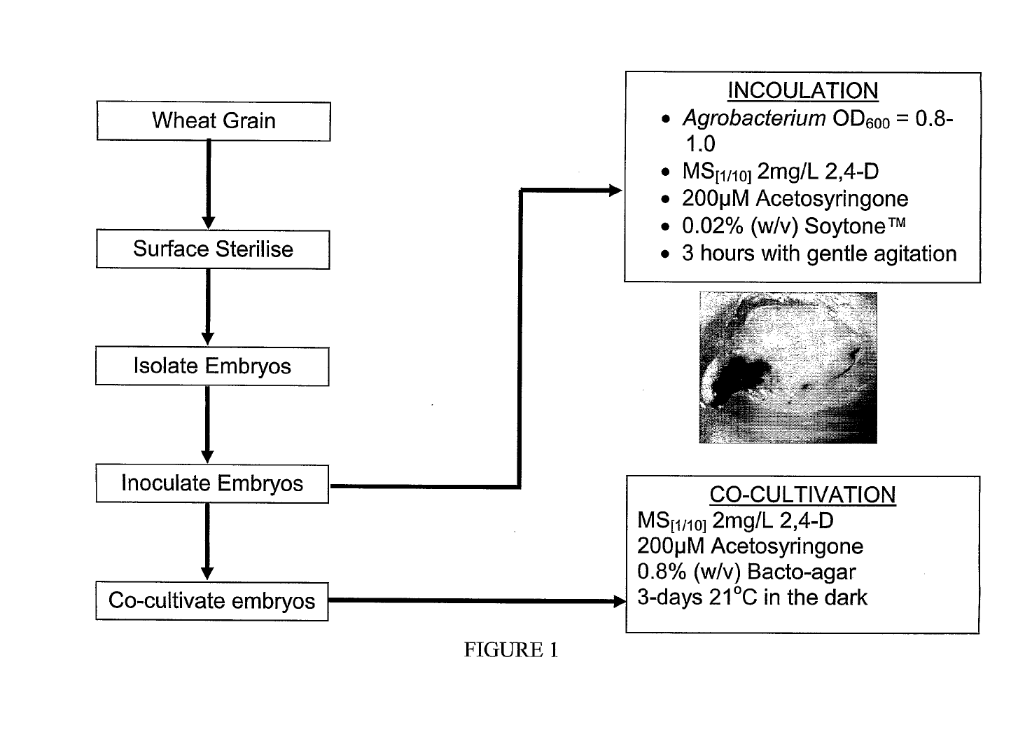

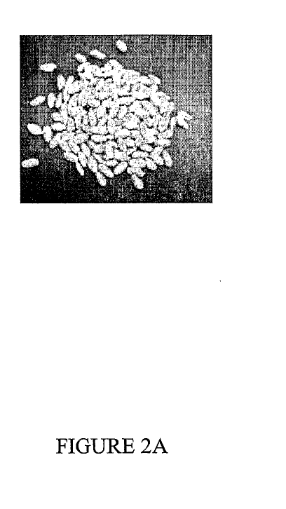

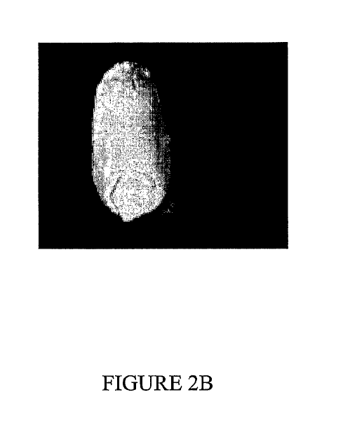

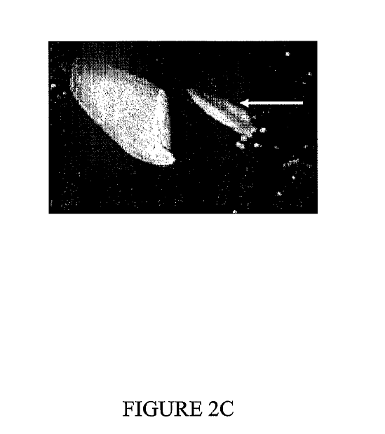

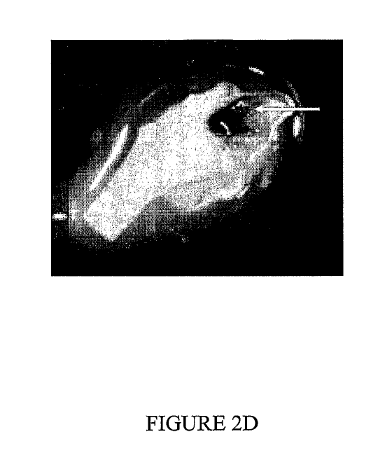

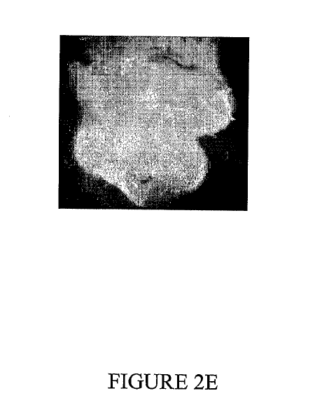

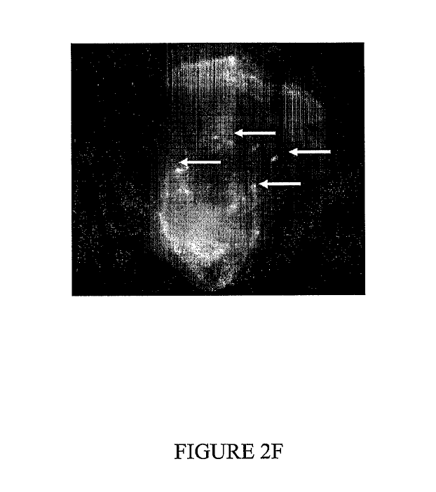

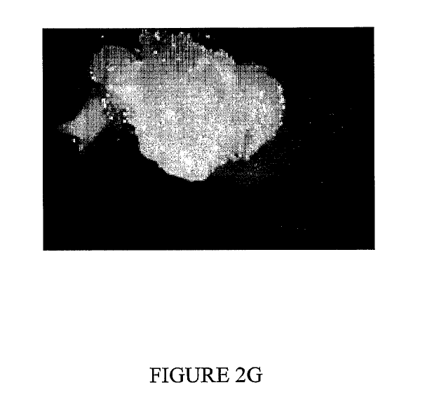

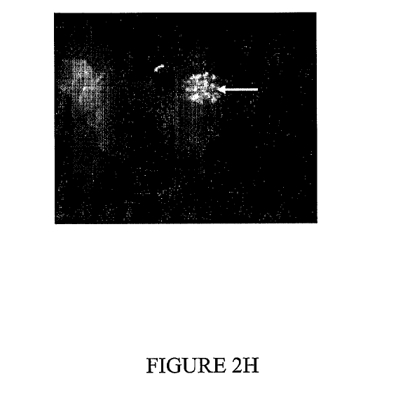

Wheat grain from Triticum aestivum (Bobwhite) was surface sterilized for 30 minutes in a 0.8% (v/v) NaOCl solution and rinsed at least four times in sterile distilled water. Mature embryos were aseptically excised from surface sterilized grain, the seed coat removed and used directly for Agrobacterium-mediated transformation. FIG. 1 summarizes the process of Agrobacterium infection of mature wheat embryos. FIG. 2 shows the isolation of embryo with intact epiblast and scutellum from dried wheat grain.

Explants were used directly for Agrobacterium-mediated transformation. Agrobacterium strain EHA105 comprising the pCAMBIA1305.2 vector (expressing the GUS reporter gene under control of the CaMV35s promoter) or pLM301 (pSB1-Ubi1::DsRed2-nos) were used to inoculate 10-15 mL of LB supplemented with 100 μg/mL of rifampicin and kanamycin in a 50 mL Falcon tube, which is incubated for 24 to 48 hours at 27-28° C. For inoculation, 100 μl of the Agrobacterium culture was used to inoculate 25 mL of fresh LB supplemented kanamycin and incubated for 24 hours. This full strength inoculum was centrifuged at 3000 rpm for 10 minutes at room temperature with the resulting pellet re-suspended in liquid inoculation medium (MS_{[1/10]}) to an OD₆₀₀=0.25-0.8. The inoculation medium consisted of 1/10 strength liquid Murashige and Skoog. (Murashige and Skoog Physiol. Plant, 15: 473-497, 1962) basal salts (MS_{[1/10]}) supplemented with 2 mg/L 2,4-D, 200 μM acetosyringone, and 0.02% (w/v) Soytone™.

Agrobacterium infection was standardized for 3 hours at room temperature with gentle agitation, followed by 3 days of co-cultivation in the dark on a medium consisting of 1× Murashige and Skoog (Murashige and Skoog Physiol. Plant, 15: 473-497, 1962) macronutrients, 1× micronutrients and organic vitamins, supplemented with 200 mg/L casein hydrolysate, 100 mg/L myo-inositol, 3% (w/v) sucrose, 2 mg/L 2,4-D supplemented with 200 μM acetosyringone and 0.8%-2.0% (w/v) Bacto Agar at 21° C. with the embryo axis preferably facing downwards.

Explants were optionally washed thoroughly with liquid MS_{(1/10)} without acetosyringone or Soytone™ but supplemented with 250 mg/L cefotaxime.

Alternatively, explants are washed in sterile water supplemented with 250 mg/L cefotaxime until no visible signs of Agrobacterium remain (i.e. wash solution remains clear after washing).

Transient gusA or DsRed2 expression was determined on explants sampled after 3 days (or as indicated otherwise) on induction medium containing Timentin, using the histochemical GUS assay (Jefferson Plant Mol. Biol. Rep. 5: 387-405 1987) or visualized using a Leica Stereomicroscope with DsRed2 optic filters (see FIG. 2).

For histochemical gusA expression, explants were incubated overnight at 37° C. in buffer containing 1 mM X-Gluc, 100 mM sodium phosphate buffer pH 7.0, potassium 0.5 mM ferricyanide, 0.5 mM potassium ferrocyanide and 0.1% (v/v) Triton X-100. Blue gusA expression foci were counted under a microscope and T-DNA delivery assessed by counting explants that had at least one gusA expression foci and then counting the number of foci per embryo. To assay for stable gusA expression calli, shoots and leaf fragments from regenerating plantlets were incubated overnight at 37° C. and, if necessary, for a further 1-2 days at 25° C. As shown in FIG. 2, gusA and DsRed2 expression is detectable in the transformed embryo 3 days after inoculation.

### Example 2

### Method 1 for Callus Induction and Regeneration of Transgenic Plants

Following co-cultivation and optional washing of transformed embryos produced as described in Example 1, explants are placed on a medium consisting of 1× Murashige and Skoog (Murashige and Skoog Physiol. Plant, 15: 473-497, 1962) macronutrients, 1× micronutrients and organic vitamins, supplemented with 200 mg/L casein hydrolysate, 100 mg/L myo-inositol, 3% (w/v) sucrose, 2 mg/L 2,4-D and 0.8%-2.0% (w/v) Bacto Agar for induction of somatic embryos and supplemented with hygromycin-B (5-15 mg/L) or 5-7.5 mM D-serine or D-alanine and antibiotics (cefotaxime 250 mg/L or timentin 150 mg/L) to control Agrobacterium growth. In some cases the application of selection is not applied until 5 weeks after inoculation.

Mature embryo explants are incubated for 3 weeks in the dark, after which they produce a callus on selection medium. Explants showing callusing on selection medium are sub-cultured regularly to fresh media supplemented with selective agents and antibiotics.

After at least 3 weeks callus induction, embryogenic calli are transferred to a regeneration medium consisting of 1× Murashige and Skoog (supra) macronutrients, 1× micronutrients and organic vitamins, supplemented with 200 mg/L casein hydrolysate, 100 mg/L myo-inositol, 3% (w/v) sucrose, 2 mg/L zeatin and 0.8% (w/v) Bacto-Agar and 10 mg/L hygromycin-B and antibiotics (cefotaxime 250 mg/L or timentin 150 mg/L) to control Agrobacterium growth.

Explants are cultured in the light for a minimum of 1 to 2 cycles of 2-3 weeks with putative transgenic plantlets/calli transferred to fresh regeneration media.

After a further 10 days, regenerated plantlets are transferred to MS_{[1/2]} supplemented with 1 mg/L NAA for root initiation. Any regenerated plantlets surviving greater than 3 weeks on root induction media with healthy root formation are potted into a nursery mix consisting of peat and sand (1:1) and kept at 22-24° C. with elevated humidity under a nursery humidity chamber system. After two weeks, plants are removed from the humidity chamber and hand watered and liquid fed Aquasol™ weekly until maturity.

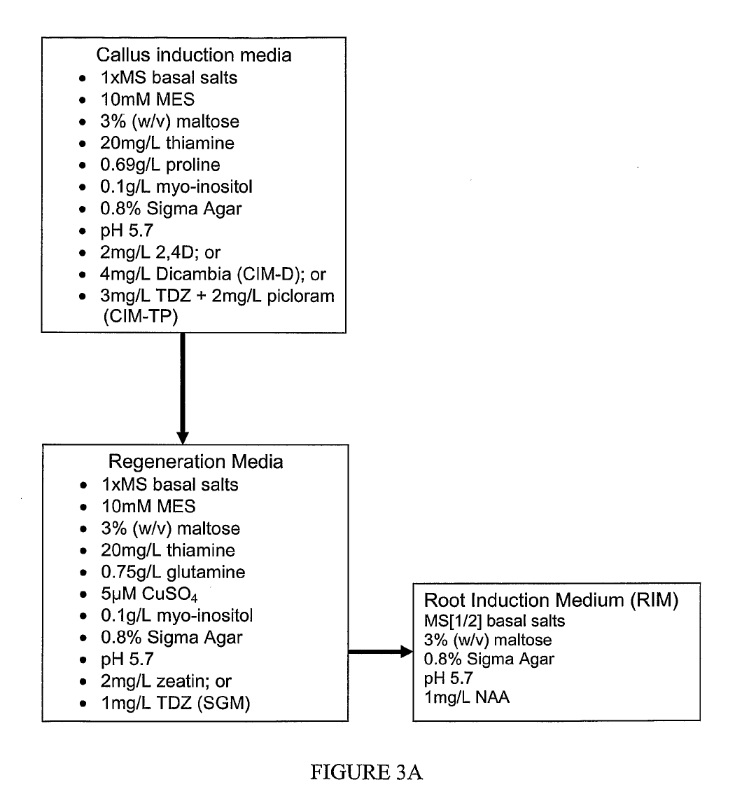

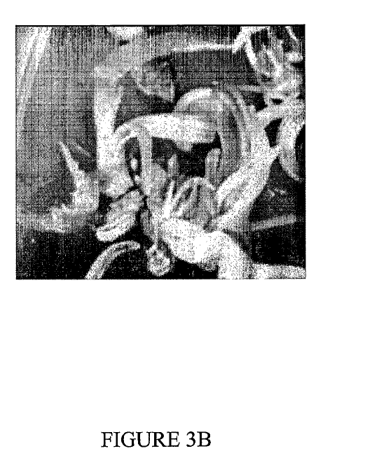

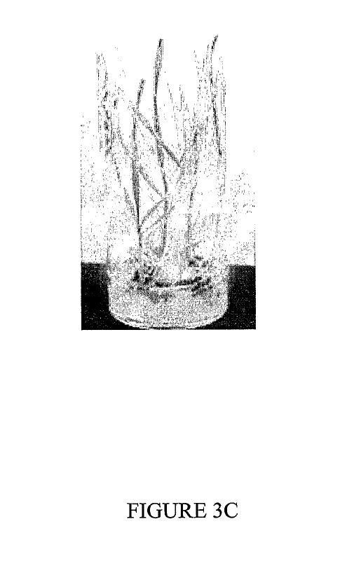

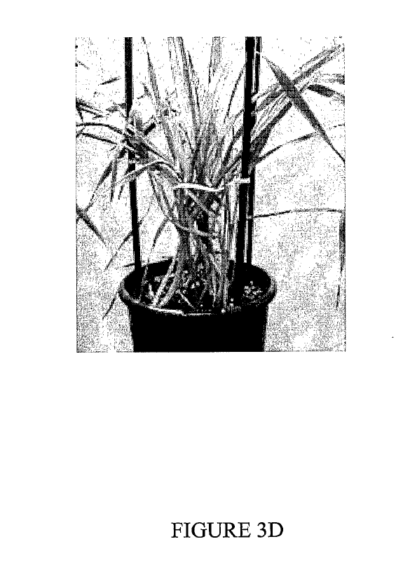

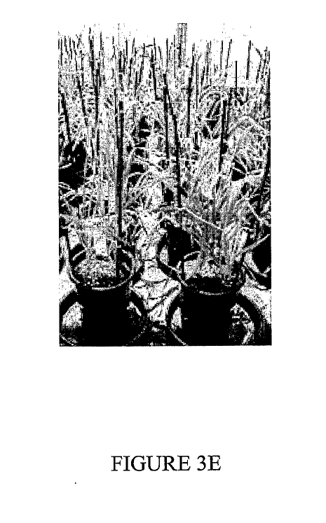

FIG. 3 shows a schematic representation of callus induction and regeneration from mature embryos and results of regeneration of transgenic wheat.

### Example 3

### Method 2 for Callus Induction and Regeneration of Transgenic Plants

Following co-cultivation and optional washing of transformed embryos produced as described in Example 1, explants are placed on a callus induction medium (CIM-D) consisting of 1× Murashige and Skoog (Murashige and Skoog Physiol. Plant, 15: 473-497, 1962) macronutrients, 1× micronutrients and organic vitamins, supplemented with 1 g/L casein hydrolysate, 100 mg/L myo-inositol, 3% (w/v) maltose, 1.95 g/L MES, 0.69 g/L proline, 20 mg/L Thiamine hydrochloride, 4 mg/L Dicamba and 0.8% (w/v) Bacto-Agar, supplemented with hygromycin-B (5-15 mg/L) or preferably 5-7.5 mM D-serine or D-alanine and antibiotics (cefotaxime 250 mg/L and/or timentin 150 mg/L) to control Agrobacterium growth. In some cases the application of selection is not applied until 5 weeks after inoculation.

Mature embryo explants are incubated for 3 weeks in the dark, after which they produce a callus on the selection medium. Explants showing callusing on the selection medium are sub-cultured regularly to fresh CIM-D supplemented with selective agents and antibiotics.

After at least 3-4 weeks callus induction, embryogenic calli are transferred to a regeneration medium (SGM) consisting of 1× Murashige and Skoog (supra) macronutrients, 1× micronutrients and organic vitamins, supplemented with 1 g/L casein hydrolysate, 100 mg/L myo-inositol, 20 mg/L Thiamine hydrochloride, 750 mg/L glutamine, 5 μM CuSO₄, 1.95 g/L MES, 3% (w/v) maltose, 1 mg/L TDZ and 0.8% (w/v) Bacto-Agar and 10 mg/L hygromycin-B and/or preferably 5-7.5 mM D-serine or D-alanine and antibiotics (cefotaxime 250 mg/L and/or timentin 150 mg/L) to control Agrobacterium growth.

Explants are cultured in the light for a minimum of 1 to 2 cycles of 2-3 weeks with putative transgenic plantlets/calli transferred to fresh regeneration media.

After a further 10 days, regenerated plantlets are transferred to RM media consisting of MS_{[1/2]} supplemented with 1 mg/L NAA and 10 mg/L hygromycin-B and/or preferably 5-7.5 mM D-serine or D-alanine and antibiotics (cefotaxime 250 mg/L and/or timentin 150 mg/L) for root initiation. Any regenerated plantlets surviving greater than 3 weeks on RM with healthy root formation are potted into a nursery mix consisting of peat and and (1:1) and kept at 22-24° C. with elevated humidity under a nursery humidity chamber system. After two weeks, plants are removed from the humidity chamber and hand watered and liquid fed Aquasol™ weekly until maturity. FIG. 3 shows a schematic representation of callus induction and regeneration from mature embryos and results of regeneration of transgenic wheat.

### Example 4

### Method 3 for Callus Induction and Regeneration of Transgenic Plants

Following co-cultivation and optional washing of transformed embryos produced as described in Example 1, explants are placed on a callus induction medium (CIM-TP) consisting of 1× Murashige and Skoog (Murashige and Skoog Physiol. Plant, 15: 473-497, 1962) macronutrients, 1× micronutrients and organic vitamins, supplemented with 1 g/L casein hydrolysate, 100 mg/L myo-inositol, 3% (w/v) maltose, 1.95 g/L MES, 0.69 g/L proline, 20 mg/L Thiamine hydrochloride, 3 mg/L TDZ, 2 mg/L picloram and 0.8% (w/v) Bacto-Agar, supplemented with hygromycin-B (5-15 mg/L) or preferably 5-7.5 mM D-serine or D-alanine and antibiotics (cefotaxime 250 mg/L and/or timentin 150 mg/L) to control Agrobacterium growth. In some cases the application of selection is not applied until 5 weeks after inoculation. Mature embryo explants are incubated for 3 weeks in the dark, after which they produce a callus on the selection medium. Explants showing callusing on the selection medium are sub-cultured regularly to fresh CIM-TP supplemented with selective agents and antibiotics.

After at least 3-4 weeks callus induction, embryogenic calli are transferred to a regeneration medium (SGM) consisting of 1× Murashige and Skoog (supra) macronutrients, 1× micronutrients and organic vitamins, supplemented with 1 g/L casein hydrolysate, 100 mg/L myo-inositol, 20 mg/L Thiamine hydrochloride, 750 mg/L glutamine, 5 □M CuSO₄, 1.95 g/L MES, 3% (w/v) maltose, 1 mg/L TDZ and 0.8% (w/v) Bacto-Agar and 10 mg/L hygromycin-B or preferably 5-7.5 mM D-serine or D-alanine and antibiotics (cefotaxime 250 mg/L and/or timentin 150 mg/L) to control Agrobacterium growth.

Explants are cultured in the light for a minimum of 1 to 2 cycles of 2-3 weeks with putative transgenic plantlets/calli transferred to fresh regeneration media.

After a further 10 days, regenerated plantlets are transferred to RM media consisting of MS_{[1/2]} supplemented with 1 mg/L NAA and 10 mg/L hygromycin-B or preferably 5-7.5 mM D-serine or D-alanine and antibiotics (cefotaxime 250 mg/L and/or timentin 150 mg/L) for root initiation. Any regenerated plantlets surviving greater than 3 weeks on RM with healthy root formation are potted into a nursery mix consisting of peat and sand (1:1) and kept at 22-24° C. with elevated humidity under a nursery humidity chamber system. After two weeks, plants are removed from the humidity chamber and hand watered and liquid fed Aquasol™ weekly until maturity. FIG. 3 shows a schematic representation of callus induction and regeneration from mature embryos and results of regeneration of transgenic wheat.

A range of concentrations of the D-amino acids D-serine and D-alanine were tested for effects on wheat regeneration and germination from embryos derived from mature dried grain of the cultivar Bobwhite. As indicated in Tables 4 and 5, both D-serine and D-alanine reduced the regeneration and germination of wheat plants with levels greater than 7.5 mM and 5 mM respectively. Similar results were observed for the wheat genotype Ventura (Tables 6 and 7).

TABLE 4
Effect of D-serine and D-alanine on regeneration of wheat
from embryos derived from mature dried grain of the cultivar
Bobwhite. Data are the proportion (%) of explants regenerating
after 3 weeks (n = 20).

| Selection | Experiment Number | | | | |
|---|---|---|---|---|---|
| (mM) | 1 | 2 | 3 | Average | stdev |
| 0 D-Amino | | | 30 | 30 | |
| acids | | | | | |
| D-serine | | | | | |
| 0.5 | 45 | 33 | 5 | 28 | 21 |
| 1 | 30 | 33 | 5 | 23 | 15 |
| 5 | 10 | 10 | 5 | 8 | 3 |
| 7.5 | 0 | 5 | 5 | 3 | 3 |
| 10 | 0 | 0 | 0 | 0 | 0 |
| 30 | 0 | 0 | 0 | 0 | 0 |
| D-alanine | | | | | |
| 0.5 | 30 | 30 | 0 | 20 | 17 |
| 1 | 35 | 21 | 5 | 20 | 15 |
| 3 | 20 | 25 | 0 | 15 | 13 |
| 5 | 5 | 5 | 0 | 3 | 3 |
| 10 | 0 | 0 | 0 | 0 | 0 |

TABLE 5
The effect of D-serine and D-alanine on the germination of wheat
mature dried grain of the cultivar Bobwhite. Data are the proportion
(%) of explants germinated after 1 week (n = 15).

| Selection | Experiment Number | | | | |
|---|---|---|---|---|---|
| (mM) | 1 | 2 | 3 | Average | stdev |
| 0 D-Amino | ND | 80 | ND | 80 | — |
| acids | | | | | |
| D-serine | | | | | |
| 0.5 | 35 | 70 | 80 | 62 | 24 |
| 1 | 30 | 80 | 60 | 57 | 25 |
| 5 | 20 | 70 | 25 | 38 | 28 |
| 7.5 | 10 | 60 | 25 | 32 | 26 |
| 10 | 0 | 35 | 20 | 18 | 18 |
| 30 | 0 | 0 | 0 | 0 | 0 |
| D-alanine | | | | | |
| 0.5 | 55 | 75 | 100 | 77 | 23 |
| 1 | 70 | 80 | 80 | 77 | 6 |
| 3 | 70 | 70 | 80 | 73 | 6 |
| 5 | 50 | 85 | 50 | 62 | 20 |
| 10 | 10 | 35 | 40 | 28 | 16 |

TABLE 6
The effect of D-serine and D-alanine on the regeneration
of wheat from embryos derived from mature dried grain of
the cultivar Ventura. Data are the proportion (%) of explants
regenerating after 3 weeks (n = 20).

| Selection | Experiment number | | | | |
|---|---|---|---|---|---|
| (mM) | 1 | 2 | 3 | Average | stdev |
| 0 D-Amino | | | | | |
| acids | | | | | |
| D-serine | | | | | |
| 0.5 | 10 | 15 | 5 | 10 | 5 |
| 1 | 10 | 5 | 5 | 7 | 3 |
| 5 | 5 | 5 | — | 5 | 0 |
| 7.5 | 0 | 0 | 0 | 0 | 0 |
| 10 | 0 | 0 | 0 | 0 | 0 |
| 30 | 0 | 0 | 0 | 0 | 0 |
| D-alanine | | | | | |
| 0.5 | 10 | 10 | 5 | 8 | 3 |
| 1 | 0 | 10 | 5 | 5 | 5 |
| 3 | 10 | 10 | 0 | 7 | 6 |
| 5 | 5 | 5 | 0 | 3 | 3 |
| 10 | 0 | 0 | 0 | 0 | 0 |
| 20 | 0 | 0 | 0 | 0 | 0 |

TABLE 7
The effect of D-serine and D-alanine on the germination of wheat
mature dried grain of the cultivar Ventura. Data are the proportion
(%) of explants regenerating after 1 week (n = 15).

| Selection | Experiment number | | | | |
|---|---|---|---|---|---|
| (mM) | 1 | 2 | 3 | Average | stdev |
| 0 D-Amino | ND | ND | 65 | 65 | |
| acids | | | | | |
| D-serine | | | | | |
| 0.5 | 65 | 60 | 85 | 70 | 13 |
| 1 | 70 | 70 | 65 | 68 | 3 |
| 5 | 60 | 45 | ND | 53 | 11 |
| 7.5 | 15 | 20 | 35 | 23 | 10 |
| 10 | ND | 30 | 55 | 43 | 18 |
| 30 | 0 | 0 | 0 | 0 | 0 |
| D-alanine | | | | | |
| 0.5 | 65 | 65 | 90 | 73 | 14 |
| 1 | 75 | 75 | 75 | 75 | 0 |
| 3 | 80 | 65 | 80 | 75 | 9 |
| 5 | 70 | 55 | 75 | 67 | 10 |
| 10 | 65 | 30 | 45 | 47 | 18 |
| 20 | 5 | 25 | 40 | 5 | 18 |

ND = Not determined

### Example 5

### Detecting Transgenic Wheat Using Q-PCR

T₀ and T₁ plants are sampled for genomic DNA for molecular analysis. All Q-PCRs are performed using Taqman® probes to detect amplification. Q-PCR Taqman® screens have been established for the gusA and DsRed2 genes and bar, dao1, dsdA and hph selectable marker genes. To ensure that positive Q-PCR signals are not due to the adventitious presence of Agrobacterium, Q-PCR Taqman® screens have been established for the presence of the vir C gene from outside of the T-DNA.

A standard real-time PCR mixture for each candidate gene contained 2× Taqman® master mix, 300 nM of each primer, 250 nM probe, 10-20 ng of genomic DNA and water to a final volume of 10 μl. The thermo-cycling conditions for the PCR were: 1 cycle of 50° C. for 2 minutes followed 1 cycle of 95° C. for 5 minutes followed by 40 cycles of 95° C. for 15 seconds, 60° C. for 1 minute. Q-PCR and data analysis were performed on a Stratagene MX3000p Real Time PCR thermocycler.

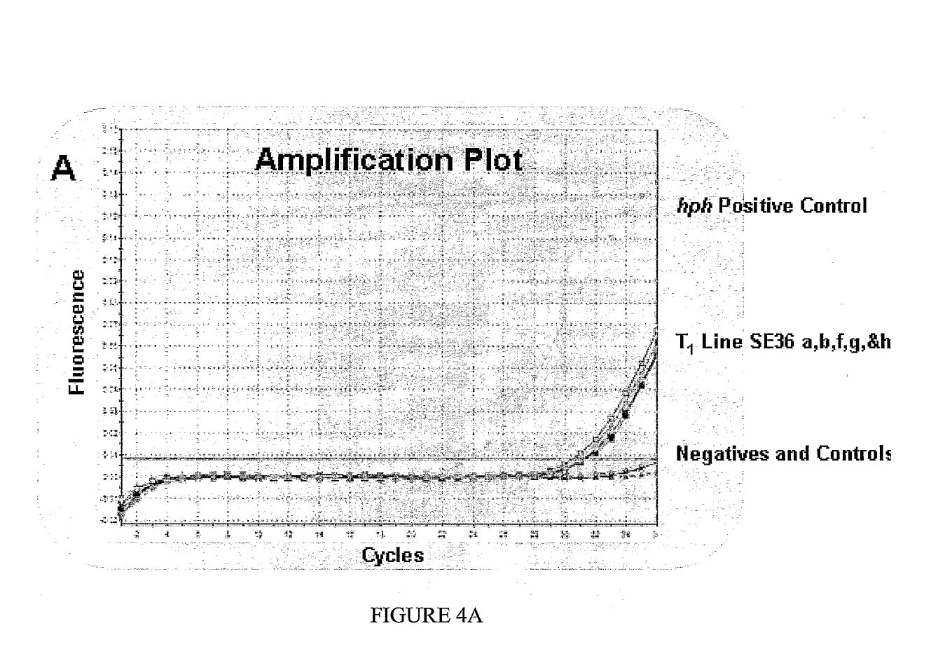

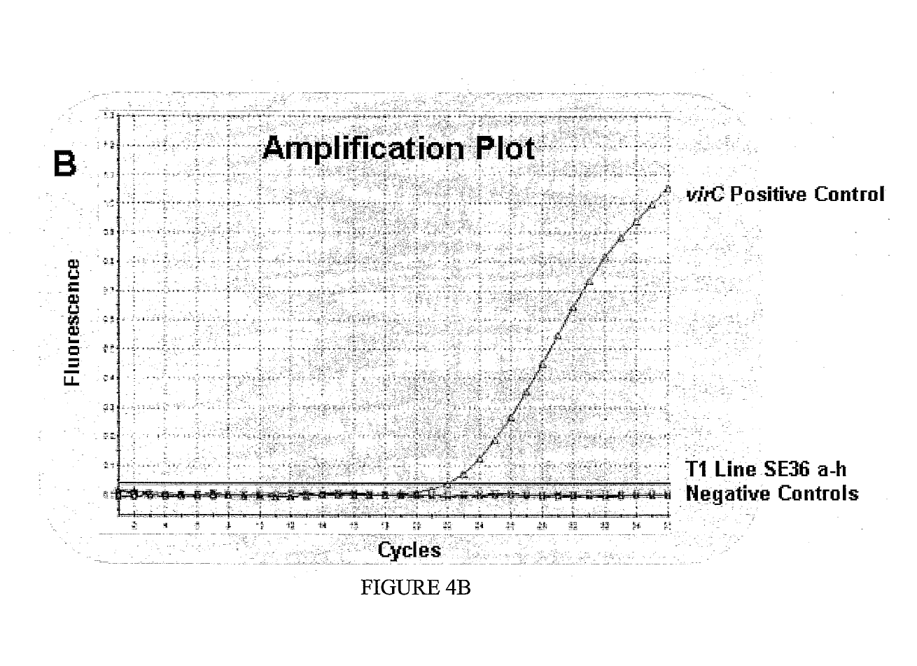

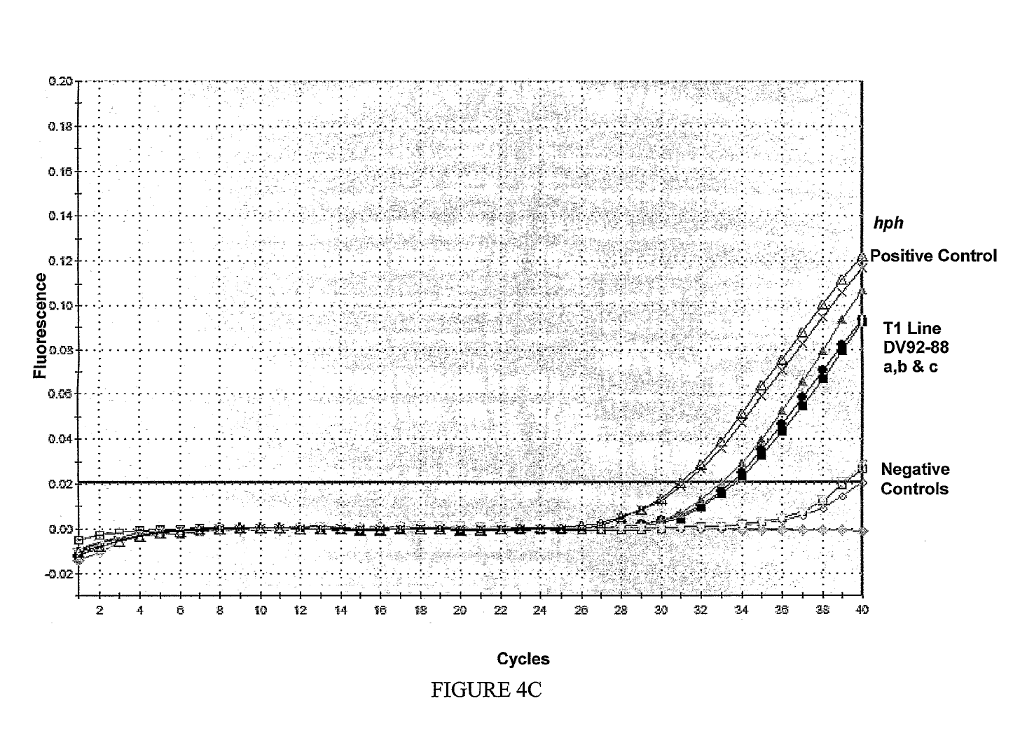

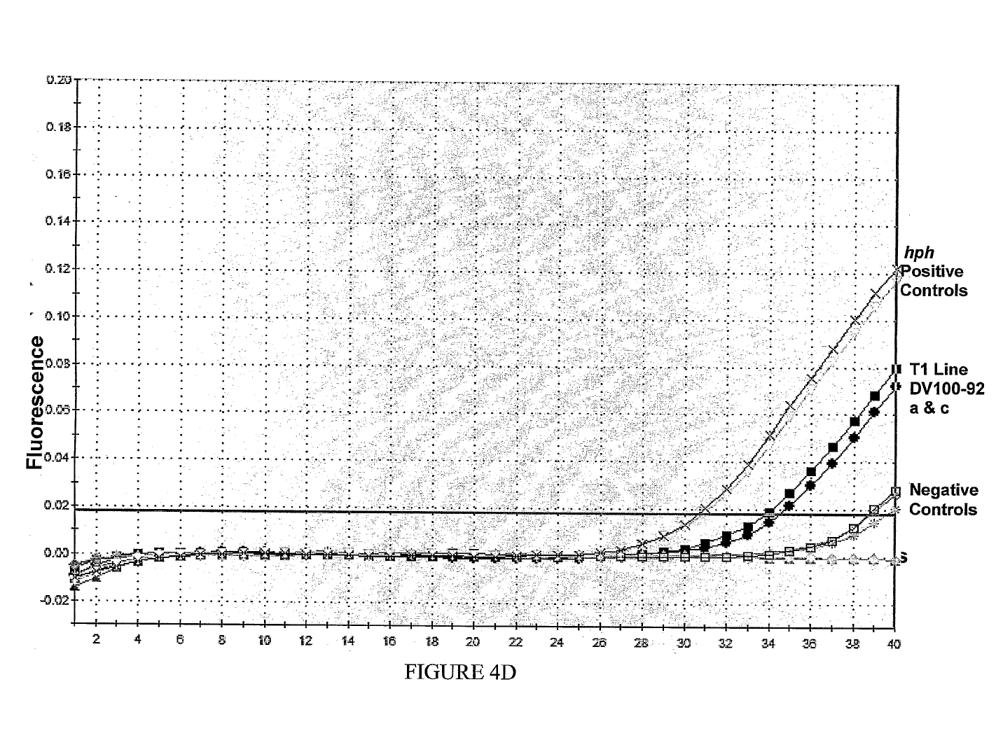

As shown in FIGS. 4A-D the presence of transgenes was detected in independent T₁ transgenic wheat lines using the Taqman® assays.

### Example 6

### Detecting Transgenic Wheat Using Southern Hybridization

Genomic DNA from T₁ or T₂ plants are analyzed by Southern blotting to detect the stable integration of transgenes and the number of copies introduced. Total genomic DNA is isolated from wheat leaves according to Dellaporta et al. Plant Mol Biol Rep., 4: 19-21, 1983. Twenty to thirty micrograms of genomic DNA is digested with appropriate restriction enzyme(s) and resolved on a 0.8-1% agarose gel and blotted onto a nylon membrane (Hybond N, Amersham, UK).

To detect transgenic wheat containing T-DNA from the vector pCAMBIA1305.2, a blot is prepared with genomic DNA digested with the restriction enzyme EcoRI, which cuts once within the T-DNA region. The blot is first probed for the presence of hph and subsequently probed for the presence of gusA. The gusA probe is PCR amplified from pCAMBIA1305.2 using the primers CAT CCT CGA CGA TAG CAC CC (SEQ ID NO: 72) and TCA TGT TTG CCA AAG CCC TT (SEQ ID NO: 73) producing a 501 bp product and the hph probe is PCR amplified from pCAMBIA1305.2 using the primers CGC ATA ACA GCG GTC ATT GAC TGG AGC (SEQ ID NO: 74) and GCT GGG GCG TCG GTT TCC ACT ATC GG (SEQ ID NO: 75) producing a 375 bp product.

For transgenic wheat containing T-DNA from the vector pMPB0057 (pUbi1::bar-nos Act1D::gusA(int)), a blot is prepared as described above with genomic DNA digested with the restriction enzyme HindIII, which cuts once within the T-DNA region. The blot is first probed for the presence of bar and subsequently probed for the presence of gusA. The gusA probe is PCR amplified from pMPB0057 using the primers ATG AAC TGT GCG TCA CAG CC (SEQ ID NO: 76) and TTG TCA CGC GCT ATC AGC C (SEQ ID NO: 77) producing a 451 bp product and the bar probe is PCR amplified from pMPBOO57 using the primers GTC TGC ACC ATC GTC AAC C (SEQ ID NO: 78) and GAA GTC CAG CTG CCA GAA AC (SEQ ID NO: 79) producing a 425 bp product.

For transgenic wheat containing T-DNA from the vector pSB1_Ubi1::dsdA-ocs_Ubi1::DsRed2-nos, a blot is prepared as described above with genomic DNA digested with the restriction enzyme SphI, which cuts once within the T-DNA region. The blot is first probed for the presence of DsRed2 and subsequently probed for the presence of dsdA. The DsRed2 probe is PCR amplified from pSB1_Ubi1::dsdA-ocs_Ubi1::DsRed2-nos using the primers CTG TCC CCC CAG TTC CAG TA (SEQ ID NO: 80) and CGA TGG TGT AGT CCT CGT TGT G (SEQ ID NO: 81) producing a 450 bp product and the dsdA probe is PCR amplified from pSB1_Ubi1::dsdA-ocs_Ubi1::DsRed2-nos using the primers GTG GGC TCA ACC GGA AAT CT (SEQ ID NO: 82) and GCA GTT GTT CTG CGC TGA AAC (SEQ ID NO: 83) producing a 750 bp product.

For transgenic wheat containing T-DNA from the vector pSB1_Ubi1::dao1A-ocs_Ubi1::DsRed2-nos, a blot is prepared as described above with genomic DNA digested with the restriction enzyme SpeI, which cuts once within the T-DNA region. The blot is first probed for the presence of DsRed2 and subsequently probed for the presence of dao1. The DsRed2 probe is PCR amplified from pSB1_Ubi1::dao1-ocs_Ubi1::DsRed2-nos using the primers CTG TCC CCC CAG TTC CAG TA (SEQ ID NO: 80) and CGA TGG TGT AGT CCT CGT TGT G (SEQ ID NO: 81) producing a 450 bp product and the dao1 probe is PCR amplified from pSB1_Ubi1::dao1-ocs_Ubi1::DsRed2-nos using the primers ACA TCA CGC CAA ATT ACC GC (SEQ ID NO: 84) and GCC CCA ACT CTG CTG GTA TC (SEQ ID NO: 85) producing a 700 bp product.

The probes described supra are radiolabeled using a Megaprime DNA Labeling kit (Amersham International Inc, UK) producing α-³²P dCTP labeled probes essentially according to manufacturer's instructions. Blots are pre-hybridized for a minimum of 4 hours in a pre-hybridization buffer consisting of 0.5M sodium phosphate buffer (pH7.5), 7% (w/v) SDS and 1 mM EDTA (pH 7.5). Hybridization with α-³²P dCTP labeled probes is performed for 16-24 h at 65° C. within a rotary hybridization oven at 40 rpm with a fresh hybridization buffer consisting of 0.5M sodium phosphate buffer (pH7.5), 7% (w/v) SDS and 1 mM EDTA (pH 7.5) at a ratio of approximately 1 ml of hybridization buffer per square centimeter of membrane. Southern hybridization blots are washed in sequence, with the following solutions: 3× with 50 mL Wash Solution #1 for 30 mins at 65° C., 2× with Wash Solution #2 for 30 mins at 65° C. Wash Solution #1 comprises 40 mM sodium phosphate buffer (pH7.5), 5% SDS and 1 mM EDTA (pH7.5) and Wash Solution #2 comprises 40 mM sodium phosphate buffer (pH7.5), 1% SDS and 1 mM EDTA (pH7.5). Membranes are removed from the hybridization bottle and placed on Whatman paper to remove excess wash solution, wrapped in plastic cling-wrap an exposed to a Phosphor-imaging screen or placed on x-ray film.

Typically Southern hybridization analysis from Agrobacterium-mediated transformation reveals a low copy integration number (1 to more than 6 copies) with a high proportion of single copy events. Fragments identified from Southern analysis using pCAMBIA1305.2 with the restriction enzyme EcoRI are typically between 2-30 Kb in size.

Segregation of transgenes in wheat plants follows normal Mendelian inheritance of transgenic loci. For single locus and two loci events, a segregation ratio of 3:1 and 15:1 respectively is expected. The segregation of transgene loci can be observed in the seeds of T₁ and T₂ progeny through germination of transgenic seeds in the presence of selective agents. For example, germination in the presence of greater than 5 mM D-serine to allow the discrimination of transgenic and non-transgenic pSB1_Ubi1::dao1-ocs_Ubi1::DsRed2-nos plants.

### Example 7

### Transformation of a Diverse Range of Wheat Genotypes

To determine the general applicability of the method of the present invention for transforming wheat mature embryos freshly isolated from dried grain from a diversity panel of wheat genotypes (Table 8) were transformed using a method essentially as described in Example 1.

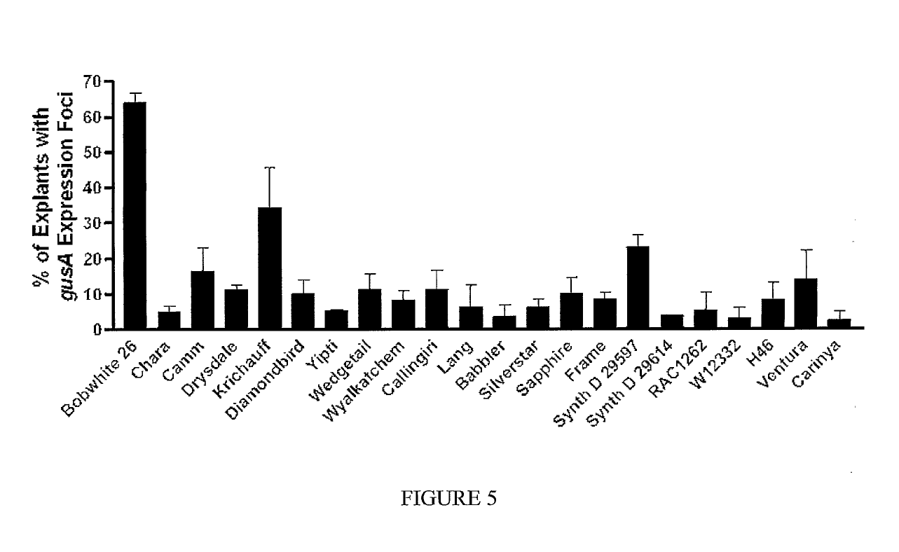

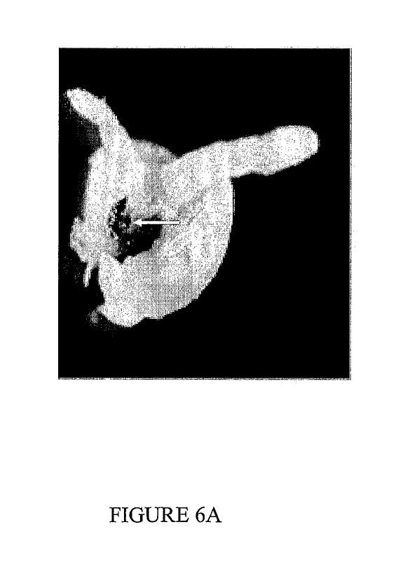

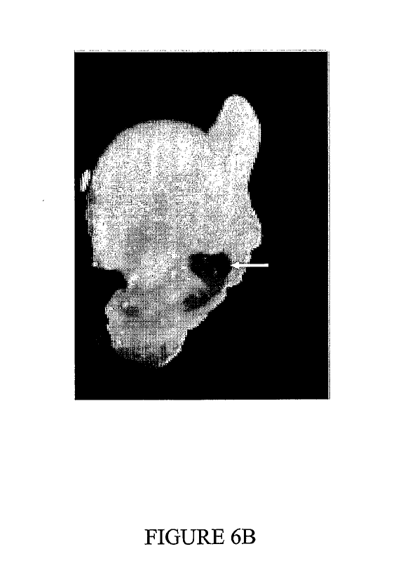

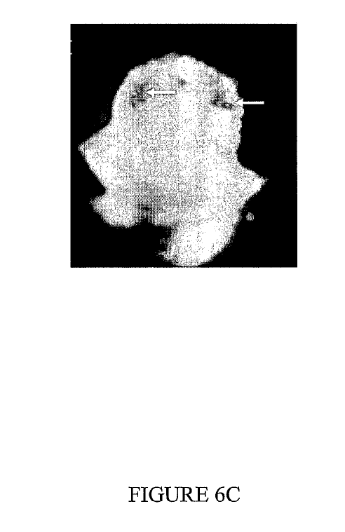

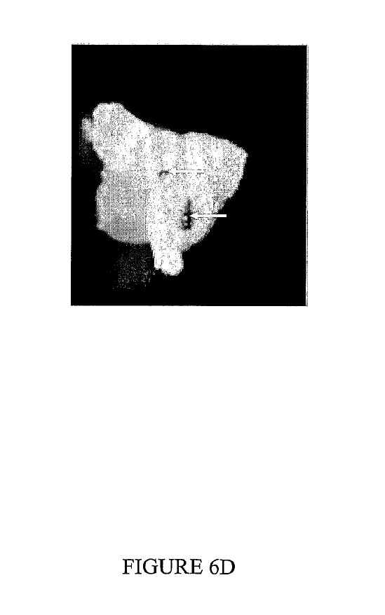

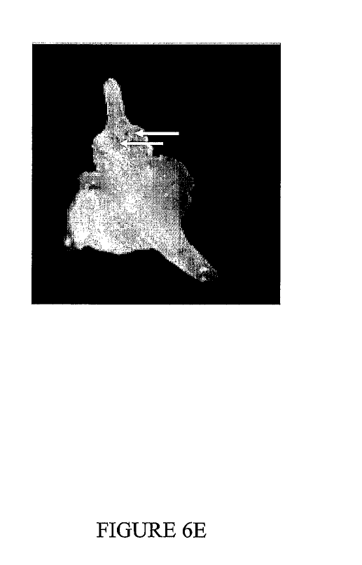

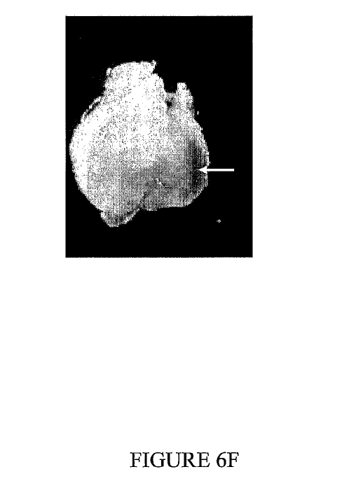

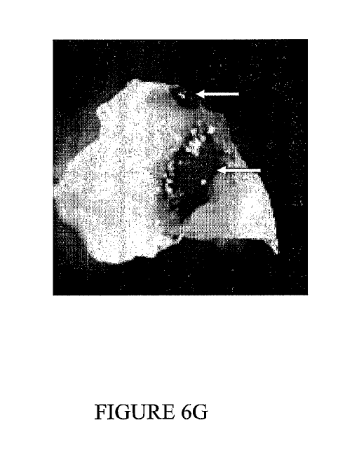

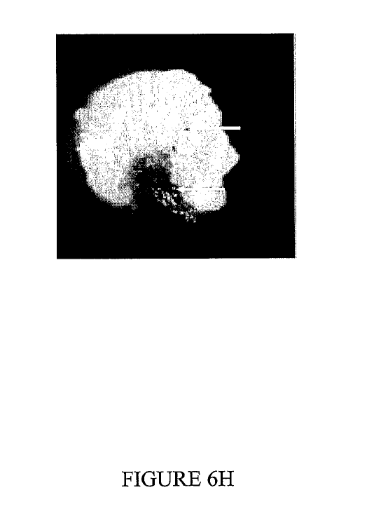

Three days following inoculation, gusA expression was determined essentially as described in Example 1. As shown in FIGS. 5 and 6 the transformation method was capable of transforming all varieties tested in this study, indicating the general applicability of the method.

TABLE 8
Wheat Genotypes Used for Agrobacterium-Mediated Transformation

| | Year | |
|---|---|---|
| Variety | Released | Characteristics |
| Bobwhite | 1970s | Bobwhite represents a group of 129 accessions in the |
| | | CIMMYT (Centro International de Milioramento de Mais y |
| | | Trigo) ex situ wheat collection. The sister lines were |
| | | generated from a cross between CM 33203 with the |
| | | pedigree ‘Aurora’//‘Kalyan’/‘Bluebird 3’/‘Woodpecker’. |
| Lang | 2000 | Similar to Sunco but generally achieves higher yields and |
| | | has stronger straw. |
| Silverstar | 1996 | Early maturity, disease resistant. |
| Wedgetail | 2002 | Prime hard winter wheat, late maturity, acid soil tolerant |
| Wyalkatchem | 2001 | Excellent yield potential, large grain, short stature, adapted |
| | | to low rainfall areas. |
| Calingiri | 1997 | Excellent yield potential, noodle wheat grown mainly in |
| | | Western Australia. Long season with good to moderate rust |
| | | resistance. |
| Sapphire | 2004 | Widely adapted with consistent yields. |
| Diamondbird | 1997 | Suited to medium-high rainfall, acid soil tolerant. |
| Frame | 1994 | Large grain, good early vigor, suited to low rain areas. |
| Yitpi | 1998 | Broad adaptation, early mid-season maturity. |
| Krichauff | 1997 | High yield potential, adapted to low rainfall areas, yellow |
| | | flour requiring specialized marketing |
| Chara | 1998 | Hard white grained wheat, high yielding, broadly adapted, |
| | | mid season maturity. |
| Drysdale | 2002 | White hard grained wheat, increased water use efficiency, |
| | | short season to maturity. |
| Babbler | 2000 | Suited to low-medium rainfall, resistant to stem rust. |
| Camm | 1998 | Zinc efficient variety but not suited to tight cereal rotations. |
| | | Stripe rust resistance, broken down to stem and leaf rust,. |
| Synthetic Derivative AU29597 | | |
| Synthetic Derivative AU29614 | | |
| RAC1262 | | Advanced Breeding Line |
| W12332 | | Advanced Breeding Line |
| H46 | | Advanced Breeding Line |
| Ventura | 2006 | Semi dwarf variety with early maturity. Resistant to the |
| | | three rusts and tolerant to root lesion nematode. Best |
| | | performance has been on acid soils. |
| Carinya | 2005 | Syn. SUN421T. Adaptation and maturity similar to Janz |
| | | with 3% higher yield in the south. Slightly shorter height |
| | | and larger grain size than Janz. |

### Example 8

### Plant Regeneration from a Variety of Wheat Genotypes

To determine the general applicability of the method of the present invention for producing transgenic wheat plants, mature embryos freshly isolated from dried grain from the wheat varieties in Example 4 were subjected to Agrobacterium-mediated transformation using a method essentially as described in Example 1 and regeneration using methods essentially as described in Examples 2 to 4.

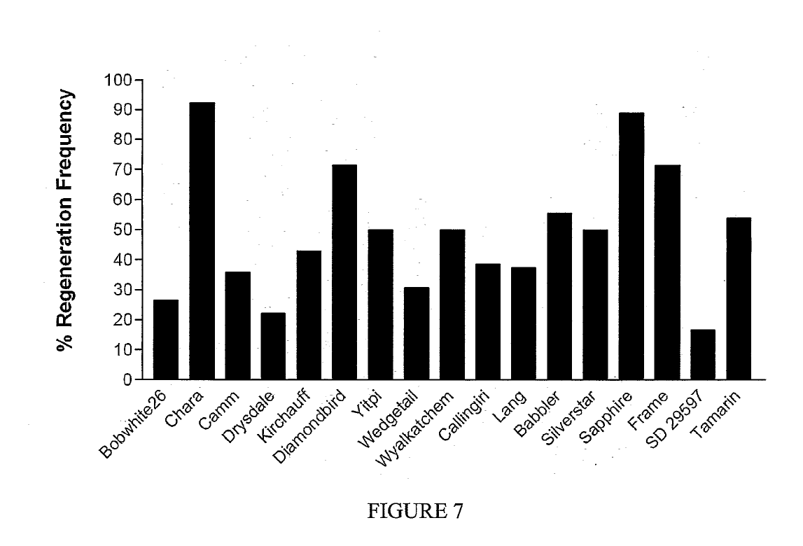

FIGS. 7 and 8 show the variability of regeneration frequency of the diversity panel of wheat genotypes.

### Example 9

### The effect of Soytone™ on Transformation Efficiency

The presence of a peptone in culture media of Agrobacterium increases the expression of genes associated with cellulose biosynthesis (Matthysse et al., Proc. Natl. Acad. Sci., USA, 101: 986-991, 2004). To test whether or not peptone assists in Agrobacterium-mediated transformation, the transformation method described in Example 1 was performed, however, the concentration of Soytone™ was varied in the inoculation and co-culture medium.

Transformation efficiency was determined by calculating the mean number of gusA expressing foci per explant 3 days after inoculation, essentially as described in Example 1.

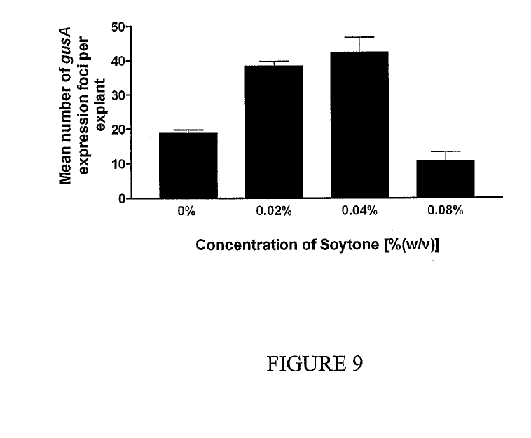

As shown in FIG. 9, even in the absence of Soytone™ the transformation method was capable of producing transgenic wheat cells. However, the presence of Soytone™ (e.g., at 0.2% or 0.4% (w/v)) dramatically increased the mean number of gusA positive foci in each explant. These concentrations approximately doubled the mean number of gusA positive foci in each explant.

### Example 10

### Factors Affecting Transformation Efficiency

To determine optimal conditions for transformation, the method described in Example 1 was modified to test a variety of conditions. For example, the effect of various concentrations of nutrients in media, the presence or absence of a seed coat on the embryo, the presence or absence of Soytone™ and/or the presence of particular sugars were tested. In particular, the effect of the following conditions was determined:
diluting Murashige and Skoog media 1:20;
diluting Murashige and Skoog media 1:10;
diluting Murashige and Skoog media 1:2 and removing the seed coat from the embryo;
adding Soytone™ and removing the seed coat;
adding 2% sorbitol;
adding 2% maltose;
adding 2% glucose; and
adding 2% sucrose.

Transformation efficiency was determined by calculating the mean proportion of explants expressing gusA foci 3 days after inoculation, essentially as described in Example 1.

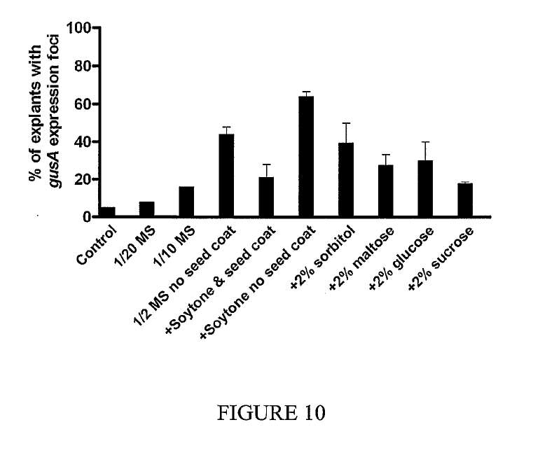

As shown in FIG. 10 all conditions tested resulted in transformation of wheat cells, demonstrating the robust nature of the method of transformation. FIG. 10 also shows that optimal transformation conditions involved the addition of Soytone™ and seed coat removal.

### Example 11

### PPT Resistant Transgenic Wheat Plants

11.1 Vector pBPS0054 and Transformation into Wheat Embryos

Vector pBPS0054 is based on the vector pPZP200 described in Hajdukiewicz et al., Plant Mol. Biol. 25: 989-94, 1994. However, the vector is modified to include the bar gene for PPT resistance under the control of the constitutive maize ubiquitin promoter. A vector map of pBPS0054 is shown in FIG. 7.

The wheat varieties transformed with the pBPS0054 using the method described in Example 1 are described in Table 8.

### 11.2 Regeneration of Transgenic Wheat Plants

Following co-cultivation and subsequent washing, explants are placed on a callus induction medium as described in Example 1 without selection but with antibiotics (cefotaxime 250 mg/L or timentin 150 mg/L) to control Agrobacterium growth. The mature embryo explants are allowed to produce calli for 3 weeks in the dark. Explants showing callusing are sub-cultured regularly to fresh media supplemented with antibiotics. During subculture non-embryogenic calli are removed leaving epiblast and the responsive regions of scutellar tissue.

After at least 3 weeks callus induction, embryogenic calli are transferred to a regeneration medium consisting of 1× Murashige and Skoog (supra) macronutrients, 1× micronutrients and organic vitamins, supplemented with 200 mg/L casein hydrolysate, 100 mg/L myo-inositol, 3% (w/v) sucrose, 2 mg/L zeatin and 0.8% (w/v) Bacto-Agar and antibiotics (cefotaxime 250 mg/L or timentin 150 mg/L) to control Agrobacterium growth. Explants are cultured in the light for 2 weeks then transferred to fresh regeneration media supplemented with 2.5-10 mg/L phosphinothricin and antibiotics. Regenerating tissues are passaged through a further 1 to 2 subculture cycles of 2-3 weeks with putative transgenic plantlets/calli transferred to fresh regeneration media supplemented with chemical selection agents.

After a further 10 days, regenerated plantlets are transferred to MS_{[1/2}] supplemented with 1 mg/L NAA (RM) for root initiation. Any regenerated plantlets surviving greater than 3 weeks on RM with healthy root formation are potted into a nursery mix consisting of peat and sand (1:1) and kept at 22-24° C. with elevated humidity under a nursery humidity chamber system. After two weeks, plants are removed from the humidity chamber and hand watered and liquid fed Aquasol™ weekly until maturity. The T₀ plants are sampled for genomic DNA and molecular analysis and mature T, seed collected.

### 11.3 Determining PPT Resistance

For each plant line produced, three healthy looking equal sized leaves from separate tillers are selected for leaf painting. PPT (at 0.2 g/l and 2 g/l) is applied in the form of BASTA herbicide (Bayer Crop Sciences) with the wetting agent Tween-20 (0.1%), using a cotton bud to paint the upper surface of the distal half of the selected leaves (7-10 cm). Tween-20 (0.1%) alone is used as a control. After 7 days, PPT resistance is determined according to the proportion of necrosis suffered over the area painted with the herbicide solution.

### 11.4 PCR Analysis of Plants to Detect the Presence of the Bar Gene

Genomic DNA is isolated using the Qiagen Mini Plant DNA extraction kit following manufacturer's instructions. DNA is quantified using a nanodrop spectrophotometer prior to PCR.

The primer sequences for PCR are: wknox4D 5′-CAA CAG GAG AGC CAG AAG GT-3′ and 5′-AGG TCA CCG GTA ACG GTA AG-3′. This primer pair acts as a positive internal PCR control amplifying 250 bp of the Knotted 1 4D allele. bar-5′-GTC TGC ACC ATC GTC AAC C-3′(SEQ ID NO: 63) and 5′-GAA GTC CAG CTG CCA GAA AC-3′ (SEQ ID NO: 64),

PCR reactions are cycled using standard techniques, with the annealing temperature for the reaction to detect the bar gene being 57° C. At least two replicates are carried out for each PCR analysis.

Reactions are electrophoresed on agarose gels and the presence of a 444 bp amplification product is indicative of the presence of the transgene in the sample tested.

### Example 12

### A Ti Vector for Plant Transformation

DNA and RNA manipulation are performed using standard techniques. The yeast R. gracilis is grown in liquid culture containing 30 mM D-alanine to induce dao1, the gene encoding DAAO. Total RNA is isolated from the yeast and used for cDNA synthesis. The PCR primers 5′-ATTAGATCTTACTACTCGAAGGACGCCATG-3′ (SEQ ID NO: 64) and 5′-ATTAGATCTACAGCCACAATTCCCGCCCTA-3′ (SEQ ID NO: 65) are used to amplify the dao1 gene from the cDNA template by PCR. The PCR fragment is sub-cloned into the pGEM-T Easy vector (Promega) and subsequently used to replace the bar resistance gene in pPZP200 ubi::bar-nos_R4R3 to produce pPZP200 ubi::dao1-nos R4R3. The vectors are analyzed using sequencing to check that they contain the correct constructs.

Nucleic acid encoding dsRED is PCR amplified using primers comprising the sequences attB1-ATGGCCTCCTCCGAGGAC (SEQ ID NO: 66) and attB2-GCCACCATCTGTTCCTTTAG (SEQ ID NO: 67) and using the pdsRED vector available from Clontech as a template. The PCR fragment is recombined into the pDONOR221 vector (Invitrogen) to produce a pDONOR/dsRED Entry Clone.

Nucleic acid comprising 2175 bp of 5′ untranslated promoter sequence, act1D (act1D) from rice is PCR amplified using primers comprising the sequences attB4-ATCGACTAGTCCCATCCCTCAGCCGCCTTTCACTATC (SEQ ID NO: 68) and attB1-ATCGGCGGCCGCCCCATCCTCGGCGCTCAGCCATCTTCTACC (SEQ ID NO: 69) The PCR fragment is recombined into the pDONORP4-P1R vector (Invitrogen) to produce a pDONOR/act1D Entry Clone. Furthermore, and nucleic acid comprising the CaMV35s polyadenylation signal is PCR amplified using primers comprising the sequences attB2-ATCGCCACCGCGGTGGAGTCCGCAAAAATCACCAGTCTC (SEQ ID NO: 70) and attB3-ATCGCCACCGCGGTGGaGGTCACTGGATTTTGGTTTTAGG (SEQ ID NO: 71) The PCR fragment is recombined into the pDONORP2R-P3 vector (Invitrogen) to produce a pDONOR/35ST Entry Clone.

The Entry clones pDONOR/act1D, pDONOR/dsRED, pDONOR/35ST are recombined into the destination vector pPZP200 ubi::dao1-nos_R4R3 to produce the vector pPZP200 ubi::dao1-nos_act1D::dsRED-35ST. The vector is analyzed using sequencing to confirm that it contains the correct constructs.

The pPZP200 ubi::dao1-nos_act1D::dsRED-35 expression vector is transformed into plant embryos essentially as described in Example 1. Sections from transformed embryos are then analyzed for dsRED expression using a Zeiss (Jena, Germany) LSM 510 CLSM implemented on an inverted microscope (Axiovert 100). Excitation is provided by a 488 nm Ar laser line, controlled by an acousto optical tuneable filter. To separate excitation from emission, two dichroic beam splitters are used. The HFT 488 dichroic beam splitter is used to reflect excitation and transmit fluorescence emission. A mirror is used to reflect the emitted fluorescence to the NFT 545 secondary beam splitter. Fluorescence transmitted by the NFT 545 splitter is filtered through a 565 to 590 nm band pass filter, resulting in the red channel. A Zeiss plan-neofluar 40× (N.A. 1.3) oil immersion objective lens is used for scanning.

Those embryo sections positive for dsRED expression are then selected for plant regeneration essentially as described in any one of Examples 2 to 3. Embryos and calli are grown on growth medium comprising 5 mM D-alanine, 5 mM D-serine. Transgenic plants are selected using D-alanine and D-serine.

### Example 13

### Transgenic Wheat Having Improved Bread Making Characteristics

### 13.1. Wheat Having Enhanced HMW-GS 1Ax1 Expression

The plasmid pHMW1Ax1 contains the HMW-GS 1Ax1 gene of wheat, the expression of which is driven by its own endosperm specific promoter (Halford et al., Theoret. Appl. Genet. 83:373-378, 1992). The HMW-GS 1Ax1 gene and promoter are excised from pHMW1Ax1 and cloned into pPZP200 ubi::dao1-nos_act1D::dsRED-35, replacing the act1D promoter and dsRED, to produce the vector pPZP200 ubi::dao1-nos HMW-35.

The pPZP200 ubi::dao1-nos HMW-35 vector is then transformed into wheat embryos from a variety of genotypes. Transgenic wheat plants are then regenerated using methods essentially as described in any one of Examples 2 to 4. To plants are grown to maturity and selfed to produce T₁ plants. Seeds are then collected from T₀ and T₁ plants.

### 13.2 Protein Analysis

Protein extracts are prepared by grinding mature dry seeds individually with a mortar and pestle. Ten to fourteen mg of the resultant flour from each seed is vortexed with 200 μl sample buffer (2% SDS, 5% β-mercaptoethanol, 0.001% Pyronin Y, 10% glycerol, 0.063 M Tris HCl pH 6.8) for 2 minutes and incubated for 2 hours on a rotary shaker at 250 rpm. The extracts are centrifuged (10 minutes, 14,000 rpm) and the supernatant boiled for 5 minutes to denature the protein. The proteins are separated by SDS-PAGE (essentially according to Laemmli, Nature 227:680-685, 1970). Briefly, 20 to 30 μl of each sample is loaded in 13 cm gels containing 10% (w/v) acrylamide, 0.8% (w/v) bis-acrylamide and run until the dye front had reached the bottom of the gel, so that the total extracted protein remained on the gel. The 1Ax1 band is resolved from the rest of the HMW-GS which are not completely separated from one other. The gels are first fixed in the staining solution without dye for 0.5 to 1 hour and then stained in Coomassie Brilliant Blue R-250 for 4 to 6 hours (essentially according to Neuhoff et al., Electrophoresis 9:255-262, 1988). Protein bands are visualized by destaining in an aqueous solution of 5% methanol and 7% acetic acid (vol/vol) until a clear background is obtained. Gels are stored in a 7% aqueous acetic acid solution (vol/vol). Stained gels are scanned using a digital imaging system, e.g., an Alpha Innotech (San Leandro, Calif.) IS-1000 Digital. Imaging System. Lane and peak values are corrected by interband background subtraction. Background intensity is determined for each individual lane from the top of each HMW-GS 1Ax1 band at approximately 140 kDa. The amount of HMW-GS 1Ax1 present is calculated relative to the corrected lane value or the corrected HMW-GS value. To calculate the total HMW-GS level, the protein contents of each lane are normalized.

### 13.3 Southern Analysis

Genomic DNA is isolated from the leaves of plants capable of growing in the presence of D-serine and D-alanine by the CTAB method (essentially as described in Lassner et al., Plant Molec. Biol. Rep. 7:116-128, 1989). Purified DNA (20 to 25 μg) is digested with XbaI, electrophoresed in 0.8% agarose gel, and blotted on Hybond-N membrane (Amersham). The probe for hybridization consists of a 2.2 kb fragment from the coding region of the HMW-GS 1Ax1 gene, derived after an EcoRI and HindIII digest of pHMW1Ax1. The probe is labeled using the random primer labeling kit (GIBCO-BRL). Hybridization is performed at 65° C. for 24 hours, and signals visualized by autoradiography.

### 13.4 Segregation Analysis

To determine the segregation ratios of transgene DAAO in the T₁ generation, 20 mature embryos from each of the transgenic lines are germinated on a medium supplemented with D-alanine and D-serine: half strength MS-salts and vitamins (supra) supplemented with 15 g/l sucrose, 2.5 g/l gelrite, 5 mM D-alanine, 5 mM D-serine, pH 5.8 (B3 medium). Lines homozygous for DAAO were identified from T₂ seeds, by testing the germinability of 20 embryos from up to 12 T₁ plants of all HMW-GS 1Ax1 accumulating lines on B3 medium. Ten seeds of each homozygous DAAO line are analyzed individually by SDS-PAGE for HMW-GS 1Ax1 to determine if co-segregation has occurred.

### Example 14

### Wheat Expressing Hepatitis B Surface Antigen (HBsAg)

### 14.1 Wheat Expressing HBsAg

The HBsAg DNA coding sequence (Cattaneo, Nature 305: 336-338, 1983) is PCR amplified from the plasmid p R/HBs-3 using primers containing the attB1 and attB2 sequences. This fragment is recombined into pDONOR221 to generate the Entry Clone pDONOR/HBsAg. This fragment is then recombined into the destination vector pPZP200 ubi::dao1-nos_R4R3 with the Entry Clones pDONOR/act1D, and pDONOR/35ST to produce pPZP200 ubi::dao1-nos_act1D::HbsAg-35ST.

14.2 Transfer of pCAMBIA:dao1/dsRED-HBsAg to A. tumefaciens

Plasmid pCAMBIA:dao1/dsRED-HBsAg is transferred to A. tumefaciens strain LBA4404 obtained from Clontech Laboratories, Inc.

A. tumefaciens is cultured in AB medium (An, Meth. Enzymol. 153: 292-305, 1987) until the optical density (O.D.) at six hundred nanometers (600 nm) of the culture reaches about 0.5. The cells are then centrifuged at 2000 g to obtain a bacterial cell pellet. The Agrobacterium pellet is resuspended in 1 ml of ice cold 20 mM CaCl₂. Plasmid (0.5 μg) is added to 0.2 ml of the calcium chloride suspension of A. tumefaciens cells in a 1.5 ml microcentrifuge tube and incubated on ice for 60 minutes. The plasmid and A. tumefaciens cell mixture is frozen in liquid nitrogen for 1 min., thawed in a 25° C. water bath, and then mixed with five volumes of rich MGL medium (An supra). The plasmid and A. tumefaciens mixture is then incubated at 25° C. for four hours with gentle shaking. The mixture is plated on Luria broth agar medium containing 100 μg/ml spectinomycin. Plates are incubated for three days at 25° C. before selection of resultant colonies which contained the transformed Agrobacterium harboring the plasmid.

### 14.3 Transformation of Wheat

The pPZP200 ubi::dao1-nos_act1D::HbsAg-35ST vector is then transformed into wheat embryos from a variety of wheat genotypes using a method essentially as described in Example 1. Transgenic wheat plants are then regenerated using methods essentially as described in any one of Examples 2 to 4. T₀ plants are grown to maturity and selfed to produce T₁ plants. Seeds are then collected from T₀ and T₁ plants.

### 14.4 Biochemical and Immunochemical Assays

Root, stem, leaf and seed samples are collected from plants. Each tissue is homogenized in 100 mM sodium phosphate, pH 7.4 containing 1.0 mM EDTA and 0.5 mM PMSF as a proteinase inhibitor. The homogenate is centrifuged at 5000×g for 10 minutes. A small aliquot of each supernatant is then reserved for protein concentration using the Lowry method. The remaining supernatant is used for the determination of the level of HBsAg expression using two standard assays: (a) a HBsAg radioimmunoassay, the reagents for which are purchased from Abbott Laboratories and (b) immunoblotting using a previously described method of Peng and Lam (Vis. Neurosci. 6: 357, 1991) with a monoclonal antibody against anti-HBsAg purchased from Zymed Laboratories.

### Example 15

### Transgenic Wheat Having Resistance Against Wheat Streak Mosaic Virus (WSMV)

### 15.1 Wheat Stably Expressing the WSMV Coat Protein

Plasmid pPZP200 ubi::dao1-nos R4R3 is engineered to introduce a nucleic acid encoding a WSMV coat protein (SEQ ID NO: 48). The resultant plasmid is designated pPZP200 ubi::dao1-nos_act1D::WSMV-35ST.

pPZP200 ubi::dao1-nos_act1D::WSMV-35ST is then transformed into wheat embryos from a variety of genotypes by performing a method essentially as described in Example 1. Transgenic wheat plants are then regenerated using a method essentially as described in any one of Examples 2 to 4. To plants are grown to maturity and selfed to produce T₁ plants. Seeds are then collected from T₀ and T₁ plants.

### 15.2 Assay to Determine Resistance to WSMV

Seeds are isolated from transgenic wheat plants and wild-type (untransformed) wheat plants. Seeds are mechanically inoculated with a solution comprising WSMV. Innoculated seeds are then planted and wild-type and transgenic seedlings grown in a growth chamber.

Following sufficient growth to allow leaf formation, leaves are observed for visual symptoms of WSMV infection, such as, for example, leaf yellowing, leaf malformation and/or leaf curling.

Provided that wild-type plants develop symptoms of WSMV infection and show expression of WSMV coat protein, it is presumed that those transgenic plants that do not demonstrate such symptoms are resistant to this pathogen.

### Example 16

### Head Scab Resistant Wheat

### 16.1 Production of Wheat Stably Expressing a Thaumatin-Like Gene

pPZP200 ubi::dao1-nos_R4R3 is modified to clone a thaumatin-like gene (SEQ ID NO: 50) in the R4R3 cassette with the act1D promoter and 35S polyadenylation signal. The thaumatin-like protein is obtained essentially as described by Kuwabara et al., Physiol. Plantarum 115: 101-110, 2002). Thaumatin-like proteins are stress response proteins that are particularly effective in the treatment of plant pathogens, as they are capable of inhibiting the infection of the plant by such a pathogen.

The resultant vector is designated pPZP200 ubi::dao1-nos_act1D::TL1-35ST

pPZP200 ubi::dao1-nos_act1D::TL1-35ST is then transformed into wheat embryos from a variety of genotypes using a method essentially as described in Example 1. Transgenic wheat plants are then regenerated using a method essentially as described in any one of Examples 2 to 4. To plants are grown to maturity and selfed to produce T₁ plants. Seeds are then collected from T₀ and T₁ plants.

### 16.2 Assay for Wheat-Scab Resistance

Seedlings of transformed wheat are grown in air-steam pasteurized (60° C. for 30 minutes) potting mix (Terra-lite Rediearth, W. R. Grace, Cambridge, Mass.) in a growth chamber at 25° C., 14 h light/day for approximately 8 weeks prior to use in bioassays. Conidial inoculum of Fusarium graminearum isolate Z3639 are produced on clarified V-8 juice agar at 25° C., 12 h light/day for 7 days while biomass of each strain of microorganism is produced on TSA/5 by inoculating plates and incubating at 25° C. for 48 h. Conidia of F. graminearum 3639 are used to inoculate the middle floret of two wheat heads per microbial strain. Inoculated wheat plants are placed in a clear plastic enclosure on greenhouse benches for 72 h to promote high relative humidity. The enclosure is then removed and wheat heads are scored for visual symptoms of Fusarium head blight 16 days after inoculation. Those that show no sign of Fusarium head blight are considered to express a protein that confers protection against head scab.

### 16.2 Greenhouse Assays of Resistance to Head Scab

Transformed and wild-type seedlings are grown two to a pot in pasteurized potting mix in a growth chamber for 8 weeks as described above. Conidia of F. graminearum isolates Z3639, DOAM, and Fg-9-96 are produced on CV-8 agar as described above. After 8 weeks, wheat plants are transferred to greenhouse benches for approximately 1 week. At the onset of wheat head flowering, generally by the end of 1 week on greenhouse benches, biocontrol bioassays are initiated. The middle floret of a wheat head is inoculated with F. graminearum. Inoculated wheat plants are then placed in a plastic enclosure on greenhouse benches for 72 h to promote high relative humidity and free moisture necessary for optimal Fusarium head blight development. Sixteen days after inoculation, wheat heads are scored for disease severity on a 0 to 100% bleached wheat head scale (Stack et al., North Dakota State University Extension Service Bulletin PP-1095, 1995), and a 0 to 100% disease incidence scale. Kernel weights are determined after heads have matured. Fully developed kernels in healthy heads have high 100 kernel weights, while shriveled kernels in heads infected by F. graminearum have lower 100 kernel weights. F. graminearum is recovered from randomly selected heads showing symptoms of disease development.

### Example 17

### Drought Tolerant Wheat

### 17.1 Wheat Stably Expressing DREB1A

pPZP200 ubi::dao1-nos R4R3 is modified to clone DREB1A cDNA (SEQ ID NO: 58) in the recombination cassette with the act1D promoter and 355 polyadenylation signal.

The DREB1A cDNA is obtained essentially as described by Wang et al., Plant Mol. Biol. 28: 605-617, 1995. DREB1A is a late-embryogenesis-abundant (LEA) protein expressed when plants are exposed to drought.

The act1D promoter in pPZP200 ubi::dao1-nos_act1D::DREB1A-35ST is also replaced with the rd29A promoter as expression under a constitutive promoter has been shown to result in severe growth retardation of plants under normal circumstances (Kasuga et al., Nature Biotechnology 17: 287-291, 1999).

The resultant protein is designated pPZP200 ubi::dao1-nos_rd29a::DREB1A-35ST.

pPZP200 ubi::dao1-nos_rd29a::DREB1A-35ST is then transformed into wheat embryos from a variety of genotypes using a method essentially as described in Example 1. Transgenic wheat plants are then regenerated using a method essentially as described in any one of Examples 2 to 4. To plants are grown to maturity and selfed to produce T₁ plants. Seeds are then collected from T₀ and T₁ plants.

### 17.2 Freezing, Drought, and High-Salt Stress Tolerance of the Transgenic Plants

Plants are grown in 9 cm pots filled with a 1:1 mixture of perlite and vermiculite. Plants are grown under continuous illumination of approximately 2500 lux at 22° C. Separate samples of the 3-week-old plants are exposed to freezing and drought stresses.

Freezing stress is created by exposing the plants to −6° C. temperatures for 2 days, then returning to 22° C. for 5 days.

Drought stress is created by withholding water for 2 weeks.

High-salt stress is created by soaking plants that are grown on agar plates and gently pulled out of the growing medium in 600 mM NaCl solution for 2 h.

The plants are then transferred to pots under normal growing conditions for 3 weeks.

The number of plants that survive and continue to grow compared to control (untransformed plants) is then determined. The statistical significance of the values is determined using chi-squared test.

### Example 18

### Wheat Having a Reduced Level of Waxy

18.1 Wheat Stably Expressing siRNA to Inhibit Expression of Granule-Bound Starch Synthase I

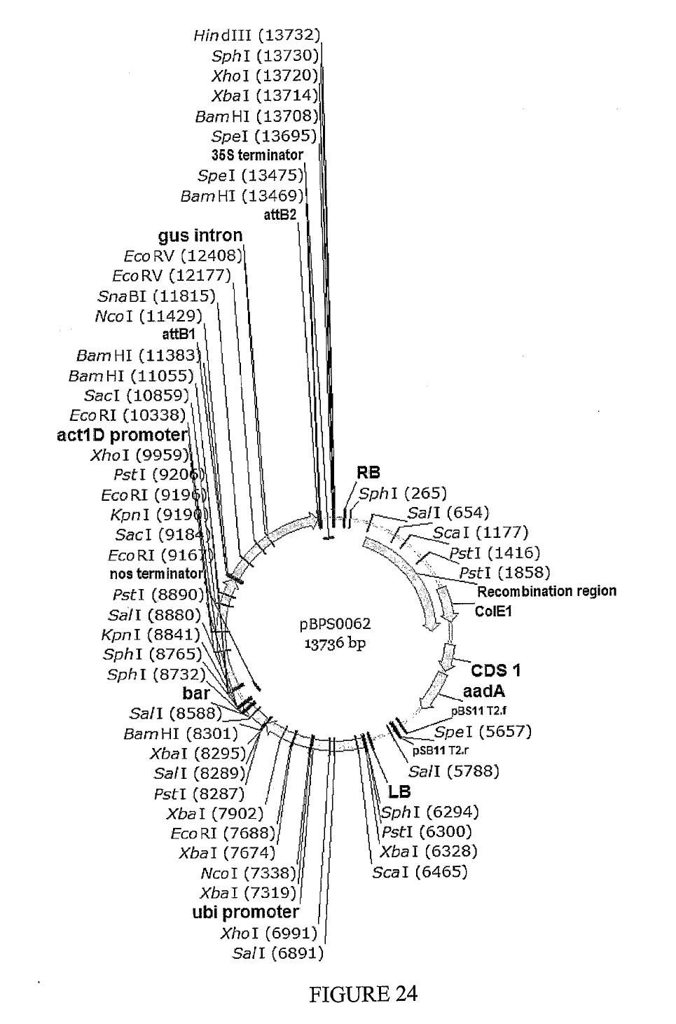

pPZP200 ubi::dao1-nos_act1D-rfa-RGA2-rfa(as)-35ST (FIG. 24) is modified to clone a nucleic acid encoding a siRNA derived from wheat granule bound starch synthase (SEQ ID NO: 60) in the recombination cassette between the act1D promoter and 35S polyadenylation signal. The resulting vector is designated pPZP200 ubi::dao1-nos_act1D::waxy-35ST.

pPZP200 ubi::dao1-nos_act1D::waxy-35ST is then transformed into wheat embryos from a variety of genotypes using a method essentially as described in Example 1 Transgenic wheat plants are then regenerated using a method essentially as described in any one of Examples 2 to 4. T₀ plants are grown to maturity and selfed to produce T₁ plants. Seeds are then collected from T₀ and T₁ plants.

### 18.2 GBSSI Expression in Wheat Seed

Levels of expression of GBSSI mRNA are determined in wheat seeds. Tissue is frozen in liquid nitrogen and ground to a fine powder, then homogenized using a polytron homogenizer. Insoluble material is removed by centrifugation at 12,000×g for 10 min, and the supernatant extracted with chloroform and precipitated with isopropyl alcohol. RNA is extracted using Trizol reagent (Life Technologies/Gibco-BRL, Cleveland) essentially according to the manufacturer's instructions.

Total RNA samples are heat denatured, then separated by electrophoresis in 1% (w/v) agarose gels containing 2.2 M formaldehyde, and transferred to GeneScreen Plus membrane (NEN Research Products, Boston) by capillary transfer. The blots are prehybridized at 42° C. in buffer containing 50% (v/v) formamide, 0.2% (w/v) polyvinylpyrrolidone, 0.2% (w/v) Ficoll, 0.2% (w/v) bovine serum albumin, 50 mm Tris, pH 7.5, 1.0 M NaCl, 0.1% sodium pyrophosphate, 1% (w/v) SDS, 10% (w/v) dextran sulfate, and 100 μg/mL denatured salmon sperm DNA, then hybridized for 1 day in the same buffer containing ³²P-labeled probe. The membranes are washed twice for 30 min in 2×SSC and 1% (w/v) SDS at 65° C., and once in 0.1×SSC at 65° C. for approximately 10 min, or until background radioactivity had dropped to near zero

### 18.3 Amylose Content of Transgenic Wheat Seed

Amylose content is measured by calorimetric method and amperometric titration as follows:

(1) Colorimetric measurement based on iodine coloration is performed following the method of Kuroda et al. (Jpn. J. Breed. 39 (Suppl. 2):142-143, 1989) using an auto-analyzer (Bran Lubbe. Co.). 35 mg of starch is gelatinized in 5 ml of 0.75 N NaOH and 25% aqueous ethanol, and neutralized by acetic acid. Absorbance at 600 nm of the starch iodine complex is measured using calorimeter. As a control, two wheat starches of known starch content are used. A first control, wheat starch purchased from Wako Pure Chemicals Ltd. contains about 31% amylose as determined by the auto-analyzer using potato amylose and amylopectin as standards, and a second control, waxy wheat starch contains about 0.6% amylose.

(2) Amperometric titration (Fukuba and Kainjima, in Starch Science Handbook (Nakamura M. and Suzuki S., eds) Tokyo: Asakura Shoten, pp 174-179, 1977) is performed using defatted starch with an iodine amperometric titration device (e.g., Model 3-05, Mitamura Riken Kogyo, Japan).. Amylose content of the starch is calculated by assuming that 20 mg of iodine can bind to 100 mg of pure wheat amylose. The starch concentration of the solution used is determined using the phenol-sulfuric acid method (e.g., essentially as described in Dubois et al., Anal. Chem. 28:350-356, 1956) with glucose as a standard.

### Example 19

### Agrobacterium-Mediated Transformation of Barley (Hordeum vulgare)

Grain from Hordeum vulgare (e.g., variety Golden Promise) was surface sterilized for 30 minutes in a 0.8% (v/v) NaOCl solution and rinsed at least four times in sterile distilled water.

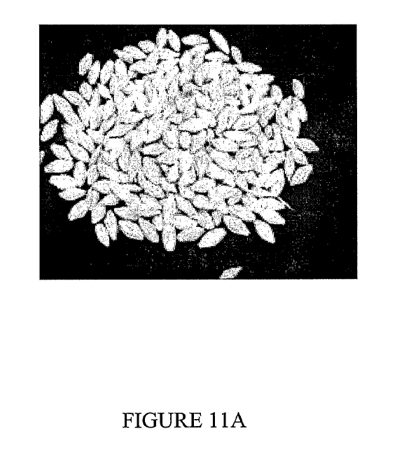

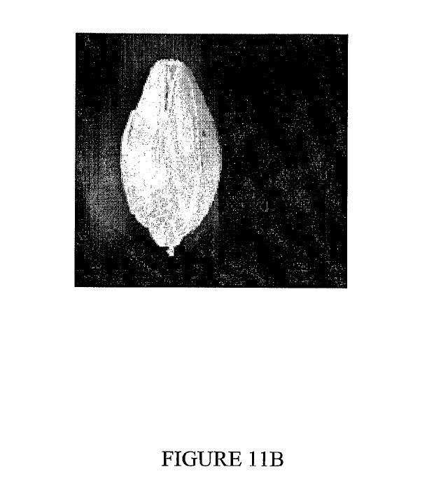

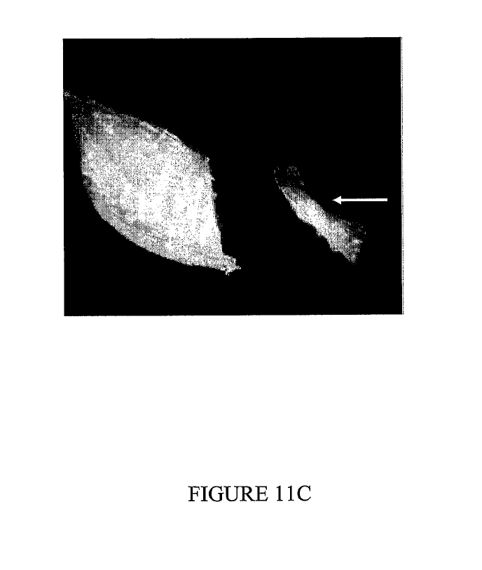

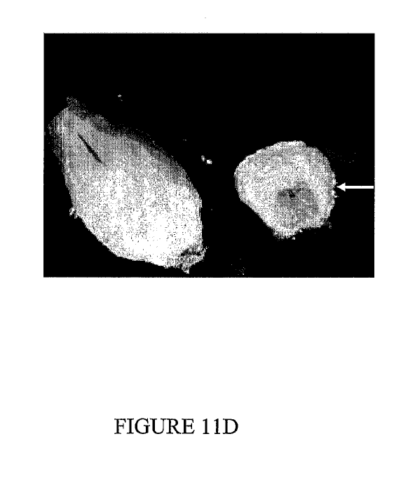

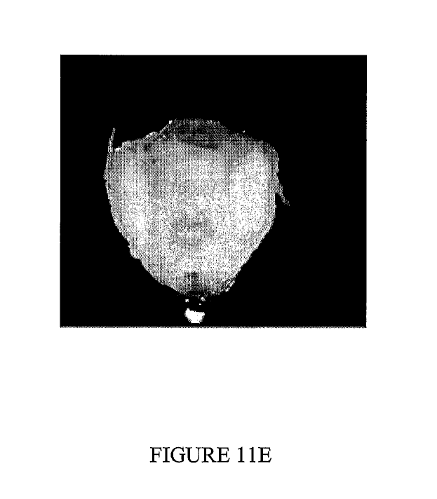

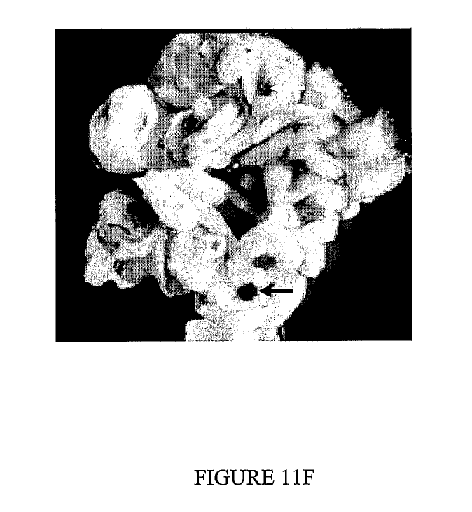

Mature embryos were aseptically excised from surface sterilized grain, the seed coat removed and used directly for Agrobacterium-mediated transformation. FIGS. 11A-E shows the isolation of embryo with intact epiblast and scutellum from dried barley grain.

Explants were used directly for Agrobacterium-mediated transformation. Agrobacterium strain EHA105 comprising the pCAMBIA1305.2 vector (expressing the GUS reporter gene under control of the CaMV35s promoter) was used to inoculate 10-15 mL of LB supplemented with 100 μg/mL of rifampicin and kanamycin in a 50 mL Falcon tube, which is incubated for 24 to 48 hours at 27-28° C. For inoculation, 100 μl of the Agrobacterium culture was used to inoculate 25 mL of fresh LB supplemented kanamycin and incubated for 24 hours. This full strength inoculum was centrifuged at 3000 rpm for 10 minutes at room temperature with the resulting pellet re-suspended in liquid inoculation medium (MS_{[1/10]}) to an OD₆₀₀=0.25-0.8. The inoculation medium consisted of 1/10 strength liquid Murashige and Skoog (1962) basal salts (MS_{[1\10]}) supplemented with 2 mg/L 2,4-D, 200 μM acetosyringone, and 0.02% (w/v) Soytone™.

Agrobacterium infection was standardised for 3 hours at room temperature with gentle agitation, followed by 3 days of co-cultivation in the dark on a medium consisting of 1× Murashige and Skoog (Murashige and Skoog Physiol. Plant, 15: 473-497, 1962) macronutrients, 1× micronutrients and organic vitamins, supplemented with 200 mg/L casein hydrolysate, 100 mg/L myo-inositol, 3% (w/v) sucrose, 2 mg/L 2,4-D supplemented with 200 μM acetosyringone and 0.8%-2.0% (w/v) Bacto Agar at 21° C. with the embryo axis preferably facing downwards.

Explants were optionally then washed thoroughly with liquid MS_{(1/10)} without acetosyringone or Soytone™ but supplemented with 250 mg/L cefotaxime. Alternatively, explants are washed in sterile water supplemented with 250 mg/L cefotaxime until no visible signs of Agrobacterium remain (i.e. wash solution remains clear after washing).

Transient gusA expression was determined on explants sampled after 3 days (or as indicated otherwise) on induction medium containing 150 mg/L timentin, using the histochemical GUS assay (Jefferson Plant Mol. Biol. Rep. 5: 387405 1987). Explants were incubated overnight at 37° C. in buffer containing 1 mM X-Gluc, 100 mM sodium phosphate buffer pH 7.0, potassium 0.5 mM ferricyanide, 0.5 mM potassium ferrocyanide and 0.1% (v/v) Triton X-100. Blue gusA expression foci were counted under a microscope and T-DNA delivery assessed by counting explants that had at least one gusA expression foci and then counting the number of foci per embryo. To assay for stable gusA expression calli, shoots and leaf fragments from regenerating plantlets were incubated overnight at 37° C. and, if necessary, for a further 1-2 days at 25° C. As shown in FIG. 11F, gusA expression is detectable in the transformed embryos 3 days after inoculation.

### Example 20

### Callus Induction and Regeneration of Transgenic Barley Plants

To determine the general applicability of the method of the present invention for transforming barley, transformed embryos from dried grain from the barley variety Golden Promise as described in Example 19 were regenerated using a method essentially as described in any one of Examples 2 to 4, respectively.

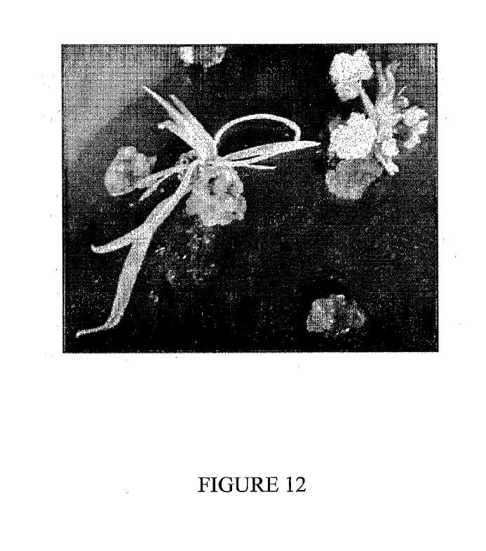

FIG. 12 shows the regeneration of barley plants derived from Agrobacterium-mediated transformation of mature embryos derived from dried grain.

### Example 21

### Agrobacterium-Mediated Transformation of Mature Rice

### Oryza sativa

Grain from Oryza sativa (e.g., Jarrah a Japonica type) was surface sterilized for 30 minutes in a 0.8% (v/v) NaOCl solution and rinsed at least four times in sterile distilled water.

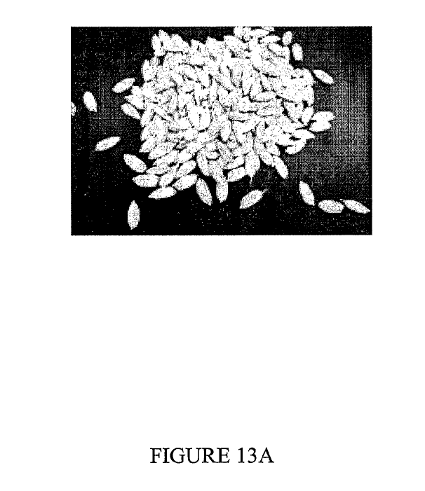

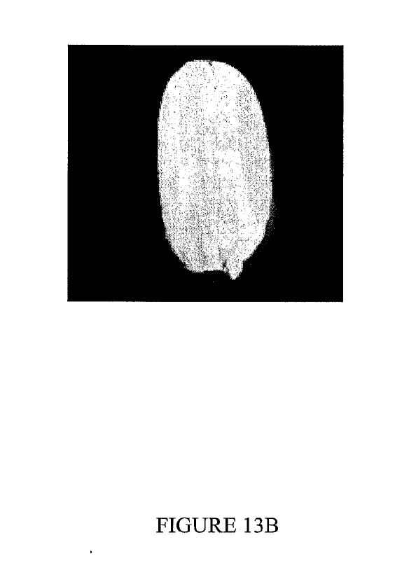

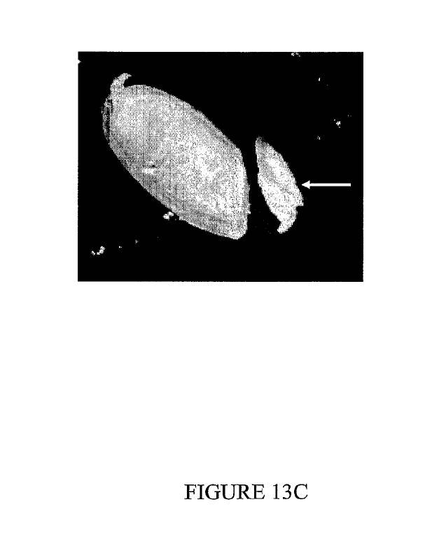

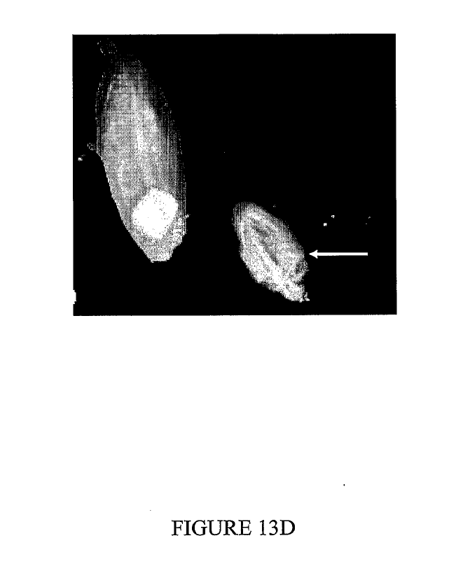

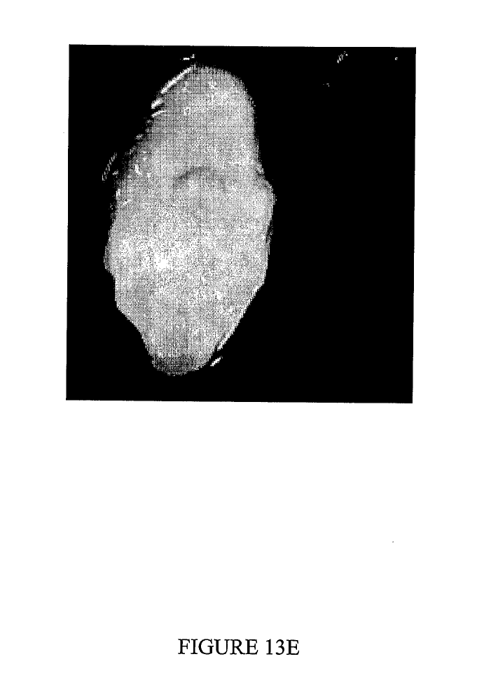

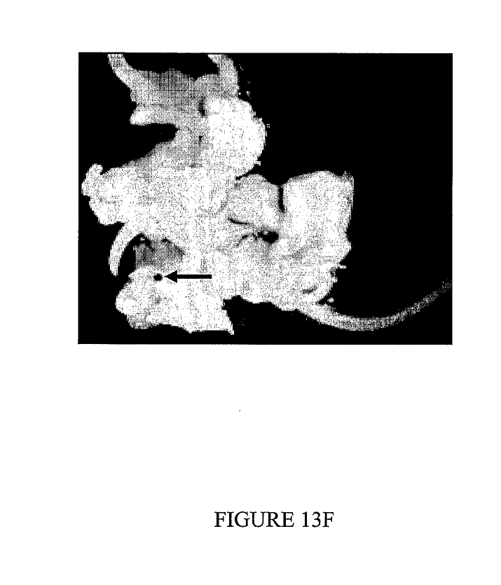

Mature embryos were aseptically excised from surface sterilized dried rice grain, the seed coat removed and used directly for Agrobacterium-mediated transformation. FIG. 13A-F shows the isolation of embryo with intact epiblast and scutellum from dried rice grain and transformation of the isolated embryo.

Explants were used directly for Agrobacterium-mediated transformation. Agrobacterium strain EHA105 comprising the pCAMBIA1305.2 vector (expressing the GUS reporter gene under control of the CaMV35s promoter) was used to inoculate 10-15 mL of LB supplemented with 100 μg/mL of rifampicin and kanamycin in a 50 mL Falcon tube, which is incubated for 24 to 48 hours at 27-28° C. For inoculation, 100 μl of the Agrobacterium culture was used to inoculate 25 mL of fresh LB supplemented kanamycin and incubated for 24 hours. This full strength inoculum was centrifuged at 3000 rpm for 10 minutes at room temperature with the resulting pellet re-suspended in liquid inoculation medium (MS_{[1/10]}) to an OD₆₀₀=0.25-0.8. The inoculation medium consisted of 1/10 strength liquid Murashige and Skoog (1962) basal salts (MS_{[1/10]}) supplemented with 2 mg/L 2,4-D, 200 μM acetosyringone, and 0.02% (w/v) Soytone™.

Agrobacterium infection was standardised for 3 hours at room temperature with gentle agitation, followed by 3 days of co-cultivation in the dark on a medium consisting of 1× Murashige and Skoog (Murashige and Skoog Physiol. Plant, 15: 473-497, 1962) macronutrients, 1× micronutrients and organic vitamins, supplemented with 200 mg/L casein hydrolysate, 100 mg/L myo-inositol, 3% (w/v) sucrose, 2 mg/L 2,4-D supplemented with 200 μM acetosyringone and 0.8%-2.0% (w/v) Bacto Agar at 21° C. with the embryo axis preferably facing downwards.

Explants are optionally then washed thoroughly with liquid MS_{(1/10)} without acetosyringone or Soytone™ but supplemented with 250 mg/L cefotaxime. Alternatively, explants can be washed in sterile water supplemented with 250 mg/L cefotaxime until no visible signs of Agrobacterium remain (i.e. wash solution remains clear after washing).

Transient gusA expression was determined on explants sampled after 3 days (or as indicated otherwise) on induction medium containing 150 mg/L timentin, using the histochemical GUS assay (Jefferson Plant Mol. Biol. Rep. 5: 387-405 1987). Explants were incubated overnight at 37° C. in buffer containing 1 mM X-Gluc, 100 μM sodium phosphate buffer pH 7.0, potassium 0.5 mM ferricyanide, 0.5 mM potassium ferrocyanide and 0.1% (v/v) Triton X-100. Blue gusA expression foci were counted under a microscope and T-DNA delivery assessed by counting explants that had at least one gusA expression foci and then counting the number of foci per embryo. To assay for stable gusA expression calli, shoots and leaf fragments from regenerating plantlets were incubated overnight at 37° C. and, if necessary, for a further 1-2 days at 25° C. As shown in FIG. 13F, gusA expression is detectable in the transformed embryo 3 days after inoculation.

### Example 22

### Agrobacterium-Mediated Transformation of Maize (Zea mays)

The Agrobacterium strain EHA 105 was transformed with the co-integrate binary vector LM227 (pSB1_Ubi1::DsdA-ocs_ScBV::DsRed2-nos) and pre-induced in a liquid infection media for approximately 3 hours before use. The OD₆₀₀ was approx 1.0 prior to inoculation.

Maize kernels were immersed in Domestos (Sodium Hypochlorite 49.9 g/l (available chlorine 4.75% m/v) Sodium hydroxide 12.0 g/l, alkaline salts 0.5 g/l) and incubated on a shaker for 30-45 minutes at 150 rpm. Kernels were rinse four times with sterile water and dispensed into a Petri dish following the fourth rinse and allow to soften for >3 hours.

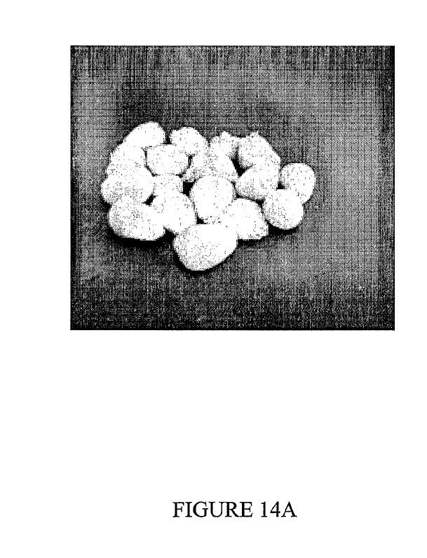

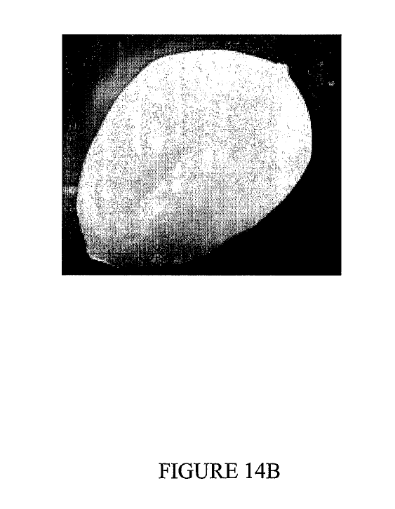

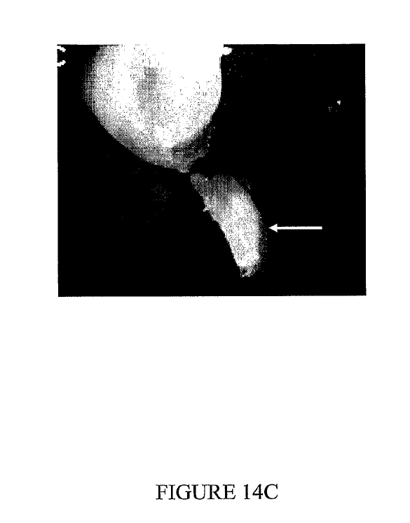

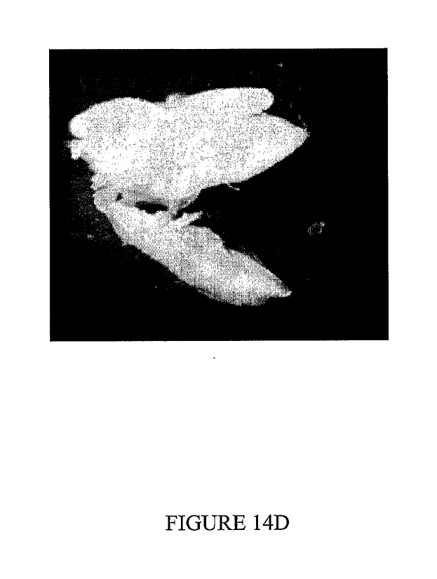

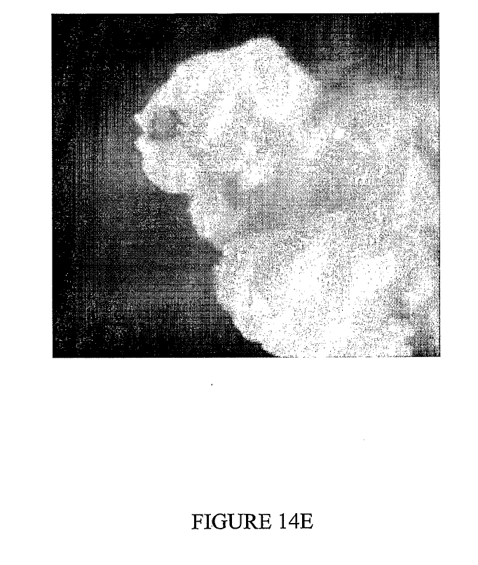

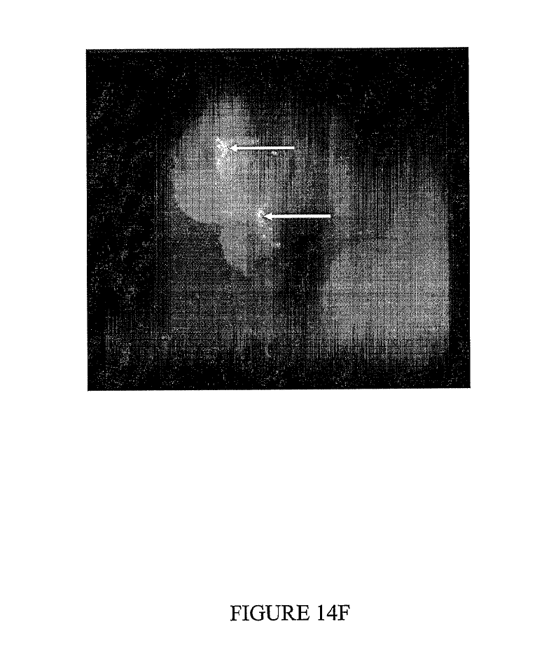

Mature embryos were isolated by holding single maize kernels with forceps whilst cutting two half moons either side of the embryo (see FIGS. 14A-D). Excised embryos were bisected and placed on an infection media ( 1/10 MS salts, 3% (w/v) sucrose, 200 μM acetosyringone, 0.04% (w/v) Soytone™, 2 mg/L 2,4-D, pH 5.7) until all explants were isolated. The infection media is removed and replaced with approximately 5 mL of Agrobacterium suspension (using 60×15 mm plates). Excised embryos were vacuum infiltrated at 27 mmHg for 5 minutes. Infection plates were incubated on a shaker at 50 rpm for 2 hours. Following inoculation, the Agrobacterium suspension was removed and explants transferred to co-culture media ( 1/10 MS salts, 3% (w/v) maltose, 200 μM acetosyringone, 2 mg/L 2,4-D, solidified with 8 μL agar, pH5.7) with the cut side facing down onto the medium. Explants were co-cultured for 3 days at 21° C. then removed to a recovery medium (MS salts, myo-inositol 0.1 g/l, thiamine hydrochloride 20 mg/L, casein hydrolysate 1 mg/L, proline 0.69 g/l, MES 1.95 g/L, maltose 30 g/L, solidified with 8 g/L agar, pH 5.7) for 7 days. The embryogenic cultures were subcultured after 7 days onto fresh recovery media supplemented with 5 mM D-serine.

Transient DsRed2 expression was determined on explants sampled after 3 or 4 days (or as indicated otherwise) on recovery media, using a Leica Stereomicroscope with DsRed2 optic filters. As shown in FIGS. 14E and F, DsRed2 was expressed in maize tissues.

1. A method for producing a transgenic graminaceous plant cell, said method comprising:
(i) obtaining embryonic cells from a mature graminaceous grain; and
(ii) contacting said embryonic cells with a bacterium capable of transforming a plant cell, said bacterium comprising transfer-nucleic acid to be introduced into the embryonic cells, said contacting being for a time and under conditions sufficient for said bacterium to introduce said transfer-nucleic acid into one or more cells thereof,
thereby producing a transgenic graminaceous plant cell.

2. The method according to claim 1 comprising obtaining the embryonic cells from a mature grain and contacting the embryonic cells with the bacterium comprising a nucleic acid construct without first inducing callus formation from said embryonic cells.

3. The method according to claim 1 comprising contacting the embryonic cells with the bacterium for a time and under conditions that are not sufficient to permit callus formation from said embryonic cells.

4. The method according to claim 1, wherein the embryonic cells are contacted with the bacterium within 3 days of obtaining said embryonic cells from the mature grain.

5. The method according to claim 1, wherein conditions sufficient for the bacterium to introduce the nucleic acid construct into a cell of the embryonic cells comprises inoculating the embryonic cells with the bacterium by performing a method comprising contacting the embryonic cells with the bacterium for a time and under conditions sufficient for said bacterium to bind to or attach to said embryonic cells.

6. The method according claim 1, wherein conditions sufficient for the bacterium to introduce the nucleic acid construct into a cell of the embryonic cells comprise co-culturing the embryonic cells and the bacterium by performing a method comprising maintaining the embryonic cells and bacterium for a time and under conditions sufficient for said bacterium to introduce the nucleic acid construct into a cell of the embryonic cells.

7. The method according to claim 1 wherein conditions sufficient for the bacterium to introduce the nucleic acid construct into a cell of the embryonic cells comprise maintaining the embryonic cells and the bacterium in the presence of a bacterial nitrogen source.

8. The method according to claim 7, wherein the bacterial nitrogen source is an enzymatic digest of a protein extract from a plant or animal is a water soluble fraction produced by partial hydrolysis of an extract from a plant or an animal.

9. The method according to claim 8, wherein the bacterial nitrogen source is from soybean.

10. The method according to claim 1 additionally comprising removing the seed coat and/or aleurone from the embryonic cells prior to contacting said tissue with the bacterium.

11. The method according to claim 1 wherein the graminaceous plant cell is a wheat cell or a barley cell or a rice cell or a maize cell.

12-19. (canceled)

20. The method according to claim 1, wherein the bacterium is an Agrobacterium.

21. A method for producing a transgenic wheat cell or a transgenic barley cell or a transgenic rice cell or a transgenic maize cell, said method comprising:
(i) obtaining embryonic cells from a mature wheat grain or from a mature barley grain or from a mature rice grain or from a mature maize kernel;
(ii) contacting the embryonic cells with an Agrobacterium comprising a nucleic acid construct that comprises transfer-nucleic acid to be introduced into the embryonic cells for a time and under conditions sufficient for said Agrobacterium to bind to or attach to said embryonic cells, wherein said contacting is performed without first inducing callus formation from said embryonic cells; and
(iii) maintaining the embryonic cells and the bound Agrobacterium for a time and under conditions sufficient for said Agrobacterium to introduce the transfer-nucleic acid into one or more cells thereof,
thereby producing a transgenic wheat cell or a transgenic barley cell or a transgenic rice cell or a transgenic maize cell.

22. A method for producing a transgenic wheat cell or a transgenic barley cell or a transgenic rice cell or a transgenic maize cell, said method comprising:
(i) obtaining embryonic cells from a mature wheat grain or from a mature barley grain or from a mature rice grain or from a mature maize kernel;
(ii) removing the seed coat and/or aleurone from the embryonic cells;
(iii) contacting the embryonic cells with an Agrobacterium comprising a nucleic acid construct that comprises transfer-nucleic acid to be introduced into the embryonic cells for a time and under conditions sufficient for said Agrobacterium to bind to or attach to said embryonic cells, wherein said contacting is performed without first inducing callus formation from said embryonic cells; and
(iv) maintaining the embryonic cells and the bound Agrobacterium for a time and under conditions sufficient for said Agrobacterium to introduce the transfer-nucleic acid into one or more cells thereof,
thereby producing a transgenic wheat cell or a transgenic barley cell or a transgenic rice cell or a transgenic maize cell.

23. A method for producing a transgenic wheat cell or a transgenic barley cell or a transgenic rice cell or a transgenic maize cell, said method comprising:
(i) obtaining embryonic cells from a mature wheat grain or from a mature barley grain or from a mature rice grain or from a mature maize kernel;
(ii) removing the seed coat and/or aleurone from the embryonic cells;
(iii) contacting the embryonic cells with an Agrobacterium comprising a nucleic acid construct that comprises transfer-nucleic acid to be introduced into the embryonic cells for a time and under conditions sufficient for said Agrobacterium to bind to or attach to said embryonic cells, wherein said contacting is performed in the presence of a peptone and wherein said contacting is performed without first inducing callus formation from said embryonic cells; and
(iv) maintaining the embryonic cells and the bound Agrobacterium for a time and under conditions sufficient for said Agrobacterium to introduce the transfer-nucleic acid into one or more cells thereof wherein said maintaining is performed in the presence of a peptone,
thereby producing a transgenic wheat cell or a transgenic barley cell or a transgenic rice cell or a transgenic maize cell.

24. A transgenic cell produced by the method according to claim 1.

25. A process for expressing a nucleic acid in a graminaceous plant cell, said process comprising:
(i) producing a transgenic graminaceous plant cell comprising a transgene in operable connection with a promoter operable in a wheat cell, said transgenic wheat cell produced by performing a method comprising:
(a) obtaining embryonic cells from a mature graminaceous grain; and
(b) contacting said embryonic cells with a bacterium capable of transforming a plant cell, said bacterium comprising transfer-nucleic acid to be introduced into the embryonic cells, said contacting being for a time and under conditions sufficient for said bacterium to introduce said transfer-nucleic acid into one or more cells thereof,
thereby producing a transgenic graminaceous plant cell; and
(ii) maintaining said transgenic cell for a time and under conditions sufficient for said transgene to be expressed.

26. A process for modulating the expression of a gene in a graminaceous plant cell, said process comprising:
(i) producing a transgenic graminaceous plant cell comprising a transgene capable of modulating the expression of the nucleic acid, said transgenic cell produced by performing a method comprising:
(a) obtaining embryonic cells from a mature graminaceous grain; and
(b) contacting said embryonic cells with a bacterium capable of transforming a plant cell, said bacterium comprising transfer-nucleic acid to be introduced into the embryonic cells, said contacting being for a time and under conditions sufficient for said bacterium to introduce said transfer-nucleic acid into one or more cells thereof,
thereby producing a transgenic graminaceous plant cell; and
(ii) maintaining said transgenic cell for a time and under conditions sufficient for the expression of the nucleic acid to be modulated.

27-46. (canceled)

47. The process according to claim 25 or 26, wherein the transgene encodes a protein associated with improved productivity of a plant.

48. The process according to claim 25 or 26, wherein the transgene encodes a protein that confers or enhances resistance to a wheat pathogen in a wheat plant in which the transgene is expressed.

49. (canceled)

50. The process according to claim 25 or 26, wherein the transgene confers drought tolerance and/or desiccation tolerance and/or salt tolerance and/or cold tolerance in a wheat plant in which the transgene is expressed.

51. (canceled)

52. The process according to claim 25 or 26, wherein the transgene encodes a protein that improves a nutritional quality of a wheat product from a wheat plant in which said transgene is expressed.

53-54. (canceled)

55. The process according to claim 25 or 26, wherein the transgene encodes a short interfering RNA or a micro-RNA.

56-57. (canceled)

58. A transgenic cell produced by the method according to claim 21.

59. A transgenic cell produced by the method according to claim 22.

60. A transgenic cell produced by the method according to claim 23.

61. A process for expressing a nucleic acid in a graminaceous plant cell or for modulating the expression of a gene in a graminaceous plant cell, said process comprising:
(i) producing a transgenic graminaceous plant cell comprising a transgene in operable connection with a promoter operable in a wheat cell, said transgenic wheat cell produced by performing a method according to claim 21; and
(ii) maintaining said transgenic cell for a time and under conditions sufficient for said transgene to be expressed or for the expression of the nucleic acid to be modulated.

62. A process for expressing a nucleic acid in a graminaceous plant cell or for modulating the expression of a gene in a graminaceous plant cell, said process comprising:
(i) producing a transgenic graminaceous plant cell comprising a transgene in operable connection with a promoter operable in a wheat cell, said transgenic wheat cell produced by performing a method according to claim 22; and
(ii) maintaining said transgenic cell for a time and under conditions sufficient for said transgene to be expressed or for the expression of the nucleic acid to be modulated.

63. A process for expressing a nucleic acid in a graminaceous plant cell or for modulating the expression of a gene in a graminaceous plant cell, said process comprising:
(i) producing a transgenic graminaceous plant cell comprising a transgene in operable connection with a promoter operable in a wheat cell, said transgenic wheat cell produced by performing a method according to claim 23; and
(ii) maintaining said transgenic cell for a time and under conditions sufficient for said transgene to be expressed or for the expression of the nucleic acid to be modulated.
